SEQUENCE LISTING (248 sequences)

SEQ ID NO: 1 - protein, 157 aa, Homo sapiens
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 2 - protein, 157 aa, Artificial Sequence
  Description of Artificial Sequence: Synthetic polypeptide
YFGKLKSKLSVIRNLNDQVLFIDQGNRPLFEDMTDSDCRDNAPRTIFIISMYADAQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 3 - protein, 157 aa, Artificial Sequence
  Description of Artificial Sequence: Synthetic polypeptide
GAGKLKSKLSVIRNLNDQVLFIDQGNRPLFEDMTDSDCRDNAPRTIFIISGYAAAQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 4 - protein, 157 aa, Artificial Sequence
  Description of Artificial Sequence: Synthetic polypeptide
YFGKLESKLSVIRNLNDQVLFIDQGNRPLFEDMTDSDCRDNAPRTIFIISMYADSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 5 - protein, 157 aa, Artificial Sequence
  Description of Artificial Sequence: Synthetic polypeptide
YFGKLESKLSVIRNLNDQVLFIDQGNRPLFEDMTDSDCRDNAPRTIFIISMYKDAQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 6 - protein, 157 aa, Artificial Sequence
  Description of Artificial Sequence: Synthetic polypeptide
YFGKLKSKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 7 - protein, 157 aa, Artificial Sequence
  Description of Artificial Sequence: Synthetic polypeptide
YFGKLKSKLSVIRNLNDQVLFIDQGNRPLFEDMTDSDCRDNAPRTIFIISMYADSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 8 - protein, 157 aa, Artificial Sequence
  Description of Artificial Sequence: Synthetic polypeptide
YFGKLKSKLSVIRNLNDQVLFIDQGNRPLFEDMTDSDCRDNAPRTIFIISMYKDAQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 9 - protein, 157 aa, Artificial Sequence
  Description of Artificial Sequence: Synthetic polypeptide
YFGKLESKLSVIRNLNDQVLFIDQGNRPLFEDMTDSDCRDNAPRTIFIISMYADAQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 10 - protein, 157 aa, Artificial Sequence
  Description of Artificial Sequence: Synthetic polypeptide
YFGKLKSKLSVIRNLNDQVLFIDQGNRPLFEDMTDSDCRDNAPRTIFIISMYADAQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 11 - protein, 157 aa, Artificial Sequence
  Description of Artificial Sequence: Synthetic polypeptide
GFGKLKSKLSVIRNLNDQVLFIDQGNRPLFEDMTDSDCRDNAPRTIFIISMYADAQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 12 - protein, 157 aa, Artificial Sequence
  Description of Artificial Sequence: Synthetic polypeptide
YAGKLKSKLSVIRNLNDQVLFIDQGNRPLFEDMTDSDCRDNAPRTIFIISMYADAQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 13 - protein, 157 aa, Artificial Sequence
  Description of Artificial Sequence: Synthetic polypeptide
YFGKLKSKLSVIRNLNDQVLFIDQGNRPLFEDMTDSDCRDNAPRTIFIISMYAAAQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 14 - protein, 157 aa, Artificial Sequence
  Description of Artificial Sequence: Synthetic polypeptide
YFGKLKSKLSVIRNLNDQVLFIDQGNRPLFEDMTDSDCRDNAPRTIFIISGYADAQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 15 - protein, 157 aa, Artificial Sequence
  Description of Artificial Sequence: Synthetic polypeptide
YFGKLESKLSVIRNLNDQVLFIDQGNRPLFEDMTDSDSRDNAPRTIFIISMYKDSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 16 - protein, 157 aa, Artificial Sequence
  Description of Artificial Sequence: Synthetic polypeptide
YFGKLESKLSVIRNLNDQVLFIDQGNRPLFEDMTDSDSRDNAPRTIFIISMYKDSQPRGMAVTISVKSECISTLSSENKIISFKEMNPPDNIKDTKSDIIFFQRSVPGHDNKMQFESSSYEGYFLASEKERDLFKLILKKEDELGDRSIMFTVQNED

SEQ ID NO: 17 - protein, 157 aa, Artificial Sequence
  Description of Artificial Sequence: Synthetic polypeptide
YFGKLKSKLSVIRNLNDQVLFIDQGNRPLFEDMTDSDSRDNAPRTIFIISMYADAQPRGMAVTISVKSECISTLSSENKIISFKEMNPPDNIKDTKSDIIFFQRSVPGHDNKMQFESSSYEGYFLASEKERDLFKLILKKEDELGDRSIMFTVQNED

SEQ ID NO: 18 - protein, 157 aa, Artificial Sequence
  Description of Artificial Sequence: Synthetic polypeptide
YFGKLKSKLSVIRNLNDQVLFIDQGNRPLFEDMTDSDCRDNAPRTIFIISMYADSQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 19 - protein, 157 aa, Artificial Sequence
  Description of Artificial Sequence: Synthetic polypeptide
YFGKLESKLSVIRNLNDQVLFIDQGNRPLFEDMTDSDCRDNAPRTIFIISMYKDSQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 20 - protein, 157 aa, Artificial Sequence
  Description of Artificial Sequence: Synthetic polypeptide
YFGKLKSKLSVIRNLNDQVLFIDQGNRPLFEDMTDSDCRDNAPRTIFIISMYKDSQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 21 - protein, 157 aa, Artificial Sequence
  Description of Artificial Sequence: Synthetic polypeptide
YFGKLESKLSVIRNLNDQVLFIDQGNRPLFEDMTDSDCRDNAPRTIFIISMYADSQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 22 - protein, 157 aa, Artificial Sequence
  Description of Artificial Sequence: Synthetic polypeptide
YFGKLKSKLSVIRNLNDQVLFIDQGNRPLFEDMTDSDSRDNAPRTIFIISMYADSQPRGMAVTISVKSECISTLSSENKIISFKEMNPPDNIKDTKSDIIFFQRSVPGHDNKMQFESSSYEGYFLASEKERDLFKLILKKEDELGDRSIMFTVQNED

SEQ ID NO: 23 - protein, 157 aa, Artificial Sequence
  Description of Artificial Sequence: Synthetic polypeptide
YFGKLESKLSVIRNLNDQVLFIDQGNRPLFEDMTDSDSRDNAPRTIFIISMYADSQPRGMAVAISVKSECISTLSSENKIISFKEMNPPDNIKDTKSDIIFFQRSVPGHDNKMQFESSSYEGYFLASEKERDLFKLILKKEDELGDRSIMFTVQNED

SEQ ID NO: 24 - protein, 157 aa, Artificial Sequence
  Description of Artificial Sequence: Synthetic polypeptide
  MOD_RES (31)..(31): Homoserine
  MOD_RES (33)..(33): Norleucine
  MOD_RES (51)..(51): Norleucine
  MOD_RES (60)..(60): Norleucine
  MOD_RES (75)..(75): Homoserine
  MOD_RES (86)..(86): Norleucine
  MOD_RES (113)..(113): Norleucine
  MOD_RES (116)..(116): Homoserine
  MOD_RES (150)..(150): Norleucine
YFGKLESKLSVIRNLNDQVLFIDQGNRPLFSDXTDSDCRDNAPRTIFIISXYKDSQPRGXAVTISVKCEKISTLSCENKIISFKEXNPPDNIKDTKSDIIFFQRSVPGHDNKXQFSSSSYEGYFLACEKERDLFKLILKKEDELGDRSIXFTVQNED

SEQ ID NO: 25 - protein, 157 aa, Artificial Sequence
  Description of Artificial Sequence: Synthetic polypeptide
  MOD_RES (31)..(31): Homoserine
  MOD_RES (33)..(33): Norleucine
  MOD_RES (51)..(51): Norleucine
  MOD_RES (60)..(60): Norleucine
  MOD_RES (75)..(75): Homoserine
  MOD_RES (86)..(86): Norleucine
  MOD_RES (113)..(113): Norleucine
  MOD_RES (116)..(116): Homoserine
  MOD_RES (150)..(150): Norleucine
YFGKLESKLSVIRNLNDQVLFIDQGNRPLFSDXTDSDSRDNAPRTIFIISXYKDSQPRGXAVTISVKSEKISTLSSENKIISFKEXNPPDNIKDTKSDIIFFQRSVPGHDNKXQFSSSSYEGYFLASEKERDLFKLILKKEDELGDRSIXFTVQNED

SEQ ID NO: 26 - protein, 157 aa, Artificial Sequence
  Description of Artificial Sequence: Synthetic polypeptide
  MOD_RES (31)..(31): Homoserine
  MOD_RES (33)..(33): Norleucine
  MOD_RES (51)..(51): Norleucine
  MOD_RES (60)..(60): Norleucine
  MOD_RES (70)..(70): (S)-47-amino-1-azido-37,41-dioxo-3,6,9,12,15, 18,21,24,27,30,33-undecaoxa-36,42-diazaoctatetracontan-48-oic acid or Azidolysine
  MOD_RES (75)..(75): Homoserine
  MOD_RES (86)..(86): Norleucine
  MOD_RES (113)..(113): Norleucine
  MOD_RES (116)..(116): Homoserine
  MOD_RES (150)..(150): Norleucine
YFGKLKSKLSVIRNLNDQVLFIDQGNRPLFSDXTDSDCRDNAPRTIFIISXYADSQPRGXAVTISVKCEXISTLSCENKIISFKEXNPPDNIKDTKSDIIFFQRSVPGHDNKXQFSSSSYEGYFLACEKERDLFKLILKKEDELGDRSIXFTVQNED

SEQ ID NO: 27 - protein, 157 aa, Artificial Sequence
  Description of Artificial Sequence: Synthetic polypeptide
  MOD_RES (31)..(31): Homoserine
  MOD_RES (33)..(33): Norleucine
  MOD_RES (51)..(51): Norleucine
  MOD_RES (60)..(60): Norleucine
  MOD_RES (63)..(63): Homoserine
  MOD_RES (86)..(86): Norleucine
  MOD_RES (113)..(113): Norleucine
  MOD_RES (116)..(116): Homoserine
  MOD_RES (150)..(150): Norleucine
YFGKLESKLSVIRNLNDQVLFIDQGNRPLFSDXTDSDCRDNAPRTIFIISXYKDSQPRGXAVSISVKCEKISTLSCENKIISFKEXNPPDNIKDTKSDIIFFQRSVPGHDNKXQFSSSSYEGYFLACEKERDLFKLILKKEDELGDRSIXFTVQNED

SEQ ID NO: 28 - protein, 157 aa, Artificial Sequence
  Description of Artificial Sequence: Synthetic polypeptide
  MOD_RES (31)..(31): Homoserine
  MOD_RES (33)..(33): Norleucine
  MOD_RES (51)..(51): Norleucine
  MOD_RES (60)..(60): Norleucine
  MOD_RES (63)..(63): Homoserine
  MOD_RES (70)..(70): (S)-47-amino-1-azido-37,41-dioxo-3,6,9,12,15, 18,21,24,27,30,33-undecaoxa-36,42-diazaoctatetracontan-48-oic acid or Azidolysine
  MOD_RES (86)..(86): Norleucine
  MOD_RES (113)..(113): Norleucine
  MOD_RES (116)..(116): Homoserine
  MOD_RES (150)..(150): Norleucine
YFGKLKSKLSVIRNLNDQVLFIDQGNRPLFSDXTDSDCRDNAPRTIFIISXYADSQPRGXAVSISVKCEXISTLSCENKIISFKEXNPPDNIKDTKSDIIFFQRSVPGHDNKXQFSSSSYEGYFLACEKERDLFKLILKKEDELGDRSIXFTVQNED

SEQ ID NO: 29 - protein, 157 aa, Artificial Sequence
  Description of Artificial Sequence: Synthetic polypeptide
  MOD_RES (31)..(31): Homoserine
  MOD_RES (33)..(33): Norleucine
  MOD_RES (51)..(51): Norleucine
  MOD_RES (60)..(60): Norleucine
  MOD_RES (63)..(63): Homoserine
  MOD_RES (86)..(86): Norleucine
  MOD_RES (113)..(113): Norleucine
  MOD_RES (116)..(116): Homoserine
  MOD_RES (150)..(150): Norleucine
YFGKLESKLSVIRNLNDQVLFIDQGNRPLFSDXTDSDSRDNAPRTIFIISXYKDSQPRGXAVSISVKSEKISTLSSENKIISFKEXNPPDNIKDTKSDIIFFQRSVPGHDNKXQFSSSSYEGYFLASEKERDLFKLILKKEDELGDRSIXFTVQNED

SEQ ID NO: 30 - protein, 157 aa, Artificial Sequence
  Description of Artificial Sequence: Synthetic polypeptide
  MOD_RES (31)..(31): Homoserine
  MOD_RES (33)..(33): Norleucine
  MOD_RES (51)..(51): Norleucine
  MOD_RES (60)..(60): Norleucine
  MOD_RES (63)..(63): Homoserine
  MOD_RES (70)..(70): (S)-47-amino-1-azido-37,41-dioxo-3,6,9,12,15, 18,21,24,27,30,33 -undecaoxa-36,42-diazaoctatetracontan-48-oic acid or Azidolysine
  MOD_RES (86)..(86): Norleucine
  MOD_RES (113)..(113): Norleucine
  MOD_RES (116)..(116): Homoserine
  MOD_RES (150)..(150): Norleucine
YFGKLKSKLSVIRNLNDQVLFIDQGNRPLFSDXTDSDSRDNAPRTIFIISXYADSQPRGXAVSISVKSEXISTLSSENKIISFKEXNPPDNIKDTKSDIIFFQRSVPGHDNKXQFSSSSYEGYFLASEKERDLFKLILKKEDELGDRSIXFTVQNED

SEQ ID NO: 31 - protein, 157 aa, Artificial Sequence
  Description of Artificial Sequence: Synthetic polypeptide
  MOD_RES (31)..(31): Homoserine
  MOD_RES (63)..(63): Homoserine
  MOD_RES (116)..(116): Homoserine
YFGKLESKLSVIRNLNDQVLFIDQGNRPLFSDMTDSDCRDNAPRTIFIISMYKDSQPRGMAVSISVKCEKISTLSCENKIISFKEMNPPDNIKDTKSDIIFFQRSVPGHDNKMQFSSSSYEGYFLACEKERDLFKLILKKEDELGDRSIMFTVQNED

SEQ ID NO: 32 - protein, 157 aa, Artificial Sequence
  Description of Artificial Sequence: Synthetic polypeptide
  MOD_RES (31)..(31): Homoserine
  MOD_RES (63)..(63): Homoserine
  MOD_RES (70)..(70): (S)-47-amino-1-azido-37,41-dioxo-3,6,9,12,15, 18,21,24,27,30,33-undecaoxa-36,42-diazaoctatetracontan-48-oic acid or Azidolysine
  MOD_RES (116)..(116): Homoserine
YFGKLKSKLSVIRNLNDQVLFIDQGNRPLFSDMTDSDCRDNAPRTIFIISMYADSQPRGMAVSISVKCEXISTLSCENKIISFKEMNPPDNIKDTKSDIIFFQRSVPGHDNKMQFSSSSYEGYFLACEKERDLFKLILKKEDELGDRSIMFTVQNED

SEQ ID NO: 33 - protein, 157 aa, Artificial Sequence
  Description of Artificial Sequence: Synthetic polypeptide
  MOD_RES (31)..(31): Homoserine
  MOD_RES (63)..(63): Homoserine
  MOD_RES (116)..(116): Homoserine
YFGKLESKLSVIRNLNDQVLFIDQGNRPLFSDMTDSDSRDNAPRTIFIISMYKDSQPRGMAVSISVKSEKISTLSSENKIISFKEMNPPDNIKDTKSDIIFFQRSVPGHDNKMQFSSSSYEGYFLASEKERDLFKLILKKEDELGDRSIMFTVQNED

SEQ ID NO: 34 - protein, 157 aa, Artificial Sequence
  Description of Artificial Sequence: Synthetic polypeptide
  MOD_RES (31)..(31): Homoserine
  MOD_RES (63)..(63): Homoserine
  MOD_RES (70)..(70): (S)-47-amino-1-azido-37,41-dioxo-3,6,9,12,15, 18,21,24,27,30,33-undecaoxa-36,42-diazaoctatetracontan-48-oic acid or Azidolysine
  MOD_RES (116)..(116): Homoserine
YFGKLKSKLSVIRNLNDQVLFIDQGNRPLFSDMTDSDSRDNAPRTIFIISMYADSQPRGMAVSISVKSEXISTLSSENKIISFKEMNPPDNIKDTKSDIIFFQRSVPGHDNKMQFSSSSYEGYFLASEKERDLFKLILKKEDELGDRSIMFTVQNED

SEQ ID NO: 35 - protein, 157 aa, Artificial Sequence
  Description of Artificial Sequence: Synthetic polypeptide
  MOD_RES (31)..(31): Homoserine
  MOD_RES (33)..(33): O-methyl-L-homoserine
  MOD_RES (51)..(51): O-methyl-L-homoserine
  MOD_RES (60)..(60): O-methyl-L-homoserine
  MOD_RES (63)..(63): Homoserine
  MOD_RES (86)..(86): O-methyl-L-homoserine
  MOD_RES (113)..(113): O-methyl-L-homoserine
  MOD_RES (116)..(116): Homoserine
  MOD_RES (150)..(150): O-methyl-L-homoserine
YFGKLESKLSVIRNLNDQVLFIDQGNRPLFSDSTDSDCRDNAPRTIFIISSYKDSQPRGSAVSISVKCEKISTLSCENKIISFKESNPPDNIKDTKSDIIFFQRSVPGHDNKSQFSSSSYEGYFLACEKERDLFKLILKKEDELGDRSISFTVQNED

SEQ ID NO: 36 - protein, 157 aa, Artificial Sequence
  Description of Artificial Sequence: Synthetic polypeptide
  MOD_RES (31)..(31): Homoserine
  MOD_RES (33)..(33): O-methyl-L-homoserine
  MOD_RES (51)..(51): O-methyl-L-homoserine
  MOD_RES (60)..(60): O-methyl-L-homoserine
  MOD_RES (63)..(63): Homoserine
  MOD_RES (70)..(70): (S)-47-amino-1-azido-37,41-dioxo-3,6,9,12,15, 18,21,24,27,30,33-undecaoxa-36,42-diazaoctatetracontan-48-oic acid or Azidolysine
  MOD_RES (86)..(86): O-methyl-L-homoserine
  MOD_RES (113)..(113): O-methyl-L-homoserine
  MOD_RES (116)..(116): Homoserine
  MOD_RES (150)..(150): O-methyl-L-homoserine
YFGKLKSKLSVIRNLNDQVLFIDQGNRPLFSDSTDSDCRDNAPRTIFIISSYADSQPRGSAVSISVKCEXISTLSCENKIISFKESNPPDNIKDTKSDIIFFQRSVPGHDNKSQFSSSSYEGYFLACEKERDLFKLILKKEDELGDRSISFTVQNED

SEQ ID NO: 37 - protein, 157 aa, Artificial Sequence
  Description of Artificial Sequence: Synthetic polypeptide
  MOD_RES (31)..(31): Homoserine
  MOD_RES (33)..(33): O-methyl-L-homoserine
  MOD_RES (51)..(51): O-methyl-L-homoserine
  MOD_RES (60)..(60): O-methyl-L-homoserine
  MOD_RES (63)..(63): Homoserine
  MOD_RES (86)..(86): O-methyl-L-homoserine
  MOD_RES (113)..(113): O-methyl-L-homoserine
  MOD_RES (116)..(116): Homoserine
  MOD_RES (150)..(150): O-methyl-L-homoserine
YFGKLESKLSVIRNLNDQVLFIDQGNRPLFSDSTDSDSRDNAPRTIFIISSYKDSQPRGSAVSISVKSEKISTLSSENKIISFKESNPPDNIKDTKSDIIFFQRSVPGHDNKSQFSSSSYEGYFLASEKERDLFKLILKKEDELGDRSISFTVQNED

SEQ ID NO: 38 - protein, 157 aa, Artificial Sequence
  Description of Artificial Sequence: Synthetic polypeptide
  MOD_RES (31)..(31): Homoserine
  MOD_RES (33)..(33): O-methyl-L-homoserine
  MOD_RES (51)..(51): O-methyl-L-homoserine
  MOD_RES (60)..(60): O-methyl-L-homoserine
  MOD_RES (63)..(63): Homoserine
  MOD_RES (70)..(70): (S)-47-amino-1-azido-37,41-dioxo-3,6,9,12,15, 18,21,24,27,30,33-undecaoxa-36,42-diazaoctatetracontan-48-oic acid or Azidolysine
  MOD_RES (86)..(86): O-methyl-L-homoserine
  MOD_RES (113)..(113): O-methyl-L-homoserine
  MOD_RES (116)..(116): Homoserine
  MOD_RES (150)..(150): O-methyl-L-homoserine
YFGKLKSKLSVIRNLNDQVLFIDQGNRPLFSDSTDSDSRDNAPRTIFIISSYADSQPRGSAVSISVKSEXISTLSSENKIISFKESNPPDNIKDTKSDIIFFQRSVPGHDNKSQFSSSSYEGYFLASEKERDLFKLILKKEDELGDRSISFTVQNED

SEQ ID NO: 39 - protein, 157 aa, Artificial Sequence
  Description of Artificial Sequence: Synthetic polypeptide
  MOD_RES (31)..(31): Homoserine
  MOD_RES (33)..(33): Norleucine
  MOD_RES (51)..(51): Norleucine
  MOD_RES (60)..(60): Norleucine
  MOD_RES (75)..(75): Homoserine
  MOD_RES (86)..(86): Norleucine
  MOD_RES (113)..(113): Norleucine
  MOD_RES (116)..(116): Homoserine
  MOD_RES (150)..(150): Norleucine
YFGKLKSKLSVIRNLNDQVLFIDQGNRPLFSDXTDSDCRDNAPRTIFIISXYADSQPRGXAVTISVKCEKISTLSCENKIISFKEXNPPDNIKDTKSDIIFFQRSVPGHDNKXQFSSSSYEGYFLACEKERDLFKLILKKEDELGDRSIXFTVQNED

SEQ ID NO: 40 - protein, 157 aa, Artificial Sequence
  Description of Artificial Sequence: Synthetic polypeptide
  MOD_RES (31)..(31): Homoserine
  MOD_RES (63)..(63): Homoserine
  MOD_RES (116)..(116): Homoserine
YFGKLKSKLSVIRNLNDQVLFIDQGNRPLFSDMTDSDCRDNAPRTIFIISMYADSQPRGMAVSISVKCEKISTLSCENKIISFKEMNPPDNIKDTKSDIIFFQRSVPGHDNKMQFSSSSYEGYFLACEKERDLFKLILKKEDELGDRSIMFTVQNED

SEQ ID NO: 41 - protein, 157 aa, Artificial Sequence
  Description of Artificial Sequence: Synthetic polypeptide
  MOD_RES (31)..(31): Homoserine
  MOD_RES (63)..(63): Homoserine
  MOD_RES (116)..(116): Homoserine
YFGKLKSKLSVIRNLNDQVLFIDQGNRPLFSDMTDSDSRDNAPRTIFIISMYADSQPRGMAVSISVKSEKISTLSSENKIISFKEMNPPDNIKDTKSDIIFFQRSVPGHDNKMQFSSSSYEGYFLASEKERDLFKLILKKEDELGDRSIMFTVQNED

SEQ ID NO: 42 - protein, 157 aa, Artificial Sequence
  Description of Artificial Sequence: Synthetic polypeptide
  MOD_RES (31)..(31): Homoserine
  MOD_RES (33)..(33): Norleucine
  MOD_RES (51)..(51): Norleucine
  MOD_RES (60)..(60): Norleucine
  MOD_RES (63)..(63): Homoserine
  MOD_RES (86)..(86): Norleucine
  MOD_RES (113)..(113): Norleucine
  MOD_RES (116)..(116): Homoserine
  MOD_RES (150)..(150): Norleucine
YFGKLKSKLSVIRNLNDQVLFIDQGNRPLFSDXTDSDCRDNAPRTIFIISXYADSQPRGXAVSISVKCEKISTLSCENKIISFKEXNPPDNIKDTKSDIIFFQRSVPGHDNKXQFSSSSYEGYFLACEKERDLFKLILKKEDELGDRSIXFTVQNED

SEQ ID NO: 43 - protein, 157 aa, Artificial Sequence
  Description of Artificial Sequence: Synthetic polypeptide
  MOD_RES (31)..(31): Homoserine
  MOD_RES (33)..(33): Norleucine
  MOD_RES (51)..(51): Norleucine
  MOD_RES (60)..(60): Norleucine
  MOD_RES (63)..(63): Homoserine
  MOD_RES (86)..(86): Norleucine
  MOD_RES (113)..(113): Norleucine
  MOD_RES (116)..(116): Homoserine
  MOD_RES (150)..(150): Norleucine
YFGKLKSKLSVIRNLNDQVLFIDQGNRPLFSDXTDSDSRDNAPRTIFIISXYADSQPRGXAVSISVKSEKISTLSSENKIISFKEXNPPDNIKDTKSDIIFFQRSVPGHDNKXQFSSSSYEGYFLASEKERDLFKLILKKEDELGDRSIXFTVQNED

SEQ ID NO: 44 - protein, 157 aa, Artificial Sequence
  Description of Artificial Sequence: Synthetic polypeptide
  MOD_RES (31)..(31): Homoserine
  MOD_RES (33)..(33): O-methyl-L-homoserine
  MOD_RES (51)..(51): O-methyl-L-homoserine
  MOD_RES (60)..(60): O-methyl-L-homoserine
  MOD_RES (63)..(63): Homoserine
  MOD_RES (86)..(86): O-methyl-L-homoserine
  MOD_RES (113)..(113): O-methyl-L-homoserine
  MOD_RES (116)..(116): Homoserine
  MOD_RES (150)..(150): O-methyl-L-homoserine
YFGKLKSKLSVIRNLNDQVLFIDQGNRPLFSDSTDSDCRDNAPRTIFIISSYADSQPRGSAVSISVKCEKISTLSCENKIISFKESNPPDNIKDTKSDIIFFQRSVPGHDNKSQFSSSSYEGYFLACEKERDLFKLILKKEDELGDRSISFTVQNED

SEQ ID NO: 45 - protein, 157 aa, Artificial Sequence
  Description of Artificial Sequence: Synthetic polypeptide
  MOD_RES (31)..(31): Homoserine
  MOD_RES (33)..(33): O-methyl-L-homoserine
  MOD_RES (51)..(51): O-methyl-L-homoserine
  MOD_RES (60)..(60): O-methyl-L-homoserine
  MOD_RES (63)..(63): Homoserine
  MOD_RES (86)..(86): O-methyl-L-homoserine
  MOD_RES (113)..(113): O-methyl-L-homoserine
  MOD_RES (116)..(116): Homoserine
  MOD_RES (150)..(150): O-methyl-L-homoserine
YFGKLKSKLSVIRNLNDQVLFIDQGNRPLFSDSTDSDSRDNAPRTIFIISSYADSQPRGSAVSISVKSEKISTLSSENKIISFKESNPPDNIKDTKSDIIFFQRSVPGHDNKSQFSSSSYEGYFLASEKERDLFKLILKKEDELGDRSISFTVQNED

SEQ ID NO: 46 - protein, 157 aa, Artificial Sequence
  Description of Artificial Sequence: Synthetic polypeptide
  MOD_RES (31)..(31): Homoserine
  MOD_RES (33)..(33): Norleucine
  MOD_RES (51)..(51): Norleucine
  MOD_RES (60)..(60): Norleucine
  MOD_RES (70)..(70): (S)-47-amino-1-azido-37,41-dioxo-3,6,9,12,15, 18,21,24,27,30,33-undecaoxa-36,42-diazaoctatetracontan-48-oic acid or Azidolysine
  MOD_RES (75)..(75): Homoserine
  MOD_RES (86)..(86): Norleucine
  MOD_RES (113)..(113): Norleucine
  MOD_RES (116)..(116): Homoserine
  MOD_RES (150)..(150): Norleucine
YFGKLESKLSVIRNLNDQVLFIDQGNRPLFSDXTDSDCRDNAPRTIFIISXYKDSQPRGXAVTISVKCEXISTLSCENKIISFKEXNPPDNIKDTKSDIIFFQRSVPGHDNKXQFSSSSYEGYFLACEKERDLFKLILKKEDELGDRSIXFTVQNED

SEQ ID NO: 47 - protein, 157 aa, Artificial Sequence
  Description of Artificial Sequence: Synthetic polypeptide
  MOD_RES (31)..(31): Homoserine
  MOD_RES (63)..(63): Homoserine
  MOD_RES (70)..(70): (S)-47-amino-1-azido-37,41-dioxo-3,6,9,12,15, 18,21,24,27,30,33-undecaoxa-36,42-diazaoctatetracontan-48-oic acid or Azidolysine
  MOD_RES (116)..(116): Homoserine
YFGKLESKLSVIRNLNDQVLFIDQGNRPLFSDMTDSDCRDNAPRTIFIISMYKDSQPRGMAVSISVKCEXISTLSCENKIISFKEMNPPDNIKDTKSDIIFFQRSVPGHDNKMQFSSSSYEGYFLACEKERDLFKLILKKEDELGDRSIMFTVQNED

SEQ ID NO: 48 - protein, 157 aa, Artificial Sequence
  Description of Artificial Sequence: Synthetic polypeptide
  MOD_RES (31)..(31): Homoserine
  MOD_RES (63)..(63): Homoserine
  MOD_RES (70)..(70): (S)-47-amino-1-azido-37,41-dioxo-3,6,9,12,15, 18,21,24,27,30,33-undecaoxa-36,42-diazaoctatetracontan-48-oic acid or Azidolysine
  MOD_RES (116)..(116): Homoserine
YFGKLESKLSVIRNLNDQVLFIDQGNRPLFSDMTDSDSRDNAPRTIFIISMYKDSQPRGMAVSISVKSEXISTLSSENKIISFKEMNPPDNIKDTKSDIIFFQRSVPGHDNKMQFSSSSYEGYFLASEKERDLFKLILKKEDELGDRSIMFTVQNED

SEQ ID NO: 49 - protein, 157 aa, Artificial Sequence
  Description of Artificial Sequence: Synthetic polypeptide
  MOD_RES (31)..(31): Homoserine
  MOD_RES (33)..(33): Norleucine
  MOD_RES (51)..(51): Norleucine
  MOD_RES (60)..(60): Norleucine
  MOD_RES (63)..(63): Homoserine
  MOD_RES (70)..(70): (S)-47-amino-1-azido-37,41-dioxo-3,6,9,12,15, 18,21,24,27,30,33-undecaoxa-36,42-diazaoctatetracontan-48-oic acid or Azidolysine
  MOD_RES (86)..(86): Norleucine
  MOD_RES (113)..(113): Norleucine
  MOD_RES (116)..(116): Homoserine
  MOD_RES (150)..(150): Norleucine
YFGKLESKLSVIRNLNDQVLFIDQGNRPLFSDXTDSDCRDNAPRTIFIISXYKDSQPRGXAVSISVKCEXISTLSCENKIISFKEXNPPDNIKDTKSDIIFFQRSVPGHDNKXQFSSSSYEGYFLACEKERDLFKLILKKEDELGDRSIXFTVQNED

SEQ ID NO: 50 - protein, 157 aa, Artificial Sequence
  Description of Artificial Sequence: Synthetic polypeptide
  MOD_RES (31)..(31): Homoserine
  MOD_RES (33)..(33): Norleucine
  MOD_RES (51)..(51): Norleucine
  MOD_RES (60)..(60): Norleucine
  MOD_RES (63)..(63): Homoserine
  MOD_RES (70)..(70): (S)-47-amino-1-azido-37,41-dioxo-3,6,9,12,15, 18,21,24,27,30,33-undecaoxa-36,42-diazaoctatetracontan-48-oic acid or Azidolysine
  MOD_RES (86)..(86): Norleucine
  MOD_RES (113)..(113): Norleucine
  MOD_RES (116)..(116): Homoserine
  MOD_RES (150)..(150): Norleucine
YFGKLESKLSVIRNLNDQVLFIDQGNRPLFSDXTDSDSRDNAPRTIFIISXYKDSQPRGXAVSISVKSEXISTLSSENKIISFKEXNPPDNIKDTKSDIIFFQRSVPGHDNKXQFSSSSYEGYFLASEKERDLFKLILKKEDELGDRSIXFTVQNED

SEQ ID NO: 51 - protein, 157 aa, Artificial Sequence
  Description of Artificial Sequence: Synthetic polypeptide
  MOD_RES (31)..(31): Homoserine
  MOD_RES (33)..(33): O-methyl-L-homoserine
  MOD_RES (51)..(51): O-methyl-L-homoserine
  MOD_RES (60)..(60): O-methyl-L-homoserine
  MOD_RES (63)..(63): Homoserine
  MOD_RES (70)..(70): (S)-47-amino-1-azido-37,41-dioxo-3,6,9,12,15, 18,21,24,27,30,33-undecaoxa-36,42-diazaoctatetracontan-48-oic acid or Azidolysine
  MOD_RES (86)..(86): O-methyl-L-homoserine
  MOD_RES (113)..(113): O-methyl-L-homoserine
  MOD_RES (116)..(116): Homoserine
  MOD_RES (150)..(150): O-methyl-L-homoserine
YFGKLESKLSVIRNLNDQVLFIDQGNRPLFSDSTDSDCRDNAPRTIFIISSYKDSQPRGSAVSISVKCEXISTLSCENKIISFKESNPPDNIKDTKSDIIFFQRSVPGHDNKSQFSSSSYEGYFLACEKERDLFKLILKKEDELGDRSISFTVQNED

SEQ ID NO: 52 - protein, 157 aa, Artificial Sequence
  Description of Artificial Sequence: Synthetic polypeptide
  MOD_RES (31)..(31): Homoserine
  MOD_RES (33)..(33): O-methyl-L-homoserine
  MOD_RES (51)..(51): O-methyl-L-homoserine
  MOD_RES (60)..(60): O-methyl-L-homoserine
  MOD_RES (63)..(63): Homoserine
  MOD_RES (70)..(70): (S)-47-amino-1-azido-37,41-dioxo-3,6,9,12,15, 18,21,24,27,30,33-undecaoxa-36,42-diazaoctatetracontan-48-oic acid or Azidolysine
  MOD_RES (86)..(86): O-methyl-L-homoserine
  MOD_RES (113)..(113): O-methyl-L-homoserine
  MOD_RES (116)..(116): Homoserine
  MOD_RES (150)..(150): O-methyl-L-homoserine
YFGKLESKLSVIRNLNDQVLFIDQGNRPLFSDSTDSDSRDNAPRTIFIISSYKDSQPRGSAVSISVKSEXISTLSSENKIISFKESNPPDNIKDTKSDIIFFQRSVPGHDNKSQFSSSSYEGYFLASEKERDLFKLILKKEDELGDRSISFTVQNED

SEQ ID NO: 53 - protein, 157 aa, Artificial Sequence
  Description of Artificial Sequence: Synthetic polypeptide
  MOD_RES (31)..(31): Homoserine
  MOD_RES (75)..(75): Homoserine
  MOD_RES (116)..(116): Homoserine
YFGKLKSKLSVIRNLNDQVLFIDQGNRPLFSDMTDSDCRDNAPRTIFIISMYADSQPRGMAVAISVKCEKISTLSCENKIISFKEMNPPDNIKDTKSDIIFFQRSVPGHDNKMQFSSSSYEGYFLACEKERDLFKLILKKEDELGDRSIMFTVQNED

SEQ ID NO: 54 - protein, 157 aa, Artificial Sequence
  Description of Artificial Sequence: Synthetic polypeptide
  MOD_RES (31)..(31): Homoserine
  MOD_RES (75)..(75): Homoserine
  MOD_RES (116)..(116): Homoserine
YFGKLKSKLSVIRNLNDQVLFIDQGNRPLFSDMTDSDSRDNAPRTIFIISMYADSQPRGMAVAISVKSEKISTLSSENKIISFKEMNPPDNIKDTKSDIIFFQRSVPGHDNKMQFSSSSYEGYFLASEKERDLFKLILKKEDELGDRSIMFTVQNED

SEQ ID NO: 55 - protein, 157 aa, Artificial Sequence
  Description of Artificial Sequence: Synthetic polypeptide
  MOD_RES (31)..(31): Homoserine
  MOD_RES (33)..(33): Norleucine
  MOD_RES (51)..(51): Norleucine
  MOD_RES (60)..(60): Norleucine
  MOD_RES (75)..(75): Homoserine
  MOD_RES (86)..(86): Norleucine
  MOD_RES (113)..(113): Norleucine
  MOD_RES (116)..(116): Homoserine
  MOD_RES (150)..(150): Norleucine
YFGKLKSKLSVIRNLNDQVLFIDQGNRPLFSDXTDSDCRDNAPRTIFIISXYADSQPRGXAVAISVKCEKISTLSCENKIISFKEXNPPDNIKDTKSDIIFFQRSVPGHDNKXQFSSSSYEGYFLACEKERDLFKLILKKEDELGDRSIXFTVQNED

SEQ ID NO: 56 - protein, 157 aa, Artificial Sequence
  Description of Artificial Sequence: Synthetic polypeptide
  MOD_RES (31)..(31): Homoserine
  MOD_RES (33)..(33): Norleucine
  MOD_RES (51)..(51): Norleucine
  MOD_RES (60)..(60): Norleucine
  MOD_RES (75)..(75): Homoserine
  MOD_RES (86)..(86): Norleucine
  MOD_RES (113)..(113): Norleucine
  MOD_RES (116)..(116): Homoserine
  MOD_RES (150)..(150): Norleucine
YFGKLKSKLSVIRNLNDQVLFIDQGNRPLFSDXTDSDSRDNAPRTIFIISXYADSQPRGXAVAISVKCEKISTLSSENKIISFKEXNPPDNIKDTKSDIIFFQRSVPGHDNKXQFSSSSYEGYFLASEKERDLFKLILKKEDELGDRSIXFTVQNED

SEQ ID NO: 57 - protein, 157 aa, Artificial Sequence
  Description of Artificial Sequence: Synthetic polypeptide
  MOD_RES (31)..(31): Homoserine
  MOD_RES (33)..(33): O-methyl-L-homoserine
  MOD_RES (51)..(51): O-methyl-L-homoserine
  MOD_RES (60)..(60): O-methyl-L-homoserine
  MOD_RES (75)..(75): Homoserine
  MOD_RES (86)..(86): O-methyl-L-homoserine
  MOD_RES (113)..(113): O-methyl-L-homoserine
  MOD_RES (116)..(116): Homoserine
  MOD_RES (150)..(150): O-methyl-L-homoserine
YFGKLKSKLSVIRNLNDQVLFIDQGNRPLFSDSTDSDCRDNAPRTIFIISSYADSQPRGSAVAISVKCEKISTLSCENKIISFKESNPPDNIKDTKSDIIFFQRSVPGHDNKSQFSSSSYEGYFLACEKERDLFKLILKKEDELGDRSISFTVQNED

SEQ ID NO: 58 - protein, 157 aa, Artificial Sequence
  Description of Artificial Sequence: Synthetic polypeptide
  MOD_RES (31)..(31): Homoserine
  MOD_RES (33)..(33): O-methyl-L-homoserine
  MOD_RES (51)..(51): O-methyl-L-homoserine
  MOD_RES (60)..(60): O-methyl-L-homoserine
  MOD_RES (75)..(75): Homoserine
  MOD_RES (86)..(86): O-methyl-L-homoserine
  MOD_RES (113)..(113): O-methyl-L-homoserine
  MOD_RES (116)..(116): Homoserine
  MOD_RES (150)..(150): O-methyl-L-homoserine
YFGKLKSKLSVIRNLNDQVLFIDQGNRPLFSDSTDSDSRDNAPRTIFIISSYADSQPRGSAVAISVKCEKISTLSSENKIISFKESNPPDNIKDTKSDIIFFQRSVPGHDNKSQFSSSSYEGYFLASEKERDLFKLILKKEDELGDRSISFTVQNED

SEQ ID NO: 59 - protein, 157 aa, Artificial Sequence
  Description of Artificial Sequence: Synthetic polypeptide
  MOD_RES (31)..(31): Homoserine
  MOD_RES (33)..(33): Norleucine
  MOD_RES (51)..(51): Norleucine
  MOD_RES (60)..(60): Norleucine
  MOD_RES (63)..(63): Homoserine
  MOD_RES (68)..(68): (S)-47-amino-1-azido-37,41-dioxo-3,6,9,12,15, 18,21,24,27,30,33-undecaoxa-36,42-diazaoctatetracontan-48-oic acid, (S)-40-amino-1-azido-37-oxo-3,6,9,12,15,18,21,24,27,30,33- undecaoxa-36-azahentetracontan-41-oic acid, (S)-39-amino-1-azido- 37-oxo-
  MOD_RES (68)..(68): CONT. FROM ABOVE: 3,6,9,12,15,18,21,24,27,30, 33-undecaoxa-36-azatetracontan-40-oic acid or (R)-41-amino-1- azido-37-oxo-3,6,9,12,15,18,21,24,27,30,33-undecaoxa-39-thia-36- azadotetracontan-42-oic acid
  MOD_RES (86)..(86): Norleucine
  MOD_RES (113)..(113): Norleucine
  MOD_RES (116)..(116): Homoserine
  MOD_RES (150)..(150): Norleucine
YFGKLKSKLSVIRNLNDQVLFIDQGNRPLFSDXTDSDCRDNAPRTIFIISXYADSQPRGXAVSISVKXEKISTLSCENKIISFKEXNPPDNIKDTKSDIIFFQRSVPGHDNKXQFSSSSYEGYFLACEKERDLFKLILKKEDELGDRSIXFTVQNED

SEQ ID NO: 60 - protein, 157 aa, Artificial Sequence
  Description of Artificial Sequence: Synthetic polypeptide
  MOD_RES (31)..(31): Homoserine
  MOD_RES (33)..(33): Norleucine
  MOD_RES (51)..(51): Norleucine
  MOD_RES (60)..(60): Norleucine
  MOD_RES (63)..(63): Homoserine
  MOD_RES (69)..(69): (S)-47-amino-1-azido-37,41-dioxo-3,6,9,12,15, 18,21,24,27,30,33-undecaoxa-36,42-diazaoctatetracontan-48-oic acid, (S)-40-amino-1-azido-37-oxo-3,6,9,12,15,18,21,24,27,30,33- undecaoxa-36-azahentetracontan-41-oic acid, (S)-39-amino-1-azido- 37-oxo-
  MOD_RES (69)..(69): CONT. FROM ABOVE: 3,6,9,12,15,18,21,24,27,30, 33-undecaoxa-36-azatetracontan-40-oic acid or (R)-41-amino-1- azido-37-oxo-3,6,9,12,15,18,21,24,27,30,33-undecaoxa-39-thia-36- azadotetracontan-42-oic acid
  MOD_RES (86)..(86): Norleucine
  MOD_RES (113)..(113): Norleucine
  MOD_RES (116)..(116): Homoserine
  MOD_RES (150)..(150): Norleucine
YFGKLKSKLSVIRNLNDQVLFIDQGNRPLFSDXTDSDCRDNAPRTIFIISXYADSQPRGXAVSISVKCXKISTLSCENKIISFKEXNPPDNIKDTKSDIIFFQRSVPGHDNKXQFSSSSYEGYFLACEKERDLFKLILKKEDELGDRSIXFTVQNED

SEQ ID NO: 61 - protein, 157 aa, Artificial Sequence
  Description of Artificial Sequence: Synthetic polypeptide
  MOD_RES (31)..(31): Homoserine
  MOD_RES (63)..(63): Homoserine
  MOD_RES (68)..(68): (S)-47-amino-1-azido-37,41-dioxo-3,6,9,12,15, 18,21,24,27,30,33-undecaoxa-36,42-diazaoctatetracontan-48-oic acid, (S)-40-amino-1-azido-37-oxo-3,6,9,12,15,18,21,24,27,30,33- undecaoxa-36-azahentetracontan-41-oic acid, (S)-39-amino-1-azido- 37-oxo-
  MOD_RES (68)..(68): CONT. FROM ABOVE: 3,6,9,12,15,18,21,24,27,30, 33-undecaoxa-36-azatetracontan-40-oic acid or (R)-41-amino-1- azido-37-oxo-3,6,9,12,15,18,21,24,27,30,33-undecaoxa-39-thia-36- azadotetracontan-42-oic acid
  MOD_RES (116)..(116): Homoserine
YFGKLKSKLSVIRNLNDQVLFIDQGNRPLFSDMTDSDCRDNAPRTIFIISMYADSQPRGMAVSISVKXEKISTLSCENKIISFKEMNPPDNIKDTKSDIIFFQRSVPGHDNKMQFSSSSYEGYFLACEKERDLFKLILKKEDELGDRSIMFTVQNED

SEQ ID NO: 62 - protein, 157 aa, Artificial Sequence
  Description of Artificial Sequence: Synthetic polypeptide
  MOD_RES (31)..(31): Homoserine
  MOD_RES (63)..(63): Homoserine
  MOD_RES (69)..(69): (S)-47-amino-1-azido-37,41-dioxo-3,6,9,12,15, 18,21,24,27,30,33-undecaoxa-36,42-diazaoctatetracontan-48-oic acid, (S)-40-amino-1-azido-37-oxo-3,6,9,12,15,18,21,24,27,30,33- undecaoxa-36-azahentetracontan-41-oic acid, (S)-39-amino-1-azido- 37-oxo-
  MOD_RES (69)..(69): CONT. FROM ABOVE: 3,6,9,12,15,18,21,24,27,30, 33-undecaoxa-36-azatetracontan-40-oic acid or (R)-41-amino-1- azido-37-oxo-3,6,9,12,15,18,21,24,27,30,33-undecaoxa-39-thia-36- azadotetracontan-42-oic acid
  MOD_RES (116)..(116): Homoserine
YFGKLKSKLSVIRNLNDQVLFIDQGNRPLFSDMTDSDCRDNAPRTIFIISMYADSQPRGMAVSISVKCXKISTLSCENKIISFKEMNPPDNIKDTKSDIIFFQRSVPGHDNKMQFSSSSYEGYFLACEKERDLFKLILKKEDELGDRSIMFTVQNED

SEQ ID NO: 63 - protein, 157 aa, Artificial Sequence
  Description of Artificial Sequence: Synthetic polypeptide
  MOD_RES (31)..(31): Homoserine
  MOD_RES (33)..(33): O-methyl-L-homoserine
  MOD_RES (51)..(51): O-methyl-L-homoserine
  MOD_RES (60)..(60): O-methyl-L-homoserine
  MOD_RES (63)..(63): Homoserine
  MOD_RES (68)..(68): (S)-47-amino-1-azido-37,41-dioxo-3,6,9,12,15, 18,21,24,27,30,33-undecaoxa-36,42-diazaoctatetracontan-48-oic acid, (S)-40-amino-1-azido-37-oxo-3,6,9,12,15,18,21,24,27,30,33- undecaoxa-36-azahentetracontan-41-oic acid, (S)-39-amino-1-azido- 37-oxo-
  MOD_RES (68)..(68): CONT. FROM ABOVE: 3,6,9,12,15,18,21,24,27,30, 33-undecaoxa-36-azatetracontan-40-oic acid or (R)-41-amino-1- azido-37-oxo-3,6,9,12,15,18,21,24,27,30,33-undecaoxa-39-thia-36- azadotetracontan-42-oic acid
  MOD_RES (86)..(86): O-methyl-L-homoserine
  MOD_RES (113)..(113): O-methyl-L-homoserine
  MOD_RES (116)..(116): Homoserine
  MOD_RES (150)..(150): O-methyl-L-homoserine
YFGKLKSKLSVIRNLNDQVLFIDQGNRPLFSDSTDSDCRDNAPRTIFIISSYADSQPRGSAVSISVKXEKISTLSCENKIISFKESNPPDNIKDTKSDIIFFQRSVPGHDNKSQFSSSSYEGYFLACEKERDLFKLILKKEDELGDRSISFTVQNED

SEQ ID NO: 64 - protein, 157 aa, Artificial Sequence
  Description of Artificial Sequence: Synthetic polypeptide
  MOD_RES (31)..(31): Homoserine
  MOD_RES (33)..(33): O-methyl-L-homoserine
  MOD_RES (51)..(51): O-methyl-L-homoserine
  MOD_RES (60)..(60): O-methyl-L-homoserine
  MOD_RES (63)..(63): Homoserine
  MOD_RES (69)..(69): (S)-47-amino-1-azido-37,41-dioxo-3,6,9,12,15, 18,21,24,27,30,33-undecaoxa-36,42-diazaoctatetracontan-48-oic acid, (S)-40-amino-1-azido-37-oxo-3,6,9,12,15,18,21,24,27,30,33- undecaoxa-36-azahentetracontan-41-oic acid, (S)-39-amino-1-azido- 37-oxo-
  MOD_RES (69)..(69): CONT. FROM ABOVE: 3,6,9,12,15,18,21,24,27,30, 33-undecaoxa-36-azatetracontan-40-oic acid or (R)-41-amino-1- azido-37-oxo-3,6,9,12,15,18,21,24,27,30,33-undecaoxa-39-thia-36- azadotetracontan-42-oic acid
  MOD_RES (86)..(86): O-methyl-L-homoserine
  MOD_RES (113)..(113): O-methyl-L-homoserine
  MOD_RES (116)..(116): Homoserine
  MOD_RES (150)..(150): O-methyl-L-homoserine
YFGKLKSKLSVIRNLNDQVLFIDQGNRPLFSDSTDSDCRDNAPRTIFIISSYADSQPRGSAVSISVKCXKISTLSCENKIISFKESNPPDNIKDTKSDIIFFQRSVPGHDNKSQFSSSSYEGYFLACEKERDLFKLILKKEDELGDRSISFTVQNED

SEQ ID NO: 65 - protein, 157 aa, Artificial Sequence
  Description of Artificial Sequence: Synthetic polypeptide
  MOD_RES (31)..(31): Homoserine
  MOD_RES (33)..(33): O-methyl-L-homoserine
  MOD_RES (51)..(51): O-methyl-L-homoserine
  MOD_RES (60)..(60): O-methyl-L-homoserine
  MOD_RES (63)..(63): Homoserine
  MOD_RES (86)..(86): O-methyl-L-homoserine
  MOD_RES (113)..(113): O-methyl-L-homoserine
  MOD_RES (116)..(116): Homoserine
  MOD_RES (150)..(150): O-methyl-L-homoserine
YFGKLKSKLSVIRNLNDQVLFIDQGNRPLFSDSTDSDSRDNAPRTIFIISSYADSQPRGSAVSISVKCEKISTLSCENKIISFKESNPPDNIKDTKSDIIFFQRSVPGHDNKSQFSSSSYEGYFLACEKERDLFKLILKKEDELGDRSISFTVQNED

SEQ ID NO: 66 - protein, 157 aa, Artificial Sequence
  Description of Artificial Sequence: Synthetic polypeptide
  MOD_RES (31)..(31): Homoserine
  MOD_RES (33)..(33): O-methyl-L-homoserine
  MOD_RES (51)..(51): O-methyl-L-homoserine
  MOD_RES (60)..(60): O-methyl-L-homoserine
  MOD_RES (63)..(63): Homoserine
  MOD_RES (86)..(86): O-methyl-L-homoserine
  MOD_RES (113)..(113): O-methyl-L-homoserine
  MOD_RES (116)..(116): Homoserine
  MOD_RES (150)..(150): O-methyl-L-homoserine
YFGKLKSKLSVIRNLNDQVLFIDQGNRPLFSDSTDSDARDNAPRTIFIISSYADSQPRGSAVSISVKCEKISTLSCENKIISFKESNPPDNIKDTKSDIIFFQRSVPGHDNKSQFSSSSYEGYFLACEKERDLFKLILKKEDELGDRSISFTVQNED

SEQ ID NO: 67 - protein, 157 aa, Artificial Sequence
  Description of Artificial Sequence: Synthetic polypeptide
  MOD_RES (31)..(31): Homoserine
  MOD_RES (63)..(63): Homoserine
  MOD_RES (116)..(116): Homoserine
YFGKLKSKLSVIRNLNDQVLFIDQGNRPLFSDMTDSDSRDNAPRTIFIISMYADSQPRGMAVSISVKCEKISTLSCENKIISFKEMNPPDNIKDTKSDIIFFQRSVPGHDNKMQFSSSSYEGYFLACEKERDLFKLILKKEDELGDRSIMFTVQNED

SEQ ID NO: 68 - protein, 157 aa, Artificial Sequence
  Description of Artificial Sequence: Synthetic polypeptide
  MOD_RES (31)..(31): Homoserine
  MOD_RES (63)..(63): Homoserine
  MOD_RES (116)..(116): Homoserine
YFGKLKSKLSVIRNLNDQVLFIDQGNRPLFSDMTDSDARDNAPRTIFIISMYADSQPRGMAVSISVKCEKISTLSCENKIISFKEMNPPDNIKDTKSDIIFFQRSVPGHDNKMQFSSSSYEGYFLACEKERDLFKLILKKEDELGDRSIMFTVQNED

SEQ ID NO: 69 - protein, 157 aa, Artificial Sequence; identical to SEQ ID NO: 22
  Description of Artificial Sequence: Synthetic polypeptide

SEQ ID NO: 70 - protein, 157 aa, Artificial Sequence
  Description of Artificial Sequence: Synthetic polypeptide
YFGKLKSKLSVIRNLNDQVLFIDQGNRPLFEDMTDSDSRDNAPRTIFIISMYADSQPRGMAVTISVKCEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 71 - protein, 157 aa, Artificial Sequence
  Description of Artificial Sequence: Synthetic polypeptide
YFGKLKSKLSVIRNLNDQVLFIDQGNRPLFEDMTDSDSRDNAPRTIFIISMYADSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 72 - protein, 157 aa, Artificial Sequence
  Description of Artificial Sequence: Synthetic polypeptide
YFGKLKSKLSVIRNLNDQVLFIDQGNRPLFEDMTDSDARDNAPRTIFIISMYADSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 73 - protein, 157 aa, Artificial Sequence
  Description of Artificial Sequence: Synthetic polypeptide
YFGKLKSKLSVIRNLNDQVLFIDQGNRPLFEDMTDSDQRDNAPRTIFIISMYADSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 74 - protein, 157 aa, Artificial Sequence
  Description of Artificial Sequence: Synthetic polypeptide
YFGKLKSKLSVIRNLNDQVLFIDQGNRPLFEDMTDSDARDNAPRTIFIISMYADSQPRGMAVTISVKCEKISTLSAENKIISFKEMNPPDNIKDTKSDIIFFQRSVPGHDNKMQFESSSYEGYFLACEKERDLFKLILKKEDELGDRSIMFTVQNED

SEQ ID NO: 75 - protein, 157 aa, Artificial Sequence
  Description of Artificial Sequence: Synthetic polypeptide
YFGKLKSKLSVIRNLNDQVLFIDQGNRPLFEDMTDSDARDNAPRTIFIISMYADSQPRGMAVTISVKCEKISTLSCENKIISFKEMNPPDNIKDTKSDIIFFQRSVPGHDNKMQFESSSYEGYFLAAEKERDLFKLILKKEDELGDRSIMFTVQNED

SEQ ID NO: 76 - protein, 157 aa, Artificial Sequence
  Description of Artificial Sequence: Synthetic polypeptide
YFGKLKSKLSVIRNLNDQVLFIDQGNRPLFEDMTDSDARDNAPRTIFIISMYADSQPRGMAVTISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 77-79 - skipped (3 entries)

SEQ ID NO: 80 - protein, 157 aa, Artificial Sequence
  Description of Artificial Sequence: Synthetic polypeptide
YFGKLKSKLSVIRNLNDQVLFIDQGNRPLFEDMTDSDARDNAPRTIFIISMYADAQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 81 - protein, 157 aa, Artificial Sequence
  Description of Artificial Sequence: Synthetic polypeptide
YFGKLKSKLSVIRNLNDQVLFIDQGNRPLFEDMTDSDQRDNAPRTIFIISMYADAQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 82 - protein, 157 aa, Artificial Sequence
  Description of Artificial Sequence: Synthetic polypeptide
YFGKLKSKLSVIRNLNDQVLFIDQGNRPLFEDMTDSDCRDNAPRTIFIISMYADSQPRGMAVTISVKCEKISTLSCENKIISFAEMNPPDNIKDTKSDIIFFQRSVPGHDNKMQFESSSYEGYFLACEKERDLFKLILKKEDELGDRSIMFTVQNED

SEQ ID NO: 83 - protein, 157 aa, Artificial Sequence
  Description of Artificial Sequence: Synthetic polypeptide
YFGKLKSKLSVIRNLNDQVLFIDQGNRPLFEDMTDSDCRDNAPRTIFIISMYADSQPRGMAVTISVKCEKISTLSCENKIISFKEMNPPDNIKDTKSAIIFFQRSVPGHDNKMQFESSSYEGYFLACEKERDLFKLILKKEDELGDRSIMFTVQNED

SEQ ID NO: 84-200 - skipped (117 entries)

SEQ ID NO: 201 - protein, 30 aa, Artificial Sequence
  Description of Artificial Sequence: Synthetic polypeptide
  MOD_RES (30)..(30): alpha-keto-phenylalanine
YFGKLESKLSVIRNLNDQVLFIDQGNRPLF

SEQ ID NO: 202 - protein, 30 aa, Artificial Sequence
  Description of Artificial Sequence: Synthetic polypeptide
  MOD_RES (30)..(30): alpha-keto-phenylalanine
YFGKLKSKLSVIRNLNDQVLFIDQGNRPLF

SEQ ID NO: 203 - protein, 30 aa, Artificial Sequence
  Description of Artificial Sequence: Synthetic polypeptide
  MOD_RES (30)..(30): alpha-keto-phenylalanine
GFGKLESKLSVIRNLNDQVLFIDQGNRPLF

SEQ ID NO: 204 - protein, 30 aa, Artificial Sequence
  Description of Artificial Sequence: Synthetic polypeptide
  MOD_RES (30)..(30): alpha-keto-phenylalanine
YAGKLESKLSVIRNLNDQVLFIDQGNRPLF

SEQ ID NO: 205 - protein, 30 aa, Artificial Sequence
  Description of Artificial Sequence: Synthetic polypeptide
  MOD_RES (30)..(30): alpha-keto-phenylalanine
GFGKLKSKLSVIRNLNDQVLFIDQGNRPLF

SEQ ID NO: 206 - protein, 30 aa, Artificial Sequence
  Description of Artificial Sequence: Synthetic polypeptide
  MOD_RES (30)..(30): alpha-keto-phenylalanine
YAGKLKSKLSVIRNLNDQVLFIDQGNRPLF

SEQ ID NO: 207 - protein, 30 aa, Artificial Sequence
  Description of Artificial Sequence: Synthetic polypeptide
  MOD_RES (30)..(30): alpha-keto-phenylalanine
GAGKLKSKLSVIRNLNDQVLFIDQGNRPLF

SEQ ID NO: 208 - protein, 30 aa, Artificial Sequence
  Description of Artificial Sequence: Synthetic polypeptide
  MOD_RES (30)..(30): alpha-keto-phenylalanine
GAGKLESKLSVIRNLNDQVLFIDQGNRPLF

SEQ ID NO: 209 - skipped

SEQ ID NO: 210 - protein, 32 aa, Artificial Sequence
  Description of Artificial Sequence: Synthetic polypeptide
  MOD_RES (1)..(1): 5-oxa-proline
  MOD_RES (32)..(32): alpha-keto-valine
PDMTDSDCRDNAPRTIFIISMYKDSQPRGMAV

SEQ ID NO: 211 - protein, 32 aa, Artificial Sequence
  Description of Artificial Sequence: Synthetic polypeptide
  MOD_RES (1)..(1): 5-oxa-proline
  MOD_RES (3)..(3): Norleucine
  MOD_RES (21)..(21): Norleucine
  MOD_RES (30)..(30): Norleucine
  MOD_RES (32)..(32): alpha-keto-valine
PDXTDSDCRDNAPRTIFIISXYKDSQPRGXAV

SEQ ID NO: 212 - protein, 32 aa, Artificial Sequence
  Description of Artificial Sequence: Synthetic polypeptide
  MOD_RES (1)..(1): 5-oxa-proline
  MOD_RES (3)..(3): O-methyl-L-homoserine
  MOD_RES (21)..(21): O-methyl-L-homoserine
  MOD_RES (30)..(30): O-methyl-L-homoserine
  MOD_RES (32)..(32): alpha-keto-valine
PDSTDSDCRDNAPRTIFIISSYKDSQPRGSAV

SEQ ID NO: 213 - protein, 32 aa, Artificial Sequence
  Description of Artificial Sequence: Synthetic polypeptide
  MOD_RES (1)..(1): 5-oxa-proline
  MOD_RES (32)..(32): alpha-keto-valine
PDMTDSDCRDNAPRTIFIISMYADSQPRGMAV

SEQ ID NO: 214 - protein, 32 aa, Artificial Sequence
  Description of Artificial Sequence: Synthetic polypeptide
  MOD_RES (1)..(1): 5-oxa-proline
  MOD_RES (3)..(3): Norleucine
  MOD_RES (21)..(21): Norleucine
  MOD_RES (30)..(30): Norleucine
  MOD_RES (32)..(32): alpha-keto-valine
PDXTDSDCRDNAPRTIFIISXYADSQPRGXAV

SEQ ID NO: 215 - protein, 32 aa, Artificial Sequence
  Description of Artificial Sequence: Synthetic polypeptide
  MOD_RES (1)..(1): 5-oxa-proline
  MOD_RES (3)..(3): O-methyl-L-homoserine
  MOD_RES (21)..(21): O-methyl-L-homoserine
  MOD_RES (30)..(30): O-methyl-L-homoserine
  MOD_RES (32)..(32): alpha-keto-valine
PDSTDSDCRDNAPRTIFIISSYADSQPRGSAV

SEQ ID NO: 216 - protein, 32 aa, Artificial Sequence
  Description of Artificial Sequence: Synthetic polypeptide
  MOD_RES (1)..(1): 5-oxa-proline
  MOD_RES (3)..(3): O-methyl-L-homoserine
  MOD_RES (21)..(21): O-methyl-L-homoserine
  MOD_RES (30)..(30): O-methyl-L-homoserine
  MOD_RES (32)..(32): alpha-keto-valine
PDSTDSDSRDNAPRTIFIISSYADSQPRGSAV

SEQ ID NO: 217 - protein, 32 aa, Artificial Sequence
  Description of Artificial Sequence: Synthetic polypeptide
  MOD_RES (1)..(1): 5-oxa-proline
  MOD_RES (3)..(3): O-methyl-L-homoserine
  MOD_RES (21)..(21): O-methyl-L-homoserine
  MOD_RES (30)..(30): O-methyl-L-homoserine
  MOD_RES (32)..(32): alpha-keto-valine
PDSTDSDSRDNAPRTIFIISSYKDSQPRGSAV

SEQ ID NO: 218 - protein, 44 aa, Artificial Sequence
  Description of Artificial Sequence: Synthetic polypeptide
  MOD_RES (1)..(1): 5-oxa-proline
  MOD_RES (44)..(44): alpha-keto-leucine
PDMTDSDCRDNAPRTIFIISMYKDSQPRGMAVTISVKCEKISTL

SEQ ID NO: 219 - protein, 44 aa, Artificial Sequence
  Description of Artificial Sequence: Synthetic polypeptide
  MOD_RES (1)..(1): 5-oxa-proline
  MOD_RES (44)..(44): alpha-keto-leucine
PDMTDSDCRDNAPRTIFIISMYADSQPRGMAVTISVKCEKISTL

SEQ ID NO: 220 - protein, 44 aa, Artificial Sequence; identical to SEQ ID NO: 219
  Description of Artificial Sequence: Synthetic polypeptide
  MOD_RES (1)..(1): 5-oxa-proline
  MOD_RES (44)..(44): alpha-keto-leucine

SEQ ID NO: 221 - protein, 44 aa, Artificial Sequence
  Description of Artificial Sequence: Synthetic polypeptide
  MOD_RES (1)..(1): 5-oxa-proline
  MOD_RES (3)..(3): Norleucine
  MOD_RES (21)..(21): Norleucine
  MOD_RES (30)..(30): Norleucine
  MOD_RES (44)..(44): alpha-keto-leucine
PDXTDSDCRDNAPRTIFIISXYKDSQPRGXAVTISVKCEKISTL

SEQ ID NO: 222 - protein, 44 aa, Artificial Sequence
  Description of Artificial Sequence: Synthetic polypeptide
  MOD_RES (1)..(1): 5-oxa-proline
  MOD_RES (3)..(3): O-methyl-L-homoserine
  MOD_RES (21)..(21): O-methyl-L-homoserine
  MOD_RES (30)..(30): O-methyl-L-homoserine
  MOD_RES (44)..(44): alpha-keto-leucine
PDSTDSDCRDNAPRTIFIISSYKDSQPRGSAVTISVKCEKISTL

SEQ ID NO: 223 - protein, 44 aa, Artificial Sequence
  Description of Artificial Sequence: Synthetic polypeptide
  MOD_RES (1)..(1): 5-oxa-proline
  MOD_RES (3)..(3): O-methyl-L-homoserine
  MOD_RES (21)..(21): O-methyl-L-homoserine
  MOD_RES (30)..(30): O-methyl-L-homoserine
  MOD_RES (44)..(44): alpha-keto-leucine
PDSTDSDCRDNAPRTIFIISSYADSQPRGSAVTISVKCEKISTL

SEQ ID NO: 224 - protein, 44 aa, Artificial Sequence
  Description of Artificial Sequence: Synthetic polypeptide
  MOD_RES (1)..(1): 5-oxa-proline
  MOD_RES (3)..(3): O-methyl-L-homoserine
  MOD_RES (21)..(21): O-methyl-L-homoserine
  MOD_RES (30)..(30): O-methyl-L-homoserine
  MOD_RES (44)..(44): alpha-keto-leucine
PDSTDSDCRDNAPRTIFIISSYADSQPRGSAVAISVKCEKISTL

SEQ ID NO: 225 - protein, 44 aa, Artificial Sequence
  Description of Artificial Sequence: Synthetic polypeptide
  MOD_RES (1)..(1): 5-oxa-proline
  MOD_RES (3)..(3): O-methyl-L-homoserine
  MOD_RES (21)..(21): O-methyl-L-homoserine
  MOD_RES (30)..(30): O-methyl-L-homoserine
  MOD_RES (44)..(44): alpha-keto-leucine
PDSTDSDSRDNAPRTIFIISSYADSQPRGSAVAISVKCEKISTL

SEQ ID NO: 226 - protein, 44 aa, Artificial Sequence
  Description of Artificial Sequence: Synthetic polypeptide
  MOD_RES (1)..(1): 5-oxa-proline
  MOD_RES (44)..(44): alpha-keto-leucine
PDMTDSDCRDNAPRTIFIISMYKDSQPRGMAVTISVKSEKISTL

SEQ ID NO: 227 - protein, 53 aa, Artificial Sequence
  Description of Artificial Sequence: Synthetic polypeptide
  MOD_RES (1)..(1): 5-oxa-proline
  MOD_RES (53)..(53): alpha-keto-phenylalanine
PISVKCEKISTLSCENKIISFKEMNPPDNIKDTKSDIIFFQRSVPGHDNKMQF

SEQ ID NO: 228 - protein, 53 aa, Artificial Sequence
  Description of Artificial Sequence: Synthetic polypeptide
  MOD_RES (1)..(1): 5-oxa-proline
  MOD_RES (24)..(24): Norleucine
  MOD_RES (51)..(51): Norleucine
  MOD_RES (53)..(53): alpha-keto-phenylalanine
PISVKCEKISTLSCENKIISFKEXNPPDNIKDTKSDIIFFQRSVPGHDNKXQF

SEQ ID NO: 229 - protein, 53 aa, Artificial Sequence
  Description of Artificial Sequence: Synthetic polypeptide
  MOD_RES (1)..(1): 5-oxa-proline
  MOD_RES (24)..(24): O-methyl-L-homoserine
  MOD_RES (51)..(51): O-methyl-L-homoserine
  MOD_RES (53)..(53): alpha-keto-phenylalanine
PISVKCEKISTLSCENKIISFKESNPPDNIKDTKSDIIFFQRSVPGHDNKSQF

SEQ ID NO: 230 - protein, 53 aa, Artificial Sequence
  Description of Artificial Sequence: Synthetic polypeptide
  MOD_RES (1)..(1): 5-oxa-proline
  MOD_RES (53)..(53): alpha-keto-phenylalanine
PISVKSEKISTLSCENKIISFKEMNPPDNIKDTKSDIIFFQRSVPGHDNKMQF

SEQ ID NO: 231 - protein, 53 aa, Artificial Sequence
  Description of Artificial Sequence: Synthetic polypeptide
  MOD_RES (1)..(1): 5-oxa-proline
  MOD_RES (24)..(24): Norleucine
  MOD_RES (51)..(51): Norleucine
  MOD_RES (53)..(53): alpha-keto-phenylalanine
PISVKSEKISTLSCENKIISFKEXNPPDNIKDTKSDIIFFQRSVPGHDNKXQF

SEQ ID NO: 232 - protein, 53 aa, Artificial Sequence
  Description of Artificial Sequence: Synthetic polypeptide
  MOD_RES (1)..(1): 5-oxa-proline
  MOD_RES (24)..(24): O-methyl-L-homoserine
  MOD_RES (51)..(51): O-methyl-L-homoserine
  MOD_RES (53)..(53): alpha-keto-phenylalanine
PISVKSEKISTLSCENKIISFKESNPPDNIKDTKSDIIFFQRSVPGHDNKSQF

SEQ ID NO: 233 - protein, 53 aa, Artificial Sequence
  Description of Artificial Sequence: Synthetic polypeptide
  MOD_RES (1)..(1): 5-oxa-proline
  MOD_RES (53)..(53): alpha-keto-phenylalanine
PISVKSEKISTLSSENKIISFKEMNPPDNIKDTKSDIIFFQRSVPGHDNKMQF

SEQ ID NO: 234 - protein, 53 aa, Artificial Sequence
  Description of Artificial Sequence: Synthetic polypeptide
  MOD_RES (1)..(1): 5-oxa-proline
  MOD_RES (53)..(53): alpha-keto-phenylalanine
PISVKCEKISTLSSENKIISFKEMNPPDNIKDTKSDIIFFQRSVPGHDNKMQF

SEQ ID NO: 235 - protein, 53 aa, Artificial Sequence
  Description of Artificial Sequence: Synthetic polypeptide
  MOD_RES (1)..(1): 5-oxa-proline
  MOD_RES (24)..(24): O-methyl-L-homoserine
  MOD_RES (51)..(51): O-methyl-L-homoserine
  MOD_RES (53)..(53): alpha-keto-phenylalanine
PISVKCEKISTLSSENKIISFKESNPPDNIKDTKSDIIFFQRSVPGHDNKSQF

SEQ ID NO: 236 - protein, 53 aa, Artificial Sequence
  Description of Artificial Sequence: Synthetic polypeptide
  MOD_RES (1)..(1): 5-oxa-proline
  MOD_RES (24)..(24): O-methyl-L-homoserine
  MOD_RES (51)..(51): O-methyl-L-homoserine
  MOD_RES (53)..(53): alpha-keto-phenylalanine
PISVKSEKISTLSSENKIISFKESNPPDNIKDTKSDIIFFQRSVPGHDNKSQF

SEQ ID NO: 237 - protein, 41 aa, Artificial Sequence
  Description of Artificial Sequence: Synthetic polypeptide
  MOD_RES (1)..(1): 5-oxa-proline
  MOD_RES (41)..(41): alpha-keto-phenylalanine
PCENKIISFKEMNPPDNIKDTKSDIIFFQRSVPGHDNKMQF

SEQ ID NO: 238 - protein, 41 aa, Artificial Sequence
  Description of Artificial Sequence: Synthetic polypeptide
  MOD_RES (1)..(1): 5-oxa-proline
  MOD_RES (41)..(41): alpha-keto-phenylalanine
PSENKIISFKEMNPPDNIKDTKSDIIFFQRSVPGHDNKMQF

SEQ ID NO: 239 - protein, 41 aa, Artificial Sequence
  Description of Artificial Sequence: Synthetic polypeptide
  MOD_RES (1)..(1): 5-oxa-proline
  MOD_RES (12)..(12): Norleucine
  MOD_RES (39)..(39): Norleucine
  MOD_RES (41)..(41): alpha-keto-phenylalanine
PCENKIISFKEXNPPDNIKDTKSDIIFFQRSVPGHDNKXQF

SEQ ID NO: 240 - protein, 41 aa, Artificial Sequence
  Description of Artificial Sequence: Synthetic polypeptide
  MOD_RES (1)..(1): 5-oxa-proline
  MOD_RES (12)..(12): Norleucine
  MOD_RES (39)..(39): Norleucine
  MOD_RES (41)..(41): alpha-keto-phenylalanine
PSENKIISFKEXNPPDNIKDTKSDIIFFQRSVPGHDNKXQF

SEQ ID NO: 241 - protein, 41 aa, Artificial Sequence
  Description of Artificial Sequence: Synthetic polypeptide
  MOD_RES (1)..(1): 5-oxa-proline
  MOD_RES (12)..(12): O-methyl-L-homoserine
  MOD_RES (39)..(39): O-methyl-L-homoserine
  MOD_RES (41)..(41): alpha-keto-phenylalanine
PCENKIISFKESNPPDNIKDTKSDIIFFQRSVPGHDNKSQF

SEQ ID NO: 242 - protein, 41 aa, Artificial Sequence
  Description of Artificial Sequence: Synthetic polypeptide
  MOD_RES (1)..(1): 5-oxa-proline
  MOD_RES (12)..(12): O-methyl-L-homoserine
  MOD_RES (39)..(39): O-methyl-L-homoserine
  MOD_RES (41)..(41): alpha-keto-phenylalanine
PSENKIISFKESNPPDNIKDTKSDIIFFQRSVPGHDNKSQF

SEQ ID NO: 243 - protein, 42 aa, Artificial Sequence
  Description of Artificial Sequence: Synthetic polypeptide
  MOD_RES (1)..(1): 5-oxa-proline
PSSSYEGYFLACEKERDLFKLILKKEDELGDRSIMFTVQNED

SEQ ID NO: 244 - protein, 42 aa, Artificial Sequence
  Description of Artificial Sequence: Synthetic polypeptide
  MOD_RES (1)..(1): 5-oxa-proline
PSSSYEGYFLASEKERDLFKLILKKEDELGDRSIMFTVQNED

SEQ ID NO: 245 - protein, 42 aa, Artificial Sequence
  Description of Artificial Sequence: Synthetic polypeptide
  MOD_RES (1)..(1): 5-oxa-proline
  MOD_RES (35)..(35): Norleucine
PSSSYEGYFLACEKERDLFKLILKKEDELGDRSIXFTVQNED

SEQ ID NO: 246 - protein, 42 aa, Artificial Sequence
  Description of Artificial Sequence: Synthetic polypeptide
  MOD_RES (1)..(1): 5-oxa-proline
  MOD_RES (35)..(35): Norleucine
PSSSYEGYFLASEKERDLFKLILKKEDELGDRSIXFTVQNED

SEQ ID NO: 247 - protein, 42 aa, Artificial Sequence
  Description of Artificial Sequence: Synthetic polypeptide
  MOD_RES (1)..(1): 5-oxa-proline
  MOD_RES (35)..(35): O-methyl-L-homoserine
PSSSYEGYFLACEKERDLFKLILKKEDELGDRSISFTVQNED

SEQ ID NO: 248 - protein, 42 aa, Artificial Sequence
  Description of Artificial Sequence: Synthetic polypeptide
  MOD_RES (1)..(1): 5-oxa-proline
  MOD_RES (35)..(35): O-methyl-L-homoserine
PSSSYEGYFLASEKERDLFKLILKKEDELGDRSISFTVQNED

(19) United States
(12) Patent Application Publication
(10) Pub. No.: US 2022/0056091 A1
(43) Pub. Date: Feb. 24, 2022

(54) MODIFIED IL-18 POLYPEPTIDES AND USES THEREOF

(71) Applicant: Bright Peak Therapeutics AG, Basel (CH)

(72) Inventors: Vijaya Raghavan PATTABIRAMAN, Volketswil (CH); Jean-Philippe CARRALOT, Saint-Louis (FR); Bertolt KREFT, Kleinmachnow (DE); Jeffrey William BODE, Zürich (CH); Régis BOEHRINGER, Zürich (CH); Matilde ARÉVALO-RUIZ, Basel (CH); Camille DELON, Mulhouse (FR); Amélie WIEDERKEHR, Blotzheim (FR); Claudia FETZ, Grüt (CH); Anna HAYDN, Zürich (CH); Alexander FLOHR, Loerrach (DE); Roberto IACONE, Basel (CH); Benoit HORNSPERGER, Altkirch (FR); Chongqing WANG, Wallisellen (CH)

(21) Appl. No.: 17/406,847

(22) Filed: Aug. 19, 2021

Related U.S. Application Data
(60) Provisional application No. 63/067,658, filed on Aug. 19, 2020.

(51) Int. Cl. C07K 14/54; A61K 38/20; A61K 47/60

(52) U.S. Cl. CPC C07K 14/54; A61K 47/60; A61K 38/20

(57) ABSTRACT

The present disclosure relates to modified IL-18 polypeptides, compositions comprising modified IL-18 polypeptides, methods of making the same, and methods of using the modified IL-18 polypeptides for treatment of diseases. In one aspect, the disclosure relates to the treatment of cancer using the modified IL-18 polypeptides. In some embodiments, the disclosed IL-18 polypeptides induce the production of IFNγ. In some embodiments, the disclosed IL-18 polypeptides induce the production of IFNγ without being neutralized by IL-18 binding protein.

### CROSS REFERENCE

This application claims the benefit of U.S. Provisional Application No. 63/067,658 filed Aug. 19, 2020, which application is incorporated herein by reference in its entirety.

### SEQUENCE LISTING

The instant application contains a Sequence Listing which has been submitted electronically in ASCII format and is hereby incorporated by reference in its entirety. Said ASCII copy, created on Aug. 16, 2021, is named 94917-0008_707201 US_SL.txt and is 182,606 bytes bytes in size.

### BACKGROUND

Immunotherapies utilize the immune system of a subject to aid in the treatment of ailments. Immunotherapies can be designed to activate or suppress the immune system depending on the nature of the disease being treated. The goal of immunotherapies for the treatment of cancer is to stimulate the immune system so that it recognizes and destroys tumors or other cancerous tissue. One method of activating the immune system to attack cancer cells in the body of a subject is cytokine therapy. Cytokines are proteins produced in the body that are important in cell signaling and in modulating the immune system. Some cytokine therapy utilizes these properties of cytokines to enhance the immune system of a subject to kill cancer cells.

### BRIEF SUMMARY

In one aspect, described herein is a modified interleukin-18 (IL-18) polypeptide, comprising a modified IL-18 polypeptide comprising E06K and K53A, wherein residue position numbering of the modified IL-18 polypeptide is based on a modified IL-18 polypeptide of SEQ ID NO: 1 as a reference sequence.) 0051 In some embodiments, the modified IL-18 polypeptide further comprises T63A. In some embodiments, the modified IL-18 polypeptide further comprises at least one of Y01X, F02X, C38X, D54X, S55X, C68X, K70X, C76X, or C127X, wherein X is an amino acid or an amino acid derivative. In some embodiments, the modified IL-18 polypeptide further comprises at least one of Y01G, F02A, C38S, D54A, S55A, C68S, K70C, C76S, or C127S. In some embodiments, the modified IL-18 polypeptide further comprises at least one of Y01X, F02X, C38X, D54X, S55X, C68X, E69X, or K70X, C76X, or C127X, wherein X is an amino acid or an amino acid derivative. In some embodiments, the modified IL-18 polypeptide further comprises at least one of Y01G, F02A, C38S, C38A, D54A, S55A, C68S, C68A, E69C, K70CC76S, C76A, C127A, or C127S.)

In some embodiments, the modified IL-18 polypeptide comprises a polymer covalently attached at residue 65, residue 66, residue 67, residue 68, residue 69, residue 70, residue 71, residue 72, residue 73, residue 74 or residue 75. In some embodiments, the modified IL-18 polypeptide comprises a polymer covalently attached at residue C68. In some embodiments, the modified IL-18 polypeptide comprises a polymer covalently attached at residue 69. In some embodiments, the modified IL-18 polypeptide comprises a polymer covalently attached at residue E69. In some embodiments, the modified IL-18 polypeptide comprises a polymer covalently attached at residue E69C. In some embodiments, the modified IL-18 polypeptide comprises a polymer covalently attached at residue 70. In some embodiments, the modified IL-18 polypeptide comprises a polymer covalently attached at residue K70. In some embodiments, the modified IL-18 polypeptide comprises a polymer covalently attached at residue K70C.

In some embodiments, the polymer has a weight average molecular weight of at most about 50,000 Daltons, at most about 25,000 Daltons, at most about 10,000 Daltons, or at most about 6,000 Daltons. In some embodiments, the polymer has a weight average molecular weight of at least about 120 Daltons, at least about 250 Daltons, at least about 300 Daltons, at least about 400 Daltons, or at least about 500 Daltons.

In some embodiments, the polymer comprises a conjugation handle or a reaction product of a conjugation handle with a complementary conjugation handle. In some embodiments, the polymer comprises an azide moiety, an alkyne moiety, or reaction product of an azide-alkyne cycloaddition reaction. In some embodiments, the polymer comprises an azide moiety. In some embodiments, the polymer is a water-soluble polymer. In some embodiments, the water-soluble polymer comprises poly(alkylene oxide), polysaccharide, poly(vinyl pyrrolidone), poly(vinyl alcohol), polyoxazoline, poly(acryloylmorpholine), or a combination thereof. In some embodiments, the water-soluble polymer comprises poly(alkylene oxide). In some embodiments, the poly(alkylene oxide) is polyethylene glycol (PEG).

In some embodiments, the polyethylene glycol has a weight average molecular weight of about 10 kDa to about 50 kDa. In some embodiments, the polyethylene glycol has a weight average molecular weight of about 10 kDa, about 20 kDa, or about 30 kDa. In some embodiments, the polyethylene glycol has a weight average molecular weight of about 30 kDa. In some embodiments, the polyethylene glycol has a weight average molecular weight of from about 1 kDa to about 10 kDa. In some embodiments, the polyethylene glycol has a weight average molecular weight of about 1 kDa, about 2 kDa, about 5 kDa, about 7.5 kDa, or about 10 kDa. In some embodiments, a half-life of the modified IL-18 polypeptide is at least 10% longer than a half-life of a corresponding wild-type IL-18 polypeptide. In some embodiments, the half-life of the modified IL-18 polypeptide is at least 30% longer than the half-life of the corresponding wild-type IL-18 polypeptide.

In some embodiments, the modified IL-18 polypeptide comprises an N-terminal extension. In some embodiments, the modified IL-18 polypeptide comprises an N-terminal truncation. 00111 In some embodiments, the modified IL-18 polypeptide comprises a polypeptide sequence having at least about 80%, at least about 85%, at least about 90%, at least about 95%, or about 100% sequence identity to SEQ ID NO: 2-58. In some embodiments, the modified IL-18 polypeptide comprises a polypeptide sequence having at least about 80% sequence identity to SEQ ID NO: 2-83. In some embodiments, the modified IL-18 polypeptide comprises a polypeptide sequence having at least about 80% sequence identity to SEQ ID NO: 2-58. In some embodiments, the polypeptide sequence is at least about 80% identical to SEQ ID NO: 2 or SEQ ID NO: 18. In some embodiments, the polypeptide sequence is at least about 80% identical to SEQ ID NO: 2. In some embodiments, the polypeptide sequence is at least about 90% identical to SEQ ID NO: 2. In some embodiments, the polypeptide sequence is at least about 95% identical to SEQ ID NO: 2. In some embodiments, the polypeptide sequence is at least about 80% identical to SEQ ID NO: 18. In some embodiments, the polypeptide sequence is at least about 90% identical to SEQ ID NO: 18. In some embodiments, the polypeptide sequence is at least about 95% identical to SEQ ID NO: 18. In some embodiments, the modified IL-18 polypeptide is recombinant.

In some embodiments, the modified IL-18 polypeptide comprises one or more amino acid substitutions selected from: (a) a homoserine residue located at any one of residues 26-36; (b) a homoserine residue located at any one of residues 60-80; (c) a homoserine residue located at any one of residues 110-120; (d) a norleucine residue located at any one of residues 28-38; (d) a norleucine residue located at any one of residues 46-56; (e) a norleucine residue located at any one of residues 54-64; (f) a norleucine residue located at any one of residues 80-90; (g) a norleucine residue located at any one of residues 108-118; and (h) a norleucine residue located at any one of residues 145-155, wherein residue position numbering of the modified IL-18 polypeptide is based on SEQ ID NO: 1 as a reference sequence. In some embodiments, the modified IL-18 polypeptide comprises one or more amino acid substitutions selected from: (a) a homoserine residue located at any one of residues 26-36; (b) a homoserine residue located at any one of residues 60-80; (c) a homoserine residue located at any one of residues 110-120; (d) a norleucine or O-methyl-homoserine residue located at any one of residues 28-38; (d) a norleucine or O-methyl-homoserine residue located at any one of residues 46-56; (e) a norleucine or O-methyl-homoserine residue located at any one of residues 54-64; (f) a norleucine or O-methyl-homoserine residue located at any one of residues 80-90; (g) a norleucine or O-methyl-homoserine residue located at any one of residues 108-118; and (h) a norleucine or O-methyl-homoserine residue located at any one of residues 145-155, wherein residue position numbering of the modified IL-18 polypeptide is based on SEQ ID NO: 1 as a reference sequence. In some embodiments, the modified IL-18 polypeptide comprises one or more amino acid substitutions selected from: (a) a homoserine residue located at any one of residues 26-36; (b) a homoserine residue located at any one of residues 60-80; (c) a homoserine residue located at any one of residues 110-120; (d) a O-methyl-homoserine residue located at any one of residues 28-38; (d) a O-methyl-homoserine residue located at any one of residues 46-56; (e) a O-methyl-homoserine residue located at any one of residues 54-64; (f) a or O-methyl-homoserine residue located at any one of residues 80-90; (g) a O-methyl-homoserine residue located at any one of residues 108-118; and (h) a O-methyl-homoserine residue located at any one of residues 145-155, wherein residue position numbering of the modified IL-18 polypeptide is based on SEQ ID NO: 1 as a reference sequence.

In some embodiments, the modified IL-18 polypeptide comprises one or more amino acid substitutions selected from homoserine (Hse) 31, norleucine (Nle) 33, Nle51, Nle59, Hse75, Nle86, Nle113, Hse116, and Nle150. In some embodiments, the modified IL-18 polypeptide comprises one or more amino acid substitutions selected from homoserine (Hse) 31, norleucine (Nle) 33, O-methyl-homoserine (Omh) 33, Nle51, Omh51, Nle60, Omh60, Hse75, Nle86, Omh86, Hse116, Nle113, Omh113, Nle150, and Omh150. In some embodiments, the modified IL-18 peptide comprises an amino acid substitution with O-methyl-L-homoserine. In some embodiments, the modified IL-18 peptide comprises an amino acid substitution with O-methyl-L-homoserine at positions Met 33, Met 51, Met 60, Met 86, Met 113, or Met 150.

In one aspect, described herein is a population of modified interleukin-18 (IL-18) polypeptides, comprising: a) a plurality of modified IL-18 polypeptides; and b) at least one polymer moiety, wherein the at least one polymer moiety is covalently linked to the modified IL-18 polypeptides and attached at residue 65, residue 66, residue 67, residue 68, residue 69, residue 70, residue 71, residue 72, residue 73, residue 74 or residue 75, wherein the amino acid residue position is based on SEQ ID NO:1 as a reference sequence; wherein at least 90% of the modified IL-18 polypeptides have a molecular weight that is within ±500 Da of the peak molecular weight of the plurality of the modified IL-18 polypeptides as determined by high resolution electrospray ionization mass spectrometry (ESI-HRMS).

In one aspect, described herein is a population of modified interleukin-18 (IL-18) polypeptides, comprising: a) a plurality of modified IL-18 polypeptides; and b) a plurality of polymer moieties, wherein the plurality of polymer moieties are covalently linked to the modified IL-18 polypeptides and attached at residue 65, residue 66, residue 67, residue 68, residue 69, residue 70, residue 71, residue 72, residue 73, residue 74 or residue 75 of the modified IL-18 polypeptide, wherein the amino acid residue position is based on SEQ ID NO: 1 as a reference sequence; wherein at least 90% of the plurality of polymer moieties have a molecular weight that is within ±500 Da of the peak molecular weight of the plurality of the modified IL-18 polypeptides as determined by high resolution electrospray ionization mass spectrometry (ESI-HRMS).

In some embodiments, at least 75% of the plurality of polymers have a molecular weight that is within ±10% of the peak molecular weight of the plurality of polymers as determined by high resolution electrospray ionization mass spectrometry (ESI-HRMS). In some embodiments, the at least one polymer moiety or the plurality of polymer moieties is covalently linked to the modified IL-18 polypeptides at amino acid residue 68, wherein the amino acid residue numbering of the modified IL-18 polypeptides is based on SEQ ID NO: 1 as a reference sequence. In some embodiments, the at least one polymer moiety or the plurality of polymer moieties is covalently linked to the modified IL-18 polypeptides at amino acid residue 69, wherein the amino acid residue numbering of the modified IL-18 polypeptides is based on SEQ ID NO: 1 as a reference sequence. In some embodiments, the at least one polymer moiety or the plurality of polymer moieties is covalently linked to the modified IL-18 polypeptides at amino acid residue 70, wherein the amino acid residue numbering of the modified IL-18 polypeptides is based on SEQ ID NO: 1 as a reference sequence. In some embodiments, the at least one polymer moiety or the plurality of polymer moieties is covalently linked to the modified IL-18 polypeptides at C68, wherein the amino acid residue numbering of the modified IL-18 polypeptides is based on SEQ ID NO: 1 as a reference sequence. In some embodiments, the at least one polymer moiety or the plurality of polymer moieties is covalently linked to the modified IL-18 polypeptides at E69, wherein the amino acid residue numbering of the modified IL-18 polypeptides is based on SEQ ID NO: 1 as a reference sequence. In some embodiments, the at least one polymer moiety or the plurality of polymer moieties is covalently linked to the modified IL-18 polypeptides at E69C, wherein the amino acid residue numbering of the modified IL-18 polypeptides is based on SEQ ID NO: 1 as a reference sequence. In some embodiments, the at least one polymer moiety or the plurality of polymer moieties is covalently linked to the modified IL-18 polypeptides at K70, wherein the amino acid residue numbering of the modified IL-18 polypeptides is based on SEQ ID NO: 1 as a reference sequence. In some embodiments, the at least one polymer moiety or the plurality of polymer moieties is covalently linked to the modified IL-18 polypeptides at K70C, wherein the amino acid residue numbering of the modified IL-18 polypeptides is based on SEQ ID NO: 1 as a reference sequence.

In some embodiments, each modified IL-18 polypeptide of the plurality of modified IL-18 polypeptides comprises one or more mutations. In some embodiments, the one or more mutations are located at residue positions selected from E06, K53, Y01, S55, F02, D54, C38, T63, C68, C76, C127, and K70, wherein residue position numbering of the modified IL-18 polypeptides are based on SEQ ID NO: 1 as a reference sequence. In some embodiments, the one or more mutations are located at residue positions selected from E06, K53, Y01, S55, F02, D54, C38, T63, C68, E69, C76, C127, and K70, wherein residue position numbering of the modified IL-18 polypeptides are based on SEQ ID NO: 1 as a reference sequence. In some embodiments, the one or more mutations are selected from E06K, K53A, Y01G, S55A, F02A, D54A, C38S, T63A, C68S, C76S, C127S, and K70C. In some embodiments, the one or more mutations are selected from E06K, K53A, Y01G, S55A, F02A, D54A, C38S, T63A, C68S, E69C, C76S, C127S, and K70C. In some embodiments, the one or more mutations are E06K and K53A. In some embodiments, the one or more mutations are E06K, K53A, and T63A.

In some embodiments, the population comprises at least 1 μg, at least 10 μg, or at least 1 mg of the modified IL-18 polypeptides. In some embodiments, the population comprises at least 100, at least 1000, or at least 1000 of the modified IL-18 polypeptides. In some embodiments, a ratio of weight average molecular weight over number average molecular weight for the population of the modified IL-18 polypeptide is at most 1.1.

In some embodiments, each of the plurality of polymers comprises a water-soluble polymer. In some embodiments, the water-soluble polymer comprises poly(alkylene oxide), polysaccharide, poly(vinyl pyrrolidone), poly(vinyl alcohol), polyoxazoline, poly(acryloylmorpholine), or a combination thereof. In some embodiments, the water-soluble polymer comprises polyethylene glycol.

In some embodiments, a weight average molecular weight of the plurality of polymers is from about 200 Da to about 50,000 Da. In some embodiments, a weight average molecular weight of the plurality of polymers is from about 10,000 Da to about 30,000 Da.

In some embodiments, the modified IL-18 polypeptide modulates IFNγ production, and wherein an EC₅₀ (nM) of the modified IL-18 polypeptide's ability to induce IFNγ is less than 10-fold higher than, less than 5-fold higher than, or less than an EC₅₀ (nM) of an IL-18 polypeptide of SEQ ID NO: 1. In some embodiments, the EC₅₀ (nM) of the modified IL-18 polypeptide's ability to induce IFNγ is less than 10-fold greater than the EC₅₀ (nM) of SEQ ID NO: 1. In some embodiments, the EC₅₀ (nM) of the modified IL-18 polypeptide's ability to induce IFNγ is less than the EC₅₀ (nM) an IL-18 polypeptide of SEQ ID NO: 1. In some embodiments, the EC₅₀ (nM) of the modified IL-18 polypeptide's ability to induce IFNγ is at least about 10-fold less than the EC₅₀ (nM) of an IL-18 polypeptide of SEQ ID NO: 1.

In some embodiments, the modified IL-18 polypeptide modulates IFNγ production, and wherein an EC₅₀ (nM) of the modified IL-18 polypeptide against IFNγ is less than an EC₅₀ (nM) of an IL-18 polypeptide of SEQ ID NO: 1. In some embodiments, the EC₅₀ (nM) of the modified IL-18 polypeptide against IFNγ is at least 10-fold less than the EC₅₀ (nM) of an IL-18 polypeptide of SEQ ID NO: 1. In some embodiments, the EC₅₀ (nM) of the modified IL-18 polypeptide against IFNγ is about 10-fold less than the EC₅₀ (nM) of an IL-18 polypeptide of SEQ ID NO: 1. In some embodiments, the EC₅₀ (nM) of the modified IL-18 polypeptide against IFNγ is about 15-fold less than the EC₅₀ (nM) of an IL-18 polypeptide of SEQ ID NO: 1.

In some embodiments, the modified IL-18 polypeptide comprises a polypeptide sequence having at least about 80%, at least about 85%, at least about 90%, at least about 95%, or about 100% sequence identity to SEQ ID NO: 2-58. In some embodiments, the modified IL-18 polypeptide comprises a polypeptide sequence having at least about 80%, at least about 85%, at least about 90%, at least about 95%, or about 100% sequence identity to SEQ ID NO: 2-83. In some embodiments, the polypeptide sequence is at least about 80% identical to SEQ ID NO: 2 or SEQ ID NO: 18. In some embodiments, the polypeptide sequence is at least about 80% identical to SEQ ID NO: 2. In some embodiments, the polypeptide sequence is at least about 90% identical to SEQ ID NO: 2. In some embodiments, the polypeptide sequence is at least about 95% identical to SEQ ID NO: 2. In some embodiments, the polypeptide sequence is at least about 80% identical to SEQ ID NO: 18. In some embodiments, the polypeptide sequence is at least about 90% identical to SEQ ID NO: 18. In some embodiments, the polypeptide sequence is at least about 95% identical to SEQ ID NO: 18.

In some embodiments, the modified IL-18 polypeptide exhibits less than a 10-fold lower affinity, less than a 5-fold lower affinity, or a greater affinity to an IL-18 receptor alpha subunit (IL-18Rα) than to IL-18 binding protein (IL-18BP) as measured by K_{D}, and wherein [K_{D} IL-18Rα]/[K_{D} IL-18BP] is greater than 0.1. In some embodiments, the modified IL-18 polypeptide binds to IL-18 receptor alpha (IL-18Rα). In some embodiments, the modified IL-18 polypeptide binds to IL-18Rα with a K_{D} of less than about 200 nM, less than about 100 nM, or less than about 50 nM. In some embodiments, the modified IL-18 polypeptide binds to IL-18Rα with a K_{D} of less than about 10 nM.

In some embodiments, the modified IL-18 polypeptide exhibits a greater affinity to an IL-18 receptor (IL-18R) than to IL-18 binding protein (IL-18BP) as measured by K_{D}, and wherein [K_{D} IL-18R]/[K_{D} IL-18BP] is less than 1. In some embodiments, the modified IL-18 polypeptide binds to IL-18 receptor alpha (IL-18Rα). In some embodiments, the modified IL-18 polypeptide binds to IL-18Rα with a K_{D} of less than about 50 nM. In some embodiments, the modified IL-18 polypeptide binds to IL-18Rα with a K_{D} of less than about 10 nM.

In some embodiments, the modified IL-18 polypeptide binds to an IL-18 receptor alpha/beta (IL-18Rα/β) heterodimer. In some embodiments, the modified IL-18 polypeptide binds to the IL-18Rα/β heterodimer with a K_{D} of less than about 10 nM. In some embodiments, the modified IL-18 polypeptide binds to the IL-18Rα/β heterodimer with a K_{D} of less than about 2 nM. In some embodiments, the modified IL-18 polypeptide is conjugated to an additional peptide.

In one aspect, described herein is a host cell comprising a modified IL-18 polypeptide.

In one aspect, described herein is a method of producing a modified IL-18 polypeptide, wherein the method comprises expressing the modified IL-18 polypeptide in a host cell.

In some embodiments, the host cell is a prokaryotic cell or a eukaryotic cell. In some embodiments, the host cell is a mammalian cell, an avian cell, a fungal cell, or an insect cell. In some embodiments, the host cell is a CHO cell, a COS cell, or a yeast cell.

In one aspect, described herein is a pharmaceutical composition comprising: a) a modified IL-18 polypeptide or a population of modified IL-18 polypeptides; and b) a pharmaceutically acceptable carrier or excipient. In some embodiments, the pharmaceutical composition is in a lyophilized form.

In one aspect, described herein is a method of treating cancer in a subject in need thereof, comprising: administering to the subject a pharmaceutically effective amount of a modified IL-18 polypeptide or a pharmaceutical composition comprising a modified IL-18 polypeptide.

In some embodiments, the cancer is a solid cancer. In some embodiments, the solid cancer is kidney cancer, skin cancer, bladder cancer, bone cancer, brain cancer, breast cancer, colorectal cancer, esophageal cancer, eye cancer, head and neck cancer, lung cancer, ovarian cancer, pancreatic cancer, or prostate cancer. In some embodiments, the solid cancer is metastatic renal cell carcinoma or melanoma. In some embodiments, the solid cancer is a carcinoma or a sarcoma.

In some embodiments, the cancer is a blood cancer. In some embodiments, the blood cancer is leukemia, non-Hodgkin lymphoma, Hodgkin lymphoma, or multiple myeloma.

In some embodiments, the method further comprises reconstituting a lyophilized form of the modified IL-18 polypeptide or the pharmaceutical composition. In some embodiments, the modified IL-18 polypeptide is conjugated to a peptide.

In one aspect, provided herein, is a synthetic IL-18 polypeptide, comprising: a synthetic IL-18 polypeptide comprising a homoserine (Hse) residue at a position selected from the region of residues 21-41, residues 60-80, and residues 106-126, wherein residue position numbering of the modified IL-18 polypeptide is based on SEQ ID NO: 1 as a reference sequence.

In some embodiments, the synthetic IL-18 polypeptide comprises a Hse residue in each of the regions of residues 21-41, residues 60-80, and residues 106-126.

In some embodiments, the synthetic IL-18 polypeptide comprises a Hse residue at position 31. In some embodiments, the synthetic IL-18 polypeptide comprises a Hse residue at position 63 or position 75. In some embodiments, the synthetic IL-18 polypeptide comprises a Hse residue at position 63. In some embodiments, the synthetic IL-18 polypeptide comprises a Hse residue at position 75. In some embodiments, the synthetic IL-18 polypeptide comprises a Hse residue at position 116. In some embodiments, the synthetic IL-18 polypeptide comprises Hse residues at positions 31, 116, and at least one of positions 63 and 75.

In some embodiments, the synthetic IL-18 polypeptide comprises an amino acid substitution of at least one methionine residue in SEQ ID NO: 1. In some embodiments, the amino acid substitution of at least one methionine residue in SEQ ID NO: 1 comprises a substitution at M33, M51, M60, M86, M113, or M150. In some embodiments, the synthetic IL-18 polypeptide comprises substitutions of at least three methionine residues. In some embodiments, the synthetic IL-18 polypeptide comprises substitutions of at least five methionine residues. In some embodiments, the synthetic IL-18 polypeptide comprises substitution of at least six methionine residues.

In some embodiments, at least one methionine residue is substituted for an O-methyl-homoserine (Omh) residue. In some embodiments, at least three methionine residues are substituted for Omh residues. In some embodiments, at least five methionine residues are substituted for Omh residues. In some embodiments, each methionine substitution is for a norleucine or Omh residue. In some embodiments, each methionine substitution is for an Omh residue. In some embodiments, each methionine residue of SEQ ID NO: 1 is substituted for an Omh residue.

In some embodiments, the synthetic IL-18 polypeptide comprises an additional mutation to SEQ ID NO: 1. In some embodiments, the synthetic IL-18 polypeptide comprises an amino acid sequence at least about 80% identical to that of SEQ ID NO: 1. In some embodiments, the synthetic IL-18 polypeptide comprises a polymer covalently attached to a residue of the synthetic IL-18 polypeptide

In one aspect, described herein is a method of making a modified IL-18 polypeptide, comprising: a) synthesizing two or more fragments of the modified IL-18 polypeptide; b) ligating the fragments; and c) folding the ligated fragments. In some embodiments, the method further comprises attaching a water-soluble polymer to the folded, ligated fragments.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawing, of which:

FIG. 1 illustrates the mechanism of action of IL-18 on IFNγ and IL-18BP production, and IL-18 inhibitory activity by IL-18BP.

FIG. 2A illustrates synthetic wild type IL-18 polypeptide.

FIG. 2B illustrates a modified synthetic IL-18 polypeptide with two modified amino acid residues (indicated by dark circles).

FIG. 2C illustrates a modified synthetic IL-18 polypeptide comprising a polymer moiety.

FIG. 3 illustrates the coupling of a dibenzocyclooctyne (DBCO) polyethylene glycol (PEG) with a modified IL-18 polypeptide comprising an azide.

FIG. 4 illustrates the binding of a modified IL-18 polypeptide comprising a polymer with IL-18Rα.

FIG. 5 shows a synthetic scheme to prepare a modified IL-18 polypeptide of SEQ ID NO: 26 comprising an azide moiety using a modified IL-18 polypeptide fragment.

FIG. 6A shows the IFNγ induction ability of a modified IL-18 polypeptide of the disclosure compared to a wild type IL-18 polypeptide.

FIG. 6B shows IL-18BP inhibition of a modified IL-18 polypeptide of the disclosure compared to a wild type IL-18 polypeptide.

FIG. 7 compares EC₅₀ values of a control (solid circles, solid line); control+IL-18BP (semi-open circles, dashed line); an IL-18 polypeptide of SEQ ID NO: 1 (solid triangles, solid line); an IL-18 polypeptide of SEQ ID NO: 1+IL-18BP (open triangles, dashed line); a modified IL-18 polypeptide of SEQ ID NO: 2 (solid diamonds, solid line); and a modified IL-18 polypeptide of SEQ ID NO: 2+IL-18BP (semi-open triangles, dashed line).

FIG. 8 shows a synthetic scheme to synthesize a modified IL-18 polypeptide of SEQ ID NO: 24 using IL-18 fragments.

FIG. 9 shows a synthetic scheme to synthesize a modified IL-18 polypeptide using IL-18 fragments comprising an azide moiety on K70.

FIG. 10 shows a synthetic scheme to synthesize a modified IL-18 polypeptide of SEQ ID NO: 25 using IL-18 fragments.

FIG. 11 shows a synthetic scheme to synthesize a modified IL-18 polypeptide of SEQ ID NO: 31 using IL-18 fragments.

FIG. 12 shows a synthetic scheme to prepare a modified IL-18 polypeptide of SEQ ID NO: 32 comprising an azide moiety using a modified IL-18 polypeptide fragment.

FIG. 13 shows a synthetic scheme to prepare a modified IL-18 polypeptide of SEQ ID NO: 33 using IL-18 fragments.

FIG. 14 shows a synthetic scheme to prepare a modified IL-18 polypeptide of SEQ ID NO: 34 comprising an azide moiety using a modified IL-18 polypeptide fragment.

FIG. 15 shows a synthetic scheme to prepare a modified IL-18 polypeptide comprising a PEG-azide moiety covalently attached at residue 70, which has been substituted to an aspartate residue using a modified IL-18 polypeptide fragment.

FIG. 16 shows a synthetic scheme to prepare a modified IL-18 polypeptide of SEQ ID NO: 62 comprising an azide moiety using a modified IL-18 polypeptide fragment.

FIG. 17 shows a generic synthetic scheme which can be used to prepare a modified IL-18 polypeptide comprising a PEG azide group covalently attached to a variety of amino acid residues.

FIG. 18 shows a schematic representation of coupling of a bifunctional probe to an IL-18 polypeptide provided herein.

FIG. 19 shows a schematic representation of coupling of a poly(ethylene glycol) moiety to an IL-18 polypeptide activated with a bifunctional probe.

FIG. 20 shows interferon gamma (IFN) levels at various time points after administration of the indicated IL-18 polypeptides in a mouse model.

FIG. 21 shows C-X-C motif chemokine ligand 10 (CXCL10) levels at various time points after administration of the indicated IL-18 polypeptides in a mouse model.

FIG. 22 shows in vitro induction of IFNγ, IL1β, TNFα, IL-6, IL-10, and IL-12p70 after 24 hr stimulation of PBMC with human IL-18.and the indicated variants.

FIG. 23 shows surface expression of CD16 on human CD3−/CD56+ NK cells upon 72 hr stimulation with human IL-18 and the indicated variants.

### DETAILED DESCRIPTION

Immune responses to tumors are primarily the function of T helper type 1 (Th1) lymphocytes. Th1 responses include the secretion of cytokines IL-2, IL-12, IL-18, IFNγ, and the generation of specific cytotoxic T lymphocytes that recognize specific tumor antigens. The Th1 response is a vital arm of host defense against many microorganisms. However, the Th1 response is also associated with autoimmune diseases and organ transplant rejection.

Interleukin 18 (IL-18) is a pro-inflammatory cytokine that elicits biological activities that initiate or promote host defense and inflammation following infection or injury. IL-18 has been implicated in autoimmune diseases, myocardial function, emphysema, metabolic syndromes, psoriasis, inflammatory bowel disease, hemophagocytic syndromes, macrophage activation syndrome, sepsis, and acute kidney injury. In some models of disease, IL-18 plays a protective role.

IL-18 also plays a major role in the production of IFNγ from T-cells and natural killer cells. IFNγ is a Th1 cytokine mainly produced by T cells, NK cells, and macrophages and is critical for innate and adaptive immunity against viral, some bacterial, and protozoal infections. IFNγ is also an important activator of macrophages and inducer of Class II major histocompatibility complex (MHC) molecule expression.

IL-18 forms a signaling complex by binding to the IL-18 alpha chain (IL-18Rα), which is the ligand binding chain for mature IL-18. However, the binding affinity of IL-18 to IL-18Rα is low. In cells that express the co-receptor, IL-18 receptor beta chain (IL-18Rβ), a high affinity heterodimer complex is formed, which then activates cell signaling.

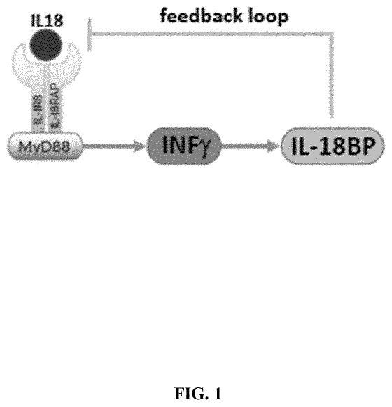

The activity of IL-18 is balanced by the presence of a high affinity, naturally occurring IL-18 binding protein (IL-18BP). IL-18BP binds IL-18 and neutralizes the biological activity of IL-18. Cell surface IL-18Rα competes with IL-18BP for IL-18 binding. Increased disease severity can be associated with an imbalance of IL-18 to IL-18BP such that levels of free IL-18 are elevated in the circulation. FIG. 1 illustrates the mechanism of action of IL-18, IFNγ production, IL-18BP production, and inhibition of IL-18 activity by IL-18BP. IL-18 induces IFNγ production, which in turn induces IL-18BP production. IL-18BP then competes with IL-18Rα to inhibit IL-18 activity.

The following description and examples illustrate embodiments of the present disclosure in detail. It is to be understood that this present disclosure is not limited to the particular embodiments described herein and as such can vary. Those of skill in the art will recognize that there are numerous variations and modifications of this present disclosure, which are encompassed within its scope.

Although various features of the present disclosure may be described in the context of a single embodiment, the features may also be provided separately or in any suitable combination. Conversely, although the present disclosure may be described herein in the context of separate embodiments for clarity, the present disclosure may also be implemented in a single embodiment.

The section headings used herein are for organizational purposes only and are not to be construed as limiting the subject matter described.

### I. Definitions

All terms are intended to be understood as they would be understood by a person skilled in the art. Unless defined otherwise, all technical and scientific terms used herein have the same meaning as commonly understood by one of ordinary skill in the art to which the disclosure pertains.

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only and is not intended to be limiting.

The terminology used herein is for the purpose of describing particular cases only and is not intended to be limiting. In this application, the use of the singular includes the plural unless specifically stated otherwise. As used herein, the singular forms “a”, “an” and “the” are intended to include the plural forms as well, unless the context clearly indicates otherwise.

In this application, the use of “or” means “and/or” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 15%, up to 10%, up to 5%, or up to 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value should be assumed.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures. To facilitate an understanding of the present disclosure, a number of terms and phrases are defined below.

As used herein, an “alpha-keto amino acid” or the phrase “alpha-keto” before the name of an amino acid refers to an amino acid or amino acid derivative having a ketone functional group positioned between the carbon bearing the amino group and the carboxylic acid of an amino acid. Alpha-keto amino acids of the instant disclosure have a structure as set forth in the following formula:

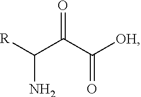

wherein R is the side chain of any natural or unnatural amino acid. The R functionality can be in either the L or D orientation in accordance with standard amino acid nomenclature. In preferred embodiments, alpha-keto amino acids are in the L orientation. When the phrase “alpha-keto” is used before the name of a traditional natural amino acid (e.g., alpha-keto leucine, alpha-keto phenylalanine, etc.) or a common unnatural amino acid (e.g., alpha-keto norleucine, alpha-keto O-methyl-homoserine, etc.), it is intended that the alpha-keto amino acid referred to matches the above formula with the side chain of the referred to amino acid. When an alpha-keto amino acid residue is set forth in a peptide or polypeptide sequence herein, it is intended that a protected version of the relevant amino acid is also encompassed (e.g., for a sequence terminating in a C-terminal alpha-keto amino acid, the terminal carboxylic acid residue may be appropriately capped with a protecting group such as a tert-butyl group).

The term “pharmaceutically acceptable” refers to approved or approvable by a regulatory agency of the Federal or a state government or listed in the U.S. Pharmacopeia or other generally recognized pharmacopeia for use in animals, including humans.

A “pharmaceutically acceptable excipient, carrier or diluent” refers to an excipient, carrier or diluent that can be administered to a subject, together with an agent, and which does not destroy the pharmacological activity thereof and is nontoxic when administered in doses sufficient to deliver a therapeutic amount of the agent.

A “pharmaceutically acceptable salt” suitable for the disclosure may be an acid or base salt that is generally considered in the art to be suitable for use in contact with the tissues of human beings or animals without excessive toxicity, irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzene sulfonic, ethane disulfonic, 2-hydroxyethyl sulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH₂)ₙ—COOH where n is 0, 2, 3, 4, or 4, and the like. Similarly, pharmaceutically acceptable cations include, but are not limited to sodium, potassium, calcium, aluminum, lithium and ammonium. Those of ordinary skill in the art will recognize from this disclosure and the knowledge in the art that further pharmaceutically acceptable salts include those listed by Remington's Pharmaceutical Sciences, 17th ed., Mack Publishing Company, Easton, Pa., p. 1418 (1985). In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in an appropriate solvent.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

The term “subject” refers to an animal which is the object of treatment, observation, or experiment. By way of example only, a subject includes, but is not limited to, a mammal, including, but not limited to, a human or a non-human mammal, such as a non-human primate, bovine, equine, canine, ovine, or feline.

The term “optional” or “optionally” denotes that a subsequently described event or circumstance can but need not occur, and that the description includes instances where the event or circumstance occurs and instances in which it does not.

The term “moiety” refers to a specific segment or functional group of a molecule. Chemical moieties are often recognized chemical entities embedded in or appended to a molecule.

As used herein, the term “number average molecular weight” (Mn) means the statistical average molecular weight of all the individual units in a sample, and is defined by Formula (1):

$$M n = \frac{\sum {N}_{i} {M}_{i}}{\sum {N}_{i}} \qquad Formula ( 1 )$$

where Mᵢ is the molecular weight of a unit and Nᵢ is the number of units of that molecular weight.

As used herein, the term “weight average molecular weight” (Mw) means the number defined by Formula (2):

$$M w = \frac{\sum {N}_{i} {M}_{i}^{2}}{\sum {N}_{i} {M}_{i}} \qquad Formula ( 2 )$$

where Mᵢ is the molecular weight of a unit and Nᵢ is the number of units of that molecular weight.

As used herein, “peak molecular weight” (Mp) means the molecular weight of the highest peak in a given analytical method (e.g. mass spectrometry, size exclusion chromatography, dynamic light scattering, analytical centrifugation, etc.).

### II. Modified IL-18 Polypeptides

The present disclosure relates to modified IL-18 polypeptides useful as therapeutic agents. Modified IL-18 polypeptides provided herein can be used as immunotherapies or as parts of other immunotherapy regimens. Such modified IL-18 polypeptides may display binding characteristics for the IL-18 receptors (IL-18R) that differ from wild-type IL-18.

In one aspect, modified IL-18 polypeptides described herein have increased affinity for IL-18Rα or IL-18Rβ. In one aspect, modified IL-18 polypeptides described herein have decreased affinity for IL-18Rα or IL-18Rβ. In some embodiments, the modified IL-18 polypeptides have an increased affinity for the IL-18Rα/β heterodimer. In one aspect, modified IL-18 polypeptides described herein have decreased affinity for the IL-18Rα/β heterodimer.

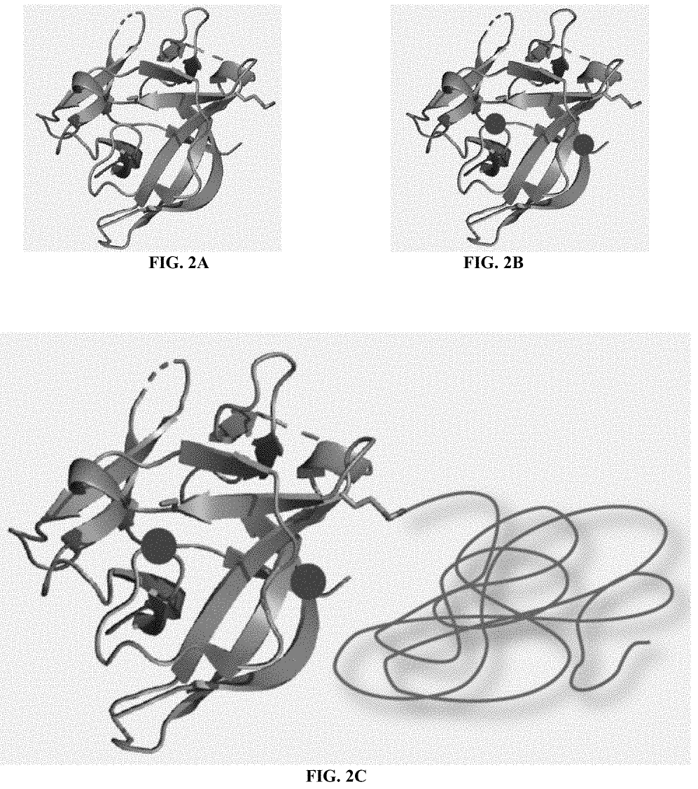

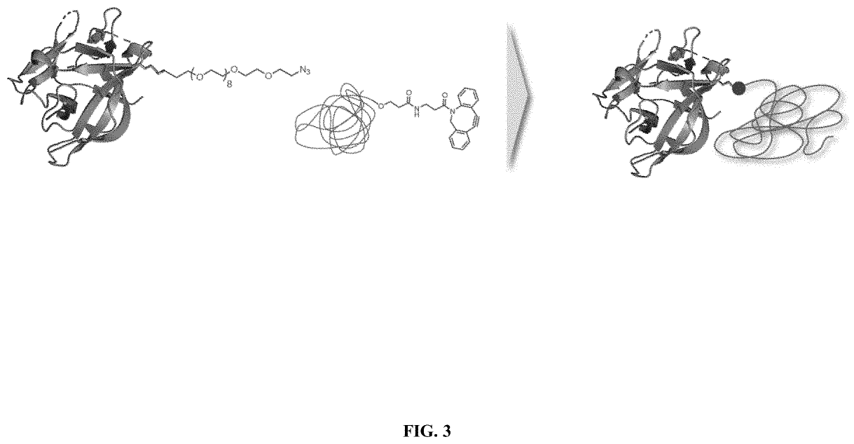

In some embodiments, the binding affinity between the modified IL-18 polypeptides and IL-18Rα is the same as or lower than the binding affinity between a wild-type IL-18 and IL-18Rα. In some embodiments, the binding affinity between the modified IL-18 polypeptides and IL-18Rα is the same as or higher than the binding affinity between a wild-type IL-18 and IL-18Rα. In some embodiments, the binding affinity between the modified IL-18 polypeptides and IL-18Rβ is the same as or lower than the binding affinity between a wild-type IL-18 and IL-18Rβ. In some embodiments, the binding affinity between the modified IL-18 polypeptides and IL-18Rβ is the same as or higher than the binding affinity between a wild-type IL-18 and IL-IL-18Rβ. In some embodiments, the binding affinity between the modified IL-18 polypeptides and the IL-18Rα/β heterodimer is the same as or lower than the binding affinity between a wild-type IL-18 and the IL-18R α/β heterodimer. In some embodiments, the binding affinity between the modified IL-18 polypeptides and the IL-18R α/β heterodimer is the same as or higher than the binding affinity between a wild-type IL-18 and the IL-18R α/β heterodimer. FIG. 2A illustrates a synthetic wild type IL-18 polypeptide.

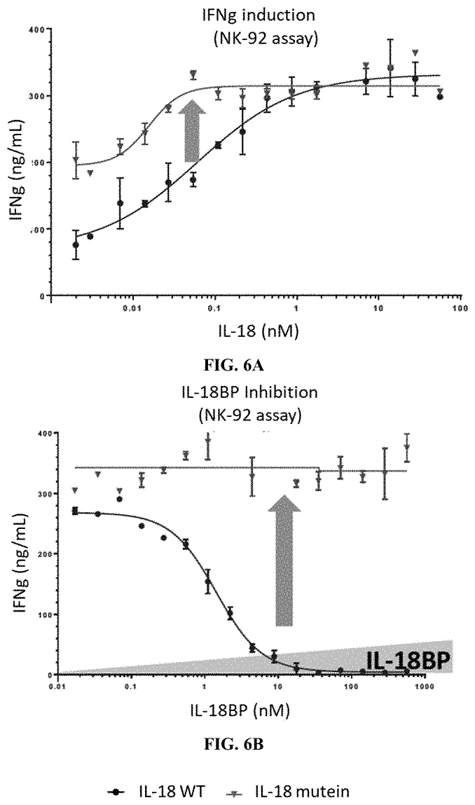

In some embodiments, a modified IL-18 polypeptide provided herein displays an ability to induce interferon gamma (IFNγ) production after administration to a subject. In some embodiments, the ability to induce IFNγ is comparable to that of a wild type IL-18 (e.g., displays an EC50 for IFNγ induction that is within about 10-fold of that of a wild type IL-18). An exemplary IL-18 polypeptide provided herein displaying this characteristic is shown in FIG. 6A, which shows a comparison of IFNγ production (ng/mL, y-axis) as a function of concentration of a wild type versus modified IL-18 polypeptide (mutein) (nM, x-axis). In some embodiments, a modified IL-18 polypeptide provided herein also display a reduced binding IL-18 binding protein (IL-18BP). In some embodiments, a modified IL-18 polypeptide provided herein can induce IFNγ even in the presence of IL-18BP (e.g., the ability of the modified IL-18 polypeptide to induce IFNγ is not substantially inhibited by the presence of IL-18BP) (nM, x-axis). An example of an IL-18 polypeptide with this property compared to wild type IL-18 is shown in FIG. 6B, which shows IFNγ production (ng/mL, y-axis) as a function of IL-18BP concentration (nM, x-axis) in a sample treated with the same level of wild type IL-18 (circles) or a modified IL-18 polypeptide provided herein (inverted triangles). Notably, the modified IL-18 polypeptide provided herein showed no inhibition in its ability to induce IFNγ in the presence of IL-18BP, whereas the wild type IL-18 displayed substantial reduction in this ability as the concentration of IL-18BP increased. In some embodiments, a modified IL-18 polypeptide provided herein displays a similar or only slightly reduced ability to induce IFNγ production compared to wild type IL-18. In some embodiments, a modified IL-18 polypeptide provided herein displays a significant reduction in inhibition of the ability to induce IFNγ production in the presence of IL-18BP compared to wild type IL-18. In some embodiments, a modified IL-18 polypeptide provided herein displays a similar or only slightly reduced ability to induce IFNγ production compared to wild type IL-18, and a significant reduction in inhibition of the ability to induce IFNγ production in the presence of IL-18BP compared to wild type IL-18.

A modified IL-18 polypeptide as described herein can comprise one or more non-canonical amino acids. “Non-canonical” amino acids can refer to amino acid residues in D- or L-form that are not among the 20 canonical amino acids generally incorporated into naturally occurring proteins. In some embodiments, one or more amino acids of the modified IL-18 polypeptides are substituted with one or more non-canonical amino acids. Non-canonical amino acids include, but are not limited to N-alpha-(9-Fluorenylmethyloxycarbonyl)-L-azidolysine (Fmoc-L-Lys(N₃)—OH), N-alpha-(9-Fluorenylmethyloxycarbonyl)-L-biphenylalanine (Fmoc-L-Bip-OH), and N-alpha-(9-Fluorenylmethyloxycarbonyl)-O-benzyl-L-tyrosine (Fmoc-L-Tyr(Bzl)-OH.

Exemplary non-canonical amino acids include azido-lysine (AzK), hydroxylysine, allo-hydroxylysine, ε-N,N,N-trimethyllysine, ε-N-acetyllysine, 5-hydroxylysine, Fmoc-Lys (Me, Boc), Fmoc-Lys (Me)₃, Fmoc-Lys (palmitoyl), Fmoc-L-photo-lysine, DL-5-hydroxylysine, H-L-photo-lysine, and/or other similar amino acids. Example non-canonical amino acids also include D-methionine, selenocysteine, and/or other similar amino acids.

Exemplary non-canonical amino acids also include p-acetyl-L-phenylalanine, p-iodo-L-phenylalanine, p-methoxyphenylalanine, O-methyl-L-tyrosine, p-propargyloxyphenylalanine, p-propargyl-phenylalanine, L-3-(2-naphthyl) alanine, 3-methyl-phenylalanine, O-4-allyl-L-tyrosine, 4-propyl-L-tyrosine, tri-O-acetyl-GlcNAcp-serine, L-Dopa, fluorinated phenylalanine, isopropyl-L-phenylalanine, p-azido-L-phenylalanine, p-acyl-L-phenylalanine, p-benzoyl-L-phenylalanine, p-Boronophenylalanine, O-propargyltyrosine, L-phosphoserine, phosphonoserine, phosphonotyrosine, p-bromophenylalanine, p-amino-L-phenylalanine, isopropyl-L-phenylalanine, an analogue of a tyrosine amino acid; an analogue of a glutamine amino acid; an analogue of a phenylalanine amino acid; an analogue of a serine amino acid; an analogue of a threonine amino acid; an alkyl, aryl, acyl, azido, cyano, halo, hydrazine, hydrazide, hydroxyl, alkenyl, alkynyl, ether, thiol, sulfonyl, seleno, ester, thioacid, borate, boronate, phospho, phosphono, phosphine, heterocyclic, enone, imine, aldehyde, hydroxylamine, keto, or amino substituted amino acid, a β-amino acid; a cyclic amino acid other than proline or histidine; an aromatic amino acid other than phenylalanine, tyrosine or tryptophan; or a combination thereof. In some embodiments, the non-canonical amino acids are selected from β-amino acids, homoamino acids, cyclic amino acids and amino acids with derivatized side chains. In some embodiments, the non-canonical amino acids comprise β-alanine, β-aminopropionic acid, piperidinic acid, aminocaprioic acid, aminoheptanoic acid, aminopimelic acid, desmosine, diaminopimelic acid, N^{α}-ethylglycine, N^{α}-ethylaspargine, isodesmosine, allo-isoleucine, ω-methylarginine, N^{α}-methylglycine, N^{α}-methylisoleucine, N^{α}-methylvaline, γ-carboxyglutamate, O-phosphoserine, N^{α}-acetylserine, N^{α}-formylmethionine, 3-methylhistidine, and/or other similar amino acids.

In some embodiments, amino acid residues of the modified IL-18 polypeptides are substituted with modified lysine residues. In some embodiments, the modified lysine residues comprise an amino, azide, allyl, ester, and/or amide functional groups. In some embodiments, the modified lysine residues contain conjugation handles which can serve as useful anchor points to attach additional moieties to the modified IL-18 polypeptides. In some embodiments, the modified lysine residues have a structure built from precursors Structure 1, Structure 2, Structure 3, or Structure 4:

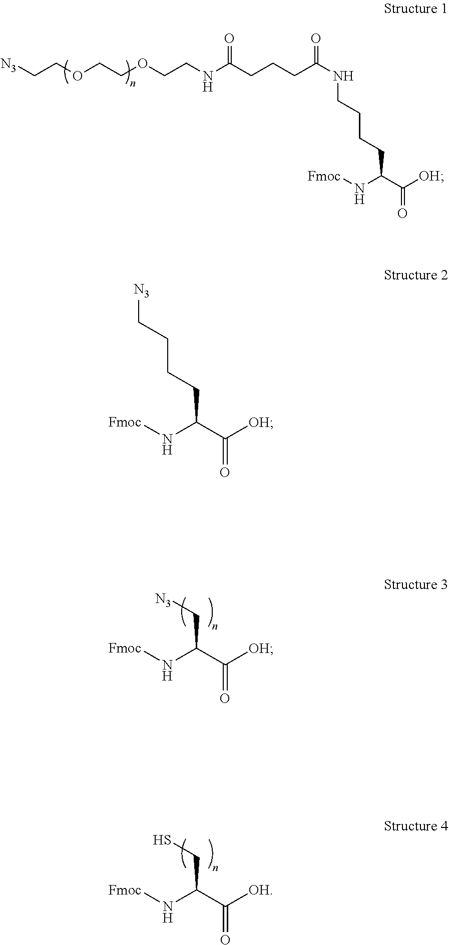

In some embodiments, the modified IL-18 polypeptide contains a substitution for modified amino acid residues which can be used for attachment of additional functional groups which can be used to facilitate conjugation reaction or attachment of various payloads to the modified IL-18 polypeptide (e.g., polymers). The substitution can be for a naturally occurring amino acid which is more amenable to attachment of additional functional groups (e.g., aspartic acid, cysteine, glutamic acid, lysine, serine, threonine, or tyrosine), a derivative of a modified version of any naturally occurring amino acid, or any unnatural amino acid (e.g., an amino acid containing a desired reactive group, such as a CLICK chemistry reagent such as an azide, alkyne, etc.). Non-limiting examples of such modified amino acid residues include the modified lysine, glutamic acid, aspartic acid, and cysteine provided below:

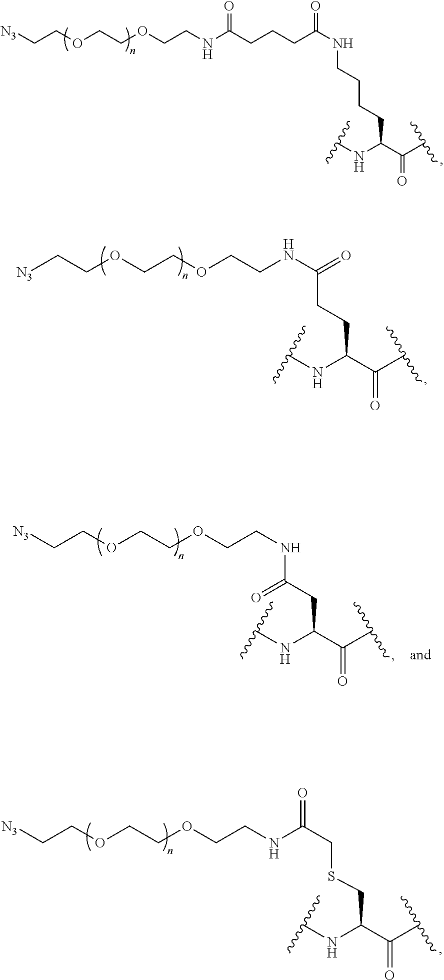

wherein each n is an integer from 1-30. These non-limiting examples of modified amino acid residues can be used at any location at which it is desirable to add an additional functionality (e.g., a polymer) to the modified IL-18 polypeptide.

### Site-Specific Modifications

In some embodiments, a modified IL-18 polypeptide described herein comprises one or more modifications at one or more amino acid residues. In some embodiments, the residue position numbering of the modified IL-18 polypeptide is based on SEQ ID NO: 1 as a reference sequence. In some embodiments, the residue position numbering of the modified IL-18 polypeptide is based on a wild-type human IL-18 polypeptide as a reference sequence.

Modifications to the polypeptides described herein encompass mutations, addition of various functionalities, deletion of amino acids, addition of amino acids, or any other alteration of the wild-type version of the protein or protein fragment. Functionalities which may be added to polypeptides include polymers, linkers, alkyl groups, detectable molecules such as chromophores or fluorophores, reactive functional groups, or any combination thereof. In some embodiments, functionalities are added to individual amino acids of the polypeptides. In some embodiments, functionalities are added site-specifically to the polypeptides.

In some embodiments, the modified IL-18 polypeptides described herein contain 1, 2, 3, 4, 5, 6, 7, 8, 9, or 10 modified amino acid residues. In some embodiments, the modified IL-18 polypeptides described herein contain 1 modified amino acid residue. In some embodiments, the modified IL-18 polypeptides described herein contain 2 modified amino acid residues. In some embodiments, the modified IL-18 polypeptides described herein contain 3 modified amino acid residues. FIG. 2B illustrates a modified synthetic IL-18 polypeptide with 2 modified amino acid residues.

In some embodiments, a modified IL-18 polypeptide provided herein comprises an amino acid sequence of any one of SEQ ID NOs: 2-58 provided herein. In some embodiments, the modified IL-18 polypeptide comprises an amino acid sequence at least 85% identical to the sequence of any one of SEQ ID NOs: 2-58. In some embodiments, a modified IL-18 polypeptide provided herein comprises an amino acid sequence of any one of SEQ ID NOs: 2-83 provided herein. In some embodiments, the modified IL-18 polypeptide comprises an amino acid sequence at least 85% identical to the sequence of any one of SEQ ID NOs: 2-83. In some embodiments, the modified IL-18 polypeptide comprises an amino acid sequence of SEQ ID NO: 2. In some embodiments, the modified IL-18 polypeptide comprises an amino acid sequence at least 85% identical to the sequence of SEQ ID NO: 2. In some embodiments, the modified IL-18 polypeptide comprises an amino acid sequence of SEQ ID NO: 7. In some embodiments, the modified IL-18 polypeptide comprises an amino acid sequence at least 85% identical to the sequence of SEQ ID NO: 7. In some embodiments, the modified IL-18 polypeptide comprises an amino acid sequence of SEQ ID NO: 18. In some embodiments, the modified IL-18 polypeptide comprises an amino acid sequence at least 85% identical to the sequence of SEQ ID NO: 18. In some embodiments, the sequence identity is measured by protein-protein BLAST algorithm using parameters of Matrix BLOSUM62, Gap Costs Existence:11, Extension:1, and Compositional Adjustments Conditional Compositional Score Matrix Adjustment.

In some embodiments, a modified IL-18 polypeptide described herein comprises at least 2, at least 3, at least 4, at least 5, at least 6, at least 7, or at least 9 amino acid substitutions. In some embodiments, the modified IL-18 polypeptide comprises 3 to 9 amino acid substitutions. In some embodiments, the modified IL-18 polypeptide comprises 3 or 4 amino acid substitutions, 3 to 5 amino acid substitutions, 3 to 6 amino acid substitutions, 3 to 7 amino acid substitutions, 3 to 9 amino acid substitutions, 4 or 5 amino acid substitutions, 4 to 6 amino acid substitutions, 4 to 7 amino acid substitutions, 4 to 9 amino acid substitutions, 5 or 6 amino acid substitutions, 5 to 7 amino acid substitutions, 5 to 9 amino acid substitutions, 6 or 7 amino acid substitutions, 6 to 9 amino acid substitutions, or 7 to 9 amino acid substitutions. In some embodiments, the modified IL-18 polypeptide comprises 3 amino acid substitutions, 4 amino acid substitutions, 5 amino acid substitutions, 6 amino acid substitutions, 7 amino acid substitutions, or 9 amino acid substitutions. In some embodiments, the modified IL-18 polypeptide comprises at most 4 amino acid substitutions, 5 amino acid substitutions, 6 amino acid substitutions, 7 amino acid substitutions, or 9 amino acid substitutions.

In some embodiments, a modified IL-18 polypeptide described herein comprises a second modification. In some embodiments, the modified IL-18 polypeptide comprises a third modification. In some embodiments, the modified IL-18 polypeptide comprises a second and a third modification.

In some embodiments, the modified IL-18 polypeptides comprise two modifications in the range of amino acid residues 1-127, based on the sequence of human IL-18³⁷⁻¹⁹³ (SEQ ID NO: 1). SEQ ID NO: 1 reflects the bioactive form of IL-18. Endogenously, IL-18 is initially expressed with an additional 36 amino acid segment at the N-terminus which is cleaved by caspases to mediate biologic activity. In some embodiments, the one modification is in the range of amino acid residues 6-63 based on SEQ ID NO: 1. In some embodiments, one modification is at amino acid residue 6. In some embodiments, one modification is in the range of amino acid residues 53-63. In some embodiments, one modification is at amino acid residue 53. In some embodiments, one modification is at amino acid residue 63.

In one aspect, described herein is a modified interleukin-18 (IL-18) polypeptide, comprising a modified IL-18 polypeptide comprising E06K and K53A, wherein residue position numbering of the modified IL-18 polypeptide is based on SEQ ID NO: 1 as a reference sequence.

In some embodiments, the modified IL-18 polypeptide further comprises T63A. In some embodiments, the modified IL-18 polypeptide further comprises at least one of Y01X, S55X, F02X, D54X, C38X, C68X, C76X, C127X, or K70X, wherein X is an amino acid or an amino acid derivative. In some embodiments, the modified IL-18 polypeptide further comprises at least one of Y01X, S55X, F02X, D54X, C38X, C68X, E69X, C76X, C127X, or K70X, wherein each X is independently an amino acid or an amino acid derivative. In some embodiments, the modified IL-18 polypeptide further comprises at least one of Y01G, S55A, F02A, D54A, C38S, C68S, C76S, C127S, or K70C. In some embodiments, the modified IL-18 polypeptide further comprises at least one of Y01G, S55A, F02A, D54A, C38S, C38A, C68S, C68A, C76S, C76A, C127S, C127A, or K70C.

In some embodiments, the modified IL-18 polypeptide comprises at least one modification to the amino acid sequence of SEQ ID NO: 1 selected from: Y01X, F02X, E06X, S10X, V11X, D17X, C38X, M51X, K53X, D54X, S55X, T63X, C68X, K70X, C76X, AND C127X, wherein X is a natural or non-natural amino acid. In some embodiments, the modified IL-18 polypeptide comprises at least one modification to the amino acid sequence of SEQ ID NO: 1 selected from: Y01X, F02X, E06X, S10X, V11X, D17X, C38X, M51X, K53X, D54X, S55X, T63X, C68X, E69X, K70X, C76X, AND C127X, wherein each X is independently a natural or non-natural amino acid. In some embodiments, the modified IL-18 polypeptide comprises at least one modification to the amino acid sequence of SEQ ID NO: 1 selected from: Y01X, F02X, E06X, S10X, V11X, D17X, C38X, M51X, K53X, D54X, S55X, T63X, C68X, K70X, C76X, AND C127X, wherein X is a natural or non-natural amino acid. In some embodiments, the modified IL-18 polypeptide comprises at least one modification to the amino acid sequence of SEQ ID NO: 1 selected from: Y01G, F02A, E06K, S10T, V11I, D17N, C38S, M51G, K53A, D54A, S55A, T63A, C68S, K70C, C76S, and C127S. In some embodiments, the modified IL-18 polypeptide comprises at least one modification to the amino acid sequence of SEQ ID NO: 1 selected from: Y01G, F02A, E06K, S10T, V11I, D17N, C38S, C38A, M51G, K53A, D54A, S55A, T63A, C68S, C68A, K70C, C76S, C76A, C127A, and C127S. In some embodiments, the modified IL-18 polypeptide comprises at least one modification to the amino acid sequence of SEQ ID NO: 1 selected from: Y01G, F02A, E06K, S10T, V11I, D17N, C38S, C38A, M51G, K53A, D54A, S55A, T63A, C68S, C68A, E69C, K70C, C76S, C76A, C127A, and C127S.

In some embodiments, the modified IL-18 peptide comprises one modification to the amino acid sequence of SEQ ID NO: 1, wherein the modification is E06X, K53X, S55X, or T63X, wherein X is a natural or non-natural amino acid. In some embodiments, the modified IL-18 peptide comprises one modification to the amino acid sequence of SEQ ID NO: 1, wherein the modification is E06X, K53X, S55X, or T63X, wherein each X is independently a natural or non-natural amino acid In some embodiments, the modified IL-18 peptide comprises two modifications to the amino acid sequence of SEQ ID NO: 1, wherein the modifications are E06X and K53X; E06X and S55X; K53X and S55X; E06X and T63X; or K53X and T63X, wherein X is a natural or non-natural amino acid. In some embodiments, the modified IL-18 peptide comprises three modifications to the amino acid sequence of SEQ ID NO: 1, wherein the modifications are E06X, K53X, and S55X; or E06X, K53X, and T63X, wherein X is a natural or non-natural amino acid. In some embodiments, the modified IL-18 peptide comprises four modifications to the amino acid sequence of SEQ ID NO: 1, wherein the modifications are E06X, K53X, S55X, and T63X; E06X, K53X, S55X, and Y01X; E06X, K53X, S55X, and F02X; E06X, K53X, S55X, and D54X; E06X, K53X, S55X, and M51X; or C38X, C68X, C76X, and C127X, wherein X is a natural or non-natural amino acid. In some embodiments, the modified IL-18 peptide comprises four modifications to the amino acid sequence of SEQ ID NO: 1, wherein the modifications are E06X, K53X, S55X, and T63X; E06X, K53X, S55X, and Y01X; E06X, K53X, S55X, and F02X; E06X, K53X, S55X, and D54X; E06X, K53X, S55X, and M51X; C38X, E69X, C76X, and C127X; or C38X, E70X, C76X, and C127X wherein X is a natural or non-natural amino acid. In some embodiments, the modified IL-18 polypeptide comprises at least 4 modification to the amino acid sequence of SEQ ID NO: 1, wherein the at least four modification are E06X, K53X, C68X, and E69X; E06X, K53X, C68X, and K70X; E06X, K53X, T63X, and E69X; or E06X, K53X, T63X, and K70X. In some embodiments, the modified IL-18 peptide comprises five modifications to the amino acid sequence of SEQ ID NO: 1, wherein the modifications are C38X, C68X, C76X, C127X, and K70X, wherein X is a natural or non-natural amino acid. In some embodiments, the modified IL-18 peptide comprises five modifications to the amino acid sequence of SEQ ID NO: 1, wherein the modifications are C38X, C68X, C76X, C127X, and E69X, wherein X is a natural or non-natural amino acid. In some embodiments, the modified IL-18 peptide comprises seven modifications to the amino acid sequence of SEQ ID NO: 1, wherein the modifications are E06X, K53X, C38X, C68X, C76X, C127X, and K70X; or K53X, T63X, C38X, C68X, C76X, C127X, and K70X, wherein X is a natural or non-natural amino acid. In some embodiments, the modified IL-18 peptide comprises seven modifications to the amino acid sequence of SEQ ID NO: 1, wherein the modifications are E06X, K53X, C38X, C68X, C76X, C127X, and E69X; or K53X, T63X, C38X, C68X, C76X, C127X, and E69X, wherein X is a natural or non-natural amino acid. In some embodiments, the modified IL-18 peptide comprises eight modifications to the amino acid sequence of SEQ ID NO: 1, wherein the modifications are Y01X, F02X, E06X, M51X, K53X, D54X, S55X, and T63X; or E06X, K53X, S55X, C38X, C68X, C76X, C127X, and K70X, wherein X is a natural or non-natural amino acid. In some embodiments, the modified IL-18 peptide comprises eight modifications to the amino acid sequence of SEQ ID NO: 1, wherein the modifications are E06X, K53X, S55X, C38X, C68X, C76X, C127X, and E69X. In some embodiments, wherein a plurality of amino acids residues are replaced with a natural or non-natural amino acid X, each X is independently the same or a different amino acid.

In some embodiments, the modified IL-18 peptide comprises one modification to the amino acid sequence of SEQ ID NO: 1, wherein the modification is E06K, K53A, S55A, or T63A. In some embodiments, the modified IL-18 peptide comprises two modifications to the amino acid sequence of SEQ ID NO: 1, wherein the modifications are E06K and K53A; E06K and S55A; K53A and S55A; E06K and T63A; or K53A and T63A. In some embodiments, the modified IL-18 peptide comprises three modifications to the amino acid sequence of SEQ ID NO: 1, wherein the modifications are E06K, K53A, and S55A; or E06K, K53A, and T63A. In some embodiments, the modified IL-18 peptide comprises four modifications to the amino acid sequence of SEQ ID NO: 1, wherein the modifications are E06K, K53A, S55A, and T63A; E06K, K53A, S55A, and Y01G; E06K, K53A, S55A, and F02A; E06K, K53A, S55A, and D54A; E06K, K53A, S55A, and M51G; or C38S, C68S, C76S, and C127S. In some embodiments, the modified IL-18 peptide comprises five modifications to the amino acid sequence of SEQ ID NO: 1, wherein the modifications are C38S, C68S, C76S, C127S, and K70C. In some embodiments, the modified IL-18 peptide comprises five modifications to the amino acid sequence of SEQ ID NO: 1, wherein the modifications are C38S, C68S, C76S, C127S, and E69C. In some embodiments, the modified IL-18 peptide comprises seven modifications to the amino acid sequence of SEQ ID NO: 1, wherein the modifications are E06K, K53A, C38S, C68S, C76S, C127S, and K70C; or K53A, T63A, C38S, C68S, C76S, C127S, and K70C. In some embodiments, the modified IL-18 peptide comprises seven modifications to the amino acid sequence of SEQ ID NO: 1, wherein the modifications are E06K, K53A, C38S, C68S, C76S, C127S, and E69C; or K53A, T63A, C38S, C68S, C76S, C127S, and E69C. In some embodiments, the modified IL-18 peptide comprises eight modifications to the amino acid sequence of SEQ ID NO: 1, wherein the modifications are Y01G, F02A, E06K, M51G, K53A, D54A, S55A, and T63A; or E06K, K53A, S55A, C38S, C68S, C76S, C127S, and K70C. In some embodiments, the modified IL-18 peptide comprises eight modifications to the amino acid sequence of SEQ ID NO: 1, wherein the modifications are Y01G, F02A, E06K, M51G, K53A, D54A, S55A, and T63A; or E06K, K53A, S55A, C38S, C68S, C76S, C127S, and E69C.

In one aspect, provided herein, is a modified IL-18 polypeptide, comprising a modified IL-18 polypeptide comprising E06K and K53A, wherein residue position numbering of the modified IL-18 polypeptide is based on SEQ ID NO: 1 as a reference sequence. In some embodiments, the modified IL-18 polypeptide has an amino acid sequence at least 80%, at least 85%, at least 90%, at least 95%, or at least 98% identical to the amino acid sequence of SEQ ID NO: 7. In some embodiments, the modified IL-18 polypeptide further comprises an amino acid substitution at one or more cysteine residues. In some embodiments, the modified IL-18 polypeptide comprises one or more cysteines substituted with either serine or alanine. In some embodiments, the modified IL-18 polypeptide comprise amino acid substitutions at each cysteine residue. In some embodiments, each cysteine residue is substituted with serine or alanine. In some embodiments, the modified IL-18 polypeptide comprises a polymer attached at residue 68, 69, or 70. In some embodiments, the modified IL-18 polypeptide comprises amino acid substitutions at 1, 2, 3, 4, 5, or 6 methionine residues. In some embodiments, each substitution at a methionine residue is for an O-methyl-L-homoserine residue. In some embodiments, each methionine residue is substituted with an O-methyl-L-homoserine residue.

In one aspect, provided herein, is a modified IL-18 polypeptide, comprising a modified IL-18 polypeptide comprising E06K and S55A, wherein residue position numbering of the modified IL-18 polypeptide is based on SEQ ID NO: 1 as a reference sequence. In some embodiments, the modified IL-18 polypeptide has an amino acid sequence at least 80%, at least 85%, at least 90%, at least 95%, or at least 98% identical to the amino acid sequence of SEQ ID NO: 8. In some embodiments, the modified IL-18 polypeptide further comprises an amino acid substitution at one or more cysteine residues. In some embodiments, the modified IL-18 polypeptide comprises one or more cysteines substituted with either serine or alanine. In some embodiments, the modified IL-18 polypeptide comprise amino acid substitutions at each cysteine residue. In some embodiments, each cysteine residue is substituted with serine or alanine. In some embodiments, the modified IL-18 polypeptide comprises a polymer attached at residue 68, 69, or 70. In some embodiments, the modified IL-18 polypeptide comprises amino acid substitutions at 1, 2, 3, 4, 5, or 6 methionine residues. In some embodiments, each substitution at a methionine residue is for an O-methyl-L-homoserine residue. In some embodiments, each methionine residue is substituted with an O-methyl-L-homoserine residue.

In one aspect, provided herein, is a modified IL-18 polypeptide, comprising a modified IL-18 polypeptide comprising E06K, K53A, S55A, and T63A, wherein residue position numbering of the modified IL-18 polypeptide is based on SEQ ID NO: 1 as a reference sequence. In some embodiments, the modified IL-18 polypeptide has an amino acid sequence at least 80%, at least 85%, at least 90%, at least 95%, or at least 98% identical to the amino acid sequence of SEQ ID NO: 10. In some embodiments, the modified IL-18 polypeptide further comprises an amino acid substitution at one or more cysteine residues. In some embodiments, the modified IL-18 polypeptide comprises one or more cysteines substituted with either serine or alanine. In some embodiments, the modified IL-18 polypeptide comprise amino acid substitutions at each cysteine residue. In some embodiments, each cysteine residue is substituted with serine or alanine. In some embodiments, the modified IL-18 polypeptide comprises a polymer attached at residue 68, 69, or 70. In some embodiments, the modified IL-18 polypeptide comprises amino acid substitutions at 1, 2, 3, 4, 5, or 6 methionine residues. In some embodiments, each substitution at a methionine residue is for an O-methyl-L-homoserine residue. In some embodiments, each methionine residue is substituted with an O-methyl-L-homoserine residue.

In one aspect, provided herein, is a modified IL-18 polypeptide, comprising a modified IL-18 polypeptide comprising E06K, K53A, and T63A, wherein residue position numbering of the modified IL-18 polypeptide is based on SEQ ID NO: 1 as a reference sequence. In some embodiments, the modified IL-18 polypeptide has an amino acid sequence at least 80%, at least 85%, at least 90%, at least 95%, or at least 98% identical to the amino acid sequence of SEQ ID NO: 18. In some embodiments, the modified IL-18 polypeptide further comprises an amino acid substitution at one or more cysteine residues. In some embodiments, the modified IL-18 polypeptide comprises one or more cysteines substituted with either serine or alanine. In some embodiments, the modified IL-18 polypeptide comprise amino acid substitutions at each cysteine residue. In some embodiments, each cysteine residue is substituted with serine or alanine. In some embodiments, the modified IL-18 polypeptide comprises a polymer attached at residue 68, 69, or 70. In some embodiments, the modified IL-18 polypeptide comprises amino acid substitutions at 1, 2, 3, 4, 5, or 6 methionine residues. In some embodiments, each substitution at a methionine residue is for an O-methyl-L-homoserine residue. In some embodiments, each methionine residue is substituted with an O-methyl-L-homoserine residue.

In one aspect, provided herein, is a modified IL-18 polypeptide, comprising a modified IL-18 polypeptide comprising T63A, wherein residue position numbering of the modified IL-18 polypeptide is based on SEQ ID NO: 1 as a reference sequence. In some embodiments, the modified IL-18 polypeptide has an amino acid sequence at least 80%, at least 85%, at least 90%, at least 95%, or at least 98% identical to the amino acid sequence of SEQ ID NO: 19. In some embodiments, the modified IL-18 polypeptide further comprises an amino acid substitution at one or more cysteine residues. In some embodiments, the modified IL-18 polypeptide comprises one or more cysteines substituted with either serine or alanine. In some embodiments, the modified IL-18 polypeptide comprise amino acid substitutions at each cysteine residue. In some embodiments, each cysteine residue is substituted with serine or alanine. In some embodiments, the modified IL-18 polypeptide comprises a polymer attached at residue 68, 69, or 70. In some embodiments, the modified IL-18 polypeptide comprises amino acid substitutions at 1, 2, 3, 4, 5, or 6 methionine residues. In some embodiments, each substitution at a methionine residue is for an O-methyl-L-homoserine residue. In some embodiments, each methionine residue is substituted with an O-methyl-L-homoserine residue.

In one aspect, provided herein, is a modified IL-18 polypeptide, comprising a modified IL-18 polypeptide comprising E06K and T63A, wherein residue position numbering of the modified IL-18 polypeptide is based on SEQ ID NO: 1 as a reference sequence. In some embodiments, the modified IL-18 polypeptide has an amino acid sequence at least 80%, at least 85%, at least 90%, at least 95%, or at least 98% identical to the amino acid sequence of SEQ ID NO: 20. In some embodiments, the modified IL-18 polypeptide further comprises an amino acid substitution at one or more cysteine residues. In some embodiments, the modified IL-18 polypeptide comprises one or more cysteines substituted with either serine or alanine. In some embodiments, the modified IL-18 polypeptide comprise amino acid substitutions at each cysteine residue. In some embodiments, each cysteine residue is substituted with serine or alanine. In some embodiments, the modified IL-18 polypeptide comprises a polymer attached at residue 68, 69, or 70. In some embodiments, the modified IL-18 polypeptide comprises amino acid substitutions at 1, 2, 3, 4, 5, or 6 methionine residues. In some embodiments, each substitution at a methionine residue is for an O-methyl-L-homoserine residue. In some embodiments, each methionine residue is substituted with an O-methyl-L-homoserine residue.

In one aspect, provided herein, is a modified IL-18 polypeptide, comprising a modified IL-18 polypeptide comprising E06K, K53A, C38S, C76S, and C127S, wherein residue position numbering of the modified IL-18 polypeptide is based on SEQ ID NO: 1 as a reference sequence. In some embodiments, the modified IL-18 polypeptide has an amino acid sequence at least 80%, at least 85%, at least 90%, at least 95%, or at least 98% identical to the amino acid sequence of SEQ ID NO: 70. In some embodiments, the modified IL-18 polypeptide comprises a polymer attached at residue 68, 69, or 70. In some embodiments, the modified IL-18 polypeptide comprises amino acid substitutions at 1, 2, 3, 4, 5, or 6 methionine residues. In some embodiments, each substitution at a methionine residue is for an O-methyl-L-homoserine residue. In some embodiments, each methionine residue is substituted with an O-methyl-L-homoserine residue.

In one aspect, provided herein, is a modified IL-18 polypeptide, comprising a modified IL-18 polypeptide comprising E06K, C38S, and K53A, wherein residue position numbering of the modified IL-18 polypeptide is based on SEQ ID NO: 1 as a reference sequence. In some embodiments, the modified IL-18 polypeptide has an amino acid sequence at least 80%, at least 85%, at least 90%, at least 95%, or at least 98% identical to the amino acid sequence of SEQ ID NO: 71. In some embodiments, the modified IL-18 polypeptide comprises a polymer attached at residue 68, 69, or 70. In some embodiments, the modified IL-18 polypeptide comprise a polymer attached at residue 68. In some embodiments, the modified IL-18 polypeptide comprises amino acid substitutions at 1, 2, 3, 4, 5, or 6 methionine residues. In some embodiments, each substitution at a methionine residue is for an O-methyl-L-homoserine residue. In some embodiments, each methionine residue is substituted with an O-methyl-L-homoserine residue.

In some embodiments, the modified IL-18 polypeptide comprises a polypeptide sequence having at least about 80%, at least about 85%, at least about 90%, at least about 95%, or about 100% sequence identity to SEQ ID NO: 2-58. In some embodiments, the modified IL-18 polypeptide comprises a polypeptide sequence having at least about 80%, at least about 85%, at least about 90%, at least about 95%, or about 100% sequence identity to SEQ ID NO: 2-83. In some embodiments, the polypeptide sequence is at least about 80% identical to SEQ ID NO: 2 or SEQ ID NO: 18. In some embodiments, the polypeptide sequence is at least about 80% identical to SEQ ID NO: 2. In some embodiments, the polypeptide sequence is at least about 90% identical to SEQ ID NO: 2. In some embodiments, the polypeptide sequence is at least about 95% identical to SEQ ID NO: 2. In some embodiments, the polypeptide sequence is at least about 80% identical to SEQ ID NO: 18. In some embodiments, the polypeptide sequence is at least about 90% identical to SEQ ID NO: 18. In some embodiments, the polypeptide sequence is at least about 95% identical to SEQ ID NO: 18. In some embodiments, the modified IL-18 polypeptide is recombinant.

In some embodiments, the modified IL-18 polypeptide comprises one or more amino acid substitutions selected from: (a) a homoserine residue located at any one of residues 26-36; (b) a homoserine residue located at any one of residues 60-80; (c) a homoserine residue located at any one of residues 110-120; (d) a norleucine residue located at any one of residues 28-38; (d) a norleucine residue located at any one of residues 46-56; (e) a norleucine residue located at any one of residues 54-64; (f) a norleucine residue located at any one of residues 80-90; (g) a norleucine residue located at any one of residues 108-118; and (h) a norleucine residue located at any one of residues 145-155, wherein residue position numbering of the modified IL-18 polypeptide is based on SEQ ID NO: 1 as a reference sequence.

In some embodiments, the modified IL-18 polypeptide comprises one or more amino acid substitutions selected from: (a) a homoserine residue located at any one of residues 26-36; (b) a homoserine residue located at any one of residues 60-80; (c) a homoserine residue located at any one of residues 110-120; (d) a norleucine or O-methyl-homoserine residue located at any one of residues 28-38; (e) a norleucine or O-methyl-homoserine residue located at any one of residues 46-56; (f) a norleucine or O-methyl-homoserine residue located at any one of residues 54-64; (g) a norleucine or O-methyl-homoserine residue located at any one of residues 80-90; (h) a norleucine or O-methyl-homoserine residue located at any one of residues 108-118; and (i) a norleucine or O-methyl-homoserine residue located at any one of residues 145-155, wherein residue position numbering of the modified IL-18 polypeptide is based on SEQ ID NO: 1 as a reference sequence.

In some embodiments, the modified IL-18 polypeptide comprises one or more amino acid substitutions selected from: (a) a homoserine residue located at any one of residues 26-36; (b) a homoserine residue located at any one of residues 60-80; (c) a homoserine residue located at any one of residues 110-120; (d) a O-methyl-homoserine residue located at any one of residues 28-38; (e) a O-methyl-homoserine residue located at any one of residues 46-56; (f) a O-methyl-homoserine residue located at any one of residues 54-64; (g) a O-methyl-homoserine residue located at any one of residues 80-90; (h) a O-methyl-homoserine residue located at any one of residues 108-118; and (i) a O-methyl-homoserine residue located at any one of residues 145-155, wherein residue position numbering of the modified IL-18 polypeptide is based on SEQ ID NO: 1 as a reference sequence.

In some embodiments, the modified IL-18 polypeptide comprises one or more amino acid substitutions selected from homoserine (Hse) 31, norleucine (Nle) 33, Nle51, Nle59, Hse75, Nle86, Nle113, Hse116, and Nle150. In some embodiments, the modified IL-18 peptide comprises an amino acid substitution with O-methyl-L-homoserine. In some embodiments, the modified IL-18 peptide comprises an amino acid substitution with O-methyl-L-homoserine at positions Met 33, Met 51, Met 60, Met 86, Met 113, or Met 150. In some embodiments, the modified IL-18 polypeptide comprises one or more amino acid substitutions selected from homoserine (Hse) 31, norleucine (Nle) 33, O-methyl-homoserine (Omh) 33, Nle51, Omh51, Nle60, Omh60, Hse75, Nle86, Omh86, Hse116, Nle113, Omh113, Nle150, and Omh150.

In some embodiments, the modified IL-18 polypeptides described herein contain a linker moiety. In some embodiments, the linker moiety includes, but is not limited to, a polymer, linker, spacer, or combinations thereof. When added to certain amino acid residues, the linker moiety can modulate the activity or other properties of the modified IL-18 polypeptide compared to wild-type IL-18.

In some embodiments, a modified IL-18 polypeptide is linked with an additional polypeptide. In some embodiments, the modified IL-18 polypeptide and the additional polypeptide form a fusion polypeptide. In some embodiments, the modified IL-18 polypeptide and the additional polypeptide are conjugated together. In some embodiments, the additional polypeptide comprises an antibody or binding fragment thereof. In some embodiments, the antibody comprises a humanized antibody, a murine antibody, a chimeric antibody, a bispecific antibody, any fragment thereof, or any combination thereof. In some embodiments, the antibody is a monoclonal antibody or any fragment thereof. In some embodiments, a modified IL-18 polypeptide is conjugated to a cytokine.

### III. Modified IL-18 Polypeptides Comprising Polymer Moieties

The modified IL-18 polypeptides described herein can contain one or more polymers. In some embodiments, a modified IL-18 polypeptide is conjugated to one polymer moiety. In some embodiments, a modified IL-18 polypeptide is conjugated to two polymer moieties. The addition of polymers to certain amino acid residues can disrupt the binding interaction of the modified IL-18 polypeptide with IL-18BP. FIG. 2C illustrates a modified synthetic IL-18 polypeptide comprising a polymer moiety.

In some embodiments, a modified IL-18 polypeptide conjugated to one or more polymer moieties can retain binding to IL-18Rα and have a reduced binding interaction with IL-18BP. In some embodiments, a modified IL-18 polypeptide conjugated to one or more polymer moieties can have increased binding to IL-18Rα and have a reduced binding interaction with IL-18BP. In some embodiments, a modified IL-18 polypeptide conjugated to one or more polymer moieties can retain binding to the IL-18Rα/β heterodimer and have a reduced binding interaction with IL-18BP. In some embodiments, a modified IL-18 polypeptide conjugated to one or more polymer moieties can have increased binding to the IL-18Rα/β heterodimer and have a reduced binding interaction with IL-18BP.

In some embodiments, the modified IL-18 polypeptide comprises a polymer covalently attached at residue 65, residue 66, residue 67, residue 68, residue 69, residue 70, residue 71, residue 72, residue 73, residue 74 or residue 75. In some embodiments, the modified IL-18 polypeptide comprises a polymer covalently attached at residue C68. In some embodiments, the modified IL-18 polypeptide comprises a polymer covalently attached at residue 68. In some embodiments, the modified IL-18 polypeptide comprises a polymer covalently attached at residue 70. In some embodiments, the modified IL-18 polypeptide comprises a polymer covalently attached at residue K70. In some embodiments, the modified IL-18 polypeptide comprises a polymer covalently attached at residue 69. In some embodiments, the modified IL-18 polypeptide comprises a polymer covalently attached at residue E69.

In some embodiments, the polymer is covalently attached through a modified amino acid α. In some embodiments, the modified amino acid α is an amino-acid-PEG-azide group. In some embodiments, the modified amino acid α is a glutamate, aspartate, lysine, cysteine, or tyrosine modified to incorporate an azide group linked to the amino acid through a PEG spacer. In some embodiments, the modified amino acid α has a structure selected from:

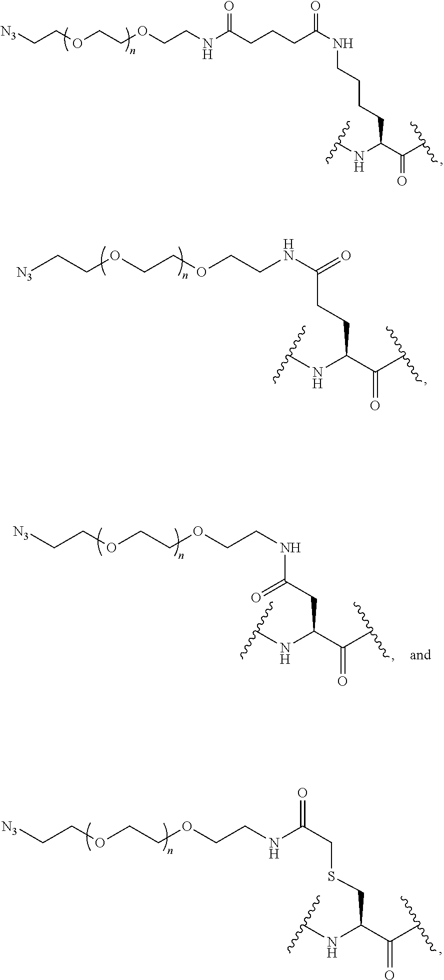

wherein each n is independently an integer from 1-30. In some embodiments, n is an integer from 1-20, 1-10, 2-30, 2-20, 2-10, 5-30, 5-20, or 5-10. In some embodiments, n is 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30. In some embodiments, n is 10. In some embodiments, n is 8. In some embodiments, n is 6. In some embodiments, n is 12.

In some embodiments, the modified amino acid a is located at a position on the modified IL-18 polypeptide selected from residue 65, residue 66, residue 67, residue 68, residue 69, residue 70, residue 71, residue 72, residue 73, residue 74 and residue 75. In some embodiments, the modified amino acid a is located at a position on the modified IL-18 polypeptide selected from residue 68, residue 69, and residue 70. In some embodiments, the modified amino acid a is located at residue 68 of the modified IL-18 polypeptide. In some embodiments, the modified amino acid a is located at residue 69 of the modified IL-18 polypeptide. In some embodiments, the modified amino acid a is located at residue 70 of the modified IL-18 polypeptide.

In some embodiments, the modified IL-18 polypeptide comprises a polymer covalently attached to a modified lysine residue. In some embodiments, the modified lysine residue comprises a conjugation handle. In some embodiments, the modified lysine residue comprises an azide. In some embodiments, the modified lysine residue has a structure of Structure B, wherein Structure B is

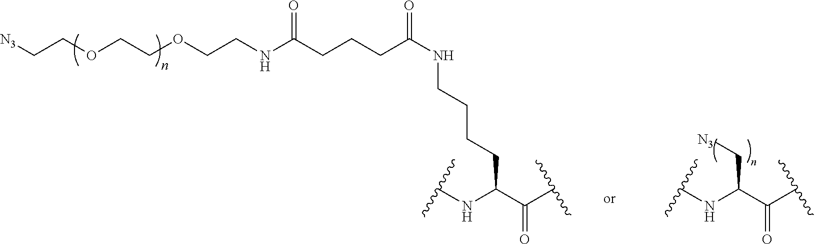

wherein each n is independently an integer from 1-30. In some embodiments, n is an integer from 1-20, 1-10, 2-30, 2-20, 2-10, 5-30, 5-20, or 5-10. In some embodiments, n is 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30. In some embodiments, n is 1, 2, 3, 4, 5, 6, 7, or 8. In some embodiments, n is 3. In some embodiments, n is 4. In some embodiments, n is 5. In some embodiments, n is 6. In some embodiments, n is 8. In some embodiments, n is 10. In some embodiments, n is 12.

In some embodiments, the modified lysine of Structure B is located at a position on the modified IL-18 polypeptide selected from residue 65, residue 66, residue 67, residue 68, residue 69, residue 70, residue 71, residue 72, residue 73, residue 74 and residue 75. In some embodiments, the modified lysine of Structure B is located at a position on the modified IL-18 polypeptide selected from residue 68, residue 69, and residue 70. In some embodiments, the modified lysine of Structure B is located at residue 68 of the modified IL-18 polypeptide. In some embodiments, the modified lysine of Structure B is located at residue 69 of the modified IL-18 polypeptide. In some embodiments, the modified lysine of Structure B is located at residue 70 of the modified IL-18 polypeptide.

In one aspect, described herein is a population of modified interleukin-18 (IL-18) polypeptides, comprising: a) a plurality of modified IL-18 polypeptides; and b) at least one polymer moiety, wherein the at least one polymer moiety is covalently linked to the modified IL-18 polypeptides and attached at residue 65, residue 66, residue 67, residue 68, residue 69, residue 70, residue 71, residue 72, residue 73, residue 74 or residue 75, wherein the amino acid residue position is based on SEQ ID NO:1 as a reference sequence; wherein at least 90% of the modified IL-18 polypeptides have a molecular weight that is within ±500 Da of the peak molecular weight of the plurality of the modified IL-18 polypeptides as determined by high resolution electrospray ionization mass spectrometry (ESI-HRMS). In some embodiments, each of the modified IL-18 polypeptides of the population comprises at least one of the polymer moieties covalently thereto.

In one aspect, described herein is a population of modified interleukin-18 (IL-18) polypeptides, comprising: a) a plurality of modified IL-18 polypeptides; and b) a plurality of polymer moieties, wherein the plurality of polymer moieties are covalently linked to the modified IL-18 polypeptides and attached at residue 65, residue 66, residue 67, residue 68, residue 69, residue 70, residue 71, residue 72, residue 73, residue 74 or residue 75 of the modified IL-18 polypeptide, wherein the amino acid residue position is based on SEQ ID NO: 1 as a reference sequence; wherein at least 90% of the plurality of polymer moieties have a molecular weight that is within ±500 Da of the peak molecular weight of the plurality of the modified IL-18 polypeptides as determined by high resolution electrospray ionization mass spectrometry (ESI-HRMS).

In some embodiments, at least 75% of the plurality of polymers have a molecular weight that is within ±10% of the peak molecular weight of the plurality of polymers as determined by high resolution electrospray ionization mass spectrometry (ESI-HRMS). In some embodiments, the at least one polymer moiety or the plurality of polymer moieties is covalently linked to the modified IL-18 polypeptides at amino acid residue 68, wherein the amino acid residue numbering of the modified IL-18 polypeptides is based on SEQ ID NO: 1 as a reference sequence. In some embodiments, the at least one polymer moiety or the plurality of polymer moieties is covalently linked to the modified IL-18 polypeptides at amino acid residue 69, wherein the amino acid residue numbering of the modified IL-18 polypeptides is based on SEQ ID NO: 1 as a reference sequence. In some embodiments, the at least one polymer moiety or the plurality of polymer moieties is covalently linked to the modified IL-18 polypeptides at amino acid residue 70, wherein the amino acid residue numbering of the modified IL-18 polypeptides is based on SEQ ID NO: 1 as a reference sequence. In some embodiments, the at least one polymer moiety or the plurality of polymer moieties is covalently linked to the modified IL-18 polypeptides at C68, wherein the amino acid residue numbering of the modified IL-18 polypeptides is based on SEQ ID NO: 1 as a reference sequence. In some embodiments, the at least one polymer moiety or the plurality of polymer moieties is covalently linked to the modified IL-18 polypeptides at K70, wherein the amino acid residue numbering of the modified IL-18 polypeptides is based on SEQ ID NO: 1 as a reference sequence.

In some embodiments, each modified IL-18 polypeptide of the plurality of modified IL-18 polypeptides comprises one or more mutations. In some embodiments, the one or more mutations are located at residue positions selected from E06, K53, Y01, S55, F02, D54, C38, T63A, C68, C76, C127, and K70, wherein residue position numbering of the modified IL-18 polypeptides are based on SEQ ID NO: 1 as a reference sequence. In some embodiments, each modified IL-18 polypeptide of the plurality of modified IL-18 polypeptides comprises one or more mutations. In some embodiments, the one or more mutations are located at residue positions selected from E06, K53, Y01, S55, F02, D54, C38, T63A, C68, C76, C127, and E69, wherein residue position numbering of the modified IL-18 polypeptides are based on SEQ ID NO: 1 as a reference sequence. In some embodiments, the one or more mutations are selected from E06K, K53A, Y01G, S55A, F02A, D54A, C38S, T63A, C68S, C76S, C127S, and K70C. In some embodiments, the one or more mutations are E06K and K53A. In some embodiments, the one or more mutations are selected from E06K, K53A, Y01G, S55A, F02A, D54A, C38S, T63A, C68S, C76S, C127S, and E69C. In some embodiments, the one or more mutations are E06K, K53A, and T63A.

In some embodiments, the polymer has a weight average molecular weight of at most about 50,000 Daltons, at most about 25,000 Daltons, at most about 10,000 Daltons, or at most about 6,000 Daltons. In some embodiments, the polymer has a weight average molecular weight of at least about 120 Daltons, at least about 250 Daltons, at least about 300 Daltons, at least about 400 Daltons, or at least about 500 Daltons.

In some embodiments, a modified IL-18 polypeptide described herein comprises a first polymer covalently attached at C68 or K70, wherein residue position numbering of the modified IL-18 polypeptide is based on SEQ ID NO: 1 as a reference sequence. In some embodiments, a modified IL-18 polypeptide described herein comprises a first polymer covalently attached at C68, E69, or K70, wherein residue position numbering of the modified IL-18 polypeptide is based on SEQ ID NO: 1 as a reference sequence. In some embodiments, a modified IL-18 polypeptide described herein comprises a first polymer covalently attached at C68, wherein residue position numbering of the modified IL-18 polypeptide is based on SEQ ID NO: 1 as a reference sequence. In some embodiments, a modified IL-18 polypeptide described herein comprises a first polymer covalently attached at E69, wherein residue position numbering of the modified IL-18 polypeptide is based on SEQ ID NO: 1 as a reference sequence. In some embodiments, a modified IL-18 polypeptide described herein comprises a first polymer covalently attached at K70, wherein residue position numbering of the modified IL-18 polypeptide is based on SEQ ID NO: 1 as a reference sequence.

In some embodiments, the polymer conjugated to the modified IL-18 polypeptide is a water-soluble polymer, such as polyethylene glycol (PEG). Polymers may be added to either one or both of residues C68 and K70 of an IL-18 polypeptide, or mutants thereof. Polymers may also be added to either one, two, or all three of residues C68, E69, and K70 of an IL-18 polypeptide, or mutants thereof. Additionally, polymers may be added to modify IL-18 polypeptides to increase the half-life of the polypeptides.

In some embodiments, a modified IL-18 polypeptide conjugated to one or more polymer moieties can retain binding to IL-18Rα, have a reduced binding interaction with IL-18BP, and exhibit an increased half life (tin). In some embodiments, a modified IL-18 polypeptide conjugated to one or more polymer moieties can have increased binding to IL-18Rα, have a reduced binding interaction with IL-18BP, and exhibit an increased half life (tin). In some embodiments, a modified IL-18 polypeptide conjugated to one or more polymer moieties can retain binding to the IL-18Rαβ heterodimer, have a reduced binding interaction with IL-18BP, and exhibit an increased half life (tin). In some embodiments, a modified IL-18 polypeptide conjugated to one or more polymer moieties can have increased binding to the IL-18Rαβ heterodimer, have a reduced binding interaction with IL-18BP, and exhibit an increased half-life (tin). In some embodiments, the half-life is a half-life in vivo in blood of a subject.

The half-life extending polymers may be of any size, including up to about 6 kDa, up to about 25 kDa, or up to about 50 kDa. In some embodiments, the half-life extending polymers are PEG polymers. In some embodiments, the half-life extending polymer has an average molecular weight of from about 200 to about 20,000, for example, PEG 200, PEG 400, PEG 600, PEG 1000, PEG 1450, PEG 1500, PEG 4000, PEG 4600, and PEG 8000.

### IIIa. Polymers

In one aspect, described herein is a modified polypeptide that comprises a modified IL-18 polypeptide, wherein the modified IL-18 polypeptide comprises a covalently attached polymer. In some embodiments, a herein described modified IL-18 polypeptide comprises one or more polymers covalently attached thereon. In some embodiments, the described modified IL-18 polypeptide comprises 1, 2, 3, 4, 5, 6, 7, 8, 9, 10, or more polymers covalently attached to the modified IL-18 polypeptide.

In some embodiments, the polymer comprises a conjugation handle which can be used to further attach an additional moiety to the modified IL-18 polypeptide. Any suitable reactive group capable of reacting with a complementary reactive group attached to another moiety can be used as the conjugation handle. In some embodiments, the conjugation handle comprises a reagent for a Cu(I)-catalyzed or “copper-free” alkyne-azide triazole-forming reaction (e.g., strain promoted cycloadditions), the Staudinger ligation, inverse-electron-demand Diels-Alder (IEDDA) reaction, “photo-click” chemistry, tetrazine cycloadditions with trans-cyclooctenes, or a metal-mediated process such as olefin metathesis and Suzuki-Miyaura or Sonogashira cross-coupling.

In some embodiments, the conjugation handle comprises a reagent for a “copper-free” alkyne azide triazole-forming reaction. Non-limiting examples of alkynes for said alkyne azide triazole forming reaction include cyclooctyne reagents (e.g., (1R,8S,9s)-Bicyclo[6.1.0]non-4-yn-9-ylmethanol containing reagents, dibenzocyclooctyne-amine reagents, difluorocyclooctynes, or derivatives thereof).

In some embodiments, the conjugation handle comprises a reactive group selected from azide, alkyne, tetrazine, halide, sulfhydryl, disulfide, maleimide, activated ester, alkene, aldehyde, ketone, imine, hydrazine, acyltrifluoroborate, hydroxylamine, phosphine, trans-cyclooctene, and hydrazide. In some embodiments, the conjugation handle and the complementary conjugation handle comprise “CLICK” chemistry reagents. Exemplary groups of click chemistry residue are shown in Hein et al., “Click Chemistry, A Powerful Tool for Pharmaceutical Sciences,” Pharmaceutical Research, volume 25, pages 2216-2230 (2008); Thirumurugan et al., “Click Chemistry for Drug Development and Diverse Chemical-Biology Applications,” Chem. Rev. 2013, 113, 7, 4905-4979; US20160107999A1; U.S. Ser. No. 10/266,502B2; and US20190204330A1, each of which is incorporated by reference in its entirety.

In some embodiments, the polymer comprises a conjugation handle or a reaction product of a conjugation handle with a complementary conjugation handle. In some embodiments, the reaction product of the conjugation handle with the complementary conjugation handle results from a KAT ligation (reaction of potassium acyltrifluoroborate with hydroxylamine), a Staudinger ligation (reaction of an azide with a phosphine), a tetrazine cycloaddition (reaction of a tetrazine with a trans-cyclooctene), or a Huisgen cycloaddition (reaction of an alkyne with an azide). In some embodiments, the polymer will comprise a reaction product of a conjugation handle with a complementary conjugation handle which was used to attach the polymer to the modified IL-18 polypeptide.

In some embodiments, the polymer comprises an azide moiety. In some embodiments, the polymer comprises an azide moiety, an alkyne moiety, or reaction product of an azide-alkyne cycloaddition reaction. In some embodiments, the reaction product of the azide-alkyne cycloaddition reaction is a 1,2,3-triazole.

In some embodiments, the polymer is attached to the modified IL-18 polypeptide through use of a bifunctional linker. In some embodiments, the bifunctional linker reacts with a reactive group of an amino acid residue on the modified IL-18 polypeptide (e.g., a cysteine sulfhydryl) to form a covalent bond. In some embodiments, in a second step, the second reactive group of the bifunctional a linker (e.g., a conjugation handle such as an azide or alkyne) is then used to attach a second moiety, such as the polymer.

In some embodiments, the polymer is a water-soluble polymer. In some embodiments, the water-soluble polymer comprises poly(alkylene oxide), polysaccharide, poly(vinyl pyrrolidone), poly(vinyl alcohol), polyoxazoline, poly(acryloylmorpholine), or a combination thereof. In some embodiments, the water-soluble polymer comprises poly(alkylene oxide). In some embodiments, the poly(alkylene oxide) is polyethylene glycol (PEG).

In some embodiments, the polymer is a first polymer. In some embodiments, the first polymer comprises a water-soluble polymer. In some embodiments, the water-soluble polymer comprises poly(alkylene oxide), polysaccharide, poly(vinyl pyrrolidone), poly(vinyl alcohol), polyoxazoline, poly(acryloylmorpholine), or a combination thereof. In some embodiments, the water-soluble polymer is poly(alkylene oxide). In some embodiments, the water-soluble polymer is polysaccharide. In some embodiments, the water-soluble polymer is poly(ethylene oxide).

In some embodiments, the polyethylene glycol has a weight average molecular weight of about 10 kDa to about 50 kDa. In some embodiments, the polyethylene glycol has a weight average molecular weight of about 10 kDa, about 20 kDa, or about 30 kDa. In some embodiments, the polyethylene glycol has a weight average molecular weight of about 30 kDa. In some embodiments, a half-life of the modified IL-18 polypeptide is at least 10% longer than a half-life of a corresponding wild-type IL-18 polypeptide. In some embodiments, the half-life of the modified IL-18 polypeptide is at least 30% longer than the half-life of the corresponding wild-type IL-18 polypeptide.

In some embodiments, the attached polymer has a weight average molecular weight of about 6,000 Daltons to about 50,000 Daltons. In some embodiments, the polymer has a weight average molecular weight of about 6,000 Daltons to about 10,000 Daltons, about 6,000 Daltons to about 25,000 Daltons, about 6,000 Daltons to about 50,000 Daltons, about 10,000 Daltons to about 25,000 Daltons, about 10,000 Daltons to about 50,000 Daltons, or about 25,000 Daltons to about 50,000 Daltons. In some embodiments, the polymer has a weight average molecular weight of about 6,000 Daltons, about 10,000 Daltons, about 25,000 Daltons, or about 50,000 Daltons. In some embodiments, the polymer has a weight average molecular weight of at least about 6,000 Daltons, about 10,000 Daltons, or about 25,000 Daltons. In some embodiments, the polymer has a weight average molecular weight of at most about 10,000 Daltons, about 25,000 Daltons, or about 50,000 Daltons.

In some embodiments, the attached polymer such as the first polymer has a weight average molecular weight of about 120 Daltons to about 1,000 Daltons. In some embodiments, the polymer has a weight average molecular weight of about 120 Daltons to about 250 Daltons, about 120 Daltons to about 300 Daltons, about 120 Daltons to about 400 Daltons, about 120 Daltons to about 500 Daltons, about 120 Daltons to about 1,000 Daltons, about 250 Daltons to about 300 Daltons, about 250 Daltons to about 400 Daltons, about 250 Daltons to about 500 Daltons, about 250 Daltons to about 1,000 Daltons, about 300 Daltons to about 400 Daltons, about 300 Daltons to about 500 Daltons, about 300 Daltons to about 1,000 Daltons, about 400 Daltons to about 500 Daltons, about 400 Daltons to about 1,000 Daltons, or about 500 Daltons to about 1,000 Daltons. In some embodiments, the polymer has a weight average molecular weight of about 120 Daltons, about 250 Daltons, about 300 Daltons, about 400 Daltons, about 500 Daltons, or about 1,000 Daltons. In some embodiments, the polymer has a weight average molecular weight of at least about 120 Daltons, about 250 Daltons, about 300 Daltons, about 400 Daltons, or about 500 Daltons. In some embodiments, the polymer has a weight average molecular weight of at most about 250 Daltons, about 300 Daltons, about 400 Daltons, about 500 Daltons, or about 1,000 Daltons.

In some embodiments, the attached polymer such as the first polymer comprises a water-soluble polymer. In some embodiments, the water-soluble polymer comprises poly(alkylene oxide), polysaccharide, poly(vinyl pyrrolidone), poly(vinyl alcohol), polyoxazoline, poly(acryloylmorpholine), or a combination thereof. In some embodiments, the water-soluble polymer is poly(alkylene oxide) such as polyethylene glycol (i.e., polyethylene oxide). In some embodiments, the water-soluble polymer is polyethylene glycol. In some embodiments, the water-soluble polymer comprises modified poly(alkylene oxide). In some embodiments, the modified poly(alkylene oxide) comprises one or more linker groups. In some embodiments, the one or more linker groups comprise bifunctional linkers such as an amide group, an ester group, an ether group, a thioether group, a carbonyl group and alike. In some embodiments, the one or more linker groups comprise an amide linker group. In some embodiments, the modified poly(alkylene oxide) comprises one or more spacer groups. In some embodiments, the spacer groups comprise a substituted or unsubstituted C₁-C₆ alkylene group. In some embodiments, the spacer groups comprise —CH₂—, —CH₂CH₂—, or —CH₂CH₂CH₂—. In some embodiments, the linker group is the product of a biorthogonal reaction (e.g., biocompatible and selective reactions). In some embodiments, the bioorthogonal reaction is a Cu(I)-catalyzed or “copper-free” alkyne-azide triazole-forming reaction, the Staudinger ligation, inverse-electron-demand Diels-Alder (IEDDA) reaction, alkyne-nitrone cycloaddition chemistry, or a metal-mediated process such as olefin metathesis and Suzuki-Miyaura or Sonogashira cross-coupling. In some embodiments, the first polymer is attached to the IL-18 polypeptide via click chemistry.

In some embodiments, a modified IL-18 polypeptide provided herein comprises one or more polymers selected from Table 1.

TABLE 1
Polymer structures for modified IL-18 polypeptides

| Poly- | | |
|---|---|---|
| mer | | |
| Iden- | | |
| tifier | Polymer Structure | MW |
| For- mula A | 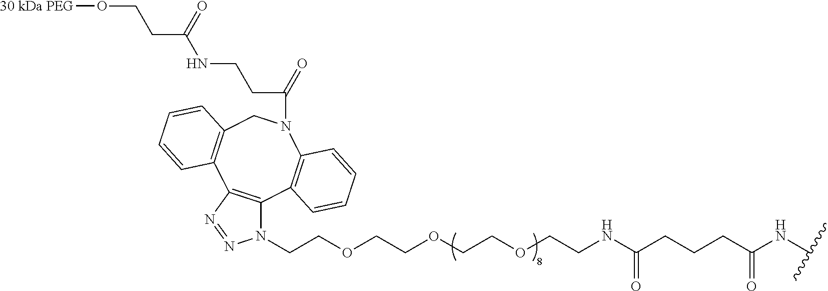 | ~30 kDa |
| For- mula B | 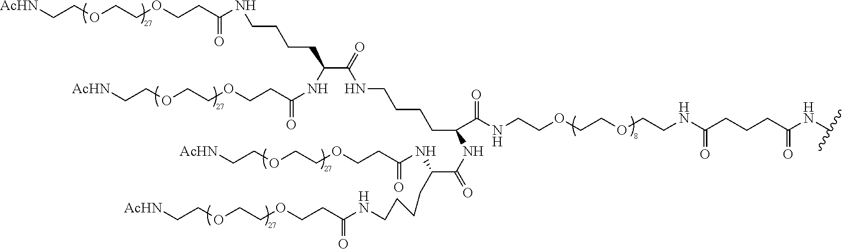 | ~6 kDa |
| For- mula C | 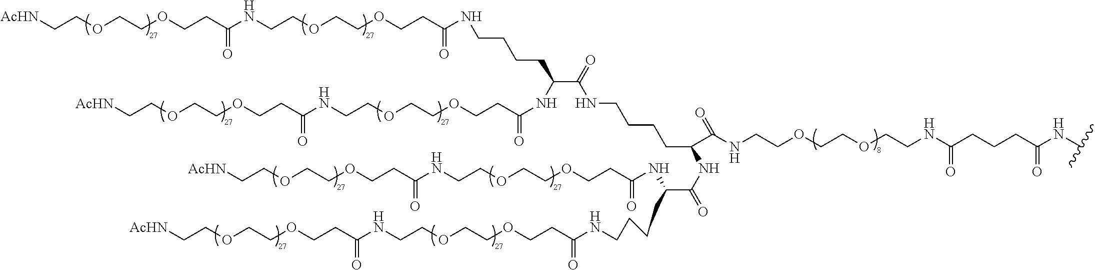 | ~11 kDa |
| For- mula D | 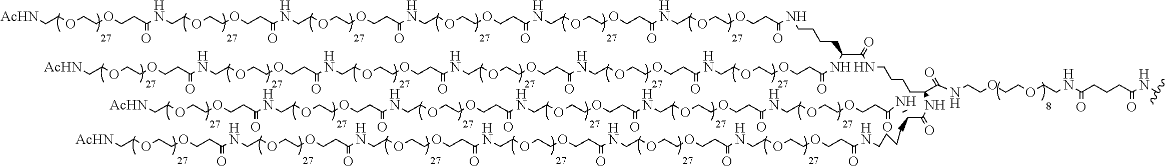 | ~30 kDa |
| For- mula E | 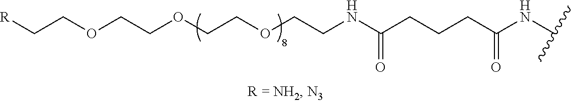 | ~500 kDa |

In some embodiments, a modified IL-18 polypeptide provided herein comprises a reaction group that facilitates the conjugation of the modified IL-18 polypeptide with a derivatized molecule or moiety such as an antibody and a polymer. In some embodiments, the reaction group comprises one or more of: carboxylic acid derived active esters, mixed anhydrides, acyl halides, acyl azides, alkyl halides, N-maleimides, imino esters, isocyanates, and isothiocyanates. In some embodiments, the reaction group comprises an azide.

In some embodiments, a modified IL-18 polypeptide provided herein comprises a chemical reagent covalently attached to a residue. In some embodiments, the chemical reagent comprises a bioorthogonal reagent. In some embodiments, the chemical reagent comprises an azide. In some embodiments, the chemical reagent comprises an alkyne. In some embodiments, the chemical reagent is attached at a residue C68 or K70, wherein the residue position numbering is based on SEQ ID NO: 1 as a reference sequence. In some embodiments, the chemical reagent is attached at a residue C68, E69, or K70, wherein the residue position numbering is based on SEQ ID NO: 1 as a reference sequence. In some embodiments, the chemical reagent is attached at a residue from C68, wherein the residue position numbering is based on SEQ ID NO: 1 as a reference sequence. In some embodiments, the chemical reagent is attached at a residue from E69, wherein the residue position numbering is based on SEQ ID NO: 1 as a reference sequence. In some embodiments, the chemical reagent is attached at residue K70, wherein the residue position numbering is based on SEQ ID NO: 1 as a reference sequence.

In some embodiments, the water-soluble polymer comprises from 1 to 10 polyethylene glycol chains. In some embodiments, the first water-soluble polymer comprises 1 polyethylene glycol chains to 10 polyethylene glycol chains. In some embodiments, the first water-soluble polymer comprises 1 polyethylene glycol chains to 2 polyethylene glycol chains, 1 polyethylene glycol chains to 4 polyethylene glycol chains, 1 polyethylene glycol chains to 6 polyethylene glycol chains, 1 polyethylene glycol chains to 10 polyethylene glycol chains, 2 polyethylene glycol chains to 4 polyethylene glycol chains, 2 polyethylene glycol chains to 6 polyethylene glycol chains, 2 polyethylene glycol chains to 10 polyethylene glycol chains, 4 polyethylene glycol chains to 6 polyethylene glycol chains, 4 polyethylene glycol chains to 10 polyethylene glycol chains, or 6 polyethylene glycol chains to 10 polyethylene glycol chains. In some embodiments, the first water-soluble polymer comprises 1 polyethylene glycol chains, 2 polyethylene glycol chains, 4 polyethylene glycol chains, 6 polyethylene glycol chains, or 10 polyethylene glycol chains. In some embodiments, the first water-soluble polymer comprises at least 1 polyethylene glycol chains, 2 polyethylene glycol chains, 4 polyethylene glycol chains, or 6 polyethylene glycol chains. In some embodiments, the first water-soluble polymer comprises at most 2 polyethylene glycol chains, 4 polyethylene glycol chains, 6 polyethylene glycol chains, or 10 polyethylene glycol chains. In some embodiments, the first water-soluble polymer comprises 4 polyethylene glycol chains. In some embodiments, the first water-soluble polymer comprises a structure of Formula (II):

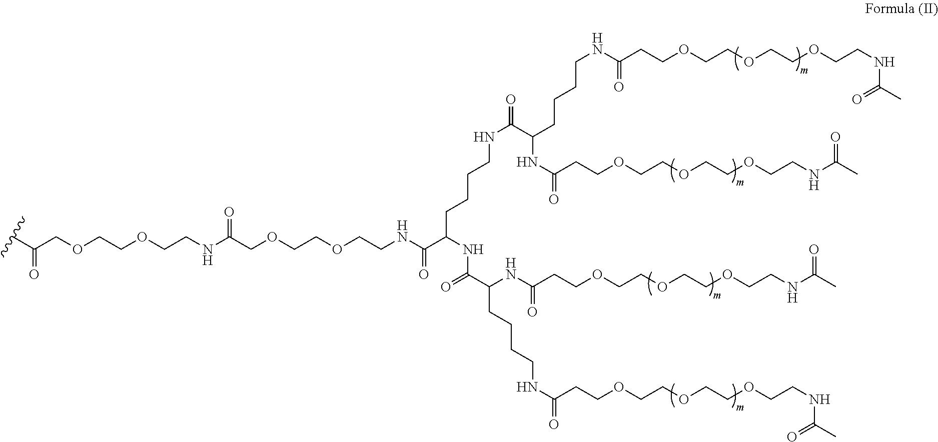

wherein each m is independently an integer from 4-30. In some embodiments, at least one polyethylene glycol chain of the first water-soluble polymer comprises the structure of Formula

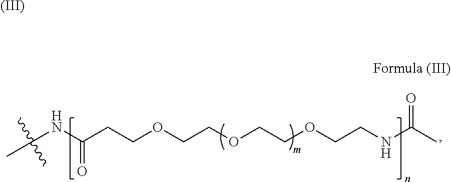

wherein each m is independently an integer from 4-30 and each n is independently an integer from 1-10. In some embodiments, each polyethylene glycol chain of the first water-soluble polymer comprises the structure of Formula (III). In some embodiments of Formula (III), m is 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40. In some embodiments of Formula (III), n is 1, 2, 3, 4, 5, 6, 7, 8, 9, or 10.

In some embodiments, a modified IL-18 polypeptide described herein further comprises a second polymer covalently attached to the modified IL-18 polypeptide. In some embodiments, the second polymer is covalently attached at an amino acid residue region from residue 68 to residue 70. In some embodiments, the second polymer is covalently attached at residue C68. In some embodiments, the second polymer is covalently attached to the N-terminus of the modified IL-18 polypeptide. In some embodiments, the second polymer is covalently attached at residue K70. In some embodiments, the second polymer is covalently attached to the N-terminus of the modified IL-18 polypeptide.

In some embodiments, the second polymer has a weight average molecular weight of about 6,000 Daltons to about 50,000 Daltons. In some embodiments, the second polymer has a weight average molecular weight of about 6,000 Daltons to about 10,000 Daltons, about 6,000 Daltons to about 25,000 Daltons, about 6,000 Daltons to about 50,000 Daltons, about 10,000 Daltons to about 25,000 Daltons, about 10,000 Daltons to about 50,000 Daltons, or about 25,000 Daltons to about 50,000 Daltons. In some embodiments, the second polymer has a weight average molecular weight of about 6,000 Daltons, about 10,000 Daltons, about 25,000 Daltons, or about 50,000 Daltons. In some embodiments, the second polymer has a weight average molecular weight of at least about 6,000 Daltons, about 10,000 Daltons, or about 25,000 Daltons. In some embodiments, the second polymer has a weight average molecular weight of at most about 10,000 Daltons, about 25,000 Daltons, or about 50,000 Daltons.

In some embodiments, the second polymer has a weight average molecular weight of about 120 Daltons to about 1,000 Daltons. In some embodiments, the second polymer has a weight average molecular weight of about 120 Daltons to about 250 Daltons, about 120 Daltons to about 300 Daltons, about 120 Daltons to about 400 Daltons, about 120 Daltons to about 500 Daltons, about 120 Daltons to about 1,000 Daltons, about 250 Daltons to about 300 Daltons, about 250 Daltons to about 400 Daltons, about 250 Daltons to about 500 Daltons, about 250 Daltons to about 1,000 Daltons, about 300 Daltons to about 400 Daltons, about 300 Daltons to about 500 Daltons, about 300 Daltons to about 1,000 Daltons, about 400 Daltons to about 500 Daltons, about 400 Daltons to about 1,000 Daltons, or about 500 Daltons to about 1,000 Daltons. In some embodiments, the second polymer has a weight average molecular weight of about 120 Daltons, about 250 Daltons, about 300 Daltons, about 400 Daltons, about 500 Daltons, or about 1,000 Daltons. In some embodiments, the second polymer has a weight average molecular weight of at least about 120 Daltons, about 250 Daltons, about 300 Daltons, about 400 Daltons, or about 500 Daltons. In some embodiments, the second polymer has a weight average molecular weight of at most about 250 Daltons, about 300 Daltons, about 400 Daltons, about 500 Daltons, or about 1,000 Daltons.

In some embodiments, the second polymer comprises a water-soluble polymer. In some embodiments, the water-soluble polymer comprises poly(alkylene oxide), polysaccharide, poly(vinyl pyrrolidone), poly(vinyl alcohol), polyoxazoline, poly(acryloylmorpholine), or a combination thereof. In some embodiments, the water-soluble polymer is poly(alkylene oxide). In some embodiments, the water-soluble polymer is poly(ethylene oxide). In some embodiments, the second polymer is attached to the IL-18 polypeptide via click chemistry.

In some embodiments, the second water-soluble polymer comprises from 1 to 10 polyethylene glycol chains. In some embodiments, the second water-soluble polymer comprises 1 polyethylene glycol chains to 10 polyethylene glycol chains. In some embodiments, the second water-soluble polymer comprises 1 polyethylene glycol chains to 2 polyethylene glycol chains, 1 polyethylene glycol chains to 4 polyethylene glycol chains, 1 polyethylene glycol chains to 6 polyethylene glycol chains, 1 polyethylene glycol chains to 10 polyethylene glycol chains, 2 polyethylene glycol chains to 4 polyethylene glycol chains, 2 polyethylene glycol chains to 6 polyethylene glycol chains, 2 polyethylene glycol chains to 10 polyethylene glycol chains, 4 polyethylene glycol chains to 6 polyethylene glycol chains, 4 polyethylene glycol chains to 10 polyethylene glycol chains, or 6 polyethylene glycol chains to 10 polyethylene glycol chains. In some embodiments, the second water-soluble polymer comprises 1 polyethylene glycol chains, 2 polyethylene glycol chains, 4 polyethylene glycol chains, 6 polyethylene glycol chains, or 10 polyethylene glycol chains. In some embodiments, the second water-soluble polymer comprises at least 1 polyethylene glycol chains, 2 polyethylene glycol chains, 4 polyethylene glycol chains, or 6 polyethylene glycol chains. In some embodiments, the second water-soluble polymer comprises at most 2 polyethylene glycol chains, 4 polyethylene glycol chains, 6 polyethylene glycol chains, or 10 polyethylene glycol chains. In some embodiments, the first water-soluble polymer comprises 4 polyethylene glycol chains. In some embodiments, the second water-soluble polymer comprises the structure of Formula (II)

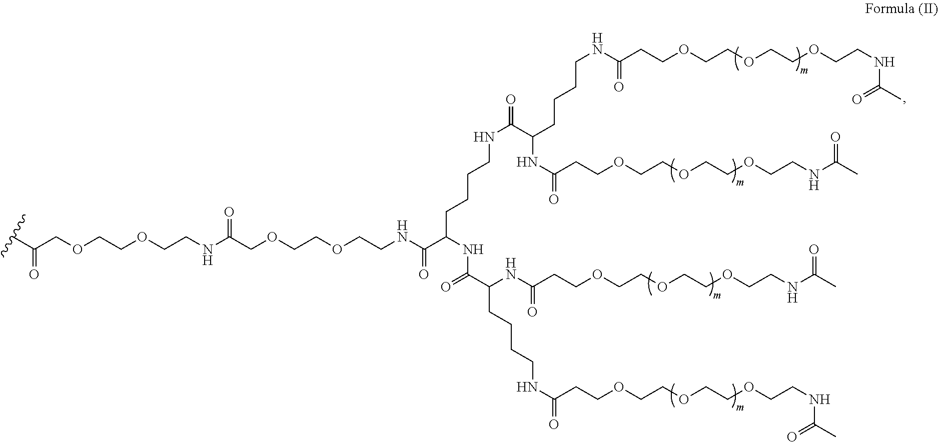

wherein each m is independently an integer from 4-30. In some embodiments, at least one polyethylene glycol chain of the second water-soluble polymer comprises the structure of Formula (III),

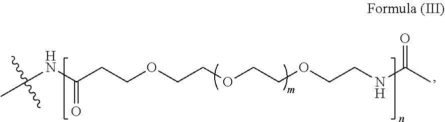

wherein each m is independently an integer from 4-30 and each n is independently an integer from 1-10. In some embodiments, each polyethylene glycol chain of the second water-soluble polymer comprises the structure of Formula (III).

In some embodiments, a modified IL-18 polypeptide described herein further comprises a third polymer covalently attached to the modified IL-18 polypeptide. In some embodiments, the third polymer has a weight average molecular weight of about 6,000 Daltons to about 50,000 Daltons. In some embodiments, the third polymer has a weight average molecular weight of about 6,000 Daltons to about 10,000 Daltons, about 6,000 Daltons to about 25,000 Daltons, about 6,000 Daltons to about 50,000 Daltons, about 10,000 Daltons to about 25,000 Daltons, about 10,000 Daltons to about 50,000 Daltons, or about 25,000 Daltons to about 50,000 Daltons. In some embodiments, the third polymer has a weight average molecular weight of about 6,000 Daltons, about 10,000 Daltons, about 25,000 Daltons, or about 50,000 Daltons. In some embodiments, the third polymer has a weight average molecular weight of at least about 6,000 Daltons, about 10,000 Daltons, or about 25,000 Daltons. In some embodiments, the third polymer has a weight average molecular weight of at most about 10,000 Daltons, about 25,000 Daltons, or about 50,000 Daltons.

In some embodiments, the third polymer has a weight average molecular weight of about 120 Daltons to about 1,000 Daltons. In some embodiments, the third polymer has a weight average molecular weight of about 120 Daltons to about 250 Daltons, about 120 Daltons to about 300 Daltons, about 120 Daltons to about 400 Daltons, about 120 Daltons to about 500 Daltons, about 120 Daltons to about 1,000 Daltons, about 250 Daltons to about 300 Daltons, about 250 Daltons to about 400 Daltons, about 250 Daltons to about 500 Daltons, about 250 Daltons to about 1,000 Daltons, about 300 Daltons to about 400 Daltons, about 300 Daltons to about 500 Daltons, about 300 Daltons to about 1,000 Daltons, about 400 Daltons to about 500 Daltons, about 400 Daltons to about 1,000 Daltons, or about 500 Daltons to about 1,000 Daltons. In some embodiments, the third polymer has a weight average molecular weight of about 120 Daltons, about 250 Daltons, about 300 Daltons, about 400 Daltons, about 500 Daltons, or about 1,000 Daltons. In some embodiments, the third polymer has a weight average molecular weight of at least about 120 Daltons, about 250 Daltons, about 300 Daltons, about 400 Daltons, or about 500 Daltons. In some embodiments, the third polymer has a weight average molecular weight of at most about 250 Daltons, about 300 Daltons, about 400 Daltons, about 500 Daltons, or about 1,000 Daltons.

In some embodiments, the modified IL-18 polypeptide comprises a third polymer having a weight average molecular weight of from about 250 Daltons to about 50,000 Daltons covalently attached thereto. In some embodiments, the modified IL-18 polypeptide comprises a third polymer having a weight average molecular weight of from about 500 Daltons to about 25,000 Daltons covalently attached thereto. In some embodiments, the modified IL-18 polypeptide comprises a third polymer having a weight average molecular weight of from about 1000 Daltons to about 10,000 Daltons covalently attached thereto.

In some embodiments, the third polymer comprises a water-soluble polymer. In some embodiments, the water-soluble polymer comprises poly(alkylene oxide), polysaccharide, poly(vinyl pyrrolidone), poly(vinyl alcohol), polyoxazoline, poly(acryloylmorpholine), or a combination thereof. In some embodiments, the water-soluble polymer is poly(alkylene oxide). In some embodiments, the water-soluble polymer is polyethylene glycol. In some embodiments, the third polymer is attached to the IL-18 polypeptide via click chemistry.

In another aspect, described herein is a modified IL-18 polypeptide, comprising: a modified IL-18 polypeptide, wherein the modified IL-18 polypeptide comprises: (a) a first polymer having a weight average molecular weight of up to about 6000 Daltons covalently attached to a first amino acid residue; (b) a second polymer having a weight average molecular weight of up to about 6000 Daltons covalently attached to a second amino acid residue; and wherein residue position numbering of the modified IL-18 polypeptide is based on SEQ ID NO: 1 as a reference sequence. In one aspect, described herein is a modified IL-18 polypeptide, comprising: a modified IL-18 polypeptide, wherein the modified IL-18 polypeptide comprises: (a) a first polymer covalently attached to a first amino acid residue; and (b) a second polymer covalently attached to a second amino acid residue, wherein one of the first polymer and the second polymer has a weight average molecular weight within a range of from about 200 Da, 300 Da, or 400 Da to about 600 Da, 1000 Da, or 6000 Da and the other polymer of the first polymer and the second polymer has a weight average molecular weight within a range of from about 5000 Da, 10,000 Da, or 20,000 Da to about 30,000 Da, 40,000 Da, or 50,000 Da, and wherein residue position numbering of the modified IL-18 polypeptide is based on SEQ ID NO: 1 as a reference sequence. In some embodiments, each of the first polymer and the second polymer independently comprises a water-soluble polymer.

In some embodiments, each polymer comprises a water-soluble polymer. In some embodiments, the water-soluble polymer comprises poly(alkylene oxide), polysaccharide, poly(vinyl pyrrolidone), poly(vinyl alcohol), polyoxazoline, poly(acryloylmorpholine), or a combination thereof. In some embodiments, each water-soluble polymer is poly(alkylene oxide). In some embodiments, each water-soluble polymer is polyethylene glycol.

In some embodiments, each of the first polymer and the second polymer independently comprises from 1 to 5 polyethylene glycol chains. In some embodiments, each of the first polymer and the second polymer independently comprise single polyethylene glycol chains.

In some embodiments, each of the first polymer and the second polymer independently comprises one polyethylene glycol chain with 3 to 25 ethylene glycol units. In some embodiments, each of the first polymer and the second polymer independently comprises one polyethylene glycol chain with 3 ethylene glycol units to 25 ethylene glycol units. In some embodiments, each of the first polymer and the second polymer independently comprises one polyethylene glycol chain with 3 ethylene glycol units to 5 ethylene glycol units, 3 ethylene glycol units to 7 ethylene glycol units, 3 ethylene glycol units to 10 ethylene glycol units, 3 ethylene glycol units to 15 ethylene glycol units, 3 ethylene glycol units to 25 ethylene glycol units, 5 ethylene glycol units to 7 ethylene glycol units, 5 ethylene glycol units to 10 ethylene glycol units, 5 ethylene glycol units to 15 ethylene glycol units, 5 ethylene glycol units to 25 ethylene glycol units, 7 ethylene glycol units to 10 ethylene glycol units, 7 ethylene glycol units to 15 ethylene glycol units, 7 ethylene glycol units to 25 ethylene glycol units, 10 ethylene glycol units to 15 ethylene glycol units, 10 ethylene glycol units to 25 ethylene glycol units, or 15 ethylene glycol units to 25 ethylene glycol units. In some embodiments, each of the first polymer and the second polymer independently comprises one polyethylene glycol chain with 3 ethylene glycol units, 5 ethylene glycol units, 7 ethylene glycol units, 10 ethylene glycol units, 15 ethylene glycol units, or 25 ethylene glycol units. In some embodiments, each of the first polymer and the second polymer independently comprises one polyethylene glycol chain with at least 3 ethylene glycol units, 5 ethylene glycol units, 7 ethylene glycol units, 10 ethylene glycol units, or 15 ethylene glycol units. In some embodiments, each of the first polymer and the second polymer independently comprises one polyethylene glycol chain with at most 5 ethylene glycol units, 7 ethylene glycol units, 10 ethylene glycol units, 15 ethylene glycol units, or 25 ethylene glycol units.

In some embodiments, the third water-soluble polymer comprises from 1 to 10 polyethylene glycol chains. In some embodiments, the third water-soluble polymer comprises 1 polyethylene glycol chains to 10 polyethylene glycol chains. In some embodiments, the third water-soluble polymer comprises 1 polyethylene glycol chains to 2 polyethylene glycol chains, 1 polyethylene glycol chains to 4 polyethylene glycol chains, 1 polyethylene glycol chains to 6 polyethylene glycol chains, 1 polyethylene glycol chains to 10 polyethylene glycol chains, 2 polyethylene glycol chains to 4 polyethylene glycol chains, 2 polyethylene glycol chains to 6 polyethylene glycol chains, 2 polyethylene glycol chains to 10 polyethylene glycol chains, 4 polyethylene glycol chains to 6 polyethylene glycol chains, 4 polyethylene glycol chains to 10 polyethylene glycol chains, or 6 polyethylene glycol chains to 10 polyethylene glycol chains. In some embodiments, the third water-soluble polymer comprises 1 polyethylene glycol chains, 2 polyethylene glycol chains, 4 polyethylene glycol chains, 6 polyethylene glycol chains, or 10 polyethylene glycol chains. In some embodiments, the third water-soluble polymer comprises at least 1 polyethylene glycol chains, 2 polyethylene glycol chains, 4 polyethylene glycol chains, or 6 polyethylene glycol chains. In some embodiments, the third water-soluble polymer comprises at most 2 polyethylene glycol chains, 4 polyethylene glycol chains, 6 polyethylene glycol chains, or 10 polyethylene glycol chains. In some embodiments, the third water-soluble polymer comprises 4 polyethylene glycol chains. In some embodiments, the third water-soluble polymer comprises the structure of Formula (II)

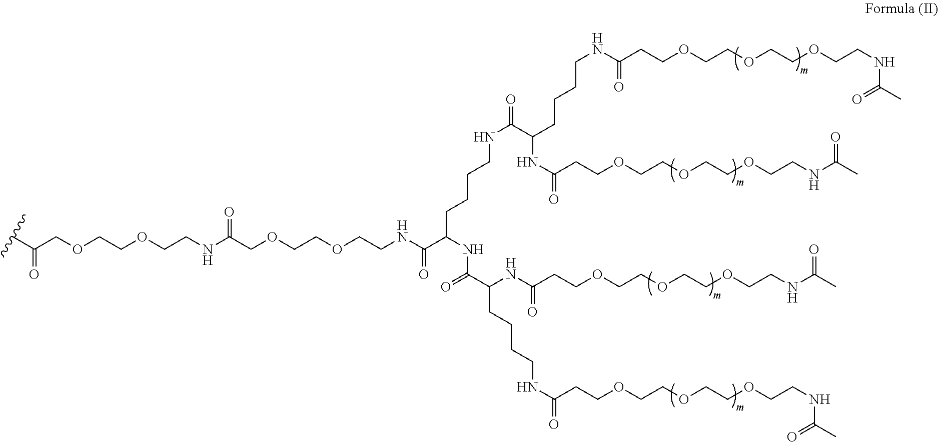

wherein each m is independently an integer from 4-30. In some embodiments, each polyethylene glycol chain of the third water-soluble polymer comprises the structure of Formula (III)

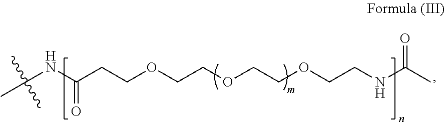

wherein each m is independently an integer from 4-30 and each n is independently an integer from 1-10.

In some embodiments, each of the polyethylene glycol chains independently comprises from about 5 to about 300, from about 10 to about 200, from about 20 to about 100, or from about 25 to about 50 ethylene glycol units. In some embodiments, each of the polyethylene glycol chains independently comprises 5 ethylene glycol units to 300 ethylene glycol units. In some embodiments, each of the polyethylene glycol chains independently comprises 5 ethylene glycol units to 10 ethylene glycol units, 5 ethylene glycol units to 20 ethylene glycol units, 5 ethylene glycol units to 25 ethylene glycol units, 5 ethylene glycol units to 50 ethylene glycol units, 5 ethylene glycol units to 100 ethylene glycol units, 5 ethylene glycol units to 200 ethylene glycol units, 5 ethylene glycol units to 300 ethylene glycol units, 10 ethylene glycol units to 20 ethylene glycol units, 10 ethylene glycol units to 25 ethylene glycol units, 10 ethylene glycol units to 50 ethylene glycol units, 10 ethylene glycol units to 100 ethylene glycol units, 10 ethylene glycol units to 200 ethylene glycol units, 10 ethylene glycol units to 300 ethylene glycol units, 20 ethylene glycol units to 25 ethylene glycol units, 20 ethylene glycol units to 50 ethylene glycol units, 20 ethylene glycol units to 100 ethylene glycol units, 20 ethylene glycol units to 200 ethylene glycol units, 20 ethylene glycol units to 300 ethylene glycol units, 25 ethylene glycol units to 50 ethylene glycol units, 25 ethylene glycol units to 100 ethylene glycol units, 25 ethylene glycol units to 200 ethylene glycol units, 25 ethylene glycol units to 300 ethylene glycol units, 50 ethylene glycol units to 100 ethylene glycol units, 50 ethylene glycol units to 200 ethylene glycol units, 50 ethylene glycol units to 300 ethylene glycol units, 100 ethylene glycol units to 200 ethylene glycol units, 100 ethylene glycol units to 300 ethylene glycol units, or 200 ethylene glycol units to 300 ethylene glycol units. In some embodiments, each of the polyethylene glycol chains independently comprises 5 ethylene glycol units, 10 ethylene glycol units, 20 ethylene glycol units, 25 ethylene glycol units, 50 ethylene glycol units, 100 ethylene glycol units, 200 ethylene glycol units, or 300 ethylene glycol units. In some embodiments, each of the polyethylene glycol chains independently comprises at least 5 ethylene glycol units, 10 ethylene glycol units, 20 ethylene glycol units, 25 ethylene glycol units, 50 ethylene glycol units, 100 ethylene glycol units, or 200 ethylene glycol units. In some embodiments, each of the polyethylene glycol chains independently comprises at most 10 ethylene glycol units, 20 ethylene glycol units, 25 ethylene glycol units, 50 ethylene glycol units, 100 ethylene glycol units, 200 ethylene glycol units, or 300 ethylene glycol units.

In some embodiments, each of the polyethylene glycol chains is independently linear or branched. In some embodiments, each of the polyethylene glycol chains is a linear polyethylene glycol. In some embodiments, each of the polyethylene glycol chains is a branched polyethylene glycol. For example, in some embodiments, each of the first and the second polymers comprises a linear polyethylene glycol chain.

In some embodiments, each of the polyethylene glycol chains is independently terminally capped with a hydroxy, an alkyl, an alkoxy, an amido, or an amino group. In some embodiments, each of the polyethylene glycol chains is independently terminally capped with an amino group. In some embodiments, each of the polyethylene glycol chains is independently terminally capped with an amido group. In some embodiments, each of the polyethylene glycol chains is independently terminally capped with an alkoxy group. In some embodiments, each of the polyethylene glycol chains is independently terminally capped with an alkyl group. In some embodiments, each of the polyethylene glycol chains is independently terminally capped with a hydroxy group. In some embodiments, one or more of the polyethylene glycol chains independently has the structure

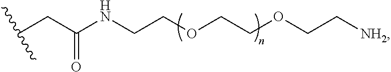

wherein n is an integer from 4-30. In some embodiments, one or more of the polyethylene glycol chains independently has the structure

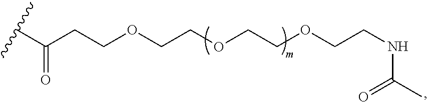

wherein m is an integer from 4-30.

In some embodiments, the modified IL-18 polypeptide comprises from 1 to 10 covalently attached water-soluble polymers. In some embodiments, the modified IL-18 polypeptide comprises 1 to 10 covalently attached water-soluble polymers. In some embodiments, the modified IL-18 polypeptide comprises 1 or 2 covalently attached water-soluble polymers, 1 to 3 covalently attached water-soluble polymers, 1 to 4 covalently attached water-soluble polymers, 1 to 6 covalently attached water-soluble polymers, 1 to 8 covalently attached water-soluble polymers, 1 to 10 covalently attached water-soluble polymers, 2 or 3 covalently attached water-soluble polymers, 2 to 4 covalently attached water-soluble polymers, 2 to 6 covalently attached water-soluble polymers, 2 to 8 covalently attached water-soluble polymers, 2 to 10 covalently attached water-soluble polymers, 3 or 4 covalently attached water-soluble polymers, 3 to 6 covalently attached water-soluble polymers, 3 to 8 covalently attached water-soluble polymers, 3 to 10 covalently attached water-soluble polymers, 4 to 6 covalently attached water-soluble polymers, 4 to 8 covalently attached water-soluble polymers, 4 to 10 covalently attached water-soluble polymers, 6 to 8 covalently attached water-soluble polymers, 6 to 10 covalently attached water-soluble polymers, or 8 to 10 covalently attached water-soluble polymers. In some embodiments, the modified IL-18 polypeptide comprises 1 covalently attached water-soluble polymer, 2 covalently attached water-soluble polymers, 3 covalently attached water-soluble polymers, 4 covalently attached water-soluble polymers, 6 covalently attached water-soluble polymers, 8 covalently attached water-soluble polymers, or 10 covalently attached water-soluble polymers. In some embodiments, the modified IL-18 polypeptide comprises at least 1 covalently attached water-soluble polymer, 2 covalently attached water-soluble polymers, 3 covalently attached water-soluble polymers, 4 covalently attached water-soluble polymers, 6 covalently attached water-soluble polymers, or 8 covalently attached water-soluble polymers. In some embodiments, the modified IL-18 polypeptide comprises at most 2 covalently attached water-soluble polymers, 3 covalently attached water-soluble polymers, 4 covalently attached water-soluble polymers, 6 covalently attached water-soluble polymers, 8 covalently attached water-soluble polymers, or 10 covalently attached water-soluble polymers. In some embodiments, the modified IL-18 polypeptide comprises from 2 to 6 covalently attached water-soluble polymers.

In some embodiments, one or more of the covalently attached polymers comprise a linker. In some embodiments, one or more of the covalently attached polymers, such as the third polymer, comprises one or more linkers. In some embodiments, the linker comprises one or more amino acids. In some embodiments, the linker comprises one or more lysines. In some embodiments, the linker comprises a spacer. In some embodiments, the linker comprises reactive functional groups or functional groups such as amide. In some embodiments, the linker has the structure of Formula (Iv)

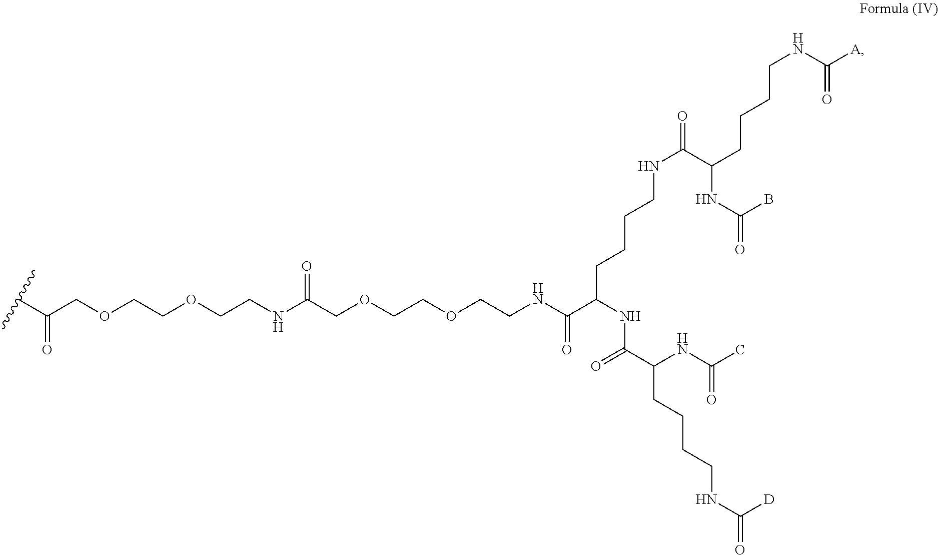

wherein A, B, C, and D are each independently polymers.

In some embodiments, the modified IL-18 polypeptide comprises one or more PEGylated lysines having a structure of formula (I),

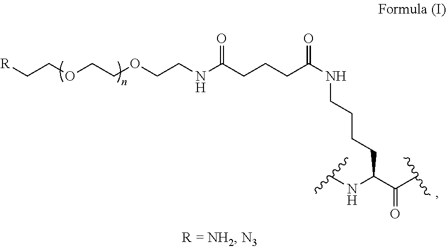

wherein n is an integer selected from 4 to 30. In some embodiments, n is 4 to 6, 4 to 8, 4 to 10, 4 to 15, 4 to 20, 4 to 25, 4 to 30, 6 to 8, 6 to 10, 6 to 15, 6 to 20, 6 to 25, 6 to 30, 8 to 10, 8 to 15, 8 to 20, 8 to 25, 8 to 30, 10 to 15, 10 to 20, 10 to 25, 10 to 30, 15 to 20, 15 to 25, 15 to 30, 20 to 25, 20 to 30, or 25 to 30. In some embodiments, n is 4, 6, 8, 10, 15, 20, 25, or 30. In some embodiments, n is at least 4, 6, 8, 10, 15, 20, or 25. In some embodiments, n is at most 6, 8, 10, 15, 20, 25, or 30. In one aspect, a modified IL-18 polypeptide as described herein comprises one or two water-soluble polymers covalently attached at one or two amino acid residues. For example, in some embodiments, the modified IL-18 polypeptide comprises one or two water-soluble polymers having the characteristics and attachment sites as shown in Table 2.

TABLE 2
Exemplary Polypeptides Structures and Water-soluble Polymer Characteristics
Exemplary

| Polypeptide | Characteristics of water-soluble | Characteristics of water-soluble |
|---|---|---|
| structures | polymer attached at residue A | polymer attached at residue B |
| 1 | Linear; | Linear; |
| | Mw: from about 15k to about 50k Da | Mw: from about 200 to about 1000 Da |
| 2 | Linear | Linear; |
| | Mw: from about 200 to about 1000 Da | Mw: from about 15k to about 50k Da |
| 3 | Branched; | Linear; |
| | Mw: from about 2k to about 10k Da | Mw: from about 200 to about 1000 Da |
| 4 | Linear; | Branched; |
| | Mw: from about 200 to about 1000 Da Mw: | from about 2k to about 10k Da |
| 5 | Linear; | Branched; |
| | Mw: from about 15k to about 50k Da | Mw: from about 200 to about 1000 Da |
| 6 | Branched; | Linear; |
| | Mw: from about 200 to about 1000 Da | Mw: from about 15k to about 50k Da |
| 7 | comprising polyethylene glycol of | None |
| | non-uniform size; | |
| | Mw is from about 15k to about 50k Da | |
| 8 | None | Linear; |
| | | Mw: from about 15k to about 50k Da |
| 9 | Linear; | None |
| | Mw: from about 15k to about 50k Da | |
| 10 | Branched; | None |
| | Mw: from about 2k to about 10k Da | |
| 11 | None | Branched; |
| | | Mw: from about 2k to about 10k Da |
| 12 | Branched; | None |
| | Mw: from about 15k to about 50k Da | |
| 13 | None | Branched; |
| | | Mw: from about 15k to about 50k Da |
| 14 | Branched; | Branched; |
| | Mw: from about 2k to about 10k Da | Mw: from about 2k to about 10k Da |
| 15 | Linear; | Branched; |
| | Mw: from about 2k to about 10k Da | Mw: from about 2k to about 10k Da |
| 16 | Linear; | Linear; |
| | Mw: from about 200 to about 1000 Da | Mw: from about 200 to about 1000 Da |
| 17 | Linear; | None |
| | Mw: from about 200 to about 1000 Da | |
| 18 | None | Linear; |
| | | Mw: from about 200 to about 1000 Da |

In some embodiments, a water-soluble polymer that can be attached to a modified IL-18 polypeptide comprises a structure of Formula (A):

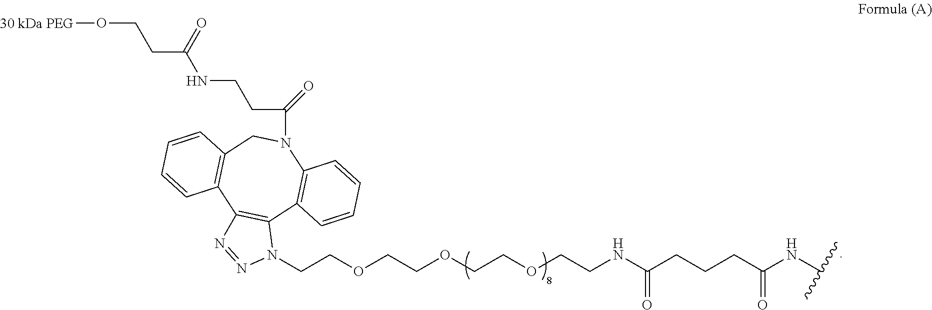

In some embodiments, a water-soluble polymer that can be attached to a modified IL-18 polypeptide comprises a structure of Formula (B):

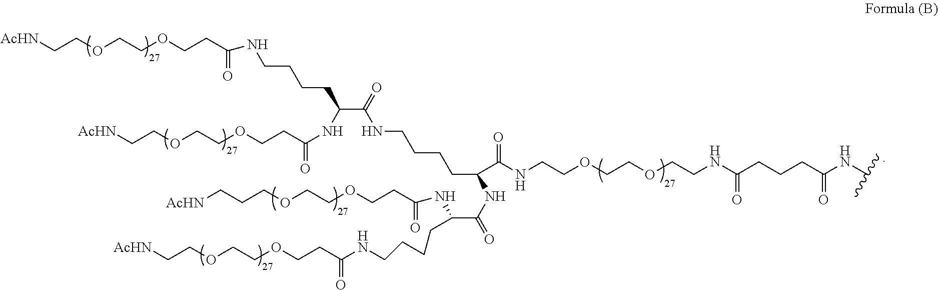

In some embodiments, a water-soluble polymer that can be attached to a modified IL-18 polypeptide comprises a structure of Formula (C):

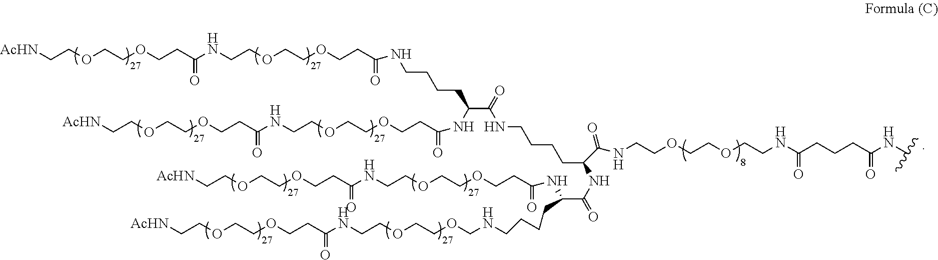

In some embodiments, a water-soluble polymer that can be attached to a modified IL-18 polypeptide comprises a structure of Formula (D):

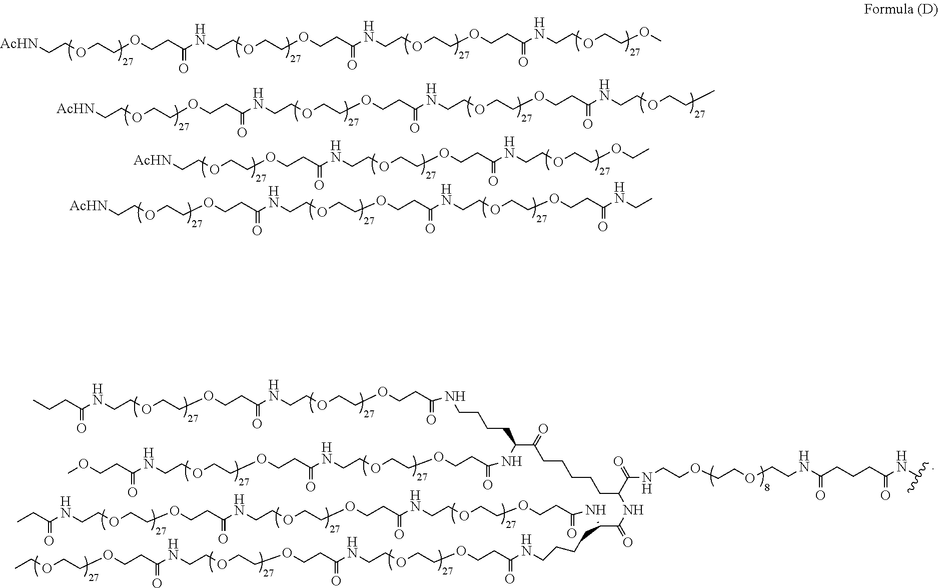

In some embodiments, a water-soluble polymer that can be attached to a modified IL-18 polypeptide comprises a structure of Formula (E):

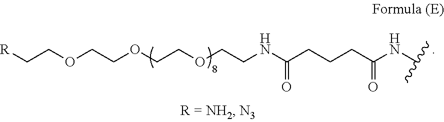

In some embodiments, the modified IL-18 polypeptide comprises one or two water-soluble polymers having the structures and attachment sites as shown in Table 3.

TABLE 3
Exemplary Polypeptide Structures and
Water-soluble Polymer Structures

| Residue A Conjugate | Residue b Conjugate |
|---|---|
| comprising a polymer | comprising a polymer |
| that comprises a | that comprises a |
| structure of Formula: | structure of Formula: |
| Formula E | Formula A |
| Formula A | Formula E |
| Formula A | Formula B |
| Formula B | Formula A |
| Formula E | Formula D |
| Formula D | Formula E |
| None | Formula A |
| Formula A | None |
| None | Formula B |
| Formula B | None |
| None | Formula D |
| Formula D | None |
| Formula B | Formula E |
| Formula E | Formula B |
| Formula E | Formula E |
| Formula E | None |
| None | Formula E |

In some embodiments, the water-soluble polymer attached at residue 68 or 70 comprises one or more linkers and/or spacers. In some embodiments, the one or more linkers comprise one or more amide groups. In some embodiments, the one or more linkers comprise one or more lysine groups. In some embodiments, the water-soluble polymer attached at residue 68 or 70 comprises a structure of Formula (II), Formula (III), Formula (IV), or a combination thereof. In some embodiments, the water-soluble polymer attached at residue 68 or 70 comprises a structure of Formula (A), Formula (B), Formula (C), Formula (D), or a combination thereof. In some embodiments, the water-soluble polymer attached at residue 68 or 70 comprises a structure of

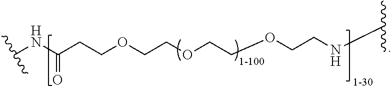

In some embodiments, the water-soluble polymer attached at residue 68 or 70 comprises one or more linkers and/or spacers. In some embodiments, the one or more linkers comprise one or more amide groups. In some embodiments, the one or more linkers comprise one or more lysine groups. In some embodiment, the water-soluble polymer attached at residue 68 comprises a structure of Formula (II), Formula (III), Formula (IV), or a combination thereof. In some embodiments, the water-soluble polymer attached at residue 70 comprises a structure of Formula (A), Formula (B), Formula (C), Formula (D), or a combination thereof. In some embodiments, the water-soluble polymer attached at residue 68 or 70 comprises a structure of

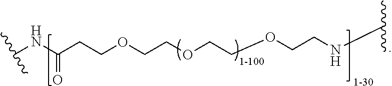

In some embodiments, the water-soluble polymer attached at residue 68, 69, or 70 comprises one or more linkers and/or spacers. In some embodiments, the one or more linkers comprise one or more amide groups. In some embodiments, the one or more linkers comprise one or more lysine groups. In some embodiments, the water-soluble polymer attached at residue 68, 69, or 70 comprises a structure of Formula (II), Formula (III), Formula (IV), or a combination thereof. In some embodiments, the water-soluble polymer attached at residue 68, 69, or 70 comprises a structure of Formula (A), Formula (B), Formula (C), Formula (D), or a combination thereof. In some embodiments, the water-soluble polymer attached at residue 68, 69, or 70 comprises a structure of

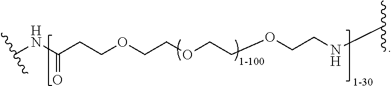

In some embodiments, the water-soluble polymer attached at residue 68, 69, or 70 comprises one or more linkers and/or spacers. In some embodiments, the one or more linkers comprise one or more amide groups. In some embodiments, the one or more linkers comprise one or more lysine groups. In some embodiment, the water-soluble polymer attached at residue 69 comprises a structure of Formula (II), Formula (III), Formula (IV), or a combination thereof. In some embodiments, the water-soluble polymer attached at residue 69 comprises a structure of Formula (A), Formula (B), Formula (C), Formula (D), or a combination thereof.

In some embodiments, the polymers are synthesized from suitable precursor materials. In some embodiments, the polymers are synthesized from the precursor materials of, Structure 6, Structure 7, Structure 8, or Structure 9, wherein Structure 6 is

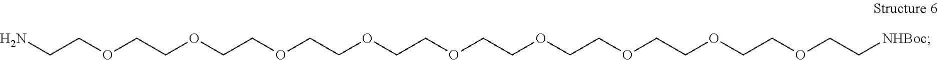 [SMILES: CC(C)(C)OC(=O)NCCOCCOCCOCCOCCOCCOCCOCCOCCOCCN]

### Structure 7 is

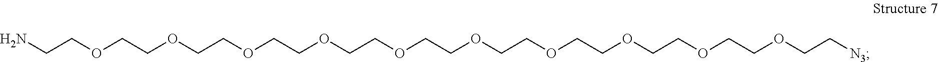 [SMILES: [N-]=[N+]=NCCOCCOCCOCCOCCOCCOCCOCCOCCOCCOCCN]

### Structure 8 is

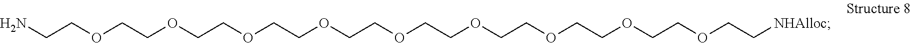

### and Structure 9 is

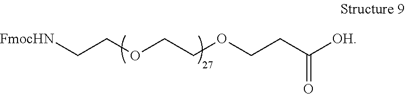

Also described herein is a modified IL-18 polypeptide population comprising, a plurality of modified IL-18 polypeptides, wherein the plurality of modified IL-18 polypeptides comprise a plurality of water-soluble polymers attached at a residue of the polypeptide. In some embodiments, at least 75%, at least 80%, at least 85%, at least 90%, or at least 95% of the plurality of water-soluble polymers attached at a residue of the polypeptide have a molecular weight that is within ±10% of the peak molecular weight of the attached plurality of water-soluble polymers as determined by matrix-assisted laser desorption/ionization mass spectroscopy (MALDI-MS). In some embodiments, at most 50%, at most 60%, at most 75%, at most 80%, at most 85%, at most 90%, or at most 95% of the plurality of water-soluble polymers attached to the polypeptide have a molecular weight that is within ±10% of the peak molecular weight of the plurality of water-soluble polymers attached at to the polypeptide as determined by MALDI-MS. In some embodiments, a ratio of weight average molecular weight over number average molecular weight for the plurality of water-soluble polymers attached to the polypeptide is from about 1.0 to about 1.5, from about 1.0 to about 1.1, from about 1.0 to about 1.2, from about 1.0 to about 1.3, from about 1.0 to about 1.25, from about 1.05 to about 1.1, from about 1.05 to about 1.2, from about 1.05 to about 1.5, from about 1.1 to about 1.2, from about 1.1 to about 1.5, or from about 1.2 to about 1.5, as determined by chromatography such as gel permeation chromatography (GPC) and high performance liquid chromatography (HPLC) or mass spectrometry such as MALDI-MS.

In some embodiments, the population comprises at least 1 μg, at least 10 μg, or at least 1 mg of the modified IL-18 polypeptides. In some embodiments, the population comprises at least 100, at least 1000, or at least 1000 of the modified IL-18 polypeptides. In some embodiments, a ratio of weight average molecular weight over number average molecular weight for the population of the modified IL-18 polypeptide is at most 1.1.

In some embodiments, each of the plurality of polymers comprises a water-soluble polymer. In some embodiments, the water-soluble polymer comprises poly(alkylene oxide), polysaccharide, poly(vinyl pyrrolidone), poly(vinyl alcohol), polyoxazoline, poly(acryloylmorpholine), or a combination thereof. In some embodiments, the water-soluble polymer comprises polyethylene glycol.

In some embodiments, a weight average molecular weight of the plurality of polymers is from about 200 Da to about 50,000 Da. In some embodiments, a weight average molecular weight of the plurality of polymers is from about 10,000 Da to about 30,000 Da.

In some embodiments, the modified IL-18 polypeptide comprises a polypeptide sequence having at least about 80%, at least about 85%, at least about 90%, at least about 95%, or about 100% sequence identity to SEQ ID NO: 2-58. In some embodiments, the modified IL-18 polypeptide comprises a polypeptide sequence having at least about 80%, at least about 85%, at least about 90%, at least about 95%, or about 100% sequence identity to SEQ ID NO: 2-83. In some embodiments, the polypeptide sequence is at least about 80% identical to SEQ ID NO: 2 or SEQ ID NO: 18. In some embodiments, the polypeptide sequence is at least about 80% identical to SEQ ID NO: 2. In some embodiments, the polypeptide sequence is at least about 90% identical to SEQ ID NO: 2. In some embodiments, the polypeptide sequence is at least about 95% identical to SEQ ID NO: 2. In some embodiments, the polypeptide sequence is at least about 80% identical to SEQ ID NO: 18. In some embodiments, the polypeptide sequence is at least about 90% identical to SEQ ID NO: 18. In some embodiments, the polypeptide sequence is at least about 95% identical to SEQ ID NO: 18.

In some embodiments, a ratio of weight average molecular weight over number average molecular weight for the plurality of water-soluble polymers attached to the polypeptide is at least 1.1, at least 1.2, at least 1.3, at least 1.5, at least 1.6, at least 1.7, at least 1.8, at least 1.9, at least 2.0, at least 2.5, or at least 3.0 as determined by chromatography such as GPC and HPLC or mass spectrometry. In some embodiments, at least 75%, at least 80%, at least 85%, at least 90%, or at least 95% of the plurality of water-soluble polymers attached to the polypeptide have a molecular weight that is within ±10% of the peak molecular weight of the plurality of water-soluble polymers attached to the polypeptide as determined by MALDI-MS. In some embodiments, at most 50%, at most 60%, at most 75%, at most 80%, at most 85%, at most 90%, or at most 95% of the plurality of water-soluble polymers attached to the polypeptide have a molecular weight that is within ±10% of the peak molecular weight of the plurality of water-soluble polymers attached to the polypeptide as determined by MALDI-MS.

In some embodiments, a ratio of weight average molecular weight over number average molecular weight for the plurality of water-soluble polymers attached to the polypeptide is from about 1.0 to about 1.5, from about 1.0 to about 1.1, from about 1.0 to about 1.2, from about 1.0 to about 1.3, from about 1.0 to about 1.25, from about 1.05 to about 1.1, from about 1.05 to about 1.2, from about 1.05 to about 1.5, from about 1.1 to about 1.2, from about 1.1 to about 1.5, or from about 1.2 to about 1.5, as determined by chromatography such as GPC and HPLC or mass spectrometry. In some embodiments, a ratio of weight average molecular weight over number average molecular weight for the plurality of water-soluble polymers attached to the polypeptide is at least 1.1, at least 1.2, at least 1.3, at least 1.5, at least 1.6, at least 1.7, at least 1.8, at least 1.9, at least 2.0, at least 2.5, or at least 3.0 as determined by chromatography such as GPC and HPLC or mass spectrometry.

### IIIb. Monodispersity

In one aspect, described herein is a population of modified IL-18 polypeptides. In some embodiments, a population of the modified IL-18 polypeptides described herein is monodispersed. In some embodiments, the population of modified IL-18 polypeptides comprises monodispersed polymers. In some embodiments, the monodispersed polymers are attached to the N-terminus or a residue of the polypeptides. In some embodiments, the monodispersed polymers are attached to a residue position of a modified IL-18 polypeptide of the disclosure.

In some embodiments, a population of modified IL-18 polypeptides described herein comprises a polymer covalently attached thereto. In some embodiments, each of the modified IL-18 polypeptides comprises a polymer covalently attached thereto. In some embodiments, the polymer is a monodisperse polymer. In some embodiments, the polymer is covalently attached to residue 68 or 70, wherein residue position numbering of the modified IL-18 polypeptide is based on SEQ ID NO: 1 as a reference sequence. In some embodiments, the polymer is covalently attached to residue 68, 69, or 70, wherein residue position numbering of the modified IL-18 polypeptide is based on SEQ ID NO: 1 as a reference sequence. In some embodiments, the polymer is covalently attached to the N-terminal region of the modified IL-18 polypeptide. In some embodiments, the polymer is covalently attached to the N-terminus of the modified IL-18 polypeptides.

In some embodiments, a population of modified IL-18 described herein comprises a second polymer covalently attached thereto. In some embodiments, the second polymer is a monodisperse polymer. In some embodiments, a population of modified IL-18 polypeptides described herein comprises a third polymer covalently attached thereto. In some embodiments, the third polymer is a monodisperse polymer.

In some embodiments, a population of the modified IL-18 polypeptides described herein is monodispersed. In some embodiments, a ratio of weight average molecular weight over number average molecular weight for the population of the modified IL-18 polypeptide is at most 1.5, at most 1.2, at most 1.1, or at most 1.05. In some embodiments, the pharmaceutical composition comprises a population of the modified IL-18 polypeptides, and wherein a ratio of weight average molecular weight over number average molecular weight for the population of the modified IL-18 polypeptide is 1.05 to 1.5. In some embodiments, the pharmaceutical composition comprises a population of the modified IL-18 polypeptides, and wherein a ratio of weight average molecular weight over number average molecular weight for the population of the modified IL-18 polypeptide is from about 1.0 to about 1.5, from about 1.0 to about 1.1, from about 1.0 to about 1.2, from about 1.0 to about 1.3, from about 1.0 to about 1.4, from about 1.05 to about 1.1, from about 1.05 to about 1.2, from about 1.05 to about 1.5, from about 1.1 to about 1.2, from about 1.1 to about 1.5, or from about 1.2 to about 1.5. In some embodiments, the pharmaceutical composition comprises a population of the modified IL-18 polypeptides, and wherein a ratio of weight average molecular weight over number average molecular weight for the population of the modified IL-18 polypeptides is about 1.05, 1.1, about 1.2, or about 1.5. In some embodiments, the pharmaceutical composition comprises a population of the modified IL-18 polypeptides, and wherein a ratio of weight average molecular weight over number average molecular weight for the population of the modified IL-18 polypeptide is at least 1.05, 1.1, or 1.2. In some embodiments, the pharmaceutical composition comprises a population of the modified IL-18 polypeptides, and wherein a ratio of weight average molecular weight over number average molecular weight for the population of the modified IL-18 polypeptide is at most 1.1, 1.2, or 1.5. In some embodiments, the ratio is determined by chromatography such as gel permeation chromatography (GPC) and high performance liquid chromatography (HPLC). In some embodiments, the ratio is determined by mass spectrum such as MALDI-MS and ESI-HRMS.

In some embodiments of a population of modified IL-18 polypeptides described herein, at least 80%, at least 85%, at least 90%, at least 95%, or at least 99% of the population of modified IL-18 polypeptides have a molecular weight that is within ±10% of the peak molecular weight as determined by mass spectrum. In some embodiments of the population of modified IL-18 polypeptides, at least 80% of the population of modified IL-18 polypeptides have a molecular weight that is within ±10% of the peak molecular weight as determined by mass spectrum. In some embodiments of the population of modified IL-18 polypeptides, at least 85% of the population of modified IL-18 polypeptides have a molecular weight that is within ±10% of the peak molecular weight as determined by mass spectrum. In some embodiments of the population of modified IL-18 polypeptides, at least 90% of the population of modified IL-18 polypeptides have a molecular weight that is within ±10% of the peak molecular weight as determined by mass spectrum. In some embodiments of the population of modified IL-18 polypeptides, at least 95% of the population of modified IL-18 polypeptides have a molecular weight that is within ±10% of the peak molecular weight as determined by mass spectrum. In some embodiments of the population of modified IL-18 polypeptides, at least 99% of the population of modified IL-18 polypeptides have a molecular weight that is within ±10% of the peak molecular weight as determined by mass spectrum. In some embodiments, the mass spectrum is a MALDI-mass spectrometry. In some embodiments, the mass spectrum is a high-resolution electrospray ionization mass spectrometry (ESI-MS or ESI-HRMS).

In some embodiments of a population of modified IL-18 polypeptides described herein, at least 80%, at least 85%, at least 90%, at least 95%, or at least 99% of the population of modified IL-18 polypeptides have a molecular weight that is within ±5% of the peak molecular weight as determined by mass spectrum. In some embodiments of the population of modified IL-18 polypeptides, at least 80% of the population of modified IL-18 polypeptides have a molecular weight that is within ±5% of the peak molecular weight as determined by mass spectrum. In some embodiments of the population of modified IL-18 polypeptides, at least 85% of the population of modified IL-18 polypeptides have a molecular weight that is within ±5% of the peak molecular weight as determined by mass spectrum. In some embodiments of the population of modified IL-18 polypeptides, at least 90% of the population of modified IL-18 polypeptides have a molecular weight that is within ±5% of the peak molecular weight as determined by mass spectrum. In some embodiments of the population of modified IL-18 polypeptides, at least 95% of the population of modified IL-18 polypeptides have a molecular weight that is within ±5% of the peak molecular weight as determined by mass spectrum. In some embodiments of the population of modified IL-18 polypeptides, at least 99% of the population of modified IL-18 polypeptides have a molecular weight that is within ±5% of the peak molecular weight as determined by mass spectrum.

In some embodiments of a population of modified IL-18 polypeptides described herein, at least 80%, at least 85%, at least 90%, at least 95%, or at least 99% of the population of modified IL-18 polypeptides have a molecular weight that is within ±2% of the peak molecular weight as determined by mass spectrum. In some embodiments of the population of modified IL-18 polypeptides, at least 80% of the population of modified IL-18 polypeptides have a molecular weight that is within ±2% of the peak molecular weight as determined by mass spectrum. In some embodiments of the population of modified IL-18 polypeptides, at least 85% of the population of modified IL-18 polypeptides have a molecular weight that is within ±2% of the peak molecular weight as determined by mass spectrum. In some embodiments of the population of modified IL-18 polypeptides, at least 90% of the population of modified IL-18 polypeptides have a molecular weight that is within ±2% of the peak molecular weight as determined by mass spectrum. In some embodiments of the population of modified IL-18 polypeptides, at least 95% of the population of modified IL-18 polypeptides have a molecular weight that is within ±2% of the peak molecular weight as determined by mass spectrum. In some embodiments of the population of modified IL-18 polypeptides, at least 99% of the population of modified IL-18 polypeptides have a molecular weight that is within ±2% of the peak molecular weight as determined by mass spectrum.

In some embodiments of a population of modified IL-18 polypeptides described herein, at least 80%, at least 85%, at least 90%, at least 95%, or at least 99% of the population of modified IL-18 polypeptides have a molecular weight that is within ±1% of the peak molecular weight as determined by mass spectrum. In some embodiments of the population of modified IL-18 polypeptides, at least 80% of the population of modified IL-18 polypeptides have a molecular weight that is within ±1% of the peak molecular weight as determined by mass spectrum. In some embodiments of the population of modified IL-18 polypeptides, at least 85% of the population of modified IL-18 polypeptides have a molecular weight that is within ±1% of the peak molecular weight as determined by mass spectrum. In some embodiments of the population of modified IL-18 polypeptides, at least 90% of the population of modified IL-18 polypeptides have a molecular weight that is within ±1% of the peak molecular weight as determined by mass spectrum. In some embodiments of the population of modified IL-18 polypeptides, at least 95% of the population of modified IL-18 polypeptides have a molecular weight that is within ±1% of the peak molecular weight as determined by mass spectrum. In some embodiments of the population of modified IL-18 polypeptides, at least 99% of the population of modified IL-18 polypeptides have a molecular weight that is within ±1% of the peak molecular weight as determined by mass spectrum.

In some embodiments of a population of modified IL-18 polypeptides described herein, at least 80%, at least 85%, at least 90%, at least 95%, or at least 99% of the population of modified IL-18 polypeptides have a molecular weight that is within ±1000 Daltons of the peak molecular weight as determined by mass spectrum. In some embodiments of the population of modified IL-18 polypeptides, at least 80% of the population of modified IL-18 polypeptides have a molecular weight that is within ±1000 Daltons of the peak molecular weight as determined by mass spectrum. In some embodiments of the population of modified IL-18 polypeptides, at least 85% of the population of modified IL-18 polypeptides have a molecular weight that is within ±1000 Daltons of the peak molecular weight as determined by mass spectrum. In some embodiments of the population of modified IL-18 polypeptides, at least 90% of the population of modified IL-18 polypeptides have a molecular weight that is within ±1000 Daltons of the peak molecular weight as determined by mass spectrum. In some embodiments of the population of modified IL-18 polypeptides, at least 95% of the population of modified IL-18 polypeptides have a molecular weight that is within ±1000 Daltons of the peak molecular weight as determined by mass spectrum. In some embodiments of the population of modified IL-18 polypeptides, at least 99% of the population of modified IL-18 polypeptides have a molecular weight that is within ±1000 Daltons of the peak molecular weight as determined by mass spectrum.

In some embodiments of a population of modified IL-18 polypeptides described herein, at least 80%, at least 85%, at least 90%, at least 95%, or at least 99% of the population of modified IL-18 polypeptides have a molecular weight that is within ±500 Daltons of the peak molecular weight as determined by mass spectrum. In some embodiments of the population of modified IL-18 polypeptides, at least 80% of the population of modified IL-18 polypeptides have a molecular weight that is within ±500 Daltons of the peak molecular weight as determined by mass spectrum. In some embodiments of the population of modified IL-18 polypeptides, at least 85% of the population of modified IL-18 polypeptides have a molecular weight that is within ±500 Daltons of the peak molecular weight as determined by mass spectrum. In some embodiments of the population of modified IL-18 polypeptides, at least 90% of the population of modified IL-18 polypeptides have a molecular weight that is within ±500 Daltons of the peak molecular weight as determined by mass spectrum. In some embodiments of the population of modified IL-18 polypeptides, at least 95% of the population of modified IL-18 polypeptides have a molecular weight that is within ±500 Daltons of the peak molecular weight as determined by mass spectrum. In some embodiments of the population of modified IL-18 polypeptides, at least 99% of the population of modified IL-18 polypeptides have a molecular weight that is within ±500 Daltons of the peak molecular weight as determined by mass spectrum.

In some embodiments of a population of modified IL-18 polypeptides described herein, at least 80%, at least 85%, at least 90%, at least 95%, or at least 99% of the population of modified IL-18 polypeptides have a molecular weight that is within ±100 Daltons of the peak molecular weight as determined by mass spectrum. In some embodiments of the population of modified IL-18 polypeptides, at least 80% of the population of modified IL-18 polypeptides have a molecular weight that is within ±100 Daltons of the peak molecular weight as determined by mass spectrum. In some embodiments of the population of modified IL-18 polypeptides, at least 85% of the population of modified IL-18 polypeptides have a molecular weight that is within ±100 Daltons of the peak molecular weight as determined by mass spectrum. In some embodiments of the population of modified IL-18 polypeptides, at least 90% of the population of modified IL-18 polypeptides have a molecular weight that is within ±100 Daltons of the peak molecular weight as determined by mass spectrum. In some embodiments of the population of modified IL-18 polypeptides, at least 95% of the population of modified IL-18 polypeptides have a molecular weight that is within ±100 Daltons of the peak molecular weight as determined by mass spectrum. In some embodiments of the population of modified IL-18 polypeptides, at least 99% of the population of modified IL-18 polypeptides have a molecular weight that is within ±100 Daltons of the peak molecular weight as determined by mass spectrum.

In some embodiments of the population of modified IL-18 polypeptides, at least 90% of the population of modified IL-18 polypeptides have a molecular weight that is within ±20 Da, ±10 Da, or ±5 Da of the peak molecular weight as determined by mass spectrum. In some embodiments of the population of modified IL-18 polypeptides, at least 95% of the population of modified IL-18 polypeptides have a molecular weight that is within ±20 Da, ±10 Da, or ±5 Da of the peak molecular weight as determined by mass spectrum. In some embodiments of the population of modified IL-18 polypeptides, at least 99% of the population of modified IL-18 polypeptides have a molecular weight that is within ±20 Da, ±10 Da, or ±5 Da of the peak molecular weight as determined by mass spectrum. In some embodiments, the mass spectrum is a MALDI-mass spectrometry. In some embodiments, the mass spectrum is ESI-HRMS.

In some embodiments of a population of modified IL-18 polypeptides described herein, at least 80%, at least 85%, at least 90%, or at least 95% of the population of modified IL-18 polypeptides have the same molecular weight as measured by mass spectrum. In some embodiments of the population of modified IL-18 polypeptides, at least 80% of the population of modified IL-18 polypeptides have the same molecular weight as measured by mass spectrum. In some embodiments of the population of modified IL-18 polypeptides, at least 85% of the population of modified IL-18 polypeptides have the same molecular weight as measured by mass spectrum. In some embodiments of the population of modified IL-18 polypeptides, at least 90% of the population of modified IL-18 polypeptides have the same molecular weight as measured by mass spectrum. In some embodiments of the population of modified IL-18 polypeptides, at least 95% of the population of modified IL-18 polypeptides have the same molecular weight as measured by mass spectrum.

In some embodiments, a population of modified IL-18 polypeptides described herein exists substantially in one apparent molecular weight form when assessed, for example, by size exclusion chromatography, dynamic light scattering, ESI-MS, MALDI-MS, or analytical ultracentrifugation. In some embodiments, the population of modified IL-18 polypeptides exists in at least about 80%, 85%, 90%, 91%, 92%, 93%, 94%, 95%, 96%, 97%, 98%, 99%, 99.5% or greater in one apparent molecular weight form when assessed, for example, by size exclusion chromatography, dynamic light scattering, ESI-MS, MALDI-MS, or analytical ultracentrifugation. In some embodiments, the population of modified IL-18 polypeptides exists substantially in one apparent molecular weight form when assessed by size exclusion chromatography. In some embodiments, the population of modified IL-18 polypeptides exists substantially in one apparent molecular weight form when assessed by dynamic light scattering. In some embodiments, the population of modified IL-18 polypeptides exists substantially in one apparent molecular weight form when assessed by MALDI-MS or ESI-MS. In some embodiments, the population of modified IL-18 polypeptides exist substantially in one apparent molecular weight form when assessed by analytical ultracentrifugation.

In one aspect, described herein is a modified IL-18 polypeptide population, comprising a plurality of modified IL-18 polypeptides, wherein the plurality of modified IL-18 polypeptides comprise a plurality of polymers (i.e., a plurality of first polymers), wherein each of the modified IL-18 polypeptides comprises one of the plurality of polymers covalently attached thereto. In some embodiments, at least 95% of the plurality of polymers have a molecular weight that is within ±10% of the peak molecular weight of the plurality of polymers as determined by mass spectrum. In some embodiments, at least 75%, at least 80%, at least 85%, at least 90%, or least 95% of the plurality of polymers have a molecular weight that is within ±10% of the peak molecular weight of the plurality of polymers as determined by mass spectrum. In some embodiments, at least 75%, at least 80%, at least 85%, at least 90%, or least 95% of the plurality of polymers have a molecular weight that is within ±5% of the peak molecular weight of the plurality of polymers as determined by MALDI-MS. In some embodiments, at most 50%, at most 60%, at most 75%, at most 80%, at most 85%, at most 90%, or most 95% of the plurality of polymers have a molecular weight that is within ±10% of the peak molecular weight of the plurality of polymers as determined by mass spectrum wherein at least 80%, at least 85%, at least 90%, at least 95%, or at least 99% of the population of modified IL-18 polypeptides have a molecular weight that is within ±10% of the peak molecular weight as determined by mass spectrum. In some embodiments, at most 50%, at most 60%, at most 75%, at most 80%, at most 85%, at most 90%, or most 95% of the plurality of polymers have a molecular weight that is within ±5% of the peak molecular weight of the plurality of polymers as determined by mass spectrum. In some embodiments, at least 80%, at least 85%, at least 90%, at least 95%, or at least 99% of the population of modified IL-18 polypeptides have a molecular weight that is within ±1% of the peak molecular weight as determined by mass spectrum. n some embodiments, at least 80%, at least 85%, at least 90%, at least 95%, or at least 99% of the population of modified IL-18 polypeptides have a molecular weight that is within ±0.5% of the peak molecular weight as determined by mass spectrum. n some embodiments, at least 80%, at least 85%, at least 90%, at least 95%, or at least 99% of the population of modified IL-18 polypeptides have a molecular weight that is within ±0.1% of the peak molecular weight as determined by mass spectrum. In some embodiments, a ratio of weight average molecular weight over number average molecular weight for the plurality of polymers is from about 1.0 to about 1.5, from about 1.0 to about 1.1, from about 1.0 to about 1.2, from about 1.0 to about 1.3, from about 1.0 to about 1.25, from about 1.05 to about 1.1, from about 1.05 to about 1.2, from about 1.05 to about 1.5, from about 1.1 to about 1.2, from about 1.1 to about 1.5, or from about 1.2 to about 1.5, as determined by chromatography such as GPC and HPLC or mass spectrometry such as MALDI-MS. In some embodiments, a ratio of weight average molecular weight over number average molecular weight for the plurality of polymers is at least 1.1, at least 1.2, at least 1.3, at least 1.5, at least 1.6, at least 1.7, at least 1.8, at least 1.9, at least 2.0, at least 2.5, or at least 3.0 as determined by chromatography such as GPC and HPLC or mass spectrometry.

In some embodiments, the weight average molecular weight of the polymers is at least about 1000 Da. In some embodiments, the weight average molecular weight of the polymers is at least about 3000 Da, at least about 6000 Da, at least about 12,000 Da, or at least about 24,000 Da. In some embodiments, the weight average molecular weight of the polymers is at least about 3000 Da. In some embodiments, the weight average molecular weight of the polymers is at least about 6000 Da. In some embodiments, the weight average molecular weight of the polymers is at least about 12,000 Da. In some embodiments, the weight average molecular weight of the polymers is at least about 24,000 Da.

In some embodiments, a plurality of modified IL-18 polypeptides described herein comprise a plurality of second polymers, wherein each of the modified IL-18 polypeptides comprises one of the plurality of second polymers covalently attached thereto. In some embodiments, at least 75%, at least 80%, at least 85%, at least 90%, or least 95% of the plurality of second polymers have a molecular weight that is within ±10% of the peak molecular weight of the plurality of second polymers as determined by mass spectrum such as MALDI-MS and ESI-MS. In some embodiments, at least 75%, at least 80%, at least 85%, at least 90%, or least 95% of the plurality of second polymers have a molecular weight that is within ±5% of the peak molecular weight of the plurality of second polymers as determined by mass spectrum. In some embodiments, at most 50%, at most 60%, at most 75%, at most 80%, at most 85%, at most 90%, or most 95% of the plurality of second polymers have a molecular weight that is within ±10% of the peak molecular weight of the plurality of second polymers as determined by mass spectrum. In some embodiments, at most 50%, at most 60%, at most 75%, at most 80%, at most 85%, at most 90%, or most 95% of the plurality of second polymers have a molecular weight that is within ±5% of the peak molecular weight of the plurality of second polymers as determined by mass spectrum. In some embodiments, a ratio of weight average molecular weight over number average molecular weight for the plurality of second polymers is from about 1.0 to about 1.5, from about 1.0 to about 1.1, from about 1.0 to about 1.2, from about 1.0 to about 1.3, from about 1.0 to about 1.25, from about 1.05 to about 1.1, from about 1.05 to about 1.2, from about 1.05 to about 1.5, from about 1.1 to about 1.2, from about 1.1 to about 1.5, or from about 1.2 to about 1.5, as determined by chromatography such as GPC and HPLC or mass spectrometry such as mass spectrum. In some embodiments, a ratio of weight average molecular weight over number average molecular weight for the plurality of second polymers is at least 1.1, at least 1.2, at least 1.3, at least 1.5, at least 1.6, at least 1.7, at least 1.8, at least 1.9, at least 2.0, at least 2.5, or at least 3.0 as determined by chromatography such as GPC and HPLC or mass spectrometry.

In some embodiments, the weight average molecular weight of the second polymers is at least about 3000 Da, at least about 6000 Da, at least about 12,000 Da, or at least about 24,000 Da. In some embodiments, the weight average molecular weight of the second polymers is at least about 3000 Da. In some embodiments, the weight average molecular weight of the second polymers is at least about 6000 Da. In some embodiments, the weight average molecular weight of the second polymers is at least about 12,000 Da. In some embodiments, the weight average molecular weight of the second polymers is at least about 24,000 Da.

In some embodiments, the plurality comprises at least 100, at least 1000, at least 10000, at least 100000, at least 1000000, at least 10000000 of the modified IL-18 polypeptides. In some embodiments, the plurality comprises at least 100 of the modified IL-18 polypeptides. In some embodiments, the plurality comprises at least 1000 of the modified IL-18 polypeptides. In some embodiments, the plurality comprises at least 10000 of the modified IL-18 polypeptides. In some embodiments, the plurality comprises at least 100000 of the modified IL-18 polypeptides. In some embodiments, the plurality comprises at least 1000000 of the modified IL-18 polypeptides. In some embodiments, the plurality comprises at least 10000000 of the modified IL-18 polypeptides. In some embodiments, the plurality comprises at least 100000000 of the modified IL-18 polypeptides.

In some embodiments, the plurality comprises about 100, about 1000, about 10000, about 100000, about 1000000, about 10000000 of the modified IL-18 polypeptides. In some embodiments, the plurality comprises about 100 of the modified IL-18 polypeptides. In some embodiments, the plurality comprises about 1000 of the modified IL-18 polypeptides. In some embodiments, the plurality comprises about 10000 of the modified IL-18 polypeptides. In some embodiments, the plurality comprises about 100000 of the modified IL-18 polypeptides. In some embodiments, the plurality comprises about 1000000 of the modified IL-18 polypeptides. In some embodiments, the plurality comprises about 10000000 of the modified IL-18 polypeptides. In some embodiments, the plurality comprises about 100000000 of the modified IL-18 polypeptides.

In some embodiments, the plurality comprises at least 1 μg, at least 10 μg, at least 100 μg, at least 1 mg, at least 10 mg, or at least 100 mg of the modified IL-18 polypeptides. In some embodiments, the plurality comprises at least 1 μg of the modified IL-18 polypeptides. In some embodiments, the plurality comprises at least 10 μg of the modified IL-18 polypeptides. In some embodiments, the plurality comprises at least 100 μg of the modified IL-18 polypeptides. In some embodiments, the plurality comprises at least 1 mg of the modified IL-18 polypeptides. In some embodiments, the plurality comprises at least 10 mg of the modified IL-18 polypeptides.

In some embodiments, the plurality comprises about 100 mg of the modified IL-18 polypeptides. In some embodiments, the plurality comprises about 1 μg, about 10 μg, about 100 μg, about 1 mg, about 10 mg, or about 100 mg of the modified IL-18 polypeptides. In some embodiments, the plurality comprises about 1 μg of the modified IL-18 polypeptides. In some embodiments, the plurality comprises about 10 μg of the modified IL-18 polypeptides. In some embodiments, the plurality comprises about 100 μg of the modified IL-18 polypeptides. In some embodiments, the plurality comprises about 1 mg of the modified IL-18 polypeptides. In some embodiments, the plurality comprises about 10 mg of the modified IL-18 polypeptides. In some embodiments, the plurality comprises about 100 mg of the modified IL-18 polypeptides.

In some embodiments, a herein described modified IL-18 polypeptide is a linear polypeptide. In some embodiments, a herein described modified IL-18 polypeptide is folded. In some embodiments, the modified polypeptide comprises one or more disulfide bonds.

In some embodiments, a modified IL-18 polypeptide described herein comprises a covalently attached polymer for half-life extension. In some embodiments, the modified IL-18 polypeptide of the disclosure comprises a covalently attached polymer for plasma or serum half-life extension. In some embodiments, a plasma or serum half-life of the modified IL-18 polypeptide of the disclosure is at least 1.5-fold, 2-fold, 3-fold, 4-fold, 5-fold, 6-fold, 7-fold, 8-fold, 9-fold, or 10-fold longer compared to a plasma or serum half-life of a wild-type IL-18 polypeptide. In some embodiments, a plasma or serum half-life of the modified IL-18 polypeptide of the disclosure is 1.5-fold to 10-fold longer compared to a plasma or serum half-life of a wild-type IL-18 polypeptide. In some embodiments, a plasma or serum half-life of the modified IL-18 polypeptide of the disclosure is 1.5-fold to 2-fold, 1.5-fold to 4-fold, 1.5-fold to 6-fold, 1.5-fold to 8-fold, 1.5-fold to 10-fold, 2-fold to 4-fold, 2-fold to 6-fold, 2-fold to 8-fold, 2-fold to 10-fold, 4-fold to 6-fold, 4-fold to 8-fold, 4-fold to 10-fold, 6-fold to 8-fold, 6-fold to 10-fold, or 8-fold to 10-fold longer compared to a plasma or serum half-life of a wild-type IL-18 polypeptide. In some embodiments, a plasma or serum half-life of the modified IL-18 polypeptide of the disclosure is 1.5-fold, 2-fold, 4-fold, 6-fold, 8-fold, or 10-fold longer compared to a plasma or serum half-life of a wild-type IL-18 polypeptide. In some embodiments, a plasma or serum half-life of the modified IL-18 polypeptide of the disclosure is at least 1.5-fold, 2-fold, 4-fold, 6-fold, or 8-fold. In some embodiments, a plasma or serum half-life of the modified IL-18 polypeptide of the disclosure is at most 2-fold, 4-fold, 6-fold, 8-fold, or 10-fold longer compared to a plasma or serum half-life of a wild-type IL-18 polypeptide.

In some embodiments, a plasma or serum half-life of a modified IL-18 polypeptide described herein is at least 1.5-fold, 2-fold, 3-fold, 4-fold, 5-fold, 6-fold, 7-fold, 8-fold, 9-fold, or 10-fold longer compared to a plasma or serum half-life of the modified IL-18 polypeptide without the half-life extending polymer. In some embodiments, a plasma or serum half-life of the modified IL-18 polypeptide of the disclosure is 1.5-fold to 10-fold longer compared to a plasma or serum half-life of the modified IL-18 polypeptide without the half-life extending polymer. In some embodiments, a plasma or serum half-life of the modified IL-18 polypeptide of the disclosure is 1.5-fold to 2-fold, 1.5-fold to 4-fold, 1.5-fold to 6-fold, 1.5-fold to 8-fold, 1.5-fold to 10-fold, 2-fold to 4-fold, 2-fold to 6-fold, 2-fold to 8-fold, 2-fold to 10-fold, 4-fold to 6-fold, 4-fold to 8-fold, 4-fold to 10-fold, 6-fold to 8-fold, 6-fold to 10-fold, or 8-fold to 10-fold longer compared to a plasma or serum half-life of the modified IL-18 polypeptide without the half-life extending polymer. In some embodiments, a plasma or serum half-life of the modified IL-18 polypeptide of the disclosure is 1.5-fold, 2-fold, 4-fold, 6-fold, 8-fold, or 10-fold longer compared to a plasma or serum half-life of the modified IL-18 polypeptide without the half-life extending polymer. In some embodiments, a plasma or serum half-life of the modified IL-18 polypeptide of the disclosure is at least 1.5-fold, 2-fold, 4-fold, 6-fold, or 8-fold. In some embodiments, a plasma or serum half-life of the modified IL-18 polypeptide of the disclosure is at most 2-fold, 4-fold, 6-fold, 8-fold, or 10-fold longer compared to a plasma or serum half-life of the modified IL-18 polypeptide without the half-life extending polymer.

### IV. Pharmaceutical Compositions

In one aspect, described herein is a pharmaceutical composition comprising: a modified IL-18 polypeptide described herein; and a pharmaceutically acceptable carrier or excipient. In some embodiments, the pharmaceutical composition comprises a plurality of the modified IL-18 polypeptides. In some embodiments, the pharmaceutical compositions further comprises one or more excipient selected from a carbohydrate, an inorganic salt, an antioxidant, a surfactant, or a buffer.

In some embodiments, the pharmaceutical composition further comprises a carbohydrate. In certain embodiments, the carbohydrate is selected from the group consisting of fructose, maltose, galactose, glucose, D-mannose, sorbose, lactose, sucrose, trehalose, cellobiose raffinose, melezitose, maltodextrins, dextrans, starches, mannitol, xylitol, maltitol, lactitol, xylitol, sorbitol (glucitol), pyranosyl sorbitol, myoinositol, cyclodextrins, and combinations thereof.

In some embodiments, the pharmaceutical composition comprises an inorganic salt. In certain embodiments, the inorganic salt is selected from the group consisting of sodium chloride, potassium chloride, magnesium chloride, calcium chloride, sodium phosphate, potassium phosphate, sodium sulfate, or combinations thereof.

In certain embodiments, the pharmaceutical composition comprises an antioxidant. In certain embodiments, the antioxidant is selected from the group consisting of ascorbyl palmitate, butylated hydroxyanisole, butylated hydroxytoluene, potassium metabisulfite, propyl gallate, sodium metabisulfite, sodium thiosulfate, vitamin E, 3,4-dihydroxybenzoic acid, and combinations thereof.

In certain embodiments, the pharmaceutical composition comprises a surfactant. In certain embodiments, the surfactant is selected from the group consisting of polysorbates, sorbitan esters, lipids, phospholipids, phosphatidylethanolamines, fatty acids, fatty acid esters, steroids, EDTA, zinc, and combinations thereof.

In certain embodiments, the pharmaceutical composition comprises a buffer. In certain embodiments, the buffer is selected from the group consisting of citric acid, sodium phosphate, potassium phosphate, acetic acid, ethanolamine, histidine, amino acids, tartaric acid, succinic acid, fumaric acid, lactic acid, tris, HEPES, or combinations thereof. In certain embodiments, the buffer is selected from the group consisting of citric acid, sodium phosphate, potassium phosphate, acetic acid, ethanolamine, histidine, amino acids, tartaric acid, succinic acid, fumaric acid, lactic acid, tris, HEPES, CHAPS, or combinations thereof.

In some embodiments, the pharmaceutical composition is formulated for parenteral or enteral administration. In some embodiments, the pharmaceutical composition is formulated for intravenous or subcutaneous administration. In some embodiments, the pharmaceutical composition is in a lyophilized form.

In one aspect, described herein is a liquid or lyophilized composition that comprises a described modified IL-18 polypeptide. In some embodiments, the modified IL-18 polypeptide is a lyophilized powder. In some embodiments, the lyophilized powder is resuspended in a buffer solution. In some embodiments, the buffer solution comprises a buffer, a sugar, a salt, a surfactant, or any combination thereof. In some embodiments, the buffer solution comprises a phosphate salt. In some embodiments, the phosphate salt is sodium Na₂HPO₄. In some embodiments, the salt is sodium chloride. In some embodiments, the buffer solution comprises phosphate buffered saline. In some embodiments, the buffer solution comprises mannitol. In some embodiments, the lyophilized powder is suspended in a solution comprising phosphate buffered saline solution (pH 7.4) with 50 mg/mL mannitol. In some embodiments, the pharmaceutical composition is a lyophilized composition which is reconstituted shortly before administration to a subject.

The modified IL-18 polypeptides described herein can be in a variety of dosage forms. In some embodiments, the modified IL-18 polypeptide is dosed as a lyophilized powder. In some embodiments, the modified IL-18 polypeptide is dosed as a suspension. In some embodiments, the modified IL-18 polypeptide is dosed as a solution. In some embodiments, the modified IL-18 polypeptide is dosed as an injectable solution. In some embodiments, the modified IL-18 polypeptides is dosed as an IV solution.

### V. Synthesis of Modified IL-18 Polypeptides

The modified IL-18 polypeptides described herein may can be synthesized chemically rather than expressed as recombinant polypeptides. The modified IL-18 polypeptides can be made by synthesizing one or more fragments of the full-length modified IL-18 polypeptides, ligating the fragments together, and folding the ligated full-length polypeptide. In some embodiments, the modified IL-18 polypeptide comprises at least one mutation in the amino acid sequence and a PEG polymer covalently attached to residue C68 or K70 of the polypeptide. In some embodiments, the modified IL-18 polypeptide comprises at least one mutation in the amino acid sequence and a PEG polymer covalently attached to residue C68, E69, or K70 of the polypeptide. In some embodiments, the PEG is attached to a cysteine residue at position 68, 69, or 70 of the modified IL-18 polypeptide. In some embodiments, the PEG polymer has a molecular weight of at least about 1 kDa, at least about 2 kDa, at least about 5 kDa, at least about 10 kDa, at least about 15 kDa, or at least about 20 kDa. In some embodiments, the PEG polymer has a molecular weight of about 1 kDa, about 2 kDa, about 5 kDa, about 10 kDa, about 15 kDa, about 20 kDa, about 25 kDa, or about 30 kDa.

In some embodiments, the modified IL-18 polypeptide comprises at least one mutation in the amino acid sequence and a PEG polymer of about 30 kDa covalently attached to residue C68 or K70 of the polypeptide. In some embodiments, the modified IL-18 polypeptide comprises at least one mutation in the amino acid sequence and a PEG polymer of about 30 kDa covalently attached to residue C68, E69, or K70 of the polypeptide.

In some embodiments, the modified IL-18 polypeptides enhance IFNγ induction when administered to a subject. In some embodiments, the modified IL-18 polypeptides enhance IFNγ induction while being resistant to IL-18BP neutralization when administered to a subject.

### VI. Host Cells

In one aspect, described herein is a host cell comprising a modified IL-18 polypeptide.

In one aspect, described herein is a method of producing a modified IL-18 polypeptide, wherein the method comprises expressing the modified IL-18 polypeptide in a host cell.

In some embodiments, the host cell is a prokaryotic cell or a eukaryotic cell. In some embodiments, the host cell is a mammalian cell, an avian cell, or an insect cell. In some embodiments, the host cell is a mammalian cell, an avian cell, a fungal cell, or an insect cell. In some embodiments, the host cell is a CHO cell, a COS cell, or a yeast cell.

### VII. Biological Activity of Modified IL-18 Polypeptides

### VIIa. Binding Affinity

In one aspect, described herein is a modified IL-18 polypeptide that exhibits a greater affinity for IL-18Rα than IL-18BP. In some embodiments, the affinity to IL-18Rα, IL-18Rα/β heterodimer, or IL-18BP is measured by a dissociation constant (K_{D}). As used herein, the phrase “the K_{D} of the modified IL-18 polypeptide/IL-18Rα” means the dissociation constant of the binding interaction of the modified IL-18 polypeptide and IL-18Rα. The phrase “the K_{D} of the modified IL-18 polypeptide/IL-18Rα/β” means the dissociation constant of the binding interaction of the modified IL-18 polypeptide and the IL-18Rα/β heterodimer. Similarly, the phrase “the K_{D} of the modified IL-18 polypeptide/IL-18BP” means the dissociation constant of the binding interaction of the modified IL-18 polypeptide and IL-18BP.

In some embodiments, the modified IL-18 polypeptide exhibits a greater affinity to an IL-18 receptor (IL-18R) than to IL-18 binding protein (IL-18BP) as measured by K_{D}, and wherein [K_{D} IL-18R]/[K_{D} IL-18BP] is lower than 1.

In some embodiments, the modified IL-18 polypeptide exhibits less than a 10-fold lower affinity, less than a 5-fold lower affinity, or a greater affinity to an IL-18 receptor alpha subunit (IL-18Rα) than to IL-18 binding protein (IL-18BP) as measured by K_{D}. In some embodiments, the modified IL-18 polypeptide exhibits less than a 10-fold lower affinity to an IL-18 receptor alpha subunit (IL-18Rα) than to IL-18 binding protein (IL-18BP) as measured by K_{D}. In some embodiments, the modified IL-18 polypeptide exhibits less than a 5-fold lower affinity, to an IL-18 receptor alpha subunit (IL-18Rα) than to IL-18 binding protein (IL-18BP) as measured by K_{D}. In some embodiments, the K_{D} is determined by a surface plasmon resonance assay (see, e.g., Example 8, Example 10, and Example 12). In some embodiments, the K_{D} is determined by an alphaLISA assay (see, e.g., Example 9).

In some embodiments, the modified IL-18 polypeptide exhibits less than a 10-fold lower affinity, less than a 5-fold lower affinity, or a greater affinity to an IL-18 receptor alpha subunit (IL-18Rα) than to IL-18 binding protein (IL-18BP) as measured by K_{D}, and wherein [K_{D} IL-18Rα]/[K_{D} IL-18BP] is greater than 0.1 In some embodiments, the [K_{D} IL-18Rα]/[K_{D} IL-18BP] is greater than about 0.1, 0.2, 0.3, 0.4, 0.5, 0.6, 0.7, 0.8, 0.9, or 1. In some embodiments, the K_{D} is determined by a surface plasmon resonance assay (see, e.g., Example 8, Example 10, and Example 12). In some embodiments, the K_{D} is determined by an alphaLISA assay (see, e.g., Example 9).

In some embodiments, the modified IL-18 polypeptide binds to IL-18 receptor alpha (IL-18Rα). In some embodiments, the modified IL-18 polypeptide binds to IL-18Rα with a K_{D} of less than about 200 nM, less than about 100 nM, or less than about 50 nM. In some embodiments, the modified IL-18 polypeptide binds to IL-18Rα with a K_{D} of less than about 200 nM. In some embodiments, the modified IL-18 polypeptide binds to IL-18Rα with a K_{D} of less than about 100 nM. In some embodiments, the modified IL-18 polypeptide binds to IL-18Rα with a K_{D} of less than about 50 nM. In some embodiments, the modified IL-18 polypeptide binds to IL-18Rα with a K_{D} of less than about 10 nM. In some embodiments, the K_{D} is determined by a surface plasmon resonance assay (see, e.g., Example 8 and Example 10).

In some embodiments, the modified IL-18 polypeptide binds to an IL-18 receptor alpha/beta (IL-18Rα/β) heterodimer. In some embodiments, the modified IL-18 polypeptide binds to the IL-18Rα/β heterodimer with a K_{D} of less than about 100 nM. In some embodiments, the modified IL-18 polypeptide binds to the IL-18Rα/β heterodimer with a K_{D} of less than about 500 nM. In some embodiments, the modified IL-18 polypeptide binds to the IL-18Rα/β heterodimer with a K_{D} of less than about 20 nM. In some embodiments, the modified IL-18 polypeptide binds to the IL-18Rα/β heterodimer with a K_{D} of less than about 10 nM. In some embodiments, the modified IL-18 polypeptide binds to the IL-18Rα/β heterodimer with a K_{D} of less than about 5 nM. In some embodiments, the modified IL-18 polypeptide binds to the IL-18Rα/β heterodimer with a K_{D} of less than about 2 nM. In some embodiments, the modified IL-18 polypeptide binds to the IL-18α/β with a K_{D} similar to that of an IL-18 polypeptide of SEQ ID No: 1. In some embodiments, the K_{D} is determined by a surface plasmon resonance assay (see, e.g., Example 8 and Example 11).

In some embodiments, the K_{D} of the modified IL-18 polypeptide/IL-18Rα is less than 1000 nM, less than 750 nM, less than 500 nM, less than 450, less than 400 nM, less than 350 nM, less than 300 nM, less than 250 nM, less than 200 nM, less than 150 nM, less than 140 nM, less than 130 nM, less than 125 nM, less than 120 nM, less than 100 nM. In some embodiments, the K_{D} of the modified IL-18 polypeptide/IL-18Rα is less than 150 nM, less than 50 nM, less than 25 nM, or less than 10 nM. In some embodiments, the K_{D} of the modified IL-18 polypeptide/IL-18Rα is less than 50 nM. In some embodiments, the K_{D} of the modified IL-18 polypeptide/IL-18Rα is less than 10 nM. In some embodiments, the K_{D} is determined by a surface plasmon resonance assay (see, e.g., Example 8 and Example 10).

In some embodiments, the K_{D} of the modified IL-18 polypeptide/IL-18Rα/β heterodimer is less than 1000 nM, less than 750 nM, less than 500 nM, less than 450, less than 400 nM, less than 350 nM, less than 300 nM, less than 250 nM, less than 200 nM, less than 150 nM, less than 140 nM, less than 130 nM, less than 125 nM, less than 120 nM, less than 100 nM. In some embodiments, the K_{D} of the modified IL-18 polypeptide/IL-18Rα/β heterodimer is less than 150 nM, less than 50 nM, less than 25 nM, less than 10 nM, less than 5 nM, or less than 2 nM. In some embodiments, the K_{D} of the modified IL-18 polypeptide/IL-18Rα/β heterodimer is less than 50 nM. In some embodiments, the K_{D} of the modified IL-18 polypeptide/IL-18Rα/β heterodimer is less than 10 nM. In some embodiments, the K_{D} of the modified IL-18 polypeptide/IL-18Rα/β heterodimer is less than 5 nM. In some embodiments, the K_{D} is determined by a surface plasmon resonance assay (see, e.g., Example 8 and Example 11).

In some embodiments, the K_{D} of the modified IL-18 polypeptide/IL-18BP is less than 1000 nM, less than 750 nM, less than 500 nM, less than 450, less than 400 nM, less than 350 nM, less than 300 nM, less than 250 nM, less than 200 nM, less than 150 nM, less than 140 nM, less than 130 nM, less than 125 nM, less than 120 nM, less than 100 nM. In some embodiments, the K_{D} of the modified IL-18 polypeptide/IL-18BP is less than 50 nM, less than 25 nM, less than 1 nM, or less than 0.5 nM. In some embodiments, the K_{D} of the modified IL-18 polypeptide/IL-18BP is about 10 nM. In some embodiments, the K_{D} of the modified IL-18 polypeptide/IL-18BP is about 2.5 nM. In some embodiments, the K_{D} of the modified IL-18 polypeptide/IL-18BP is about 1 nM. In some embodiments, the K_{D} is determined by a surface plasmon resonance assay (see, e.g., Example 8 and Example 12). In some embodiments, the K_{D} is determined by an alphaLISA assay (see, e.g., Example 9).

In some embodiments, the K_{D} of a modified IL-18 polypeptide/IL-18Rα is substantially the same as the K_{D} of wild-type IL-18/IL-18Rα. In some embodiments, the K_{D} of the modified IL-18 polypeptide/IL-18Rα is greater than the K_{D} of wild-type IL-18/IL-18Rα. In some embodiments, the K_{D} of the modified IL-18 polypeptide/IL-18Rα is lower than the K_{D} of wild-type IL-18/IL-18Rα. In some embodiments, the K_{D} of the modified IL-18 polypeptide/IL-18Rα is at most 10%, at most 20%, at most 30%, at most 40%, at most 50%, at most 60%, at most 70%, at most 80%, at most 85%, or at most 90% greater than the K_{D} of wild-type IL-18/IL-18Rα. In some embodiments, the K_{D} of the modified IL-18 polypeptide/IL-18Rα is at least 20% greater than the K_{D} of wild-type IL-18/IL-18Rα. In some embodiments, the K_{D} of the modified IL-18 polypeptide/IL-18Rα is at least 25% greater than the K_{D} of wild-type IL-18/IL-18Rα. In some embodiments, the K_{D} is determined by a surface plasmon resonance assay (see, e.g., Example 8 and Example 10).

In some embodiments, the K_{D} of the modified IL-18 polypeptide/IL-18Rα is at most 100%, at most 200%, at most 300%, at most 400%, at most 500%, or at most 600% greater than the K_{D} of wild-type IL-18/IL-18Rα. In some embodiments, the K_{D} of the modified IL-18 polypeptide/IL-18Rα is at most 500% greater than the K_{D} of wild-type IL-18/IL-18Rα. In some embodiments, the K_{D} of the modified IL-18 polypeptide/IL-18Rα is about 500% greater than the K_{D} of wild-type IL-18/IL-18Rα. In some embodiments, the K_{D} is determined by a surface plasmon resonance assay (see, e.g., Example 8 and Example 10).

In some embodiments, the K_{D} of a modified IL-18 polypeptide/IL-18Rα/β heterodimer is substantially the same as the K_{D} of wild-type IL-18/IL-18Rα/β. In some embodiments, the K_{D} of the modified IL-18 polypeptide/IL-18Rα/β heterodimer is greater than the K_{D} of wild-type IL-18/IL-18Rα/β heterodimer. In some embodiments, the K_{D} of the modified IL-18 polypeptide/IL-18Rα/β heterodimer is at most 10%, at most 20%, at most 30%, at most 40%, at most 50%, at most 60%, at most 70%, at most 80%, or at most 90% greater than the K_{D} of wild-type IL-18/IL-18Rα/β heterodimer. In some embodiments, the K_{D} of the modified IL-18 polypeptide/IL-18Rα/β heterodimer is at most 25% greater than the K_{D} of wild-type IL-18/IL-18Rα/β heterodimer. In some embodiments, the K_{D} of the modified IL-18 polypeptide/IL-18Rα/β heterodimer is about 25% greater than the K_{D} of wild-type IL-18/IL-18Rα/β heterodimer. In some embodiments, the K_{D} is determined by a surface plasmon resonance assay (see, e.g., Example 8 and Example 11).

In some embodiments, the K_{D} of the modified IL-18 polypeptide/IL-18Rα/β heterodimer is at most 100%, at most 200%, at most 300%, at most 400%, at most 500% greater than the K_{D} of wild-type IL-18/IL-18Rα/β heterodimer. In some embodiments, the K_{D} of the modified IL-18 polypeptide/IL-18Rα/β heterodimer is at most 350% greater than the K_{D} of wild-type IL-18/IL-18Rα/β heterodimer. In some embodiments, the K_{D} is determined by a surface plasmon resonance assay (see, e.g., Example 8 and Example 11).

In some embodiments, the K_{D} of a modified IL-18 polypeptide/IL-18BP is substantially the same as the K_{D} of wild-type IL-18/IL-18BP. In some embodiments, the K_{D} of the modified IL-18 polypeptide/IL-18BP is greater than the K_{D} of wild-type IL-18/IL-18BP. In some embodiments, the K_{D} of the modified IL-18 polypeptide/IL-18BP is at least 10%, at least 20%, at least 30%, at least 40%, at least 50%, at least 60%, at least 70%, at least 80%, or at least 90% greater than the K_{D} of wild-type IL-18/IL-18BP. In some embodiments, the K_{D} of the modified IL-18 polypeptide/IL-18BP is at least 25% greater than the K_{D} of wild-type IL-18/IL-18BP. In some embodiments, the K_{D} of the modified IL-18 polypeptide/IL-18BP is about 25% greater than the K_{D} of wild-type IL-18/IL-18BP. In some embodiments, the K_{D} is determined by a surface plasmon resonance assay (see, e.g., Example 8 and Example 12). In some embodiments, the K_{D} is determined by an alphaLISA assay (see, e.g., Example 9).

In some embodiments, the K_{D} of the modified IL-18 polypeptide/IL-18BP is greater than the K_{D} of wild-type IL-18/IL-18BP. In some embodiments, the K_{D} of the modified IL-18 polypeptide/IL-18BP is at least 2 times, 3 times, 5 times, 10 times, 15 times, 20 times, 30 times, 40 times, or 50 times greater than the K_{D} of wild-type IL-18/IL-18BP. In some embodiments, the K_{D} of the modified IL-18 polypeptide/IL-18BP is at least 5 times greater than the K_{D} of wild-type IL-18/IL-18BP. In some embodiments, the K_{D} of the modified IL-18 polypeptide/IL-18BP is at least 30 times greater than the K_{D} of wild-type IL-18/IL-18BP. In some embodiments, the K_{D} of the modified IL-18 polypeptide/IL-18BP is about 8 times greater than the K_{D} of wild-type IL-18/IL-18BP. In some embodiments, the K_{D} of the modified IL-18 polypeptide/IL-18BP is about 35 times greater than the K_{D} of wild-type IL-18/IL-18BP. In some embodiments, the K_{D} of the modified IL-18 polypeptide/IL-18BP is about 40 times greater than the K_{D} of wild-type IL-18/IL-18BP. In some embodiments, the K_{D} is determined by a surface plasmon resonance assay (see, e.g., Example 8 and Example 12). In some embodiments, the K_{D} is determined by an alphaLISA assay (see, e.g., Example 9).

### VIIb. Half-Maximal Effective Concentrations (EC₅₀)

In some embodiments, the modified IL-18 polypeptide modulates IFN production. In some embodiments, an EC₅₀ (nM) of the modified IL-18 polypeptide's ability to induce IFNγ is less than 10-fold higher than, less than 5-fold higher than, or less than an EC₅₀ (nM) of an IL-18 polypeptide of SEQ ID NO: 1. In some embodiments, the EC₅₀ of the modified IL-18 polypeptide's ability to induce IFNγ is less than 10-fold higher than an EC₅₀ (nM) of an IL-18 polypeptide of SEQ ID NO: 1. In some embodiments, the EC₅₀ of the modified IL-18 polypeptide's ability to induce IFNγ is less than 5-fold higher than an EC₅₀ (nM) of an IL-18 polypeptide of SEQ ID NO: 1. In some embodiments, the EC₅₀ of the modified IL-18 polypeptide's ability to induce IFNγ is less than an EC₅₀ (nM) of an IL-18 polypeptide of SEQ ID NO: 1. In some embodiments, the EC₅₀ of the modified IL-18 polypeptide's ability to induce IFNγ is less than 10-fold higher than, less than 8-fold higher than, less than 6-fold higher than, less than 5-fold higher than, less than 4-fold higher than, less than 3-fold higher than, or less than 2-fold higher than an EC₅₀ (nM) of an IL-18 polypeptide of SEQ ID NO: 1. In some embodiments, the EC₅₀ of the modified IL-18 polypeptide's ability to induce IFNγ is measured by an IFNγ induction cellular assay (see, e.g., Example 13).

In some embodiments, the modified IL-18 polypeptide modulates IFNγ production, and wherein an EC₅₀ (nM) of the modified IL-18 polypeptide against IFNγ is less than an EC₅₀ (nM) of an IL-18 polypeptide of SEQ ID NO: 1. In some embodiments, the EC₅₀ (nM) of the modified IL-18 polypeptide against IFNγ is at least 10-fold less than the EC₅₀ (nM) of an IL-18 polypeptide of SEQ ID NO: 1. In some embodiments, the EC₅₀ (nM) of the modified IL-18 polypeptide against IFNγ is about 10-fold less than the EC₅₀ (nM) of an IL-18 polypeptide of SEQ ID NO: 1. In some embodiments, the EC₅₀ (nM) of the modified IL-18 polypeptide against IFNγ is about 15-fold less than the EC₅₀ (nM) of a n IL-18 polypeptide of SEQ ID NO: 1. In some embodiments, the EC₅₀ of the modified IL-18 polypeptide's ability to induce IFNγ is measured by an IFNγ induction cellular assay (see, e.g., Example 13).

### VIII. Method of Treatment

In one aspect, described herein, is a method of treating cancer in a subject in need thereof, comprising: administering to the subject an effective amount of a modified IL-18 polypeptide or a pharmaceutical composition as described herein.

In another aspect, described herein, is a modified IL-18 polypeptide provided herein for use in treatment of cancer in a subject in need thereof. In another aspect, described herein, is a modified IL-18 polypeptide provided herein for in the manufacture of a medicament for treatment of cancer in a subject in need thereof.

In some embodiments, the cancer is a solid cancer. In some embodiments, the solid cancer is kidney cancer, skin cancer, bladder cancer, bone cancer, brain cancer, breast cancer, colorectal cancer, esophageal cancer, eye cancer, head and neck cancer, lung cancer, ovarian cancer, pancreatic cancer, or prostate cancer. In some embodiments, the solid cancer is metastatic renal cell carcinoma (metastatic RCC) or melanoma. In some embodiments, the cancer is a solid cancer. In some embodiments, the solid cancer is a carcinoma or a sarcoma.

In some embodiments, the cancer is a liquid cancer. In some embodiments, the cancer is a blood cancer. In some embodiments, the liquid cancer is a myeloma or a leukemia. In some embodiments, the liquid cancer is leukemia, non-Hodgkin lymphoma, Hodgkin lymphoma, or multiple myeloma.

In some embodiments, the modified IL-18 polypeptide is administered in a single dose of the effective amount of the modified IL-18 polypeptide, including further embodiments in which (i) the modified IL-18 polypeptide is administered once a day; or (ii) the modified IL-18 polypeptide is administered to the subject multiple times over the span of one day. In some embodiments, the modified IL-18 polypeptide is administered daily, every other day, 3 times a week, once a week, every 2 weeks, every 3 weeks, every 4 weeks, every 5 weeks, every 3 days, every 4 days, every 5 days, every 6 days, bi-weekly, 3 times a week, 4 times a week, 5 times a week, 6 times a week, once a month, twice a month, 3 times a month, once every 2 months, once every 3 months, once every 4 months, once every 5 months, or once every 6 months. In some embodiments, the modified IL-18 polypeptide is administered daily. In some embodiments, the modified IL-18 polypeptide is administered every other day. In some embodiments, the modified IL-18 polypeptide is administered every other day. In some embodiments, the modified IL-18 polypeptide is administered 3 times a week. In some embodiments, the modified IL-18 polypeptide is administered once a week. In some embodiments, the modified IL-18 polypeptide is administered every 2 weeks. In some embodiments, the modified IL-18 polypeptide is administered every 3 weeks. In some embodiments, the modified IL-18 polypeptide is administered every 4 weeks. In some embodiments, the modified IL-18 polypeptide is administered every 5 weeks. In some embodiments, the modified IL-18 polypeptide is administered every 3 days. In some embodiments, the modified IL-18 polypeptide is administered every 4 days. In some embodiments, the modified IL-18 polypeptide is administered every 5 days. In some embodiments, the modified IL-18 polypeptide is administered every 6 days. In some embodiments, the modified IL-18 polypeptide is administered bi-weekly. In some embodiments, the modified IL-18 polypeptide is administered 3 times a week. In some embodiments, the modified IL-18 polypeptide is administered 4 times a week. In some embodiments, the modified IL-18 polypeptide is administered 5 times a week. In some embodiments, the modified IL-18 polypeptide is administered 6 times a week. In some embodiments, the modified IL-18 polypeptide is administered once a month. In some embodiments, the modified IL-18 polypeptide is administered twice a month. In some embodiments, the modified IL-18 polypeptide is administered 3 times a month. In some embodiments, the modified IL-18 polypeptide is administered once every two months. In some embodiments, the modified IL-18 polypeptide is administered once every 3 months. In some embodiments, the modified IL-18 polypeptide is administered once every 4 months. In some embodiments, the modified IL-18 polypeptide is administered once every 5 months. In some embodiments, the modified IL-18 polypeptide is administered once every 6 months.

In some embodiments, the subject is 5 to 75 years old. In some embodiments, the subject is 5 to 10, 5 to 15, 5 to 18, 5 to 25, 5 to 35, 5 to 45, 5 to 55, 5 to 65, 5 to 75, 10 to 15, 10 to 18, 10 to 25, 10 to 35, 10 to 45, 10 to 55, 10 to 65, 10 to 75, 15 to 18, 15 to 25, 15 to 35, 15 to 45, 15 to 55, 15 to 65, 15 to 75, 18 to 25, 18 to 35, 18 to 45, 18 to 55, 18 to 65, 18 to 75, 25 to 35, 25 to 45, 25 to 55, 25 to 65, 25 to 75, 35 to 45, 35 to 55, 35 to 65, 35 to 75, 45 to 55, 45 to 65, 45 to 75, 55 to 65, 55 to 75, or 65 to 75 years old. In some embodiments, the subject is at least 5, 10, 15, 18, 25, 35, 45, 55, or 65 years old. In some embodiments, the subject is at most 10, 15, 18, 25, 35, 45, 55, 65, or 75 years old.

In some embodiments, the method further comprises reconstituting a lyophilized form of the modified IL-18 polypeptide or the pharmaceutical composition. In some embodiments, the modified IL-18 polypeptide or the pharmaceutical composition is reconstituted before administration. In some embodiments, the composition is reconstituted immediately before administration, up to about 5 minutes before administration, up to about 20 minutes before administration, up to about 40 minutes before administration, up to an hour before administration, or up to about four hours before administration.

### IX. Method of Manufacturing (Synthesis)

Also provided herein is a method synthesizing a modified IL-18 polypeptide. In some cases, the modified IL-18 polypeptide is synthized chemically rather than recombinantly expressed. In some instances, several fragment peptide precursors of the modified IL-18 polypeptide are synthesized and subsequently ligated together using a suitable ligation methodology (e.g., alpha-keto acid hydroxylamine (KAHA) ligation). In some cases, after ligation, the resulting modified IL-18 polypeptide is folded to produce a modified IL-18 polypeptide having a secondary and tertiary structure substantially identical to that of a recombinant or wild type IL-18 polypeptide.

In some instances, methionine residues of the modified IL-18 polypeptide are substituted for stability purposes and to aid in the folding of the linear modified IL-18 polypeptide to produce the final modified IL-18 polypeptide. The side chain of methionine is prone to oxidation during the synthesis process (e.g., peptide synthesis and protein folding), thus resulting, in some cases, in a finalized IL-18 polypeptide of insufficient quality for certain uses due to a lack of uniformity.

In some cases, in order to combat these limitations, all methionine residues of the modified IL-18 polypeptide were replaced with norleucine residues. In some cases, synthesis of the linear peptide was successful, but the resulting showed signs of instability, such as increased hydrophobicity and propensity to precipitate, and detuned biological activity, potentially because of misfolding resulting in altered secondary/tertiary structure of the modified IL-18 polypeptide relative to wild type or recombinant IL-18.

In some cases, modified IL-18 polypeptides were synthesized to directly incorporate oxidized methionine during the synthesis of the precursor peptides in an attempt to create a uniform linear protein without a complex mixture of partial methionine oxidation. In some cases, the modified linear IL-18 polypeptides were successfully synthesized, but difficulty was encountered in reducing the methionine back to the unoxidized form.

In order to combat these challenges, new modified IL-18 polypeptide variants were designed which replaced the methionine residues with O-methyl-L-homoserine (Omh) residues. Omh is a structural analog of natural methionine with the sulfur atom of methionine replaced with an oxygen. Due to the lack of the sulfur atom, Omh residues are less prone to oxidation and thus are predicted to give the modified IL-18 polypeptide greater stability and ease of synthesis/purification. Additionally, the increased hydrophilicity of the Omh residue compared to norleucine residues, along Omh's greater structural homology to the native methionine residues, is predicted to facilitate proper folding and greater stability of the modified IL-18 polypeptide as compared to a variant with norleucine residues in place of the methionines. Thus, it is predicted that a chemically synthesized modified IL-18 polypeptide which replaces methionine residues with Omh residues will provide several advantages over other synthesized modified IL-18 polypeptides.

In one aspect, described herein, is a method of making a modified IL-18 polypeptide. In another aspect, described herein, is a method of making a modified IL-18 polypeptide comprising synthesizing two or more fragments of the modified IL-18 polypeptide and ligating the fragments. In another aspect, described herein, is a method of making a modified IL-18 polypeptide comprising a. synthesizing two or more fragments of the modified IL-18 polypeptide, b. ligating the fragments; and c. folding the ligated fragments. In another aspect, described herein, is a method of making a modified IL-18 polypeptide comprising providing two or more fragments of the modified IL-18 polypeptide and ligating the fragments. In another aspect, described herein, is a method of making a modified IL-18 polypeptide comprising a. providing two or more fragments of the modified IL-18 polypeptide, b. ligating the fragments; and c. folding the ligated fragments. In another aspect, described herein, is a method of making a modified IL-18 polypeptide comprising ligating two or more fragments of the modified IL-18 polypeptide, wherein at least one the two or more fragments of the modified IL-18 polypeptide are synthesized, and folding the ligated fragments.

In some embodiments, the two or more fragments of the modified IL-18 polypeptide are synthesized chemically. In some embodiments, the two or more fragments of the modified IL-18 polypeptide are synthesized by solid phase peptide synthesis. In some embodiments, the two or more fragments of the modified IL-18 polypeptide are synthesized on an automated peptide synthesizer.

In some embodiments, the modified IL-18 polypeptide is ligated from 2, 3, 4, 5, 6, 7, 8, 9, 10, or more peptide fragments. In some embodiments, the modified peptide is ligated from 2 peptide fragments. In some embodiments, the modified IL-18 polypeptide is ligated from 3 peptide fragments. In some embodiments, the modified IL-18 polypeptide is ligated from 4 peptide fragments. In some embodiments, the modified IL-18 polypeptide is ligated from 2 to 10 peptide fragments.

In some embodiments, the two or more fragments comprise an N-terminal fragment, a C-terminal fragment, and optionally one or more interior fragments, wherein the N-terminal fragment comprises the N-terminus of the modified IL-18 polypeptide and the C-terminal fragment comprises the C-terminus of the modified IL-18 polypeptide. In some embodiments, each of the N-terminal fragment and the one or more interior fragments comprise an alpha-keto amino acid as the C-terminal residue of each fragment. In some embodiments, each alpha-keto amino acid is selected from alpha-keto-phenylalanine, alpha-keto-tyrosine, alpha-keto-valine, alpha-keto-leucine, alpha-keto-isoleucine, alpha-keto-norleucine, and alpha-keto-O-methylhomoserine.

In some embodiments, each of the C-terminal fragment and the one or more interior fragments comprise a residue having a hydroxylamine or a cyclic hydroxylamine functionality as the N-terminal residue of each fragment. In some embodiments, each residue having the hydroxylamine or the cyclic hydroxylamine functionality is a 5-oxaproline residue.

In some embodiments, the two or more fragments of the modified IL-18 polypeptide are ligated together. In some embodiments, three or more fragments of the modified IL-18 polypeptide are ligated in a sequential fashion. In some embodiments, three or more fragments of the modified IL-18 polypeptide are ligated in a one-pot reaction.

In some embodiments, synthesizing two or more fragments of the modified IL-18 polypeptide comprises synthesizing four fragments. In some embodiments, providing two or more fragments of the modified IL-18 polypeptide comprises providing four fragments. In some embodiments, the four fragments include four fragments each having at least about 80% sequence identity to any sequence independently selected from those provided in Table 13. In some embodiments, the four fragments include four fragments having at least about 85% sequence identity to those provided in Table 13. In some embodiments, the four fragments include four fragments having at least about 90% sequence identity to those provided in Table 13. In some embodiments, the four fragments include four fragments having at least about 95% sequence identity to those provided in Table 13. In some embodiments, the four fragments include four fragments provided in Table 13.

TABLE 13
Exemplary Peptides used to synthesize IL-18

| SEQ | | | |
|---|---|---|---|
| ID | | | |
| NO | Comment | Sequence | Key |
| 201 | Peptide 1 (1-30) | YFGKLESKLS VIRNLNDQVL FIDQGNRPL(Akf) | Akf = alpha-keto- |
| | | | phenylalanine |
| 202 | Peptide 1 E6K | YFGKLKSKLS VIRNLNDQVL FIDQGNRPL(Akf) | Akf = alpha-keto- |
| | | | phenylalanine |
| 203 | Peptide 1 Y1G | GFGKLESKLS VIRNLNDQVL FIDQGNRPL(Akf) | Akf = alpha-keto- |
| | | | phenylalanine |
| 204 | Peptide 1 F2A | YAGKLESKLS VIRNLNDQVL FIDQGNRPL(Akf) | Akf = alpha-keto- |
| | | | phenylalanine |
| 205 | Peptide 1 Y1G E6K | GFGKLKSKLS VIRNLNDQVL FIDQGNRPL(Akf) | Akf = alpha-keto- |
| | | | phenylalanine |
| 206 | Peptide 1 F2A E6K | YAGKLKSKLS VIRNLNDQVL FIDQGNRPL(Akf) | Akf = alpha-keto- |
| | | | phenylalanine |
| 207 | Peptide 1 Y1G F2A | GAGKLKSKLS VIRNLNDQVL FIDQGNRPL(Akf) | Akf = alpha-keto- |
| | E6K | | phenylalanine |
| 208 | Peptide 1 Y1G F2A | GAGKLESKLS VIRNLNDQVL FIDQGNRPL(Akf) | Akf = alpha-keto- |
| | | | phenylalanine |
| 210 | Peptide 2A (31-62) | (Opr)DMTDSDCRD NAPRTIFIIS | Opr = 5-oxa-proline; |
| | | MYKDSQPRGM A(Akv) | Akv = alpha-keto- |
| | | | valine |
| 211 | Peptide 2A M33X, | (Opr)DXTDSDCRD NAPRTIFIIS | Opr = 5-oxa-proline; |
| | M51X, M60X | XYKDSQPRGX A(Akv) | Akv = alpha-keto- |
| | | | valine |
| 212 | Peptide 2A M33J, | (Opr)DJTDSDCRD NAPRTIFIIS | Opr = 5-oxa-proline; |
| | M51J, M60J | JYKDSQPRGJ A(Akv) | Akv = alpha-keto- |
| | | | valine |
| 213 | Peptide 2A K53A | (Opr)DMTDSDCRD NAPRTIFIIS | Opr = 5-oxa-proline; |
| | | MYADSQPRGM A(Akv) | Akv = alpha-keto- |
| | | | valine |
| 214 | Peptide 2A M33X, | (Opr)DXTDSDCRD NAPRTIFIIS | Opr = 5-oxa-proline; |
| | M51X, K53A, M60X | XYADSQPRGX A(Akv) | Akv = alpha-keto- |
| | | | valine |
| 215 | Peptide 2A M33J, | (Opr)DJTDSDCRD NAPRTIFIIS | Opr = 5-oxa-proline; |
| | M51J, K53A, M60J | JYADSQPRGJ A(Akv) | Akv = alpha-keto- |
| | | | valine |
| 216 | Peptide 2A M33J, | (Opr)DJTDSDSRD NAPRTIFIIS | Opr = 5-oxa-proline; |
| | C38S, M51J, K53A, | JYADSQPRGJ A(Akv) | Akv = alpha-keto- |
| | M60J | | valine |
| 217 | Peptide 2A M33J, | (Opr)DJTDSDSRD NAPRTIFIIS | Opr = 5-oxa-proline; |
| | C38S, M51J, M60J | JYKDSQPRGJ A(Akv) | Akv = alpha-keto- |
| | | | valine |
| 218 | Peptide 2B (31-74) | (Opr)DMTDSDCRD NAPRTIFIIS | Opr = 5-oxa-proline; |
| | | MYKDSQPRGM AVTISVKCEK | Akl = alpha-keto- |
| | | IST(Akl) | leucine |
| 219 | Peptide 2B K53A | (Opr)DMTDSDCRD NAPRTIFIIS | Opr = 5-oxa-proline; |
| | | MYADSQPRGM AVTISVKCEK | Akl = alpha-keto- |
| | | IST(Akl) | leucine |
| 220 | Peptide 2B K53A, | (Opr)DMTDSDCRD NAPRTIFIIS | Opr = 5-oxa-proline; |
| | T63A | MYADSQPRGM AVTISVKCEK | Akl = alpha-keto- |
| | | IST(Akl) | leucine |
| 221 | Peptide 2B M33X, | (Opr)DXTDSDCRD NAPRTIFIIS | Opr = 5-oxa-proline; |
| | M51X, M60X | XYKDSQPRGX AVTISVKCEK | Akl = alpha-keto- |
| | | IST (Akl) | leucine |
| 222 | Peptide 2B M33J, | (Opr)DJTDSDCRD NAPRTIFIIS | Opr = 5-oxa-proline; |
| | M51J, M60J | JYKDSQPRGJ AVTISVKCEK | Akl = alpha-keto- |
| | | IST (Akl) | leucine |
| 223 | Peptide 2B M33J, | (Opr)DJTDSDCRD NAPRTIFIIS | Opr = 5-oxa-proline; |
| | M51J, K53A, M60J | JYADSQPRGJ AVTISVKCEK | Akl = alpha-keto- |
| | | IST(Akl) | leucine |
| 224 | Peptide 2B M33J, | (Opr)DJTDSDCRD NAPRTIFIIS | Opr = 5-oxa-proline; |
| | M51J, K53A, M60J, | JYADSQPRGJ AVAISVKCEK | Akl = alpha-keto- |
| | T63A | IST(Akl) | leucine |
| 225 | Peptide 2B M33J, | (Opr)DJTDSDSRD NAPRTIFIIS | Opr = 5-oxa-proline; |
| | C38S, M51J, K53A, | JYADSQPRGJ AVAISVKCEK | Akl = alpha-keto- |
| | M60J, T63A | IST(Akl) | leucine |
| 226 | Peptide 2B C68S | (Opr)DMTDSDCRD NAPRTIFIIS | Opr = 5-oxa-proline; |
| | | MYKDSQPRGM AVTISVKSEK | Akl = alpha-keto- |
| | | IST(Akl) | leucine |
| 227 | Peptide 3A (63-115) | (Opr)ISVKCEK ISTLSCENKI | Opr = 5-oxa-proline; |
| | | ISFKEMNPPD NIKDTKSDII | Akf = alpha-keto- |
| | | FFQRSVPGHD NKMQ(Akf) | phenylalanine |
| 228 | Peptide 3A M86X, | (Opr)ISVKCEK ISTLSCENKI | Opr = 5-oxa-proline; |
| | M113X | ISFKEXNPPD NIKDTKSDII | Akf = alpha-keto- |
| | | FFQRSVPGHD NKXQ(Akf) | phenylalanine |
| 229 | Peptide 3A M86J, | (Opr)ISVKCEK ISTLSCENKI | Opr = 5-oxa-proline; |
| | M113J | ISFKEJNPPD NIKDTKSDII | Akf = alpha-keto- |
| | | FFQRSVPGHD NKJQ(Akf) | phenylalanine |
| 230 | Peptide 3A C68S | (Opr)ISVKSEK ISTLSCENKI | Opr = 5-oxa-proline; |
| | | ISFKEMNPPD NIKDTKSDII | Akf = alpha-keto- |
| | | FFQRSVPGHD NKMQ(Akf) | phenylalanine |
| 231 | Peptide 3A C68S, | (Opr)ISVKSEK ISTLSCENKI | Opr = 5-oxa-proline; |
| | M86X, M113X | ISFKEXNPPD NIKDTKSDII | Akf = alpha-keto- |
| | | FFQRSVPGHD NKXQ(Akf) | phenylalanine |
| 232 | Peptide 3A C68S, | (Opr)ISVKSEK ISTLSCENKI | Opr = 5-oxa-proline; |
| | M86J, M113J | ISFKEJNPPD NIKDTKSDII | Akf = alpha-keto- |
| | | FFQRSVPGHD NKJQ(Akf) | phenylalanine |
| 233 | Peptide 3A C68S, | (Opr)ISVKSEK ISTLSSENKI | Opr = 5-oxa-proline; |
| | C76S | ISFKEMNPPD NIKDTKSDII | Akf = alpha-keto- |
| | | FFQRSVPGHD NKMQ(Akf) | phenylalanine |
| 234 | Peptide 3A C76S | (Opr)ISVKCEK ISTLSSENKI | Opr = 5-oxa-proline; |
| | | ISFKEMNPPD NIKDTKSDII | Akf = alpha-keto- |
| | | FFQRSVPGHD NKMQ(Akf) | phenylalanine |
| 235 | Peptide 3A C76S, | (Opr)ISVKCEK ISTLSSENKI | Opr = 5-oxa-proline; |
| | M86J, M113J | ISFKEJNPPD NIKDTKSDII | Akf = alpha-keto- |
| | | FFQRSVPGHD NKJQ(Akf) | phenylalanine |
| 236 | Peptide 3A C68S, | (Opr)ISVKSEK ISTLSSENKI | Opr = 5-oxa-proline; |
| | C76S, M86J, M113J | ISFKEJNPPD NIKDTKSDII | Akf = alpha-keto- |
| | | FFQRSVPGHD NKJQ(Akf) | phenylalanine |
| 237 | Peptide 3B (75-115) | (Opr)CENKI ISFKEMNPPD | Opr = 5-oxa-proline; |
| | | NIKDTKSDII FFQRSVPGHD | Akf = alpha-keto- |
| | | NKMQ(Akf) | phenylalanine |
| 238 | Peptide 3B C76S | (Opr)SENKI ISFKEMNPPD | Opr = 5-oxa-proline; |
| | | NIKDTKSDII FFQRSVPGHD | Akf = alpha-keto- |
| | | NKMQ(Akf) | phenylalanine |
| 239 | Peptide 3B M86X, | (Opr)CENKI ISFKEXNPPD | Opr = 5-oxa-proline; |
| | M113X | NIKDTKSDII FFQRSVPGHD | Akf = alpha-keto- |
| | | NKXQ(Akf) | phenylalanine |
| 240 | Peptide 3B C76S, | (Opr)SENKI ISFKEXNPPD | Opr = 5-oxa-proline; |
| | M86X, M113X | NIKDTKSDII FFQRSVPGHD | Akf = alpha-keto- |
| | | NKXQ (Akf) | phenylalanine |
| 241 | Peptide 3B M86J, | (Opr)CENKI ISFKEJNPPD | Opr = 5-oxa-proline; |
| | M113J | NIKDTKSDII FFQRSVPGHD | Akf = alpha-keto- |
| | | NKJQ(Akf) | phenylalanine |
| 242 | Peptide 3B C76S, | (Opr)SENKI ISFKEJNPPD | Opr =5-oxa-proline; |
| | M86J, M113J | NIKDTKSDII FFQRSVPGHD | Akf = alpha-keto- |
| | | NKJQ(Akf) | phenylalanine |
| 243 | Peptide 4 (116-157) | (Opr)SSSY EGYFLACEKE | Opr = 5-oxaproline |
| | | RDLFKLILKK EDELGDRSIM | |
| | | FTVQNED | |
| 244 | Peptide 4 C127S | (Opr)SSSY EGYFLASEKE | Opr = 5-oxaproline |
| | | RDLFKLILKK EDELGDRSIM | |
| | | FTVQNED | |
| 245 | Peptide 4 M150X | (Opr)SSSY EGYFLACEKE | Opr = 5-oxaproline |
| | | RDLFKLILKK EDELGDRSIX | |
| | | FTVQNED | |
| 246 | Peptide 4 C127S, | (Opr)SSSY EGYFLASEKE | Opr = 5-oxaproline |
| | M150X | RDLFKLILKK EDELGDRSIX | |
| | | FTVQNED | |
| 247 | Peptide 4 M150J | (Opr)SSSY EGYFLACEKE | Opr = 5-oxaproline |
| | | RDLFKLILKK EDELGDRSIJ | |
| | | FTVQNED | |
| 248 | Peptide 4 C127S, | (Opr)SSSY EGYFLASEKE | Opr = 5-oxaproline |
| | M150J | RDLFKLILKK EDELGDRSIJ | |
| | | FTVQNED | |

Table 13 - X = Norleucine; J = O-methyl-L-homoserine

In some embodiments, the four fragments comprise an N-terminal fragment, a first interior fragment, a second interior fragment, and a C-terminal fragment.

In some embodiments, the N-terminal fragment comprises residues which correspond to amino acids 1-30 of the modified IL-18 polypeptide, wherein residue position numbering of the modified IL-18 polypeptide is based on SEQ ID NO: 1 as a reference sequence. In some embodiments, the N-terminal fragment comprises an N-terminal extension as compared to the sequence of SEQ ID NO: 1. In some embodiments, the N-terminal fragment comprises an amino acid sequence having at least 80% sequence identity with the amino acid sequence as set forth in SEQ ID NO: 201. In some embodiments, the N-terminal fragment comprises an amino acid sequence as set forth in any one of SEQ ID Nos: 201-209.

In some embodiments, the first interior fragment comprises residues which correspond to amino acids 31-62 of the modified IL-18 polypeptide, wherein residue position numbering of the modified IL-18 polypeptide is based on SEQ ID NO: 1 as a reference sequence. In some embodiments, the first interior fragment comprises an amino acid sequence having at least 80% sequence identity with the amino acid sequence as set forth in SEQ ID NO: 210. In some embodiments, the first interior fragment comprises an amino acid sequence as set forth in any one of SEQ ID Nos: 210-217.

In some embodiments, the second interior fragment comprises residues which correspond to amino acids 63-115 of the modified IL-18 polypeptide, wherein residue position numbering of the modified IL-18 polypeptide is based on SEQ ID NO: 1 as a reference sequence. In some embodiments, the second interior fragment comprises an amino acid sequence having at least 80% sequence identity with the amino acid sequence as set forth in SEQ ID NO: 227. In some embodiments, the second interior fragment comprises an amino acid sequence as set forth in any one of SEQ ID NOs: 227-236.

In some embodiments, the first interior fragment comprises residues which correspond to amino acids 31-74 of the modified IL-18 polypeptide, wherein residue position numbering of the modified IL-18 polypeptide is based on SEQ ID NO: 1 as a reference sequence. In some embodiments, the first interior fragment comprises an amino acid sequence having at least 80% sequence identity with the amino acid sequence as set forth in SEQ ID NO: 218. In some embodiments, the first interior fragment comprises an amino acid sequence as set forth in any one of SEQ ID NOs: 218-226.

In some embodiments, the second interior fragment comprises residues which correspond to amino acids 75-115 of the modified IL-18 polypeptide, wherein residue position numbering of the modified IL-18 polypeptide is based on SEQ ID NO: 1 as a reference sequence. In some embodiments, the second interior fragment comprises an amino acid sequence having at least 80% sequence identity with the amino acid sequence as set forth in SEQ ID NO: 237. In some embodiments, the second interior fragment comprises an amino acid sequence as set forth in any one of SEQ ID NOs: 237-242.

In some embodiments, the C-terminal fragment comprises residues which correspond to amino acids 116-157 of the modified IL-18 polypeptide, wherein residue position numbering of the modified IL-18 polypeptide is based on SEQ ID NO: 1 as a reference sequence. In some embodiments, the C-terminal fragment comprises an amino acid sequence having at least 80% sequence identity with the amino acid sequence as set forth in SEQ ID NO: 243. In some embodiments, the C-terminal fragment comprises an amino acid sequence as set forth in any one of SEQ ID NOs: 243-248.

In some embodiments, the N-terminal fragment, the first interior fragment, the second interior fragment, and the C-terminal fragment are arranged from the N-terminus to the C-terminus, respectively, in the modified IL-18 polypeptide.

In some embodiments, the method further comprises rearranging the ligated fragments. In some embodiments, rearranging the ligated fragments involves rearranging one or more depsipeptide bonds of the linear IL-18 polypeptide. In some embodiments, the one or more depsipeptide bonds are rearranged to form one or more amide bonds. In some embodiments, the depsipeptide bonds are formed as a result of the ligation of the fragments. In some embodiments, the depsipeptide bonds are between the hydroxyl moiety of a homoserine residue and an amino acid adjacent to the homoserine residue. In some embodiments, rearranging the ligated fragments occurs after each of the fragments have been ligated.

In some embodiments, ligated fragments are folded. In some embodiments, folding comprises forming one or more disulfide bonds within the modified IL-18 polypeptide. In some embodiments, the ligated fragments are subjected to a folding process. In some embodiments, the ligated fragments are folded using methods well known in the art. In some embodiments, the ligated polypeptide or the folded polypeptide are further modified by attaching one or more polymers thereto. In some embodiments, the ligated polypeptide or the folded polypeptide are further modified by PEGylation.

In some embodiments, the modified IL-18 polypeptide is synthetic.

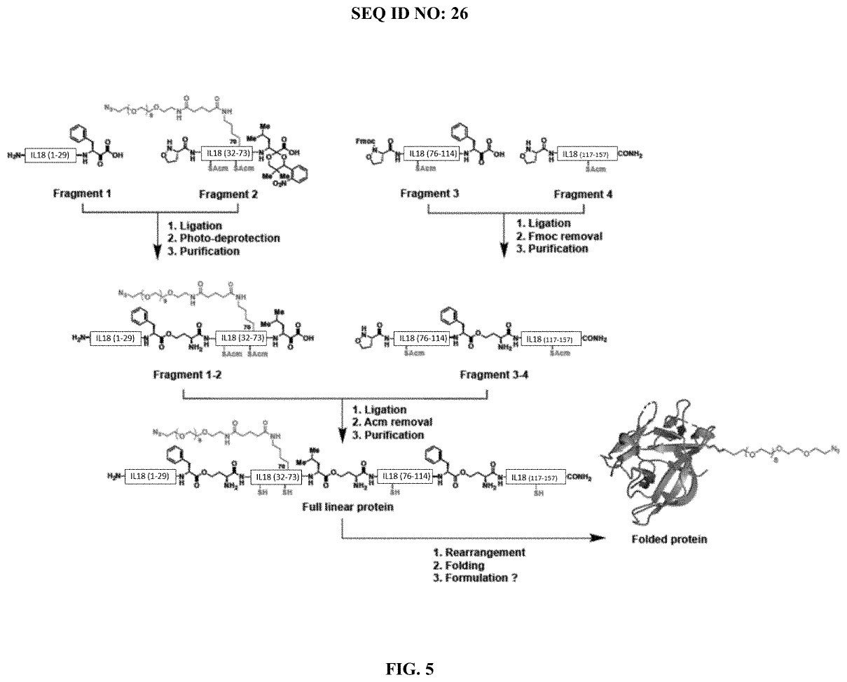

Exemplary, non-limiting synthetic schemes of particular modified IL-18 polypeptides provided herein are shown in FIGS. 5 and 9-17. In general, in some embodiments, a first fragment (“Segment 1”) containing amino acids or amino acid precursors corresponding to residue numbers 1-30 of the modified IL-18 polypeptide is prepared (e.g., by solid phase peptide synthesis (SPPS)), as compared to the amino acid sequence set for in SEQ ID NO: 1. This is coupled to a second fragment (“Segment 2”) containing, in some embodiments, amino acids or amino precursors corresponding to either residue numbers 31-74 or residue numbers 31-62 of the modified IL-18 polypeptide to produce a single fragment (“Segment 12”). This second fragment is in some embodiments also prepared by SPPS. Similarly, a third fragment is prepared, in some embodiments by SPPS, having amino acids or amino acid precursors corresponding to either residue numbers 63-115 or 75-115 of the modified IL-18 polypeptide. This third fragment is coupled to a fourth fragment (“Segment 4”), in some embodiments prepared by SPPS, which contains amino acids or amino acid precursors corresponding to residue numbers 116-157 of the modified IL-18 polypeptide to produce a single fragment (“Segment 34”). Segment 12 and Segment 34 are then coupled to produce a full length fragment (“Segment 1234”). In embodiments where KAHA ligation is used to ligate the fragments, the site residues are then rearranged to produce amide bonds at the ligation points (e.g., depsipeptide homoserine rearrangement to amide bond). Finally, the full length linear fragment is then folded to produce a synthetic IL-18 polypeptide.

FIG. 5 shows an exemplary synthetic scheme for a synthesis of a modified IL-18 polypeptide having an amino acid sequence as set forth in SEQ ID NO: 26. This modified IL-18 polypeptide incorporates an azide functionality appended to residue K70 via a PEG linker in the synthesis of Fragment 2. This azide functionality later acts as a conjugation handle to attach a larger PEG group.

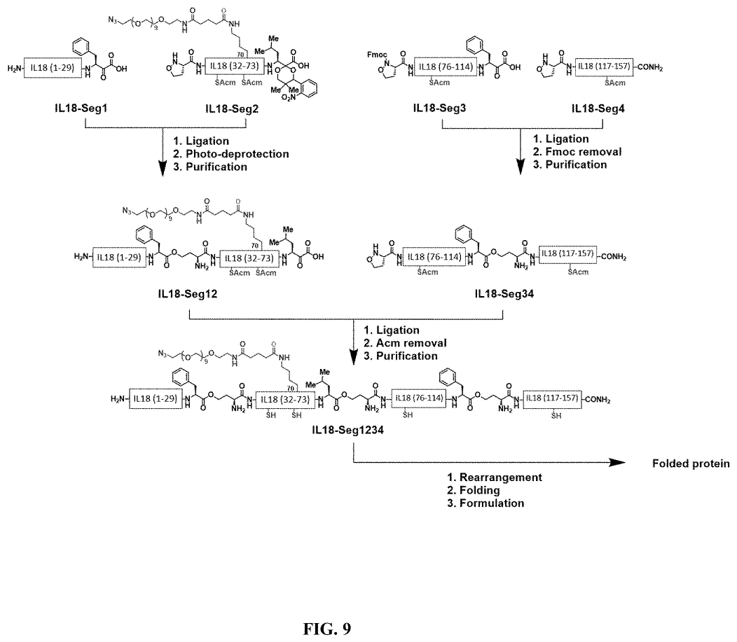

FIG. 9 shows an exemplary synthetic scheme for a synthesis of a modified IL-18 polypeptide. The synthesis depicted in this figure also incorporates an azide-bearing PEG lysine residue at position 70, similar to FIG. 5.

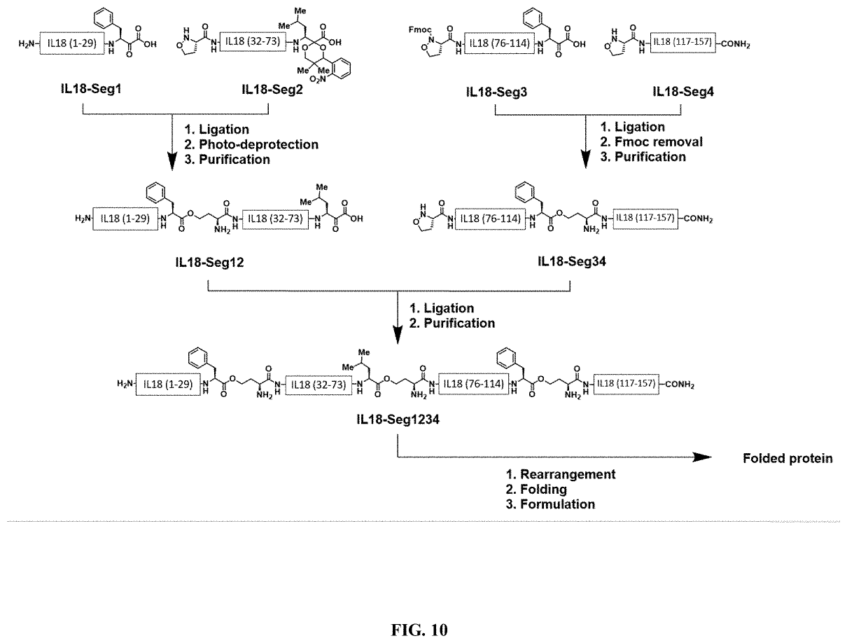

FIG. 10 shows an additional exemplary synthetic scheme for a synthesis of a modified IL-18 polypeptide. The modified IL-18 polypeptide has an amino acid sequence as set forth in SEQ ID NO: 25. The IL-18 polypeptide depicted contains no cysteine residues and is not modified to incorporate a conjugation handle, and thus is incompetent for site-specific PEGylation using the techniques provided herein.

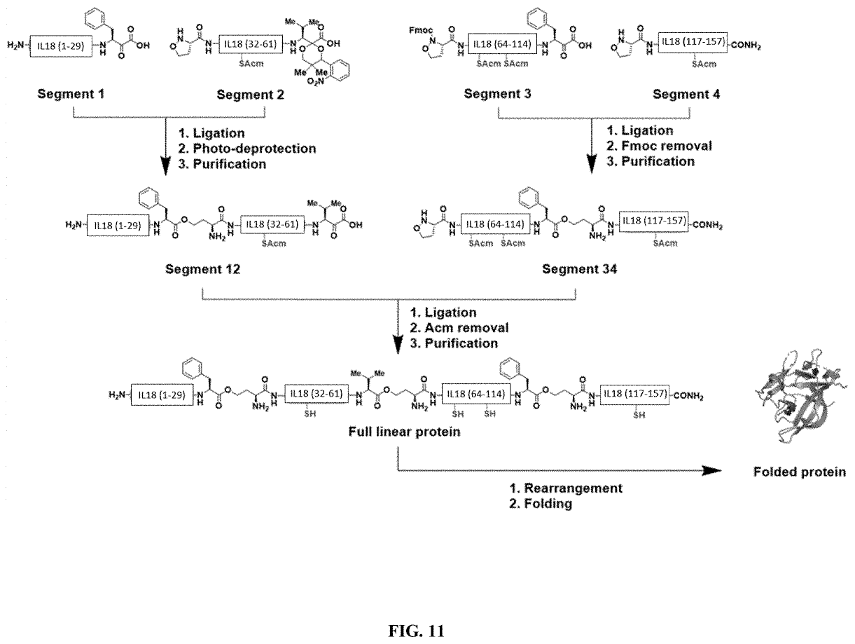

FIG. 11 shows an exemplary synthetic scheme for a synthesis of a modified IL-18 polypeptide having an amino acid sequence as set forth in SEQ ID NO: 31. Compared to the syntheses set forth in FIGS. 5, 9, and 10, the modified IL-18 is ligated at position 62/63 rather than 74/75.

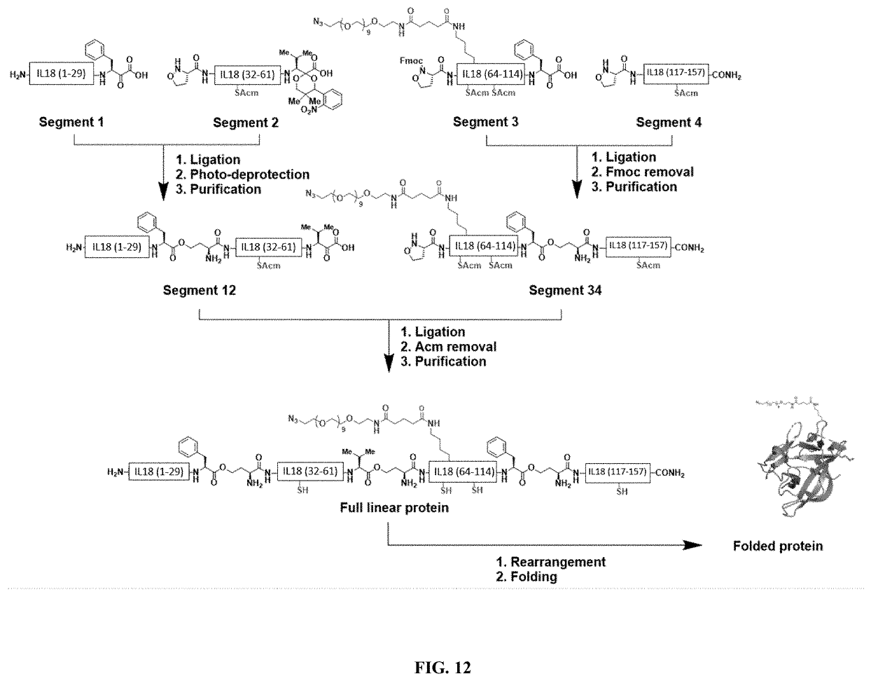

FIG. 12 shows an exemplary synthetic scheme for a synthesis of a modified IL-18 polypeptide having an amino acid sequence as set forth in SEQ ID NO: 32. The modified IL-18 polypeptide comprises a modified lysine residue bearing an azide functionality.

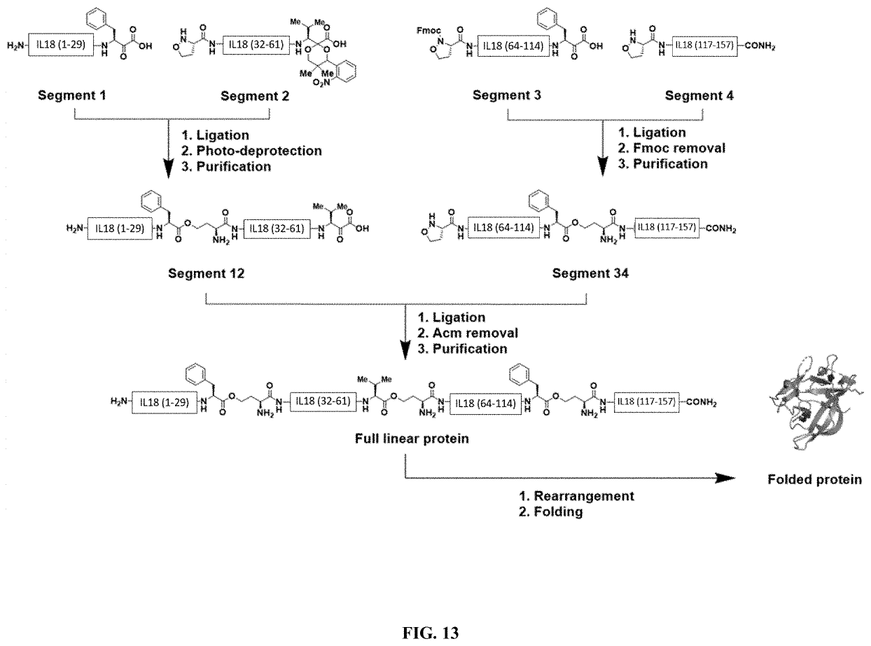

FIG. 13 shows an exemplary synthetic scheme for a synthesis of a modified IL-18 polypeptide having an amino acid sequence as set forth in SEQ ID NO: 33. The IL-18 polypeptide depicted contains no cysteine residues and is not modified to incorporate a conjugation handle, and thus is incompetent for site-specific PEGylation using the techniques provided herein.

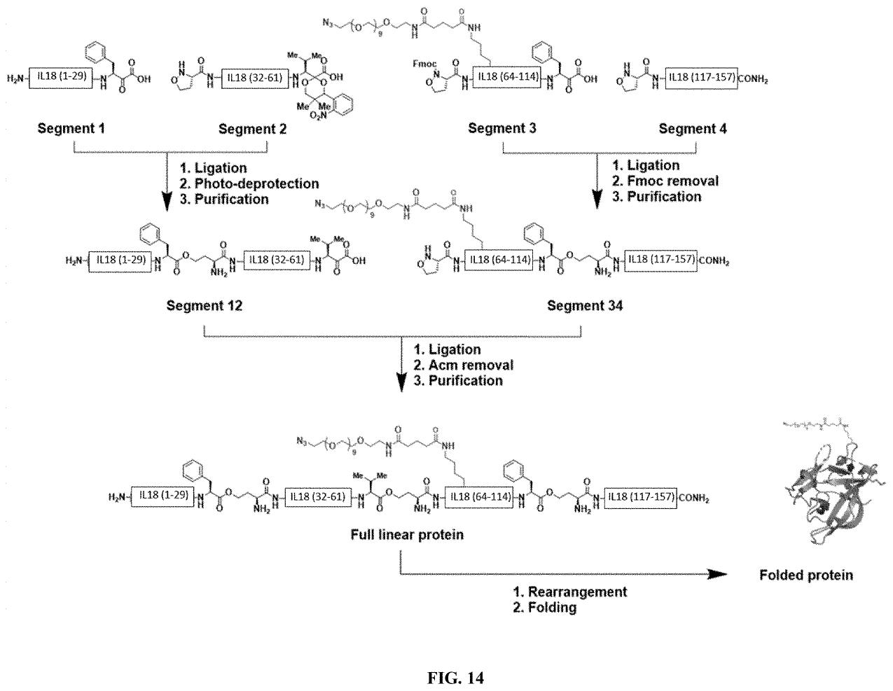

FIG. 14 shows an exemplary synthetic scheme for a synthesis of a modified IL-18 polypeptide having an amino acid sequence as set forth in SEQ ID NO: 34. The modified IL-18 polypeptide comprises a modified lysine residue bearing an azide functionality.

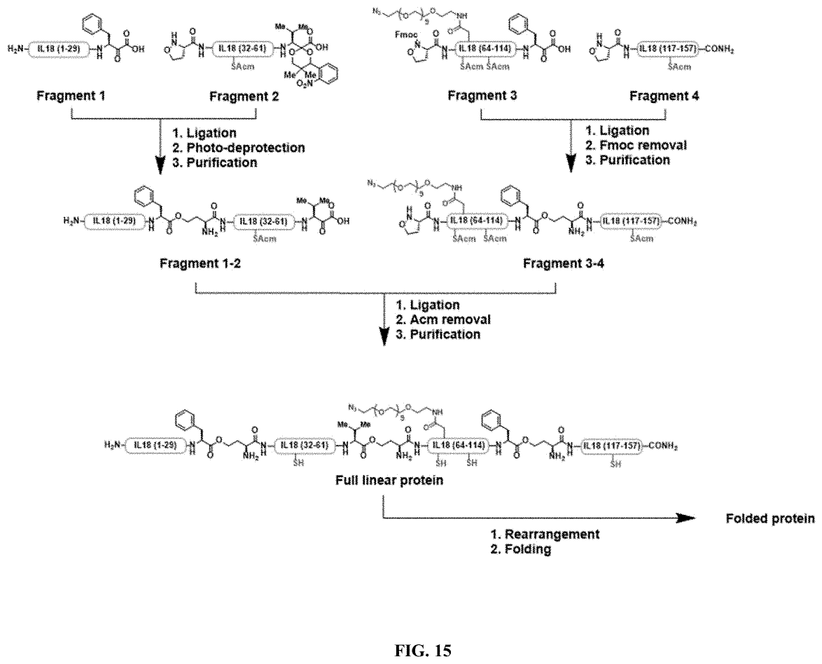

FIG. 15 shows an exemplary synthetic scheme for a synthesis of a modified IL-18 polypeptide which comprises a modified aspartate residue which has an azide moiety appended to it via a PEG group. The modified aspartate residue can be placed at any desired positions (e.g., residue 68, 69, or 70).

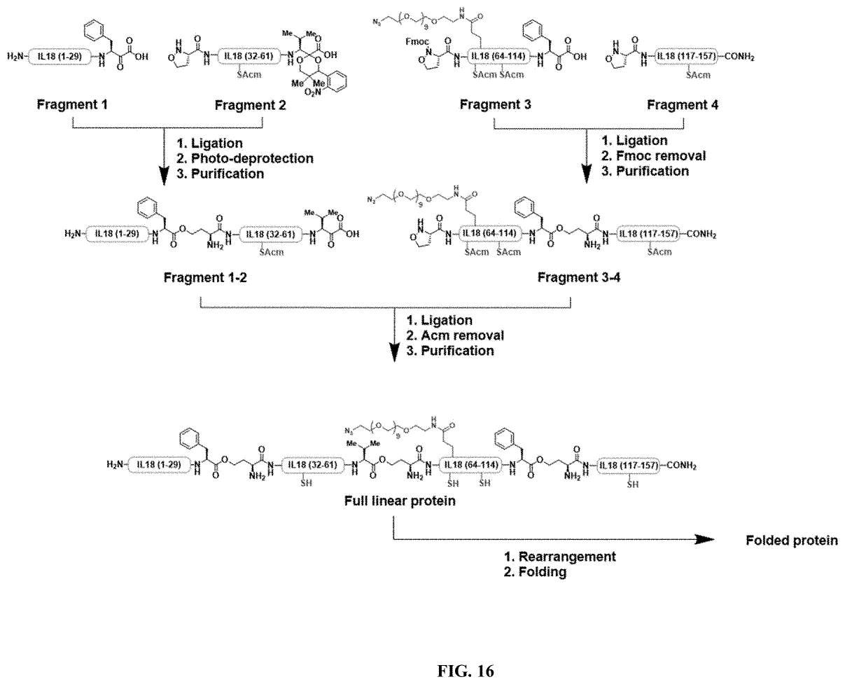

FIG. 16 shows an exemplary synthetic scheme for a synthesis of a modified IL-18 polypeptide which comprises a modified glutamate residue which has an azide moiety appended to it via a PEG group. The modified glutamate residue can be placed at any desired positions (e.g., residue 68, 69, or 70).

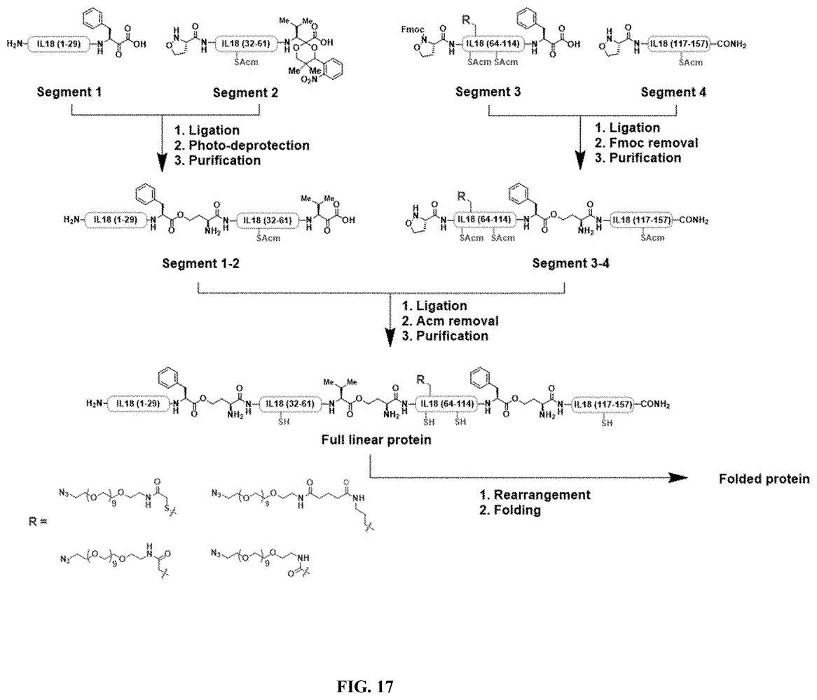

FIG. 17 shows a generic synthetic scheme used for the preparation of a modified IL-18 polypeptide which comprises an azide group appended to an amino acid residue on segment 3 (e.g., residue 68, 69, or 70). The R group shown indicates that the azide functionality can be attached through a variety of modified residues, including cysteine, lysine, aspartate, and glutamate.

In some embodiments, the modified IL-18 polypeptides are expressed as recombinant polypeptides. In some embodiments, the modified IL-18 polypeptides are expressed using Escherichia coli.

Although the present disclosure and its advantages have been described in detail, it should be understood that various changes, substitutions and alterations can be made herein without departing from the spirit and scope of the disclosure as defined in the appended claims.

### X. Synthetic IL-18

Also provided herein are chemically synthesized IL-18s. In some embodiments, the chemically synthesized IL-18s display a biological activity substantially identical to a recombinant IL-18 of SEQ ID NO: 1. In some embodiments, the chemically synthesized IL-18s contain modifications as provided herein. In some embodiments, the modifications provided herein modulate the biological activity of the modified IL-18 polypeptide as provided herein.

Chemically synthesized IL-18 provides advantages over recombinant IL-18 because it can be synthesized to include any desired modification with ease in a site-specific manner, allowing ready modulation of the biological activity.

In one aspect, provided herein, is a synthetic IL-18 polypeptide, comprising a synthetic IL-18 polypeptide comprising a homoserine (Hse) residue at a position selected from the region of residues 21-41, residues 60-80, and residues 106-126, wherein residue position numbering of the modified IL-18 polypeptide is based on SEQ ID NO: 1 as a reference sequence. In some embodiments, the synthetic IL-18 polypeptide comprises a Hse residue in each of the regions of residues 21-41, residues 60-80, and residues 106-126.

In some embodiments, the synthetic IL-18 polypeptide comprises a Hse residue at position 31. In some embodiments, the synthetic IL-18 polypeptide comprises a Hse residue at position 63 or position 75. In some embodiments, the synthetic IL-18 polypeptide comprises a Hse residue at position 63. In some embodiments, the synthetic IL-18 polypeptide comprises a Hse residue at position 75. In some embodiments, the synthetic IL-18 polypeptide comprises a Hse residue at position 116. In some embodiments, the synthetic IL-18 polypeptide comprises Hse residues at positions 31, 116, and at least one of positions 63 and 75.

In some embodiments, the synthetic IL-18 polypeptide comprises an amino acid substitution of at least one methionine residue in SEQ ID NO: 1. In some embodiments, the amino acid substitution of at least one methionine residue in SEQ ID NO: 1 comprises a substitution at M33, M51, M60, M86, M113, or M150. In some embodiments, the synthetic IL-18 polypeptide comprises substitutions of at least three methionine residues. In some embodiments, the synthetic IL-18 polypeptide comprises substitutions of at least five methionine residues. In some embodiments, the synthetic IL-18 polypeptide comprises substitution of at least six methionine residues.

In some embodiments, at least one methionine residue is substituted for an O-methyl-homoserine (Omh) residue. In some embodiments, at least three methionine residues are substituted for Omh residues. In some embodiments, at least five methionine residues are substituted for Omh residues. In some embodiments, each methionine substitution is for a norleucine or Omh residue. In some embodiments, each methionine substitution is for an Omh residue. In some embodiments, each methionine residue of SEQ ID NO: 1 is substituted for an Omh residue.

In some embodiments, the synthetic IL-18 polypeptide comprises an additional mutation to SEQ ID NO: 1. In some embodiments, the synthetic IL-18 polypeptide comprises an amino acid sequence at least about 80% identical to that of SEQ ID NO: 1. In some embodiments, the synthetic IL-18 polypeptide comprises a polymer covalently attached to a residue of the synthetic IL-18 polypeptide.

The present disclosure is further illustrated in the following Examples which are given for illustration purposes only and are not intended to limit the disclosure in any way.

### EXAMPLES

### Example 1—Synthesis of Modified IL-18 Polypeptides

A modified IL-18 polypeptide having an amino acid sequence of SEQ ID NO: 7, was prepared by ligating individual peptides synthesized using solid phase peptide synthesis (SPPS). Individual peptides were synthesized on an automated peptide synthesizer using the methods described below.

Commercially available reagents were purchased from Sigma-Aldrich, Acros, Merck or TCI Europe and used without further purification. Fluorenylmethoxycarbonyl (Fmoc) amino acids with suitable side-chain protecting groups for solid phase peptide synthesis were purchased from Novabiochem, Christof Senn Laboratories AG or PeptART and used as supplied. The polyethylene glycol derivatives used for peptide synthesis were purchased by Polypure. HPLC grade CH₃CN from Sigma Aldrich was used for analytical and preparative HPLC purification.

Peptides and proteins were characterized by high resolution Fourier-transform mass spectrometry (FTMS) using a Bruker solariX (9.4T magnet) spectrometer equipped with a dual ESI/MALDI-FTICR source using 4-hydroxy-α-cyanocinnamic acid (HCCA) as matrix. CD spectra were recorded with a Jasco J-715 spectrometer with a 1.0 mm path length cell. CD spectra were collected at 25° C. in continuous scanning mode with standard sensitivity (100 mdeg), 0.5 nm data pitch, 50 nm/min scanning speed and 1 nm bandwidth. CD curves were obtained by averaging 5 scans and subtracting the background signal.

Peptide segments, ligated peptides, and linear proteins were analyzed and purified by reverse phase high performance liquid chromatography (RP-HPLC). The peptide analysis and reaction monitoring were performed on analytical Jasco instruments with dual pumps, mixer and in-line degasser, autosampler, a variable wavelength UV detector (simultaneous monitoring of the eluent at 220 nm and 254 nm), and an injector fitted with a 100 μL injection loop. The purification of the peptide segments was performed on a Gilson preparative instrument or Jasco semi-preparative instrument with 10-20 mL injection loop. In all cases, the mobile phase was MilliQ-H₂O with 0.1% TFA (v/v) (Buffer A) and HPLC grade CH₃CN with 0.1% TFA (v/v) (Buffer B). Analytical HPLC was performed on bioZen™ Intact C4 column (3.6 μm, 150×4.6 mm) at room temperature or Aeris WIDEPORE XB-C18 column (3.6 μm, 150×4.6 mm) with a flow rate of 1 mL/min at 60° C. Preparative HPLC was performed on a Gemini NX-C18 110 Å column (5 μm, 250×50 mm) or on a Shiseido capcell Pak UG80 C18 column (5 μm, 250×50 mm) at a flow rate of 40 mL/min at 40° C. or 60° C. Semi-preparative HPLC was performed on a Shiseido capcell Pak C18 column (5 μm, 250×20 mm) at a flow rate of 10 mL/min at 60° C.

The peptide segments were synthesized on an automated peptide synthesizer using Fmoc-SPPS chemistry. The following Fmoc-amino acids with side-chain protecting groups were used: Fmoc-Ma-OH, Fmoc-Arg(Pbf)-OH, Fmoc-Asn(Trt)-OH, Fmoc-Asp(OtBu)-OH, Fmoc-Asp(OBno)-OH, Fmoc-Asp(OAll)-OH, Fmoc-Cys(Acm)-OH, Fmoc-Gln(Trt)-OH, Fmoc-Glu(OtBu)-OH, Fmoc-Gly-OH, Fmoc-His(Trt)-OH, Fmoc-Ile-OH, Fmoc-Leu-OH, Fmoc-Lys(Boc)-OH, Fmoc-Lys(alloc)-OH, Fmoc-Met-OH, Fmoc-Met(O)-OH, Fmoc-Hse(Me)-OH, Fmoc-Nle-OH, Fmoc-Phe-OH, Fmoc-Pro-OH, Fmoc-Ser(tBu)-OH, Fmoc-Thr(tBu)-OH, Fmoc-Trp(Boc)-OH, Fmoc-Tyr(tBu)-OH, Fmoc-Val-OH. Fmoc-pseudoproline dipeptides were incorporated in the synthesis if necessary. Fmoc deprotections were performed with 20% piperidine in DMF (2×8 min) or 25% piperidine in DMF containing 0.1 M Cl-HOBt (2×8 min) or 20% piperidine in DMF containing 0.1 M Cl-HOBt (2×8 min), and monitored by UV at 304 nm with a feedback loop to ensure complete Fmoc removal. Couplings were performed with Fmoc-amino acid (3.0-5.0 eq to resin substitution), HCTU or HATU (2.9-4.9 eq) as coupling reagents and DIPEA or NMM (6-10 eq) in DMF at room temperature or at 50° C. After pre-activating for 3 min, the solution was added to the resin and allowed to react for 15 min, 30 min or 2 h depending on the amino acid. In some cases, double couplings were required. In some cases, the resin was treated with 20% acetic anhydride in DMF for capping any unreacted free amine. LiCl washings were performed if required. The allyloxycarbonyl (Alloc) deprotection was performed under nitrogen using phenylsilane (24 eq) and tetrakis(triphenylphosphine)palladium(0) (0.5 eq) in nitrogen purged dichloromethane at room temperature for 30 min.

The synthesis of the peptide segments by SPPS was monitored by microcleavage using the following sample protocol: 10 mg of peptidyl resin were treated with a cleavage cocktail (200 μL) at room temperature for 1.5 h. The resin was filtered off and the filtrate was concentrated and treated with cold diethyl ether, triturated and centrifuged. The ether layer was carefully decanted, and the residue was suspended again in diethyl ether, triturated and centrifuged. Ether washings were repeated twice. The resulting paste was resolubilized in 1:1 CH₃CN/H₂O with 0.1% TFA (v/v) and analyzed by analytical HPLC using an Aeris WIDEPORE XB-C18 column (3.6 μm, 150×4.6 mm) at 60° C. and MALDI-TOF.

Once the peptide synthesis was completed, the peptide was cleaved from the resin using a cleavage cocktail at room temperature for 2 h. The resin was filtered off, and the filtrate was concentrated and treated with cold diethyl ether, triturated and centrifuged. The ether layer was carefully decanted, and the residue was suspended again in diethyl ether, triturated and centrifuged. Ether washings were repeated twice. The resulting crude peptide was dried under vacuum and stored at −20° C.

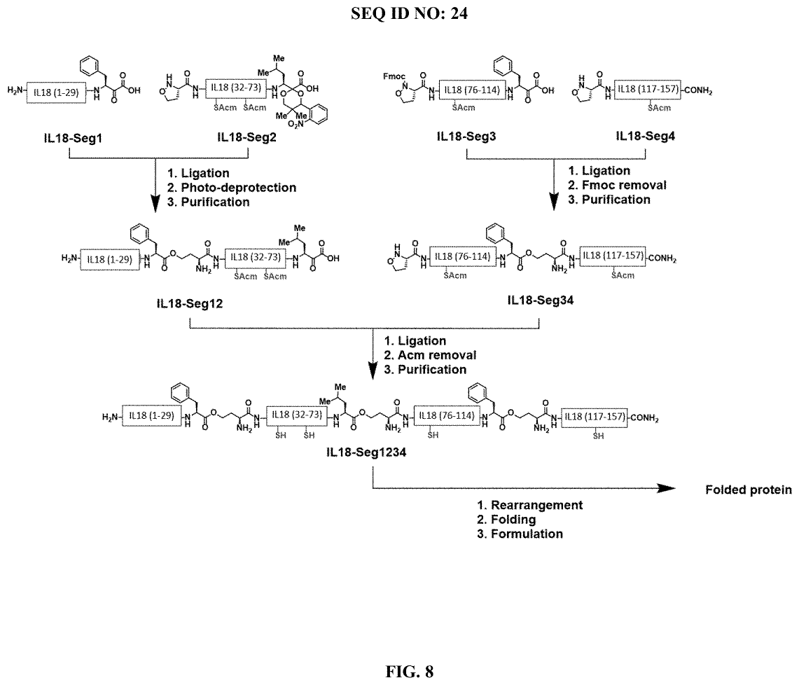

The general synthesis scheme used to produce modified IL-18 polypeptides provided herein is shown in FIG. 8. Briefly, linear peptide fragments (Fragments 1-4 as shown in FIG. 8) were prepared using SPPS, and any desired modification to the amino acid sequence of wild-type IL-18 (SEQ ID NO: 1) was incorporated during the syntheses. After purification of the individual segments, Segments 1 and 2 were ligated together, and Segments 3 and 4 were ligated together separately. Then, resulting Segments 1-2 and 3-4 were ligated together and universally deprotected to afford crude synthetic IL-18 polypeptide.

The Acm groups of IL18-Seg1234-Acm were then universally deprotected and purified to afford synthetic IL18 linear protein.

### 1.1 General Procedure for Synthesis of IL-18 Fragments

### 1.1.1. Segment 1: IL18(1-29)-Leu-α-Ketoacid

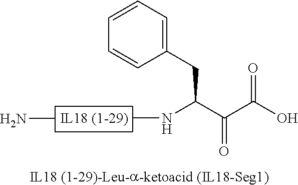

IL18(1-29)-Phe-α-ketoacid segment is synthesized on Rink Amide MBHA resin pre-loaded with protected Fmoc-α-Phe-ketoacid with a substitution capacity of 0.25 mmol/g. The synthesis is performed up to Tyr 1 by automated Fmoc-SPPS using the procedure described in the general methods section.

Variants of segment 1: In some cases, Glu 6 was substituted with Lys.

The progress of the peptide synthesis is monitored by performing a microcleavage analysis as described in the general methods section. The cleavage cocktail is composed of a mixture of 95:2.5:2.5 TFA/DODT/H₂O.

Once the synthesis is complete, the peptide is cleaved from the resin by stirring the resin in a mixture of 95:2.5:2.5 TFA/DODT/H₂O (10 mL/g resin) at room temperature for 2 h, as described in the general methods. Purification of crude IL18(1-29)-Phe-α-ketoacid segment is performed by preparative HPLC using a Shiseido capcell Pak UG80 C18 column (5 μm, 250×50 mm) at a flow rate of 40 mL/min at 60° C. with a gradient of 10 to 60% CH₃CN with 0.1% TFA (v/v) in 25 min. The fractions containing the purified product are pooled and lyophilized to obtain IL18(1-29)-Phe-α-ketoacid segment (IL18-Seg1). Analytical HPLC and ESI-HRMS are used to confirm the purity and mass of the product.

### 1.1.2 Segment 2: Opr-IL18(32-61)-Photoprotected-Val-α-Ketoacid and Opr-IL18(32-73)-Photoprotected-Leu-α-Ketoacid

### 1.1.2.1 Opr-IL18(32-61)-Photoprotected-Val-α-Ketoacid

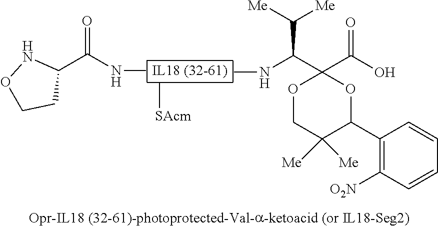

The Opr-IL18(32-61)-Val-photoprotected-α-ketoacid segment is synthesized on a 0.2 mmol scale on Rink Amide MBHA resin pre-loaded with Fmoc-Val-photoprotected-α-ketoacid with a substitution capacity of 0.24 mmol/g. The synthesis is performed up to Asp 32 by automated Fmoc-SPPS using the procedure described in the general methods section. Pseudoproline dipeptides are required for the synthesis of this segment and were manually coupled at positions 54-55, 49-50 and 35-36. Boc-5-(S)-oxaproline is manually coupled at the end of the sequence. Aspartic acid residues with non-conventional side-chain protecting groups are manually added at positions 32, 37 and 40. In some case, these protecting groups required an additional deprotection step after cleaving the peptide from the resin.

Variants of segment 2: In some cases, Lys 53 is substituted with Ala. In some cases, Cys(Acm) 38 is substituted with Ser. In some cases, Met 33, Met 51, and Met 60 are substituted with Nle or O-methyl-L-homoserine.

The progress of the peptide synthesis is monitored by performing a microcleavage described in the general methods section. The cleavage cocktail is composed of a mixture of 95:2.5:2.5 TFA/DODT/H₂O. Once the synthesis is complete, the peptide is cleaved from the resin using a mixture of 95:2.5:2.5 TFA/DODT/H₂O (15 mL/g resin) at room temperature for 2 h. The crude Opr-IL18(32-61)-photoprotected-Val-α-ketoacid segment is purified by preparative HPLC using Gemini NX-C18 110 Å column (5 μm, 250×50 mm) at a flow rate of 40 mL/min at 40° C. with a gradient of 10 to 60% CH₃CN with 0.1% TFA (v/v) in 30 min. The fractions with the purified product are pooled and lyophilized to obtain Opr-IL18(32-61)-photoprotected-Val-α-ketoacid (IL18-Seg2). Analytical HPLC and ESI-HRMS are used to confirm the purity and mass of the product. The fractions containing the purified product are pooled and lyophilized to obtain Opr-IL18(32-61)-photoprotected-Val-α-ketoacid (IL18-Seg2) as a white solid in >98% purity.

### 1.1.2.1 Opr-IL18(32-73)-Photoprotected-Leu-α-Ketoacid

Variations in segment 2 length: In some cases, the sequence of segment 2 of IL-18 is longer by a few amino acids and would comprise IL-18 sequence from position 31 to 74.

The segment Opr-IL18(32-73)-photoprotected-Phe-α-ketoacid segment is prepared on Rink Amide MBHA resin preloaded with Fmoc-Phe-photoprotected-α-ketoacid with a substitution capacity of 0.21 mmol/g. The synthesis is performed up to Asp 32 by automated Fmoc-SPPS using the procedure described in the general methods section. Boc-5-(S)-oxaproline is manually coupled to the sequence.

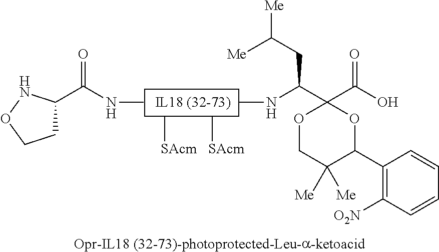

Variants of segment 2: In some cases, Lys 53 is substituted with Ala and Lys 70 was substituted with non-canonical N-α-(9-Fluorenylmethyloxycarbonyl)-ε-azido-L-lysine (Fmoc-Lys(N₃)-OH). In some cases, the side chain of Lys 70 is protected with an alloc group. The alloc group is then removed during an on-resin deprotection step, and the resulting free amine coupled with glutaric anhydride. The resulting free acid is then coupled to the corresponding desired group, for example a PEG group or PEG group bearing an azide functionality. In some cases, Cys(Acm) 38 and Cys(Acm) 68 are substituted with Ser. In some cases, Met 33, Met 51, and Met 60 are substituted with Nle or O-methyl-L-homoserine.

### 1.1.3 Segment 3: Fmoc-Opr-IL18(64-114)-Phe-α-Ketoacid and Fmoc-Opr-IL18(76-114)-Phe-α-Ketoacid

### 1.1.3.1 Fmoc-Opr-IL18(64-114)-Phe-α-Ketoacid

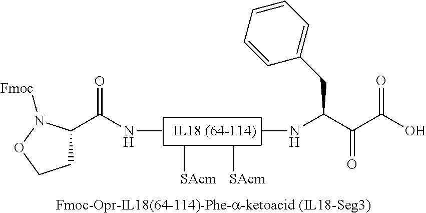

The Fmoc-Opr-IL18(64-114)-Phe-α-ketoacid segment is synthesized on a 0.1 mmol scale on Rink Amide ChemMatrix© resin pre-loaded with Fmoc-Phe-protected-α-ketoacid with a substitution capacity of 0.47 mmol/g. The synthesis is performed up to Ile 64 by automated Fmoc-SPPS using the procedure described in the general methods section. Pseudoproline dipeptides are required for the synthesis of this segment and are manually coupled at positions 81-82 and 71-72. Fmoc-5-(S)-oxaproline is manually coupled at the end of the sequence.

The progress of the peptide synthesis is monitored by performing a microcleavage described in the general methods section. The cleavage cocktail is composed of a mixture of 95:2.5:2.5 TFA/DODT/H₂O. Once the synthesis was complete, the peptide is cleaved from the resin using a mixture of 95:2.5:2.5 TFA/DODT/H₂O (15 mL/g resin) for 2 h. The crude Fmoc-Opr-IL18(76-114)-Phe-α-ketoacid segment is purified by preparative HPLC using a Gemini NX-C18 110 Å column (5 μm, 250×50 mm) at a flow rate of 40 mL/min at 40° C., with a gradient of 10 to 50% CH₃CN with 0.1% TFA (v/v) in 40 min. The fractions containing the purified product are pooled and lyophilized to obtain Fmoc-Opr-IL18(76-114)-Phe-α-ketoacid (IL18-Seg3). Analytical HPLC and ESI-HRMS are used to confirm the purity and mass of the product.

Variants of segment 3: In some cases, Cys(Acm) 68 and Cys(Acm) 76 are substituted with Ser. In some cases, Met86 and Met113 are substituted with Nle or O-methyl-L-homoserine. In some cases, Lys 70 is substituted with non-canonical N-α-(9-Fluorenylmethyloxycarbonyl)-ε-azido-L-lysine (Fmoc-Lys(N₃)-OH). In some cases, the side chain of Lys 70 is protected with an alloc group. The alloc group is then removed during an on-resin deprotection step, and the resulting free amine coupled with glutaric anhydride. The resulting free acid is then coupled to the corresponding desired group, for example a PEG group or PEG group bearing an azide functionality.

### 1.1.3.2 Fmoc-Opr-IL18(76-114)-Phe-α-Ketoacid

Variations in segment 3 length: In some cases, the sequence of segment 3 of IL-18 is shorter by a few amino acids and would comprise IL-18 sequence from position 75 to 115. The segment Fmoc-Opr-IL18(74-114)-Phe-α-ketoacid is then synthesized on Rink Amide ChemMatrix® resin pre-loaded with Fmoc-Phe-protected-α-ketoacid with a substitution capacity of 0.47 mmol/g. Automated Fmoc-SPPS is performed using the procedure described in the general methods section up to Cys(Acm) 76. Fmoc-5-(S)-oxaproline is manually coupled to the sequence.

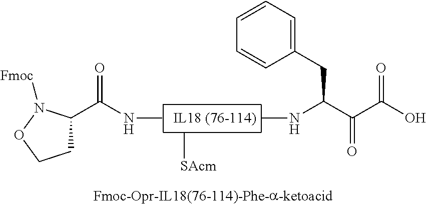

Variants of segment 3: In some cases, Cys(Acm) 76 is substituted with Ser. In some cases, Met86 and Met113 are substituted with Nle or O-methyl-L-homoserine.

### 1.1.4 Segment 4: Opr-IL18(117-157).

Preloading of Fmoc-Asp(OtBu)-OH is performed on a Fmoc-Rink-Amide MBHA resin. 4 g of resin (loading: 0.56 mmol/g, 2.24 mmol scale) is swollen in DMF for 15 min. The resin is treated with 20% in DMF (v/v) at r.t. for 20 min. The resin is washed several times with DMF. Fmoc-Asp(OtBu)-OH (691 mg, 1.68 mmol, 0.75 equiv) and HATU (638 mg, 1.68 mmol, 0.75 equiv) are dissolved in DMF (12 mL). Pre-activation is performed at r.t. for 3 min by addition of DIPEA (585 μL, 3.36 mmol, 1.5 equiv). The reaction mixture is added to the swollen resin. It is let to react overnight at r.t. under gentle agitation. The resin is rinsed thoroughly with DMF. Capping of unreacted amines on the resin is initiated by addition of a solution of acetic anhydride (1.17 mL) and DIPEA (2.34 mL) in DMF (12 mL). It is let to react at r.t. for 15 min under gentle agitation. The resin is rinsed thoroughly with DCM and dried. The loading of the resin is measured (0.34 mmol/g).

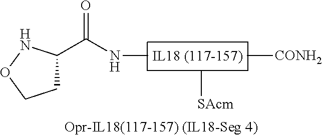

The Opr-IL18(117-157) segment is synthesized on Rink Amide MBHA resin pre-loaded with Fmoc-Asp(OtBu)-OH with a substitution capacity of 0.34 mmol/g. Automated Fmoc-SPPS is performed using the procedure described in the general methods section up to Ser 117. Boc-5-(S)-oxaproline is coupled to the sequence.

The progress of the peptide synthesis is monitored by performing a microcleavage described in the general methods section. The cleavage cocktail is composed of a mixture of 92.5:2.5:2.5:2.5 TFA/TIPS/DODT/H₂O. Once the synthesis is complete, the peptide is cleaved from the resin using a mixture of 92.5:2.5:2.5:2.5 TFA/TIPS/DODT/H₂O (10 mL/g resin) for 2 h. The crude Opr-IL18(117-157) segment is purified by preparative HPLC using a Gemini NX-C18 110 Å column (5 μm, 250×50 mm) at a flow rate of 40 mL/min at 40° C., with a gradient of 10 to 55% CH₃CN with 0.1% TFA (v/v) in 45 min. The fractions containing the purified product are pooled and lyophilized to obtain Opr-IL18(117-157) (IL18-Seg4). Analytical HPLC and ESI-HRMS are used to confirm the purity and mass of the product.

Variants of segment 4: In some cases, Cys(Acm) 127 is substituted with Ser. In some cases, Met 150 is substituted with Nle or O-methyl-L-homoserine

### 1.2 KAHA Ligations for the Preparation of IL18 Linear Protein.

### 1.2.1. KAHA Ligation for the Synthesis of Segment 12 (IL18-Seg12)

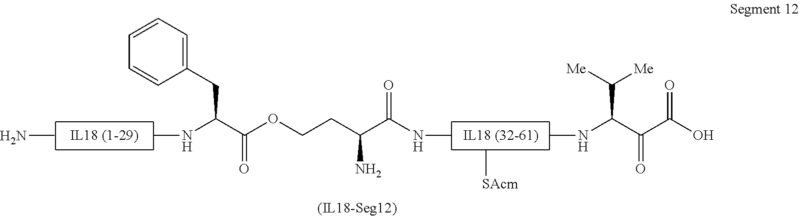

Ligation: IL18-Seg1 (1.2 eq) and IL18-Seg2 (1 eq) are dissolved in 9:1 DMSO/H₂O containing 0.1 M oxalic acid (20 mM peptide concentration for the limiting agent) and reacted at 60° C. for 15 h. The ligation vial is protected from light by wrapping the vial in aluminum foil. The progress of the KAHA ligation is monitored by HPLC using an Aeris WIDEPORE XB-C18 column (3.6 μm, 150×4.6 mm) at a flow rate of 1 mL/min at 60° C. with a gradient of 20 to 95% CH₃CN in 7 min.

Photodeprotection: After completion of the ligation, the mixture is diluted with 1:1 CH₃CN/H₂O with 0.1% TFA (v/v) and irradiated at a wavelength of 365 nm for 1.5 h. Completion of the photolysis reaction was confirmed by HPLC and MALDI-TOF MS analysis.

Purification: The photo-deprotected sample is purified by preparative HPLC using a Shiseido capcell Pak UG80 C18 column (5 μm, 250×50 mm) kept at 60° C., with a 2-step gradient: 10 to 60% CH₃CN with 0.1% TFA (v/v) in 25 min, then hold 60% CH₃CN for 5 min, with a flow of 40 mL/min. The fractions containing the purified product are pooled and lyophilized to obtain IL18-Seg12. The purity and identity of the segment is confirmed by HPLC and ESI-HRMS analysis.

### 1.2.2 KAHA Ligation for the Synthesis of Segment 34 (IL18-Seg34)

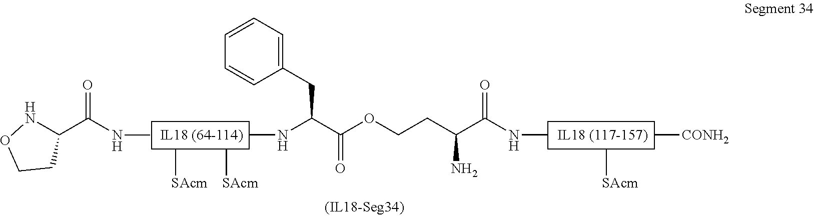

Ligation: IL18-Seg3 (1 eq) and IL18-Seg4 (1.2 eq) are dissolved in 97.5:2.5 DMSO/H₂O containing 0.1 M oxalic acid (20 mM peptide concentration for the limiting agent) and reacted for 16 h at 60° C. The progress of the KAHA ligation is monitored by HPLC using an Aeris WIDEPORE (3.6 μm, 150×4.6 mm) column with a flow rate of 1 mL/min at 60° C. with a gradient of 5 to 65% CH₃CN in 7 min.

Fmoc deprotection: After completion of ligation, the reaction mixture is diluted with DMSO (6.7 mM peptide concentration). Diethylamine is added (5%, v/v) and the reaction mixture is shaken at room temperature for 15 min. The reaction mixture is diluted a second time with DMSO (3.3 mM peptide concentration). Diethylamine is added (2.5%, v/v) and the reaction mixture is shaken at room temperature for another 15 min. The reaction mixture is then diluted with 1:1 CH₃CN/H₂O with 0.1% TFA (v/v).

Purification: The sample is purified by preparative HPLC on a Gemini NX-C18 110 Å column (5 μm, 250×50 mm) at a flow rate of 40 mL/min at 40° C., with a gradient of 10 to 50% CH₃CN with 0.1% TFA (v/v) in 40 min. The fractions containing the purified product are pooled and lyophilized to obtain IL18-Seg34 (Seg34). Analytical HPLC and ESI-HRMS are used to confirm the purity and mass of the product.

### 1.2.3 KAHA Ligation for the Synthesis of IL18 Segment 1234 (IL18-Seg1234-Acm).

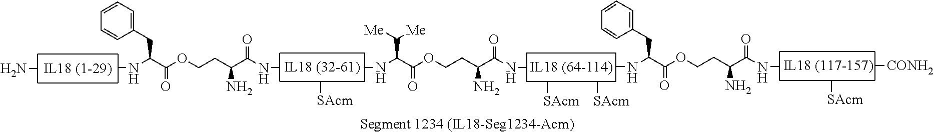

Ligation: IL18-Seg12 (1.2 eq) and IL18-Seg34 (1.0 eq) are dissolved in 9:1 DMSO/H₂O containing 0.1 M oxalic acid (15 mM peptide concentration), and the reaction is stirred for 24 hat 60° C. The progress of the KAHA ligation is monitored by analytical HPLC using an Aeris WIDEPORE (3.6 μm, 150×4.6 mm) column with a flow rate of 1 mL/min at 60° C. using CH₃CN/H₂O with 0.1% TFA (v/v) as mobile phase, with a gradient of 20 to 95% CH₃CN in 7 min. After completion of ligation, the reaction mixture is diluted with DMSO followed by further dilution with a mixture of 1:1 CH₃CN/H₂O with 0.1% TFA (v/v).

Purification: The sample is purified by preparative HPLC on a Gemini NX-C18 110 Å column (5 μm, 250×50 mm) at a flow rate of 40 mL/min at 60° C., with a gradient of 10 to 60% CH₃CN with 0.1% TFA (v/v) in 30 min. The fractions containing the purified product are pooled and lyophilized to obtain IL18-Seg1234 with cysteine residues protected with an Acm group (IL18-Seg1234-Acm). Analytical HPLC and ESI-HRMS are used to confirm the purity and mass of the product.

### 1.2.4 Rearrangement and Acm Deprotection for the Synthesis of IL18 Linear Protein

Rearrangement: IL18-Seg1234-Acm is dissolved in 6 M Gu.HCl containing 0.1 M Tris (pH 8.1) (1.5 mL, 0.13 mM protein concentration). The pH is adjusted to 8.0. It is let to react for 2 hat 50° C. After completion of reaction, the sample is diluted with 6 M Gu.HCl containing 0.1% TFA (v/v, 10 mL), and purified by preparative HPLC using a Proteonavi S5 column (250×20 mm) at a flow rate of 10 mL/min at 60° C. using CH₃CN/H₂O with 0.1% TFA (v/v) as mobile phase, with a gradient of 20 to 40% (in 19 min) and 40 to 50% (in 11 min) CH₃CN with 0.1% TFA (v/v). The fractions containing the product are pooled and lyophilized to obtain IL18 linear protein with Acm. Analytical HPLC and ESI-HRMS are used to confirm the purity and mass of the product.

Acm deprotection: IL18 linear protein with Acm is dissolved in 1:1 AcOH/H₂O (0.25 mM protein concentration), and silver acetate (1%, m/v) is added to the solution. The mixture is shaken for 2.5 h at 50° C. protected from light. The progress of the Acm deprotection reaction is monitored by analytical HPLC using an Aeris WIDEPORE (3.6 μm, 150×4.6 mm) column with a flow rate of 1 mL/min at 60° C. using CH₃CN/H₂O with 0.1% TFA (v/v) as mobile phase, with a gradient of 20 to 95% CH₃CN in 7 min. After completion of the reaction, the sample is diluted with 1:1 CH₃CN/H₂O with 0.1% TFA (v/v).

Purification: The sample is purified by preparative HPLC on a Shiseido capcell Pak UG80 C18 column (250×20 mm) at a flow rate of 10 mL/min at room temperature using CH₃CN/H₂O with 0.1% TFA (v/v) as mobile phase, with a two-step gradient: 10 to 30% CH₃CN in 5 min and 30 to 95% CH₃CN in 20 min. The fractions containing the purified product are pooled and lyophilized to obtain the desired IL18 linear protein. Analytical HPLC and HRMS are used to confirm the purity and mass of the product.

### Example 2—Folding of Modified IL-18 Polypeptides

The synthesized modified IL-18 polypeptides are dissolved in buffered solutions and subjected to specific buffer and pH conditions to promote folding of the polypeptides. The folded protein is confirmed using analytical techniques, such as HPLC, ESI-MS and/or MALDI-TOF. Several conditions are screened and tested by varying the composition of the folding buffers (Buffers A and B) and formulation buffers. Exemplary folding conditions and buffer compositions are shown below in Table 14. One or more conditions which result in the desired analytical and biochemical properties of the modified IL-18 polypeptide is selected for scale up folding protocols.

Step 1: The linear protein is dissolved in Buffer A (2 to 4 mg/mL protein concentration). The protein solution is gently shaken at 20° C. for up to 1 h.

Step 2: The solution of protein is slowly diluted in a dropwise fashion with Buffer B. A clear solution obtained at a concentration of 0.2 to 0.4 mg/mL is incubated at 4° C., 10° C. or 20° C. for 18 to 48 h.

Step 3: The solution is centrifuged at 10 000 RPM at 10° C. for 10 min. It is then dialyzed against PBS (pH 7.4) containing 0.02% Tween 80 and 5-6% sucrose at r.t. for 2 h. This step is repeated a second time. It is then dialyzed a third time at r.t. for 18 h with the same buffer.

The purity and identity of the pure folded protein is further confirmed by analytical HPLC and MALDI-TOF.

TABLE 14
Composition of Buffers A and B

| Buffer A | Buffer B |
|---|---|
| Tris buffer (50 mM, pH 8.0) containing 8 M | Tris buffer (50 mM, pH 7.8) containing 2 mM |
| urea, 2 mM DTT and 0.02% (m/v) Tween 80 | EDTA, 137 mM NaC1, 2.7 mM KC1, 400 mM |
| | Arginine HC1, 2 mM DTT and 0.02% (m/v) |
| | Tween 80 |
| Tris buffer (50 mM, pH 8.0) containing 8 M | Tris buffer (50 mM, pH 7.8) containing 2 mM |
| urea, 2 mM DTT and 0.1% (m/v) Tween 80 | EDTA, 137 mM NaC1, 2.7 mM KC1, 400 mM |
| | ArginineHC1, 2 mM DTT and 0.1% (m/v) |
| | Tween 80 |
| PBS (pH 7.4) containing 6 M urea, 0.02% | PBS (pH 7.4) containing 0.02% Tween20 |
| Tween20 and 1 mM | TCEP |
| PBS (pH 7.4) containing 8 M urea | PBS (pH 7.4) |
| Tris buffer (50 mM, pH 8.0) containing 8 M | Tris buffer (50 mM, pH 7.8) containing 2 mM |
| urea | EDTA, 240 mM NaC1, 10 mM KC1, 200 mM |
| | ArginineHC1, 200 mM sucrose, 2 mM DTT |
| | and 0.02% (m/v) Tween 80 |
| HEPES buffer (50 mM, pH 8.0) containing 8 | HEPES buffer (50 mM, pH 7.8) containing 2 |
| M urea, 2 mM DTT and 0.02% (m/v) Tween | mM EDTA, 137 mM NaC1, 2.7 mM KC1, 400 |
| 80 | mM ArginineHC1, 2 mM DTT and 0.02% |
| | (m/v) Tween 80 |
| HEPES buffer (100 mM, pH 7.5) containing 8 | HEPES buffer (100 mM, pH 7.5) containing 1 |
| mM cysteine, 0.1 mM cystine, 1 mM EDTA | M urea, 1 mM cysteine, 0.1 mM cystine, 1 mM |
| EDTA and 0.02% (m/v) Tween 80 | and 0.02% (m/v) Tween 80 |

### Example 3—Further Modification of the Folded IL-18

The folded IL-18 is further modified by reaction with a polyethylene glycol polymer and formulated in appropriate buffers.

### Example 4—Synthesis of a Modified IL-18 Polypeptide of for SEQ ID NO: 24

A linear peptide of SEQ ID NO: 24 was prepared according to the protocol described below.

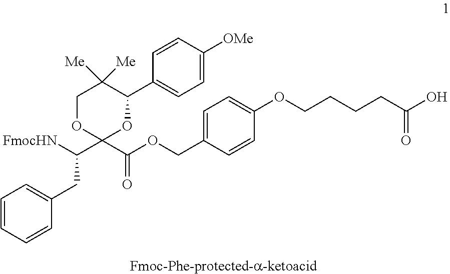

Segment 1 (IL-18 (1-29)-Phe-α-ketoacid): Preloading of Fmoc-Phe-protected-α-ketoacid 1 was performed on a Fmoc-Rink Amide MBHA resin. 5 g of resin (loading: 0.56 mmol/g, 1.8 mmol scale) was swollen in DMF for 20 min. The resin was treated twice with 20% piperidine in DMF (v/v) at room temperature for 10 min. and was washed several times with DMF. Ketoacid 1 (1.46 g, 1.8 mmol, 1.0 eq) and HATU (650 mg, 1.71 mmol, 0.95 eq) were dissolved in DMF (20 mL). Pre-activation was performed at room temperature for 3 min by adding NMM (396 μL, 3.6 mmol, 2 eq). The reaction mixture was added to the swollen resin and gently agitated at room temperature for 2.5 h. The resin was rinsed thoroughly with DMF. Capping of unreacted amines on the resin was initiated by adding a solution of acetic anhydride (1.17 mL) and DIPEA (2.34 mL) in DMF (20 mL) and gently agitating the reaction at room temperature for 15 min. The resin was rinsed thoroughly with DCM followed by diethyl ether and dried. The loading of the resin was measured (0.30 mmol/g).

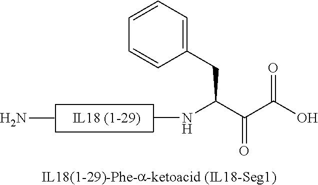

The IL18(1-29)-Phe-α-ketoacid segment was synthesized on a 0.45 mmol scale on Rink Amide MBHA resin pre-loaded with Fmoc-Phe-protected-α-ketoacid (1.5 g) with a substitution capacity of ˜0.30 mmol/g.

Automated Fmoc-SPPS of IL18(1-29)-Phe-α-ketoacid: The coupling reactions were performed at room temperature for 30 min by adding a solution of Fmoc-amino acids dissolved in DMF (10.0 mL, 0.4 M, 4 eq), HCTU in DMF (10.0 mL, 0.38 M, 3.8 eq) and NMM in DMF (10.0 mL, 0.8 M, 8 eq) to the resin. For position 14 to 1, double couplings were required. Washing with a solution of lithium chloride (0.8 M) in DMF was performed every five amino acids before the Fmoc deprotection reaction. When required, capping was performed at room temperature for 10 min by adding a 20% (v/v) acetic anhydride solution in DMF (10.0 mL) and NMM in DMF (0.8 M, 10.0 mL). The Fmoc deprotection reaction was performed using 20% (v/v) piperidine in DMF containing Cl-HOBt (0.1 M) at room temperature for 8 min.

The resin was washed with DCM and dried under vacuum. The mass of the dried peptidyl resin was 4.6 g. The peptide was cleaved from the resin using a mixture of 95:2.5:2.5 TFA/DODT/H₂O (10 mL/g resin) at room temperature for 2.0 h. The resin was filtered off from the cleavage cocktail, and the filtrate was concentrated and diluted 20-fold with cold diethyl ether (20° C.), allowing the peptide to precipitate. After centrifugation, the ether layer was carefully decanted, and the peptide precipitate was resuspended in diethyl ether, triturated and centrifuged. Ether washings were repeated twice, and the resulting peptide precipitate was dried. The mass of crude peptide was 1.8 g. Purification of crude IL18(1-29)-Phe-α-ketoacid segment was performed by preparative HPLC using Shiseido Capcell Pak UG80 C18 column (5 μm, 250×50 mm) at a flow rate of 40 mL/min at 60° C. using CH₃CN/H₂O with 0.1% TFA (v/v) as mobile phase, with a gradient of 10 to 60% CH₃CN with 0.1% TFA (v/v) in 25 min. The fractions containing the purified product were pooled and lyophilized to obtain IL18(1-29)-Phe-α-ketoacid (IL18-Seg1) as a white solid in >98% purity. The isolated yield based on the resin loading was 472 mg (28%). MS (ESI): C₂₃₃H₃₄₈N₅₈O₆₉S; Average isotope calculated 3550.8936 Da [M]; found: 3550.8948 Da.

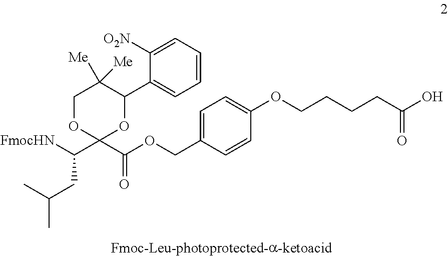

Segment 2 (Opr-IL18(32-73)-Leu-photoprotected-α-ketoacid): Preloading of Fmoc-Leu-photoprotected-α-ketoacid 2 was performed on a Fmoc-Rink-Amide MBHA resin. 5 g of resin (loading: 0.56 mmol/g, 2.25 mmol scale) was swollen in DMF for 20 min. Ketoacid 2 (1.79 g, 2.25 mmol, 1 eq) and HATU (813 mg, 2.14 mmol, 0.95 eq) were dissolved in DMF (25 mL). Pre-activation was performed at room temperature for 2 min by adding NMM (495 μL, 4.5 mmol, 2 eq). The reaction mixture was added to the swollen resin and gently agitated for 6 h at room temperature. The resin was rinsed thoroughly with DMF. Capping of unreacted amines on the resin was initiated by adding a solution of acetic anhydride (2.0 mL) and DIPEA (2.0 mL) in DMF (20 mL) and gently agitating the mixture at room temperature for 15 min. The resin was rinsed thoroughly with DCM and diethyl ether and dried. The loading of the resin was measured (0.34 mmol/g).

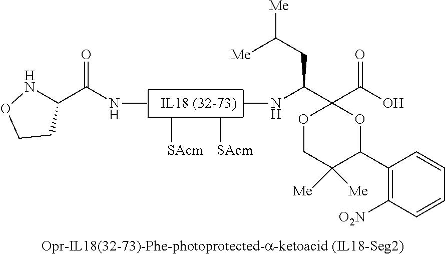

Opr-IL18(32-73)-Phe-photoprotected-α-ketoacid segment was synthesized on a 0.2 mmol scale on Rink Amide MBHA resin pre-loaded with Fmoc-Phe-Leu-photoprotected-α-ketoacid with a substitution capacity of ˜0.34 mmol/g.

Automated Fmoc-SPPS from position 73 to 66: The coupling reactions were performed at room temperature for 30 min by adding the Fmoc-amino acids dissolved in DMF (2.0 mL, 0.4 M, 4 eq), HCTU in DMF (2.0 mL, 0.38 M, 3.8 eq) and NMM in DMF (2.0 mL, 0.8 M, 8 eq) to the resin. The Fmoc deprotection reaction was performed twice for each coupling cycle using 20% (v/v) piperidine in DMF containing Cl-HOBt (0.1 M) at room temperature for 7 min.

Manual coupling of Fmoc-L-Ile-L-Ser[Ψ(Me,Me)Pro]-OH was then performed. A solution of Fmoc-L-Ile-L-Ser[Ψ(Me,Me)Pro]-OH (384 mg, 0.8 mmol, 4 eq), HATU (290 mg, 0.76 mmol, 3.8 eq) and NMM (176 μL, 1.6 mmol, 8 eq) in 3 mL of DMF was prepared (3 min of pre-activation at room temperature) and added to the resin. The reaction was gently agitated at room temperature for 1 h.

Automated Fmoc-SPPS for position 63: The coupling reactions were performed using the conditions described above. Triple coupling was required for position 63.

Automated Fmoc-SPPS from position 64 to 56: The coupling reactions were performed using the conditions described above.

Manual coupling of Fmoc-L-Asp(tBu)-L-Ser[Ψ(Me,Me)Pro]-OH was then performed. A solution of Fmoc-L-Asp(tBu)-L-Ser[Ψ(Me,Me)Pro]-OH (430 mg, 0.8 mmol, 4 eq), HATU (290 mg, 0.76 mmol, 3.8 eq) and NMM (176 μL, 1.6 mmol, 8 eq) in 3 mL of DMF was prepared (3 min of pre-activation at room temperature) and added to the resin. The reaction was gently agitated at room temperature for 1 h.

Automated Fmoc-SPPS from position 53 to 51: The coupling reactions were performed using the same conditions as previously mentioned for the beginning of the sequence.

Manual coupling of Fmoc-L-Ile-L-Ser[Ψ(Me,Me)Pro]-OH was then performed. A solution of Fmoc-L-Ile-L-Ser[Ψ(Me,Me)Pro]-OH (384 mg, 0.8 mmol, 4 eq), HATU (290 mg, 0.76 mmol, 3.8 eq) and NMM (176 μL, 1.6 mmol, 8 eq) in 3 mL of DMF was prepared (3 min of pre-activation at room temperature) and added to the resin. The reaction was gently agitated at room temperature for 1 h.

Automated SPPS from position 48 to 37: The coupling reactions were performed using the conditions described above. Double couplings were required for each position.

Manual coupling of Fmoc-L-Asp(tBu)-L-Ser[Ψ(Me,Me)Pro]-OH was then performed. A solution of Fmoc-L-Asp(tBu)-L-Ser[Ψ(Me,Me)Pro]-OH (430 mg, 0.8 mmol, 4 eq), HATU (290 mg, 0.76 mmol, 3.8 eq) and NMM (176 μL, 1.6 mmol, 8 eq) in 3 mL of DMF was prepared (3 min of pre-activation at room temperature) and added to the resin. The reaction was gently agitated at room temperature for 1 h.

Automated Fmoc-SPPS from position 34 to 32: The coupling reactions were performed using the conditions described above. Double couplings were required for each position. Capping was performed at room temperature for 10 min at each position by adding 20% (v/v) acetic anhydride in DMF (2 mL) and NMM in DMF (0.8 M, 2 mL). Fmoc deprotection was performed using 20% (v/v) piperidine in DMF containing Cl-HOBt (0.1 M) at room temperature for 7 min.

Manual coupling of Boc-5-(S)-oxaproline was then performed. A solution of Boc-5-(S)-oxaproline (217 mg, 1.0 mmol, 5 eq), HATU (361 mg, 0.95 mmol, 4.8 eq) and NMM (220 μL, 2.0 mmol, 10 eq) in 7 mL of DMF was prepared (3 min of pre-activation at room temperature) and added to the resin. The reaction was gently agitated at room temperature for 2.5 h. The resin was washed with DCM and diethyl ether and dried under vacuum. The mass of the dried peptidyl resin was 1.8 g.

The peptide was cleaved from the resin using a mixture of 95:2.5:2.5 TFA/DODT/H₂O (15 mL/g resin) and gently agitating the mixture at room temperature for 2.0 h. The resin was filtered off from the cleavage cocktail, and the filtrate was concentrated and diluted 20-fold with cold diethyl ether (20° C.), allowing the peptide to precipitate. After centrifugation, the ether layer was carefully decanted, and the peptide precipitate was resuspended in diethyl ether, triturated and centrifuged. Ether washings were repeated twice, and the resulting peptide precipitate was dried. The mass of crude peptide was 1.2 g. Purification of the crude Opr-IL18(32-73)-Phe-photoprotected-α-ketoacid segment was performed by preparative HPLC using Gemini NX-C18 110 Å column (5 μm, 250×50 mm) at a flow rate of 40 mL/min at 40° C. using CH₃CN/H₂O with 0.1% TFA (v/v) as mobile phase, with a gradient of 10 to 60% CH₃CN with 0.1% TFA (v/v) in 30 min. The fractions containing the purified product were pooled and lyophilized to obtain Opr-IL18(32-73)-Phe-photoprotected-α-ketoacid (IL18-Seg2) as a white solid in >98% purity. The isolated yield based on the resin loading was 148 mg (14%). LC-MS (ESI): 4.88 min; C₂₃₃H₃₄₈N₅₈O₆₉S; m/z calculated: 1315.4233 Da [M+4H]; found: 1315.4231 Da [M+4H].

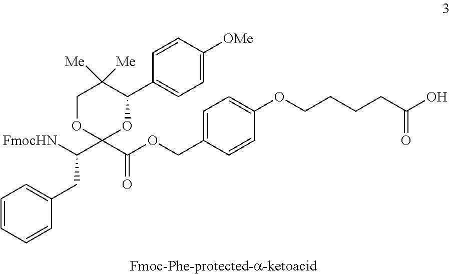

Segment 3 (Fmoc-Opr-IL18(76-114)-Phe-α-ketoacid): 222 mg of resin (loading: 0.47 mmol/g, 0.1 mmol scale) was swollen in DMF for 15 min. Ketoacid 3 (163 mg, 0.2 mmol, 2 eq) and HATU (76 mg, 0.2 mmol, 2 eq) were dissolved in DMF (2 mL). Pre-activation was performed at room temperature for 2 min by adding DIPEA (100 μL, 0.6 mmol, 6 eq). The reaction mixture was added to the swollen resin. The reaction was gently agitated overnight at room temperature. The resin was rinsed thoroughly with DMF. Capping of unreacted amines on the resin was initiated by adding a solution of acetic anhydride (100 μL) and DIPEA (100 μL) in DMF (2 mL). The reaction was gently agitated at room temperature for 15 min. The resin was rinsed thoroughly with DMF. The final loading of the resin was not calculated and was estimated to be unchanged (0.47 mmol/g).

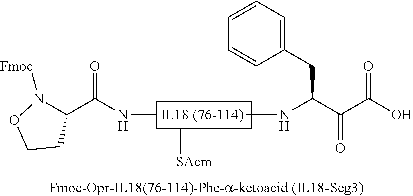

The Fmoc-Opr-IL18(76-114)-Phe-α-ketoacid segment was synthesized on a 0.1 mmol scale on Rink Amide ChemMatrix© resin pre-loaded with Fmoc-Phe-protected-α-ketoacid with a substitution capacity of ˜0.47 mmol/g.

Automated Fmoc-SPPS from position 96 to 114: The coupling reactions were performed at room temperature for 30 min by adding Fmoc-amino acids dissolved in DMF (1.0 mL, 0.5 M, 5 eq), HCTU in DMF (1.0 mL, 0.48 M, 4.8 eq) and DIPEA in NMP (0.4 mL, 0.2 M, 8 eq) to the resin. Fmoc deprotection was performed using 20% (v/v) piperidine in DMF at room temperature for 15 min.

Manual coupling of Fmoc-L-Asp(tBu)-L-Thr[Ψ(Me,Me)Pro]-OH was then performed. A solution of Fmoc-L-Asp(tBu)-L-Thr[Ψ(Me,Me)Pro]-OH (166 mg, 0.3 mmol, 3 eq), HATU (114 mg, 0.3 mmol, 3 eq) and DIPEA (100 μL, 0.6 mmol, 6 eq) in 3 mL of DMF was prepared (3 min of pre-activation at room temperature) and added to the resin. The reaction was gently agitated at room temperature for 2 h.

Automated Fmoc-SPPS from position 76 to 93: The coupling reactions were performed using the conditions described above. Double couplings were required for each position. Capping was performed at room temperature for 10 min at each position by adding 20% (v/v) acetic anhydride in DMF (1 mL) and DIPEA in DMF (0.2 M, 1 mL). Fmoc deprotection reactions were performed using 25% (v/v) piperidine in DMF containing Cl-HOBt (0.1 M) at room temperature for 15 min.

Manual coupling of Fmoc-5-(S)-oxaproline was then performed. A solution of Fmoc-5-(S)-oxaproline (102 mg, 0.3 mmol, 3 eq), HATU (114 mg, 0.3 mmol, 3 eq) and DIPEA (100 μL, 0.6 mmol, 6 eq) in 3 mL of DMF was prepared (3 min of pre-activation at room temperature) and added to the resin. The reaction was gently agitated at room temperature for 2 h. The resin was washed with DCM and dried under vacuum. The mass of the dried peptidyl resin was 1.0 g.

The peptide was cleaved from the resin by stirring the resin in a mixture of 95:2.5:2.5 TFA/DODT/H₂O (15 mL/g resin) at room temperature for 2.0 h. The resin was filtered off from the cleavage cocktail, and the filtrate was concentrated and diluted 20-fold with cold diethyl ether (20° C.), allowing the peptide to precipitate. After centrifugation, the ether layer was carefully decanted, and the peptide precipitate was resuspended in diethyl ether, triturated and centrifuged. Ether washings were repeated twice, and the resulting peptide precipitate was dried. Mass of crude peptide was 540 mg. Purification of crude Fmoc-Opr-IL18(76-114)-Phe-α-ketoacid segment was performed by preparative HPLC using Gemini NX-C18 110 Å column (5 μm, 250×250 mm) at a flow rate of 40 mL/min at 40° C. using CH₃CN/H₂O with 0.1% TFA (v/v) as mobile phase, with a gradient of 10 to 50% CH₃CN with 0.1% TFA (v/v) in 40 min. The fractions containing the purified product were pooled and lyophilized to obtain the Fmoc-Opr-IL18(76-114)-Phe-α-ketoacid (IL18-Seg3) as a white solid. The isolated yield based on the resin loading was 128 mg (25%). MS (ESI): C₂₃₃H₃₄₈N₅₈O⁶⁹S; Average isotope calculated 5094.5226 Da [M]; found: 5094.5224 Da.

Segment 4 (Opr-IL18 (117-157)): Preloading of Fmoc-Asp(OtBu)-OH was performed on a Fmoc-Rink-Amide MBHA resin. 4 g of resin (loading: 0.56 mmol/g, 2.24 mmol scale) was swollen in DMF for 15 min. The resin was treated with 20% in DMF (v/v) at room temperature for 20 min. The resin was washed several times with DMF. Fmoc-Asp(OtBu)-OH (691 mg, 1.68 mmol, 0.75 eq) and HATU (638 mg, 1.68 mmol, 0.75 eq) were dissolved in DMF (12 mL). Pre-activation was performed at room temperature for 3 min by adding DIPEA (585 μL, 4.48 mmol, 2 eq). The reaction mixture was added to the swollen resin and gently agitated overnight at room temperature. The resin was rinsed thoroughly with DMF. Capping of unreacted amines on the resin was initiated by adding a solution of acetic anhydride (1.17 mL) and DIPEA (2.34 mL) in DMF (12 mL) and gently agitating the mixture at room temperature for 15 min. The resin was rinsed thoroughly with DCM and dried. The loading of the resin was measured (0.34 mmol/g).

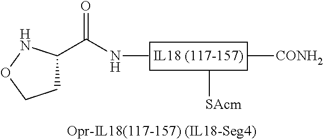

Opr-IL18(117-157) segment was synthesized on a 0.1 mmol scale on Rink Amide MBHA resin pre-loaded with Fmoc-Asp(OtBu)-OH with a substitution capacity of −0.34 mmol/g. 294 mg of resin was swollen in DMF for 15 min.

Automated Fmoc-SPPS from position 147 to 157: The coupling reactions were performed at room temperature for 30 min by adding Fmoc-amino acids dissolved in DMF (1.0 mL, 0.5 M, 5 eq), HCTU in DMF (1.0 mL, 0.48 M, 4.8 eq) and DIPEA in NMP (0.4 mL, 0.2 M, 8 eq) to the resin. Fmoc deprotection was performed using 20% (v/v) piperidine in DMF at room temperature for 15 min. Double coupling was required from position 117 to 146 as well as capping steps. Capping was performed at room temperature for 10 min at each position by adding 20% (v/v) acetic anhydride in DMF (1 mL) and DIPEA in DMF (0.2 M, 1 mL).

Manual coupling of Boc-5-(S)-oxaproline was then performed. A solution of Boc-5-(S)-oxaproline (65 mg, 0.3 mmol, 3 eq), HATU (114 mg, 0.3 mmol, 3 eq) and DIPEA (100 μL, 0.6 mmol, 6 eq) in 3 mL of DMF was prepared (3 min of pre-activation at room temperature) and added to the resin. The mixture was reacted at room temperature for 2 h.

The resin was washed with DCM and dried under vacuum. The mass of the dried peptidyl resin was 1.2 g. The peptide was cleaved from the resin using a mixture of 92.5:2.5:2.5:2.5 TFA/TIPS/DODT/H₂O (10 mL/g resin) at room temperature for 2 h. The resin was filtered off from the cleavage cocktail, and the filtrate was concentrated and diluted 20-fold with cold diethyl ether (20° C.), allowing the peptide to precipitate. After centrifugation, the ether layer was carefully decanted, and the peptide precipitate was resuspended in diethyl ether, triturated and centrifuged. Ether washings were repeated twice, and the resulting peptide precipitate was dried. Mass of crude peptide was 770 mg. Purification of crude Opr-IL18(117-157) segment was performed by preparative HPLC using Gemini NX-C18 110 Å column (5 μm, 250×50 mm) at a flow rate of 40 mL/min at 40° C. using CH₃CN/H₂O with 0.1% TFA (v/v) as mobile phase, with a gradient of 10 to 50% CH₃CN with 0.1% TFA (v/v) in 40 min. The fractions containing the purified product were pooled and lyophilized to obtain Opr-IL18(117-157) (IL18-Seg4) as a white solid. The isolated yield based on the resin loading was 106 mg (21%). MS (ESI): C₂₂₂H₃₄₆N₅₆O₇₃S; Average isotope calculated 1250.9051 Da [M+4H⁺]; found: 1250.6293 Da.

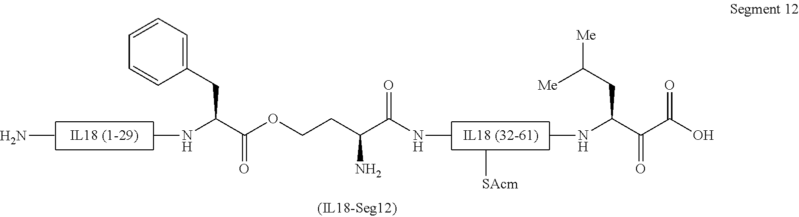

Peptide photoprotected ketoacid IL18-Seg12 (28.4 mg; 7.98 μmol; 1.2 equiv) and hydroxylamine peptide IL18-Seg2 (25.9 mg; 6.65 μmol; 1.0 equiv) were in dissolved in 9:1 DMSO/H₂O containing 0.1 M oxalic acid (333 μL). A homogeneous liquid solution was obtained. The ligation vial was protected from light by wrapping the vial in aluminum foil, and the reaction was left overnight at 60° C. After completion of the ligation the mixture was diluted with 1:1 CH₃CN/H₂O with 0.1% TFA (v/v) (1780 μL) and irradiated at a wavelength of 365 nm for 1.5 h to allow photodeprotection of the C-terminal ketoacid. The reaction mixture was further diluted with 1:1 CH₃CN/H₂O (q.s. 10 mL) with TFA (0.1%, v/v). The diluted mixture was filtered and injected into preparative HPLC. Crude ligated peptide was purified by preparative HPLC using Gemini NX-C18 110 Å column (5 μm, 250×250 mm) at a flow rate of 40 mL/min at 60° C., with a gradient of 10 to 60% CH₃CN with 0.1% TFA (v/v) in 30 min. The fractions containing the purified product were pooled and lyophilized to obtain IL18-Seg12 as a white solid in >98% purity. The isolated yield was 23.9 mg (50%).

LC-MS (ESI): 4.63 min; C₃₂₂H₅₁₅N₈₉O₉₆S; m/z calculated: 7196.7874 Da [M]; found: 7196.7476 Da [M].

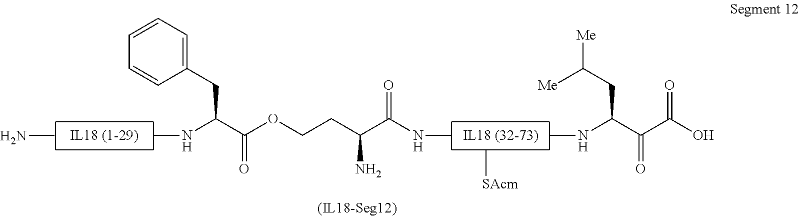

IL18-Seg12 preparation: Peptide photo-protected ketoacid IL18-Seg1 (18.1 mg; 5.09 μmol; 1.2 eq) and hydroxylamine peptide IL18-Seg2 (22.3 mg; 4.24 μmol; 1.0 eq) were in dissolved in a 9:1 DMSO/H₂O solution containing 0.1 M oxalic acid (220 μL). A very homogeneous liquid solution was obtained. The ligation vial was protected from light by wrapping the vial in aluminum foil and gently agitated overnight at 60° C. After completion of the ligation, the mixture was diluted with 1:1 CH₃CN/H₂O with 0.1% TFA (v/v) (1780 μL) and irradiated at a wavelength of 365 nm for 1.5 h to allow photo deprotection of the C-terminal ketoacid. The mixture was further diluted with 1:1 CH₃CN/H₂O (q.s. 10 mL) with TFA (0.1%, v/v). The diluted mixture was filtered and injected into preparative HPLC. Crude ligated peptide was purified by preparative HPLC using Gemini NX-C18 110 Å column (5 μm, 250×250 mm) at a flow rate of 40 mL/min at 60° C., with a gradient of 10% to 60% CH₃CN with 0.1% TFA (v/v) in 25 min. The fractions containing the purified product were pooled and lyophilized to obtain IL18-Seg12 as a white solid in >98% purity. The isolated yield was 16.9 mg (47%).

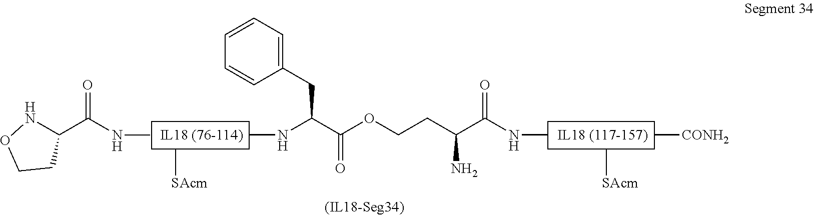

IL18-Seg34 preparation: Peptide ketoacid IL18-Seg3 (54.6 mg; 10.9 μmol; 1.0 eq) and hydroxylamine peptide IL18-Seg4 (66.8 mg; 13.1 μmol; 1.2 eq) were in dissolved in 9:1 DMSO/H₂O containing 0.1 M oxalic acid (546 μL). A very homogeneous liquid solution was obtained, which was gently agitated overnight at 60° C. Upon completion of the ligation reaction, the mixture was diluted with DMSO (1092 μL). Fmoc deprotection was initiated by the adding diethylamine (82 μL, 5%, v/v) and gently agitated at room temperature for 15 min. A second solution of diethylamine (82 μL) in DMSO (1638 μL) was added to the reaction mixture and gently agitated at room temperature for another 15 min. Gel formation was expected. Trifluoroacetic acid (200 μL) was added to neutralize the reaction mixture. A homogeneous and colorless liquid solution was obtained, which was further diluted with 1:1 CH₃CN/H₂O (q.s. 17 mL) with TFA (0.1%, v/v). The diluted mixture was directly injected into preparative HPLC. The crude ligated peptide solution was filtered and purified by preparative HPLC using Gemini NX-C18 110 Å column (5 μm, 250×50 mm) at a flow rate of 40 mL/min at 40° C. using CH₃CN/H₂O with 0.1% TFA (v/v) as mobile phase, with a gradient of 10% to 50% CH₃CN with 0.1% TFA (v/v) in 40 min. The diluted mixture was directly injected into preparative HPLC. The fractions containing the purified product were pooled and lyophilized to obtain IL18-Seg34 as a white solid. The isolated yield was 60.1 mg (56%). MS (ESI): C₄₃₉H₆₈₄N₁₁₄O₁₃₈S₂; Average isotope calculated 9831.0810 Da [M]; found: 9830.9439 Da.

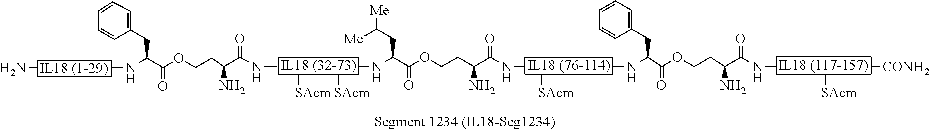

IL18-Seg1234 with Acm preparation: Peptide ketoacid IL18-Seg12 (16.1 mg; 1.97 μmol; 1.2 eq) and hydroxylamine peptide IL18-Seg34 (16.1 mg; 1.64 μmol; 1.0 eq) were in dissolved in 9:1 DMSO/H₂O containing 0.1 M oxalic acid (110 μL). A homogeneous liquid solution was obtained, which was reacted overnight at 60° C. After completion of the ligation reaction, the mixture was diluted first with DMSO (1890 μL). The mixture was further diluted with 1:1 H₂O/CH₃CN (q.s. 8 mL) containing TFA (0.1%, v/v). The diluted mixture was filtered and injected into preparative HPLC. Crude ligated peptide was purified by preparative HPLC using Gemini NX-C18 110 Å column (5 μm, 250×50 mm) at a flow rate of 40 mL/min at 60° C. using CH₃CN/H₂O with 0.1% TFA (v/v) as mobile phase, with a gradient of 10 to 60% CH₃CN with 0.1% TFA (v/v) in 30 min. The fractions containing the purified product were pooled and lyophilized to obtain IL18-Seg12 as a white solid in >98% purity. The isolated yield was 10.0 mg (33%).

Table 4 shows modified IL-18 polypeptides which may be prepared according to the methods provided herein.

TABLE 4
Modified IL-18 polypeptides

| SEQ | | |
|---|---|---|
| ID | | |
| NO: | Sequence modifications* | Sequence |
| 1 | Native sequence | YFGKLESKLS VIRNLNDQVL FIDQGNRPLF |
| | | EDMTDSDCRD NAPRTIFIIS MYKDSQPRGM |
| | | AVTISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 2 | E06K, K53A, S55A | YFGKLKSKLS VIRNLNDQVL FIDQGNRPLF |
| | | EDMTDSDCRD NAPRTIFIIS MYADAQPRGM |
| | | AVTISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 3 | Y01G, F02A, E06K, M51G, | GAGKLKSKLS VIRNLNDQVL FIDQGNRPLF |
| | K53A, D54A, S55A, T63A | EDMTDSDCRD NAPRTIFIIS GYAAAQPRGM |
| | | AVAISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 4 | K53A | YFGKLESKLS VIRNLNDQVL FIDQGNRPLF |
| | | EDMTDSDCRD NAPRTIFIIS MYADSQPRGM |
| | | AVTISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 5 | S55A | YFGKLESKLS VIRNLNDQVL FIDQGNRPLF |
| | | EDMTDSDCRD NAPRTIFIIS MYKDAQPRGM |
| | | AVTISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 6 | E06K | YFGKLKSKLS VIRNLNDQVL FIDQGNRPLF |
| | | EDMTDSDCRD NAPRTIFIIS MYKDSQPRGM |
| | | AVTISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 7 | E06K, K53A | YFGKLKSKLS VIRNLNDQVL FIDQGNRPLF |
| | | EDMTDSDCRD NAPRTIFIIS MYADSQPRGM |
| | | AVTISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 8 | E06K, S55A | YFGKLKSKLS VIRNLNDQVL FIDQGNRPLF |
| | | EDMTDSDCRD NAPRTIFIIS MYKDAQPRGM |
| | | AVTISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 9 | K53A, S55A | YFGKLESKLS VIRNLNDQVL FIDQGNRPLF |
| | | EDMTDSDCRD NAPRTIFIIS MYADAQPRGM |
| | | AVTISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 10 | E06K, K53A, S55A, T63A | YFGKLKSKLS VIRNLNDQVL FIDQGNRPLF |
| | | EDMTDSDCRD NAPRTIFIIS MYADAQPRGM |
| | | AVAISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 11 | E06K, K53A, S55A, Y01G | GFGKLKSKLS VIRNLNDQVL FIDQGNRPLF |
| | | EDMTDSDCRD NAPRTIFIIS MYADAQPRGM |
| | | AVTISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 12 | E06K, K53A, S55A, F02A | YAGKLKSKLS VIRNLNDQVL FIDQGNRPLF |
| | | EDMTDSDCRD NAPRTIFIIS MYADAQPRGM |
| | | AVTISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 13 | E06K, K53A, S55A, D54A | YFGKLKSKLS VIRNLNDQVL FIDQGNRPLF |
| | | EDMTDSDCRD NAPRTIFIIS MYAAAQPRGM |
| | | AVTISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 14 | E06K, K53A, S55A, M51G | YFGKLKSKLS VIRNLNDQVL FIDQGNRPLF |
| | | EDMTDSDCRD NAPRTIFIIS GYADAQPRGM |
| | | AVTISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 15 | C38S, C68S, C76S, C127S | YFGKLESKLS VIRNLNDQVL FIDQGNRPLF |
| | | EDMTDSDSRD NAPRTIFIIS MYKDSQPRGM |
| | | AVTISVKSEK ISTLSSENKI ISFKEMNPPD |
| | | NIKDTKSDII FFQRSVPGHD NKMQFESSSY |
| | | EGYFLASEKE RDLFKLILKK EDELGDRSIM |
| | | FTVQNED |
| 16 | C38S, C68S, C76S, C127S, | YFGKLESKLS VIRNLNDQVL FIDQGNRPLF |
| | K70C | EDMTDSDSRD NAPRTIFIIS MYKDSQPRGM |
| | | AVTISVKSEC ISTLSSENKI ISFKEMNPPD |
| | | NIKDTKSDII FFQRSVPGHD NKMQFESSSY |
| | | EGYFLASEKE RDLFKLILKK EDELGDRSIM |
| | | FTVQNED |
| 17 | E06K, K53A, S55A, C38S, | YFGKLKSKLS VIRNLNDQVL FIDQGNRPLF |
| | C68S, C76S, C127S, K70C | EDMTDSDSRD NAPRTIFIIS MYADAQPRGM |
| | | AVTISVKSEC ISTLSSENKI ISFKEMNPPD |
| | | NIKDTKSDII FFQRSVPGHD NKMQFESSSY |
| | | EGYFLASEKE RDLFKLILKK EDELGDRSIM |
| | | FTVQNED |
| 18 | E06K, K53A, T63A | YFGKLKSKLS VIRNLNDQVL FIDQGNRPLF |
| | | EDMTDSDCRD NAPRTIFIIS MYADSQPRGM |
| | | AVAISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 19 | T63A | YFGKLESKLS VIRNLNDQVL FIDQGNRPLF |
| | | EDMTDSDCRD NAPRTIFIIS MYKDSQPRGM |
| | | AVAISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 20 | E06K, T63A | YFGKLKSKLS VIRNLNDQVL FIDQGNRPLF |
| | | EDMTDSDCRD NAPRTIFIIS MYKDSQPRGM |
| | | AVAISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 21 | K53A, T63A | YFGKLESKLS VIRNLNDQVL FIDQGNRPLF |
| | | EDMTDSDCRD NAPRTIFIIS MYADSQPRGM |
| | | AVAISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 22 | E06K, K53A, C38S, C68S, | YFGKLKSKLS VIRNLNDQVL FIDQGNRPLF |
| | C76S, C127S, K70C | EDMTDSDSRD NAPRTIFIIS MYADSQPRGM |
| | | AVTISVKSEC ISTLSSENKI ISFKEMNPPD |
| | | NIKDTKSDII FFQRSVPGHD NKMQFESSSY |
| | | EGYFLASEKE RDLFKLILKK EDELGDRSIM |
| | | FTVQNED |
| 23 | K53A, T63A, C38S, C68S, C76S, | YFGKLESKLS VIRNLNDQVL FIDQGNRPLF |
| | C127S, K70C | EDMTDSDSRD NAPRTIFIIS MYADSQPRGM |
| | | AVAISVKSEC ISTLSSENKI ISFKEMNPPD |
| | | NIKDTKSDII FFQRSVPGHD NKMQFESSSY |
| | | EGYFLASEKE RDLFKLILKK EDELGDRSIM |
| | | FTVQNED |
| 24 | E31Hse, M33Nle, M51Nle, | YFGKLESKLS VIRNLNDQVL FIDQGNRPLF |
| | M60Nle, S75Hse, M86Nle, | ZDXTDSDCRD NAPRTIFIIS XYKDSQPRGX |
| | M113Nle, E116Hse, M150Nle | AVTISVKCEK ISTLZCENKI ISFKEXNPPD |
| | | NIKDTKSDII FFQRSVPGHD NKXQFZSSSY |
| | | EGYFLACEKE RDLFKLILKK EDELGDRSIX |
| | | FTVQNED |
| | | X = Nle, Z = Hse |
| 25 | E31Hse, M33Nle, C38S, M51Nle, | YFGKLESKLS VIRNLNDQVL FIDQGNRPLF |
| | M60Nle, C68S, S75Hse, C76S, | ZDXTDSDSRD NAPRTIFIIS XYKDSQPRGX |
| | M86Nle, M113Nle, E116Hse, | AVTISVKSEK ISTLZSENKI ISFKEXNPPD |
| | C127S, M150Nle | NIKDTKSDII FFQRSVPGHD NKXQFZSSSY |
| | | EGYFLASEKE RDLFKLILKK EDELGDRSIX |
| | | FTVQNED |
| | | X = Nle, Z = Hse |
| 26 | E6K, E31Hse, M33Nle, M51Nle, | YFGKLKSKLS VIRNLNDQVL FIDQGNRPLF |
| | K53A, M60Nle, S75Hse, K70PEG, | ZDXTDSDCRD NAPRTIFIIS XYADSQPRGX |
| | M86Nle, M113Nle, E116Hse, | AVTISVKCEB ISTLZCENKI ISFKEXNPPD |
| | M150Nle | NIKDTKSDII FFQRSVPGHD NKXQFZSSSY |
| | | EGYFLACEKE RDLFKLILKK EDELGDRSIX |
| | | FTVQNED |
| | | X = Nle, Z = Hse, B = Azido lysine, |
| | | Lysino-PEG etc. |
| 27 | E31Hse, M33Nle, M51Nle, | YFGKLESKLS VIRNLNDQVL FIDQGNRPLF |
| | M60Nle, T63Hse, M86Nle, | ZDXTDSDCRD NAPRTIFIIS XYKDSQPRGX |
| | M113Nle, E116Hse, M150Nle | AVZISVKCEK ISTLSCENKI ISFKEXNPPD |
| | | NIKDTKSDII FFQRSVPGHD NKXQFZSSSY |
| | | EGYFLACEKE RDLFKLILKK EDELGDRSIX |
| | | FTVQNED |
| | | X = Nle, Z = Hse |
| 28 | E6K, E31Hse, M33Nle, M51Nle, | YFGKLKSKLS VIRNLNDQVL FIDQGNRPLF |
| | K53A, M60Nle, T63Hse, | ZDXTDSDCRD NAPRTIFIIS XYADSQPRGX |
| | K70PEG, M86Nle, M113Nle, | AVZISVKCEB ISTLSCENKI ISFKEXNPPD |
| | E116Hse, M150Nle | NIKDTKSDII FFQRSVPGHD NKXQFZSSSY |
| | | EGYFLACEKE RDLFKLILKK EDELGDRSIX |
| | | FTVQNED |
| | | X = Nle, Z = Hse, B = Azido lysine, |
| | | Lysino-PEG etc. |
| 29 | E31Hse, M33Nle, C38S, M51Nle, | YFGKLESKLS VIRNLNDQVL FIDQGNRPLF |
| | M60Nle, T63Hse, C68S, C76S, | ZDXTDSDSRD NAPRTIFIIS XYKDSQPRGX |
| | M86Nle, M113Nle, E116Hse, | AVZISVKSEK ISTLSSENKI ISFKEXNPPD |
| | C127S, M150Nle | NIKDTKSDII FFQRSVPGHD NKXQFZSSSY |
| | | EGYFLASEKE RDLFKLILKK EDELGDRSIX |
| | | FTVQNED |
| | | X = Nle, Z = Hse |
| 30 | E6K, E31Hse, M33Nle, C38S, | YFGKLKSKLS VIRNLNDQVL FIDQGNRPLF |
| | M51Nle, K53A, M60Nle, T63Hse, | ZDXTDSDSRD NAPRTIFIIS XYADSQPRGX |
| | C68S, K70PEG, C76S, M86Nle, | AVZISVKSEB ISTLSSENKI ISFKEXNPPD |
| | M113Nle, E116Hse, C127S, | NIKDTKSDII FFQRSVPGHD NKXQFZSSSY |
| | M150Nle | EGYFLASEKE RDLFKLILKK EDELGDRSIX |
| | | FTVQNED |
| | | X = Nle, Z = Hse, B = Azido lysine, |
| | | Lysino-PEG etc. |
| 31 | E31Hse, T63Hse, E116Hse | YFGKLESKLS VIRNLNDQVL FIDQGNRPLF |
| | | ZDMTDSDCRD NAPRTIFIIS MYKDSQPRGM |
| | | AVZISVKCEK ISTLSCENKI ISFKEMNPPD |
| | | NIKDTKSDII FFQRSVPGHD NKMQFZSSSY |
| | | EGYFLACEKE RDLFKLILKK EDELGDRSIM |
| | | FTVQNED |
| | | Z = Hse |
| 32 | E6K, E31Hse, K53A, T63Hse, | YFGKLKSKLS VIRNLNDQVL FIDQGNRPLF |
| | K70PEG, E116Hse | ZDMTDSDCRD NAPRTIFIIS MYADSQPRGM |
| | | AVZISVKCEB ISTLSCENKI ISFKEMNPPD |
| | | NIKDTKSDII FFQRSVPGHD NKMQFZSSSY |
| | | EGYFLACEKE RDLFKLILKK EDELGDRSIM |
| | | FTVQNED |
| | | Z = Hse, B = Azido lysine, Lysino-PEG |
| | | etc |
| 33 | E31Hse, C38S, T63Hse, C68S, | YFGKLESKLS VIRNLNDQVL FIDQGNRPLF |
| | C76S, E116Hse, C127S | ZDMTDSDSRD NAPRTIFIIS MYKDSQPRGM |
| | | AVZISVKSEK ISTLSSENKI ISFKEMNPPD |
| | | NIKDTKSDII FFQRSVPGHD NKMQFZSSSY |
| | | EGYFLASEKE RDLFKLILKK EDELGDRSIM |
| | | FTVQNED |
| | | Z = Hse |
| 34 | E6K, E31Hse, C38S, K53A, | YFGKLKSKLS VIRNLNDQVL FIDQGNRPLF |
| | T63Hse, C68S, K70PEG, C76S, | ZDMTDSDSRD NAPRTIFIIS MYADSQPRGM |
| | E116Hse, C127S | AVZISVKSEB ISTLSSENKI ISFKEMNPPD |
| | | NIKDTKSDII FFQRSVPGHD NKMQFZSSSY |
| | | EGYFLASEKE RDLFKLILKK EDELGDRSIM |
| | | FTVQNED |
| | | Z = Hse, B = Azido lysine, Lysino-PEG |
| | | etc |
| 35 | E31Hse, M33J, M51J, M60J, | YFGKLESKLS VIRNLNDQVL FIDQGNRPLF |
| | T63Hse, M86J, M113J, E116Hse, | ZDJTDSDCRD NAPRTIFIIS JYKDSQPRGJ |
| | M150J | AVZISVKCEK ISTLSCENKI ISFKEJNPPD |
| | | NIKDTKSDII FFQRSVPGHD NKJQFZSSSY |
| | | EGYFLACEKE RDLFKLILKK EDELGDRSIJ |
| | | FTVQNED |
| | | Z = Hse, J = O-methyl-L-homoserine |
| 36 | E6K, E31Hse, M33J, M51J, K53A, | YFGKLKSKLS VIRNLNDQVL FIDQGNRPLF |
| | M60J, T63Hse, K70PEG, M86J, | ZDJTDSDCRD NAPRTIFIIS JYADSQPRGJ |
| | M113J, E116Hse, M150J | AVZISVKCEB ISTLSCENKI ISFKEJNPPD |
| | | NIKDTKSDII FFQRSVPGHD NKJQFZSSSY |
| | | EGYFLACEKE RDLFKLILKK EDELGDRSIJ |
| | | FTVQNED |
| | | Z = Hse, J = O-methyl-L-homoserine, |
| | | B = Azido lysine, Lysino-PEG etc |
| 37 | E31Hse, M33J, C38S, M51J, | YFGKLESKLS VIRNLNDQVL FIDQGNRPLF |
| | M60J, T63Hse, C68S, C76S, | ZDJTDSDSRD NAPRTIFIIS JYKDSQPRGJ |
| | M86J, M113J, E116Hse, C127S, | AVZISVKSEK ISTLSSENKI ISFKEJNPPD |
| | M150J | NIKDTKSDII FFQRSVPGHD NKJQFZSSSY |
| | | EGYFLASEKE RDLFKLILKK EDELGDRSIJ |
| | | FTVQNED |
| | | Z = Hse, J = O-methyl-L-homoserine |
| 38 | E6K, E31Hse, M33J, C38S, M51J, | YFGKLKSKLS VIRNLNDQVL FIDQGNRPLF |
| | K53A, M60J, T63Hse, C68S, | ZDJTDSDSRD NAPRTIFIIS JYADSQPRGJ |
| | K70PEG, C76S, M86J, M113J, | AVZISVKSEB ISTLSSENKI ISFKEJNPPD |
| | E116Hse, C127S, M150J | NIKDTKSDII FFQRSVPGHD NKJQFZSSSY |
| | | EGYFLASEKE RDLFKLILKK EDELGDRSIJ |
| | | FTVQNED |
| | | Z = Hse, J = O-methyl-L-homoserine, |
| | | B = Azido lysine, Lysino-PEG etc |
| 39 | E6K, E31Hse, M33Nle, M51Nle, | YFGKLKSKLS VIRNLNDQVL FIDQGNRPLF |
| | K53A, M60Nle, S75Hse, M86Nle, | ZDXTDSDCRD NAPRTIFIIS XYADSQPRGX |
| | M113Nle, E116Hse, M150Nle | AVTISVKCEK ISTLZCENKI ISFKEXNPPD |
| | | NIKDTKSDII FFQRSVPGHD NKXQFZSSSY |
| | | EGYFLACEKE RDLFKLILKK EDELGDRSIX |
| | | FTVQNED |
| | | X = Nle, Z = Hse |
| 40 | E6K, E31Hse, K53A, T63Hse, | YFGKLKSKLS VIRNLNDQVL FIDQGNRPLF |
| | E116Hse | ZDMTDSDCRD NAPRTIFIIS MYADSQPRGM |
| | | AVZISVKCEK ISTLSCENKI ISFKEMNPPD |
| | | NIKDTKSDII FFQRSVPGHD NKMQFZSSSY |
| | | EGYFLACEKE RDLFKLILKK EDELGDRSIM |
| | | FTVQNED |
| | | Z = Hse |
| 41 | E6K, E31Hse, C38S, K53A, | YFGKLKSKLS VIRNLNDQVL FIDQGNRPLF |
| | T63Hse, C68S, C76S, E116Hse, | ZDMTDSDSRD NAPRTIFIIS MYADSQPRGM |
| | C127S | AVZISVKSEK ISTLSSENKI ISFKEMNPPD |
| | | NIKDTKSDII FFQRSVPGHD NKMQFZSSSY |
| | | EGYFLASEKE RDLFKLILKK EDELGDRSIM |
| | | FTVQNED |
| | | Z = Hse |
| 42 | E6K, E31Hse, M33Nle, M51Nle, | YFGKLKSKLS VIRNLNDQVL FIDQGNRPLF |
| | K53A, M60Nle, T63Hse, M86Nle, | ZDXTDSDCRD NAPRTIFIIS XYADSQPRGX |
| | M113Nle, E116Hse, M150Nle | AVZISVKCEK ISTLSCENKI ISFKEXNPPD |
| | | NIKDTKSDII FFQRSVPGHD NKXQFZSSSY |
| | | EGYFLACEKE RDLFKLILKK EDELGDRSIX |
| | | FTVQNED |
| | | X = Nle, Z = Hse |
| 43 | E6K, E31Hse, M33Nle, C38S, | YFGKLKSKLS VIRNLNDQVL FIDQGNRPLF |
| | M51Nle, K53A, M60Nle, T63Hse, | ZDXTDSDSRD NAPRTIFIIS XYADSQPRGX |
| | C68S, C76S, M86Nle, M113Nle, | AVZISVKSEK ISTLSSENKI ISFKEXNPPD |
| | E116Hse, C127S, M150Nle | NIKDTKSDII FFQRSVPGHD NKXQFZSSSY |
| | | EGYFLASEKE RDLFKLILKK EDELGDRSIX |
| | | FTVQNED |
| | | X = Nle, Z = Hse |
| 44 | E6K, E31Hse, M33J, M51J, K53A, | YFGKLKSKLS VIRNLNDQVL FIDQGNRPLF |
| | M60J, T63Hse, M86J, M113J, | ZDJTDSDCRD NAPRTIFIIS JYADSQPRGJ |
| | E116Hse, M150J | AVZISVKCEK ISTLSCENKI ISFKEJNPPD |
| | | NIKDTKSDII FFQRSVPGHD NKJQFZSSSY |
| | | EGYFLACEKE RDLFKLILKK EDELGDRSIJ |
| | | FTVQNED |
| | | Z = Hse, J =O-methyl-L-homoserine |
| 45 | E6K, E31Hse, M33J, C38S, M51J, | YFGKLKSKLS VIRNLNDQVL FIDQGNRPLF |
| | K53A, M60J, T63Hse, C68S, | ZDJTDSDSRD NAPRTIFIIS JYADSQPRGJ |
| | C76S, M86J, M113J, E116Hse, | AVZISVKSEK ISTLSSENKI ISFKEJNPPD |
| | C127S, M150J | NIKDTKSDII FFQRSVPGHD NKJQFZSSSY |
| | | EGYFLASEKE RDLFKLILKK EDELGDRSIJ |
| | | FTVQNED |
| | | Z = Hse, J = O-methyl-L-homoserine |
| 46 | E31Hse, M33Nle, M51Nle, | YFGKLESKLS VIRNLNDQVL FIDQGNRPLF |
| | M60Nle, K70PEG, S75Hse, | ZDXTDSDCRD NAPRTIFIIS XYKDSQPRGX |
| | M86Nle, M113Nle, E116Hse, | AVTISVKCEB ISTLZCENKI ISFKEXNPPD |
| | M150Nle | NIKDTKSDII FFQRSVPGHD NKXQFZSSSY |
| | | EGYFLACEKE RDLFKLILKK EDELGDRSIX |
| | | FTVQNED |
| | | X = Nle, Z = Hse, B = Azido lysine, |
| | | Lysino-PEG etc. |
| 47 | E31Hse, T63Hse, K70PEG, | YFGKLESKLS VIRNLNDQVL FIDQGNRPLF |
| | E116Hse | ZDMTDSDCRD NAPRTIFIIS MYKDSQPRGM |
| | | AVZISVKCEB ISTLSCENKI ISFKEMNPPD |
| | | NIKDTKSDII FFQRSVPGHD NKMQFZSSSY |
| | | EGYFLACEKE RDLFKLILKK EDELGDRSIM |
| | | FTVQNED |
| | | Z = Hse, B = Azido lysine, Lysino-PEG |
| | | etc |
| 48 | E31Hse, C38S, T63Hse, C68S, | YFGKLESKLS VIRNLNDQVL FIDQGNRPLF |
| | K70PEG, C76S, E116Hse, C127S | ZDMTDSDSRD NAPRTIFIIS MYKDSQPRGM |
| | | AVZISVKSEB ISTLSSENKI ISFKEMNPPD |
| | | NIKDTKSDII FFQRSVPGHD NKMQFZSSSY |
| | | EGYFLASEKE RDLFKLILKK EDELGDRSIM |
| | | FTVQNED |
| | | Z = Hse, B = Azido lysine, Lysino-PEG |
| | | etc |
| 49 | E31Hse, M33Nle, M51Nle, | YFGKLESKLS VIRNLNDQVL FIDQGNRPLF |
| | M60Nle, T63Hse, K70PEG, | ZDXTDSDCRD NAPRTIFIIS XYKDSQPRGX |
| | M86Nle, M113Nle, E116Hse, | AVZISVKCEB ISTLSCENKI ISFKEXNPPD |
| | M150Nle | NIKDTKSDII FFQRSVPGHD NKXQFZSSSY |
| | | EGYFLACEKE RDLFKLILKK EDELGDRSIX |
| | | FTVQNED |
| | | X = Nle, Z = Hse, B = Azido lysine, |
| | | Lysino-PEG etc. |
| 50 | E31Hse, M33Nle, C38S, M51Nle, | YFGKLESKLS VIRNLNDQVL FIDQGNRPLF |
| | M60Nle, T63Hse, C68S, K70PEG, | ZDXTDSDSRD NAPRTIFIIS XYKDSQPRGX |
| | C76S, M86Nle, M113Nle, | AVZISVKSEB ISTLSSENKI ISFKEXNPPD |
| | E116Hse, C127S, M150Nle | NIKDTKSDII FFQRSVPGHD NKXQFZSSSY |
| | | EGYFLASEKE RDLFKLILKK EDELGDRSIX |
| | | FTVQNED |
| | | X = Nle, Z = Hse, B = Azido lysine, |
| | | Lysino-PEG etc. |
| 51 | E31Hse, M33J, M51J, M60J, | YFGKLESKLS VIRNLNDQVL FIDQGNRPLF |
| | T63Hse, K70PEG, M86J, M113J, | ZDJTDSDCRD NAPRTIFIIS JYKDSQPRGJ |
| | E116Hse, M150J | AVZISVKCEB ISTLSCENKI ISFKEJNPPD |
| | | NIKDTKSDII FFQRSVPGHD NKJQFZSSSY |
| | | EGYFLACEKE RDLFKLILKK EDELGDRSIJ |
| | | FTVQNED |
| | | Z = Hse, J = O-methyl-L-homoserine, |
| | | B = Azido lysine, Lysino-PEG etc |
| 52 | E31Hse, M33J, C38S, M51J, | YFGKLESKLS VIRNLNDQVL FIDQGNRPLF |
| | M60J, T63Hse, C68S, K70PEG, | ZDJTDSDSRD NAPRTIFIIS JYKDSQPRGJ |
| | C76S, M86J, M113J, E116Hse, | AVZISVKSEB ISTLSSENKI ISFKEJNPPD |
| | C127S, M150J | NIKDTKSDII FFQRSVPGHD NKJQFZSSSY |
| | | EGYFLASEKE RDLFKLILKK EDELGDRSIJ |
| | | FTVQNED |
| | | Z = Hse, J = O-methyl-L-homoserine, |
| | | B = Azido lysine, Lysino-PEG etc |
| 53 | E6K, E31Hse, K53A, T63A, | YFGKLKSKLS VIRNLNDQVL FIDQGNRPLF |
| | S75Hse, E116Hse | ZDMTDSDCRD NAPRTIFIIS MYADSQPRGM |
| | | AVAISVKCEK ISTLZCENKI ISFKEMNPPD |
| | | NIKDTKSDII FFQRSVPGHD NKMQFZSSSY |
| | | EGYFLACEKE RDLFKLILKK EDELGDRSIM |
| | | FTVQNED |
| | | Z = Hse |
| 54 | E6K, E31Hse, C38S, K53A, T63A, | YFGKLKSKLS VIRNLNDQVL FIDQGNRPLF |
| | C68S, S75Hse, C76S, E116Hse, | ZDMTDSDSRD NAPRTIFIIS MYADSQPRGM |
| | C127S | AVAISVKSEK ISTLZSENKI ISFKEMNPPD |
| | | NIKDTKSDII FFQRSVPGHD NKMQFZSSSY |
| | | EGYFLASEKE RDLFKLILKK EDELGDRSIM |
| | | FTVQNED |
| | | Z = Hse |
| 55 | E6K, E31Hse, M33Nle, M51Nle, | YFGKLKSKLS VIRNLNDQVL FIDQGNRPLF |
| | K53A, M60Nle, T63A, S75Hse, | ZDXTDSDCRD NAPRTIFIIS XYADSQPRGX |
| | M86Nle, M113Nle, E116Hse, | AVAISVKCEK ISTLZCENKI ISFKEXNPPD |
| | M150Nle | NIKDTKSDII FFQRSVPGHD NKXQFZSSSY |
| | | EGYFLACEKE RDLFKLILKK EDELGDRSIX |
| | | FTVQNED |
| | | X = Nle, Z = Hse |
| 56 | E6K, E31Hse, M33Nle, C38S, | YFGKLKSKLS VIRNLNDQVL FIDQGNRPLF |
| | M51Nle, K53A, M60Nle, T63A, | ZDXTDSDSRD NAPRTIFIIS XYADSQPRGX |
| | C68S, S75Hse, C76S, M86Nle, | AVAISVKCEK ISTLZSENKI ISFKEXNPPD |
| | M113Nle, E116Hse, C127S, | NIKDTKSDII FFQRSVPGHD NKXQFZSSSY |
| | M150Nle | EGYFLASEKE RDLFKLILKK EDELGDRSIX |
| | | FTVQNED |
| | | X = Nle, Z = Hse |
| 57 | E6K, E31Hse, M33J, M51J, K53A, | YFGKLKSKLS VIRNLNDQVL FIDQGNRPLF |
| | M60J, T63A, S75Hse, M86J, | ZDJTDSDCRD NAPRTIFIIS JYADSQPRGJ |
| | M113J, E116Hse, M150J | AVAISVKCEK ISTLZCENKI ISFKEJNPPD |
| | | NIKDTKSDII FFQRSVPGHD NKJQFZSSSY |
| | | EGYFLACEKE RDLFKLILKK EDELGDRSIJ |
| | | FTVQNED |
| | | Z = Hse, J = O-methyl-L-homoserine |
| 58 | E6K, E31Hse, M33J, C38S, M51J, | YFGKLKSKLS VIRNLNDQVL FIDQGNRPLF |
| | K53A, M60J, 163A, C68S, | ZDJTDSDSRD NAPRTIFIIS JYADSQPRGJ |
| | S75Hse, C76S, M86J, M113J, | AVAISVKCEK ISTLZSENKI ISFKEJNPPD |
| | E116Hse, C127S, M150J | NIKDTKSDII FFQRSVPGHD NKJQFZSSSY |
| | | EGYFLASEKE RDLFKLILKK EDELGDRSIJ |
| | | FTVQNED |
| | | Z = Hse, J = O-methyl-L-homoserine |
| 59 | E6K, E31Hse, M33Nle, M51Nle, | YFGKLKSKLS VIRNLNDQVL FIDQGNRPLF |
| | K53A, M60Nle, T63Hse, C68PEG, | ZDXTDSDCRD NAPRTIFIIS XYADSQPRGX |
| | M86Nle, M113Nle, E116Hse, | AVZISVKαEK ISTLSCENKI ISFKEXNPPD |
| | M150Nle | NIKDTKSDII FFQRSVPGHD NKXQFZSSSY |
| | | EGYFLACEKE RDLFKLILKK EDELGDRSIX |
| | | FTVQNED |
| | | X = Nle, Z = Hse, α = Amino Acid-PEG-azide |
| 60 | E6K, E31Hse, M33Nle, M51Nle, | YFGKLKSKLS VIRNLNDQVL FIDQGNRPLF |
| | K53A, M60Nle, T63Hse, E69PEG, | ZDXTDSDCRD NAPRTIFIIS XYADSQPRGX |
| | M86Nle, M113Nle, E116Hse, | AVZISVKCαK ISTLSCENKI ISFKEXNPPD |
| | M150Nle | NIKDTKSDII FFQRSVPGHD NKXQFZSSSY |
| | | EGYFLACEKE RDLFKLILKK EDELGDRSIX |
| | | FTVQNED |
| | | X = Nle, Z = Hse, α = Amino Acid-PEG-azide |
| 61 | E6K, E31Hse, K53A, T63Hse, | YFGKLKSKLS VIRNLNDQVL FIDQGNRPLF |
| | C68PEG, E116Hse | ZDMTDSDCRD NAPRTIFIIS MYADSQPRGM |
| | | AVZISVKαEK ISTLSCENKI ISFKEMNPPD |
| | | NIKDTKSDII FFQRSVPGHD NKMQFZSSSY |
| | | EGYFLACEKE RDLFKLILKK EDELGDRSIM |
| | | FTVQNED |
| | | Z = Hse, α = Amino Acid-PEG-azide |
| 62 | E6K, E31Hse, K53A, T63Hse, | YFGKLKSKLS VIRNLNDQVL FIDQGNRPLF |
| | E69PEG, E116Hse | ZDMTDSDCRD NAPRTIFIIS MYADSQPRGM |
| | | AVZISVKCαK ISTLSCENKI ISFKEMNPPD |
| | | NIKDTKSDII FFQRSVPGHD NKMQFZSSSY |
| | | EGYFLACEKE RDLFKLILKK EDELGDRSIM |
| | | FTVQNED |
| | | Z = Hse, α = Amino Acid-PEG-azide |
| 63 | E6K, E31Hse, M33J, M51J, K53A, | YFGKLKSKLS VIRNLNDQVL FIDQGNRPLF |
| | M60J, T63Hse, C68PEG, M86J, | ZDJTDSDCRD NAPRTIFIIS JYADSQPRGJ |
| | M113J, E116Hse, M150J | AVZISVKαEK ISTLSCENKI ISFKEJNPPD |
| | | NIKDTKSDII FFQRSVPGHD NKJQFZSSSY |
| | | EGYFLACEKE RDLFKLILKK EDELGDRSIJ |
| | | FTVQNED |
| | | Z = Hse, J = O-methyl-L-homoserine, |
| | | α = Amino Acid-PEG-azide |
| 64 | E6K, E31Hse, M33J, M51J, K53A, | YFGKLKSKLS VIRNLNDQVL FIDQGNRPLF |
| | M60J, T63Hse, E69PEG, M86J, | ZDJTDSDCRD NAPRTIFIIS JYADSQPRGJ |
| | M113J, E116Hse, M150J | AVZISVKCαK ISTLSCENKI ISFKEJNPPD |
| | | NIKDTKSDII FFQRSVPGHD NKJQFZSSSY |
| | | EGYFLACEKE RDLFKLILKK EDELGDRSIJ |
| | | FTVQNED |
| | | Z = Hse, J = O-methyl-L-homoserine, |
| | | α = Amino Acid-PEG-azide |
| 65 | E6K,E31Hse, M33J, C38S, M51J, | YFGKLKSKLS VIRNLNDQVL FIDQGNRPLF |
| | K53A, M60J, T63Hse, M86J, | ZDJTDSDSRD NAPRTIFIIS JYADSQPRGJ |
| | M113J, E116Hse, M150J | AVZISVKCEK ISTLSCENKI ISFKEJNPPD |
| | | NIKDTKSDII FFQRSVPGHD NKJQFZSSSY |
| | | EGYFLACEKE RDLFKLILKK EDELGDRSIJ |
| | | FTVQNED |
| | | Z = Hse, J = O-methyl-L-homoserine |
| 66 | E6K, E31Hse, M33J, C38A, M51J, | YFGKLKSKLS VIRNLNDQVL FIDQGNRPLF |
| | K53A, M60J, T63Hse, M86J, | ZDJTDSDARD NAPRTIFIIS JYADSQPRGJ |
| | M113J, E116Hse, M150J | AVZISVKCEK ISTLSCENKI ISFKEJNPPD |
| | | NIKDTKSDII FFQRSVPGHD NKJQFZSSSY |
| | | EGYFLACEKE RDLFKLILKK EDELGDRSIJ |
| | | FTVQNED |
| | | Z = Hse, J = O-methyl-L-homoserine |
| 67 | E6K, E31Hse, C38S, K53A, | YFGKLKSKLS VIRNLNDQVL FIDQGNRPLF |
| | T63Hse, E116Hse | ZDMTDSDSRD NAPRTIFIIS MYADSQPRGM |
| | | AVZISVKCEK ISTLSCENKI ISFKEMNPPD |
| | | NIKDTKSDII FFQRSVPGHD NKMQFZSSSY |
| | | EGYFLACEKE RDLFKLILKK EDELGDRSIM |
| | | FTVQNED |
| | | Z = Hse |
| 68 | E6K, E31Hse, C38A, K53A, | YFGKLKSKLS VIRNLNDQVL FIDQGNRPLF |
| | T63Hse, E116Hse | ZDMTDSDARD NAPRTIFIIS MYADSQPRGM |
| | | AVZISVKCEK ISTLSCENKI ISFKEMNPPD |
| | | NIKDTKSDII FFQRSVPGHD NKMQFZSSSY |
| | | EGYFLACEKE RDLFKLILKK EDELGDRSIM |
| | | FTVQNED |
| | | Z = Hse |
| 69 | E6K, K53A, C38S, C68S, C76S, | YFGKLKSKLS VIRNLNDQVL FIDQGNRPLF |
| | C127S, K70C | EDMTDSDSRD NAPRTIFIIS MYADSQPRGM |
| | | AVTISVKSEC ISTLSSENKI ISFKEMNPPD |
| | | NIKDTKSDII FFQRSVPGHD NKMQFESSSY |
| | | EGYFLASEKE RDLFKLILKK EDELGDRSIM |
| | | FTVQNED |
| 70 | E6K, K53A, C38S, C76S, C127S | YFGKLKSKLS VIRNLNDQVL FIDQGNRPLF |
| | | EDMTDSDSRD NAPRTIFIIS MYADSQPRGM |
| | | AVTISVKCEK ISTLSSENKI ISFKEMNPPD |
| | | NIKDTKSDII FFQRSVPGHD NKMQFESSSY |
| | | EGYFLASEKE RDLFKLILKK EDELGDRSIM |
| | | FTVQNED |
| 71 | E6K, C38S, K53A | YFGKLKSKLS VIRNLNDQVL FIDQGNRPLF |
| | | EDMTDSDSRD NAPRTIFIIS MYADSQPRGM |
| | | AVTISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 72 | E6K, C38A, K53A | YFGKLKSKLS VIRNLNDQVL FIDQGNRPLF |
| | | EDMTDSDARD NAPRTIFIIS MYADSQPRGM |
| | | AVTISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 73 | E6K, C38Q, K53A | YFGKLKSKLS VIRNLNDQVL FIDQGNRPLF |
| | | EDMTDSDQRD NAPRTIFIIS MYADSQPRGM |
| | | AVTISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 74 | E6K, C38A, K53A, C76A | YFGKLKSKLS VIRNLNDQVL FIDQGNRPLF |
| | | EDMTDSDARD NAPRTIFIIS MYADSQPRGM |
| | | AVTISVKCEK ISTLSAENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 75 | E6K, C38A, K53A, C127A | YFGKLKSKLS VIRNLNDQVL FIDQGNRPLF |
| | | EDMTDSDARD NAPRTIFIIS MYADSQPRGM |
| | | AVTISVKCEK ISTLSCENKI ISFKEMNPPD |
| | | NIKDTKSDII FFQRSVPGHD NKMQFESSSY |
| | | EGYFLAAEKE RDLFKLILKK EDELGDRSIM |
| | | FTVQNED |
| 76 | E6K, C38A, K53A, C76A, C127A | YFGKLKSKLS VIRNLNDQVL FIDQGNRPLF |
| | | EDMTDSDARD NAPRTIFIIS MYADSQPRGM |
| | | AVTISVKCEK ISTLSAENKI ISFKEMNPPD |
| | | NIKDTKSDII FFQRSVPGHD NKMQFESSSY |
| | | EGYFLAAEKE RDLFKLILKK EDELGDRSIM |
| | | FTVQNED |
| 80 | E6K, K53A, C38A, S55A, T63A | YFGKLKSKLS VIRNLNDQVL FIDQGNRPLF |
| | | EDMTDSDARD NAPRTIFIIS MYADAQPRGM |
| | | AVAISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 81 | E6K, C38Q, K53A, S55A, T63A | YFGKLKSKLS VIRNLNDQVL FIDQGNRPLF |
| | | EDMTDSDQRD NAPRTIFIIS MYADAQPRGM |
| | | AVAISVKCEK ISTLSCENKI ISFKEMNPPD |
| | | NIKDTKSDII FFQRSVPGHD NKMQFESSSY |
| | | EGYFLACEKE RDLFKLILKK EDELGDRSIM |
| | | FTVQNED |
| 82 | E6K, K53A, K84A | YFGKLKSKLS VIRNLNDQVL FIDQGNRPLF |
| | | EDMTDSDCRD NAPRTIFIIS MYADSQPRGM |
| | | AVTISVKCEK ISTLSCENKI ISFAEMNPPD |
| | | NIKDTKSDII FFQRSVPGHD NKMQFESSSY |
| | | EGYFLACEKE RDLFKLILKK EDELGDRSIM |
| | | FTVQNED |
| 83 | E6K, K53A, D98A | YFGKLKSKLS VIRNLNDQVL FIDQGNRPLF |
| | | EDMTDSDCRD NAPRTIFIIS MYADSQPRGM |
| | | AVTISVKCEK ISTLSCENKI ISFKEMNPPD |
| | | NIKDTKSAII FFQRSVPGHD NKMQFESSSY |
| | | EGYFLACEKE RDLFKLILKK EDELGDRSIM |
| | | FTVQNED |

*Residue position numbering based on SEQ ID NO: 1 as a reference sequence

### Example 4A—Synthesis of a Modified IL-18 Polypeptide of SEQ ID NO: 42

A modified linear IL-18 polypeptide of SEQ ID NO: 42 was prepared according to the protocol provided below.

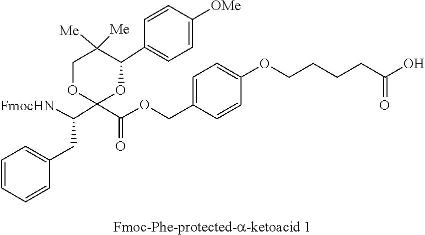

Segment 1A (IL18 (1-29)-Phe-α-ketoacid: Preloading of Fmoc-Phe-protected-α-ketoacid 1 was performed on a Fmoc-Rink Amide MBHA resin. 5 g of resin (loading: 0.56 mmol/g, 2.8 mmol scale) was swollen in DMF for 20 min. The resin was treated twice with 20% 4-methylpiperidine in DMF (v/v) at room temperature for 10 min. and was washed several times with DMF. Ketoacid 1 (1.7 g, 2.1 mmol, 0.75 eq) and HATU (800 mg, 2.1 mmol, 0.75 eq) were dissolved in DMF (15 mL). Pre-activation was performed at room temperature for 3 min by adding DIPEA (730 μL, 4.2 mmol, 1.5 eq). The reaction mixture was added to the swollen resin and gently agitated at room temperature for 3 h. The resin was rinsed thoroughly with DMF. Capping of unreacted amines on the resin was initiated by adding a solution of acetic anhydride (2.1 mL) and DIPEA (3.9 mL) in DMF (15 mL) and gently agitating the reaction at room temperature for 15 min. The resin was rinsed thoroughly with DCM followed by diethyl ether and dried. The loading of the resin was measured (0.27 mmol/g).

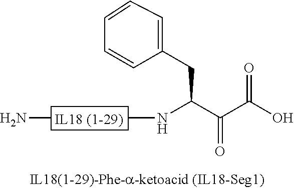

The IL18(1-29)-Phe-α-ketoacid segment was synthesized on a 0.2 mmol scale on Rink Amide MBHA resin pre-loaded with Fmoc-Phe-protected-α-ketoacid (741 mg) with a substitution capacity of ˜0.27 mmol/g.

Automated Fmoc-SPPS of IL18(1-29)-Phe-α-ketoacid: The coupling reactions were performed at room temperature for 30 min by adding a solution of Fmoc-amino acids dissolved in DMF (2.0 mL, 0.4 M, 4 eq), OxymaPure in DMF (2 mL, 0.4 M, 4 eq) and DIC in DMF (2 mL, 0.4 M, 4 eq) to the resin. For position 14 to 1, double couplings were required. Capping was performed after each amino acid at room temperature for 6 min by adding a 20% (v/v) acetic anhydride solution in DMF (2 mL) and NMM in DMF (0.8 M, 2.0 mL). The Fmoc deprotection reaction was performed using 20% (v/v) 4-methylpiperidine in DMF containing Cl-HOBt (0.1 M) at room temperature for 8 min.

The resin was washed with DCM and dried under vacuum. The mass of the dried peptidyl resin was 3.4 g. The peptide was cleaved from the resin using a mixture of 95:2.5:2.5 TFA/DODT/H₂O (10 mL/g resin) at room temperature for 2.0 h. The resin was filtered off from the cleavage cocktail, and the filtrate was concentrated and diluted 20-fold with cold diethyl ether (20° C.), allowing the peptide to precipitate. After centrifugation, the ether layer was carefully decanted, and the peptide precipitate was resuspended in diethyl ether, triturated and centrifuged. Ether washings were repeated twice, and the resulting peptide precipitate was dried. The mass of crude peptide was 1.07 g. Purification of crude IL18(1-29)-Phe-α-ketoacid segment 1A was performed by preparative HPLC using Shiseido Capcell Pak C18 column (5 μm, 250×20 mm) at a flow rate of 10 mL/min at 60° C. using CH₃CN/H₂O with 0.1% TFA (v/v) as mobile phase, with a gradient of 10 to 60% CH₃CN with 0.1% TFA (v/v) in 40 min. The fractions containing the purified product were pooled and lyophilized to obtain IL18(1-29)-Phe-α-ketoacid (IL18-Seg1) as a white solid in >90% purity. The isolated yield based on the resin loading was 348 mg (29%). MS (ESI): C₁₆₄H₂₆₀N₄₄O₄₄; Average isotope calculated 3551.9517 Da [M]; found: 3551.9644 Da [M].

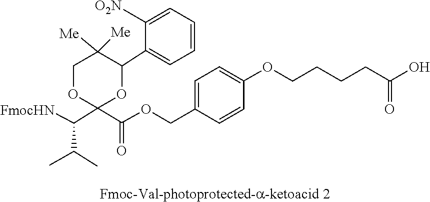

Segment 2A (Opr-IL18(32-61)-Val-photoprotected-α-ketoacid): Preloading of Fmoc-Val-photoprotected-α-ketoacid 2 was performed on a Fmoc-Rink-Amide MBHA resin. 3.05 g of resin (loading: 0.56 mmol/g, 1.71 mmol scale) was swollen in DMF for 20 min. Ketoacid 2 (1.0 g, 1.28 mmol, 0.75 eq) and HATU (487 mg, 1.28 mmol, 0.75 eq) were dissolved in DMF (10 mL). Pre-activation was performed at room temperature for 2 min by adding NMM (280 μL, 2.56 mmol, 1.5 eq). The reaction mixture was added to the swollen resin and gently agitated for 15 h at room temperature. The resin was rinsed thoroughly with DMF. Capping of unreacted amines on the resin was initiated by adding a solution of acetic anhydride (1.29 mL) and DIPEA (2.38 mL) in DMF (10 mL) and gently agitating the mixture at room temperature for 15 min. The resin was rinsed thoroughly with DCM and diethyl ether and dried. The loading of the resin was measured (0.307 mmol/g).

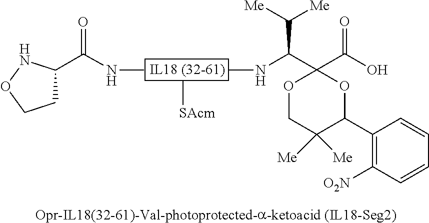

Opr-IL18(32-61)-Val-photoprotected-α-ketoacid segment was synthesized on a 0.2 mmol scale on Rink Amide MBHA resin pre-loaded with Fmoc-Val-photoprotected-α-ketoacid with a substitution capacity of ˜0.307 mmol/g.

Automated Fmoc-SPPS from position 61 to 56: The coupling reactions were performed at room temperature for 30 min by adding the Fmoc-amino acids dissolved in DMF (2.0 mL, 0.4 M, 4 eq), OxymaPure in DMF (2 mL, 0.4 M, 4 eq) and DIC in DMF (2 mL, 0.4 M, 4 eq) to the resin. Capping was performed after each amino acid at room temperature for 6 min by adding a 20% (v/v) acetic anhydride solution in DMF (2 mL) and NMM in DMF (0.8 M, 2.0 mL). Fmoc deprotection reaction was performed using 20% (v/v) 4-methylpiperidine in DMF at room temperature for 8 min.

Manual coupling of Fmoc-L-Asp(tBu)-L-Ser[Ψ(Me,Me)Pro]-OH was then performed. A solution of Fmoc-L-Asp(tBu)-L-Ser[Ψ(Me,Me)Pro]-OH (322 mg, 0.6 mmol, 3 eq), HATU (228 mg, 0.6 mmol, 3 eq) and DIPEA (209 μL, 1.2 mmol, 6 eq) in 6 mL of DMF was prepared (3 min of pre-activation at room temperature) and added to the resin. The reaction was gently agitated at room temperature for 2 h.

Automated Fmoc-SPPS from position 53 to 51: The coupling reactions were performed using the same conditions as previously mentioned for the beginning of the sequence.

Manual coupling of Fmoc-L-Ile-L-Ser[Ψ(Me,Me)Pro]-OH was then performed. A solution of Fmoc-L-Ile-L-Ser[Ψ(Me,Me)Pro]-OH (288 mg, 0.6 mmol, 3 eq), HATU (228 mg, 0.6 mmol, 3 eq) and DIPEA (209 μL, 1.2 mmol, 6 eq) in 6 mL of DMF was prepared (3 min of pre-activation at room temperature) and added to the resin. The reaction was gently agitated at room temperature for 2 h.

Automated SPPS from position 48 to 37: The coupling reactions were performed using the conditions described above. Double couplings were required for each position.

Manual coupling of Fmoc-L-Asp(tBu)-L-Ser[Ψ(Me,Me)Pro]-OH was then performed. A solution of Fmoc-L-Asp(tBu)-L-Ser[Ψ(Me,Me)Pro]-OH (322 mg, 0.6 mmol, 3 eq), HATU (228 mg, 0.6 mmol, 3 eq) and DIPEA (209 μL, 1.2 mmol, 6 eq) in 6 mL of DMF was prepared (3 min of pre-activation at room temperature) and added to the resin. The reaction was gently agitated at room temperature for 2 h.

Automated Fmoc-SPPS from position 34 to 32: The coupling reactions were performed using the conditions described above. Double couplings were required for each position.

Manual coupling of Boc-5-(S)-oxaproline was then performed. A solution of Boc-5-(S)-oxaproline (130 mg, 0.6 mmol, 3 eq), HATU (228 mg, 0.6 mmol, 3 eq) and DIPEA (209 μL, 1.2 mmol, 6 eq) in 6 mL of DMF was prepared (3 min of pre-activation at room temperature) and added to the resin. The reaction was gently agitated at room temperature for 2 h. The resin was washed with DCM and diethyl ether and dried under vacuum. The mass of the dried peptidyl resin was 1.2 g.

The peptide was cleaved from the resin using a mixture of 95:2.5:2.5 TFA/DODT/H₂O (10 mL/g resin) and gently agitating the mixture at room temperature for 2.0 h. The resin was filtered off from the cleavage cocktail, and the filtrate was concentrated and diluted 20-fold with cold diethyl ether (20° C.), allowing the peptide to precipitate. After centrifugation, the ether layer was carefully decanted, and the peptide precipitate was resuspended in diethyl ether, triturated and centrifuged. Ether washings were repeated twice, and the resulting peptide precipitate was dried. The mass of crude peptide was 520 mg. Purification of the crude Opr-IL18(32-61)-Val-photoprotected-α-ketoacid segment 2A was performed by preparative HPLC using Shiseido Capcell Pak C18 column (5 μm, 250×20 mm) at a flow rate of 10 mL/min at 60° C. using CH₃CN/H₂O with 0.1% TFA (v/v) as mobile phase, with a gradient of 10 to 70% CH₃CN with 0.1% TFA (v/v) in 40 min. The fractions containing the purified product were pooled and lyophilized to obtain Opr-IL18(32-61)-Val-photoprotected-α-ketoacid (IL18-Seg2) as a white solid in >95% purity. The isolated yield based on the resin loading was 200 mg (24%). C₁₆₆H₂₅₉N₄₅O₅₇S; Average isotope calculated: 3828.8527 Da [M]; found: 3829.1116 Da [M].

Segment 3A (Fmoc-Opr-IL18(64-114)-Phe-α-ketoacid): 488 mg of resin (loading: 0.40 mmol/g, 0.2 mmol scale) was swollen in DMF for 15 min. Ketoacid 1 (326 mg, 0.4 mmol, 2 eq) and HATU (152 mg, 0.4 mmol, 2 eq) were dissolved in DMF (6 mL). Pre-activation was performed at room temperature for 2 min by adding DIPEA (174 μL, 1 mmol, 5 eq). The reaction mixture was added to the swollen resin. The reaction was gently agitated for 3 hours at room temperature. The resin was rinsed thoroughly with DMF. Capping of unreacted amines on the resin was initiated by adding a solution of acetic anhydride (200 μL) and DIPEA (200 μL) in DMF (5 mL). The reaction was gently agitated at room temperature for 15 min. The resin was rinsed thoroughly with DMF. The final loading of the resin was not calculated and was estimated to be unchanged (0.40 mmol/g).

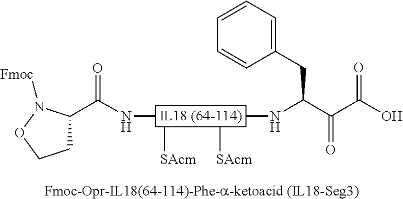

The Fmoc-Opr-IL18(64-114)-Phe-α-ketoacid segment was synthesized on a 0.2 mmol scale on Rink Amide ChemMatrix© resin pre-loaded with Fmoc-Phe-protected-α-ketoacid with a substitution capacity of ˜0.40 mmol/g.

Automated Fmoc-SPPS from position 96 to 114: The coupling reactions were performed at room temperature for 30 min by adding the Fmoc-amino acids dissolved in DMF (2.0 mL, 0.4 M, 4 eq), OxymaPure in DMF (2 mL, 0.4 M, 4 eq) and DIC in DMF (2 mL, 0.4 M, 4 eq) to the resin. Double couplings were required for each position. Capping was performed after each amino acid at room temperature for 6 min by adding a 20% (v/v) acetic anhydride solution in DMF (2 mL) and NMM in DMF (0.8 M, 2.0 mL). Fmoc deprotection reaction was performed using 20% (v/v) 4-methylpiperidine in DMF at room temperature for 8 min.

Manual coupling of Fmoc-L-Asp(tBu)-L-Thr[Ψ(Me,Me)Pro]-OH was then performed. A solution of Fmoc-L-Asp(tBu)-L-Thr[Ψ(Me,Me)Pro]-OH (332 mg, 0.6 mmol, 3 eq), HATU (228 mg, 0.6 mmol, 3 eq) and DIPEA (209 μL, 1.2 mmol, 6 eq) in 6 mL of DMF was prepared (3 min of pre-activation at room temperature) and added to the resin. The reaction was gently agitated at room temperature for 2 h.

Automated Fmoc-SPPS from position 73 to 93: The coupling reactions were performed using the conditions described above. Double couplings were required for each position. Fmoc deprotection reactions were performed using 20% (v/v) 4-methylpiperidine in DMF containing Cl-HOBt (0.1 M) at room temperature for 8 min.

Manual coupling of Fmoc-L-Ile-L-Ser[Ψ(Me,Me)Pro]-OH was then performed. A solution of Fmoc-L-Ile-L-Ser[Ψ(Me,Me)Pro]-OH (288 mg, 0.6 mmol, 3 eq), HATU (228 mg, 0.6 mmol, 3 eq) and DIPEA (209 μL, 1.2 mmol, 6 eq) in 6 ml of DMF was prepared (3 min of pre-activation at room temperature) and added to the resin. It was let to react at room temperature for 2 h.

Automated Fmoc-SPPS from position 64 to 70: The coupling reactions were performed using the same conditions used for position 73 to 93.

Manual coupling of Fmoc-5-(S)-oxaproline was then performed. A solution of Fmoc-5-(S)-oxaproline (204 mg, 0.6 mmol, 3 eq), HATU (228 mg, 0.6 mmol, 3 eq) and DIPEA (209 μL, 0.6 mmol, 6 eq) in 6 mL of DMF was prepared (3 min of pre-activation at room temperature) and added to the resin. The reaction was gently agitated at room temperature for 2 h. The resin was washed with DCM and dried under vacuum. The mass of the dried peptidyl resin was 2.0 g. The peptide was cleaved from the resin by stirring the resin in a mixture of 95:2.5:2.5 TFA/DODT/H₂O (10 mL/g resin) at room temperature for 2.0 h. The resin was filtered off from the cleavage cocktail, and the filtrate was concentrated and diluted 20-fold with cold diethyl ether (20° C.), allowing the peptide to precipitate. After centrifugation, the ether layer was carefully decanted, and the peptide precipitate was resuspended in diethyl ether, triturated and centrifuged. Ether washings were repeated twice, and the resulting peptide precipitate was dried. Mass of crude peptide was 1.05 g. Purification of crude Fmoc-Opr-IL18(64-114)-Phe-α-ketoacid segment 3A was performed by preparative HPLC using Shiseido Capcell Pak C18 column (5 μm, 250×20 mm) at a flow rate of 10 mL/min at 60° C. using CH₃CN/H₂O with 0.1% TFA (v/v) as mobile phase, with a gradient of 10 to 45% CH₃CN with 0.1% TFA (v/v) in 40 min. The fractions containing the purified product were pooled and lyophilized to obtain the Fmoc-Opr-IL18(64-114)-Phe-α-ketoacid (IL18-Seg3) as a white solid in >95% purity. The isolated yield based on the resin loading was 350 mg (27.1%). MS (MALDI-TOF): C₂₉₂H₄₅₃N₇₃O₈₈S₂; Average isotope calculated: 6458.3790 Da [M]; found: 6459.476 Da [M+H⁺].

Segment 4A (Opr-IL18 (117-157)): Preloading of Fmoc-Asp(OtBu)-OH was performed on a Fmoc-Rink-Amide MBHA resin. 4 g of resin (loading: 0.56 mmol/g, 2.24 mmol scale) was swollen in DMF for 15 min. The resin was treated with 20% in DMF (v/v) at room temperature for 20 min. The resin was washed several times with DMF. Fmoc-Asp(OtBu)-OH (691 mg, 1.68 mmol, 0.75 eq) and HATU (638 mg, 1.68 mmol, 0.75 eq) were dissolved in DMF (12 mL). Pre-activation was performed at room temperature for 3 min by adding DIPEA (585 μL, 4.48 mmol, 2 eq). The reaction mixture was added to the swollen resin and gently agitated overnight at room temperature. The resin was rinsed thoroughly with DMF. Capping of unreacted amines on the resin was initiated by adding a solution of acetic anhydride (1.17 mL) and DIPEA (2.34 mL) in DMF (12 mL) and gently agitating the mixture at room temperature for 15 min. The resin was rinsed thoroughly with DCM and dried. The loading of the resin was measured (0.34 mmol/g).

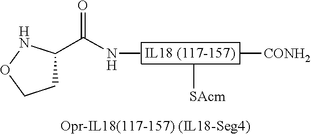

Opr-IL18(117-157) segment was synthesized on a 0.1 mmol scale on Rink Amide MBHA resin pre-loaded with Fmoc-Asp(OtBu)-OH with a substitution capacity of −0.34 mmol/g. 294 mg of resin was swollen in DMF for 15 min.

Automated Fmoc-SPPS from position 147 to 157: The coupling reactions were performed at room temperature for 30 min by adding Fmoc-amino acids dissolved in DMF (1.0 mL, 0.5 M, 5 eq), HCTU in DMF (1.0 mL, 0.48 M, 4.8 eq) and DIPEA in NMP (0.4 mL, 0.2 M, 8 eq) to the resin. Fmoc deprotection was performed using 20% (v/v) piperidine in DMF at room temperature for 15 min. Double coupling was required from position 117 to 146 as well as capping steps. Capping was performed at room temperature for 10 min at each position by adding 20% (v/v) acetic anhydride in DMF (1 mL) and DIPEA in DMF (0.2 M, 1 mL).

Manual coupling of Boc-5-(S)-oxaproline was then performed. A solution of Boc-5-(S)-oxaproline (65 mg, 0.3 mmol, 3 eq), HATU (114 mg, 0.3 mmol, 3 eq) and DIPEA (100 μL, 0.6 mmol, 6 eq) in 3 mL of DMF was prepared (3 min of pre-activation at room temperature) and added to the resin. The mixture was reacted at room temperature for 2 h.

The resin was washed with DCM and dried under vacuum. The mass of the dried peptidyl resin was 1.2 g. The peptide was cleaved from the resin using a mixture of 92.5:2.5:2.5:2.5 TFA/TIPS/DODT/H₂O (10 mL/g resin) at room temperature for 2 h. The resin was filtered off from the cleavage cocktail, and the filtrate was concentrated and diluted 20-fold with cold diethyl ether (20° C.), allowing the peptide to precipitate. After centrifugation, the ether layer was carefully decanted, and the peptide precipitate was resuspended in diethyl ether, triturated and centrifuged. Ether washings were repeated twice, and the resulting peptide precipitate was dried. Mass of crude peptide was 770 mg. Purification of crude Opr-IL18(117-157) segment 4A was performed by preparative HPLC using Gemini NX-C18 110 Å column (5 μm, 250×50 mm) at a flow rate of 40 mL/min at 40° C. using CH₃CN/H₂O with 0.1% TFA (v/v) as mobile phase, with a gradient of 10 to 50% CH₃CN with 0.1% TFA (v/v) in 40 min. The fractions containing the purified product were pooled and lyophilized to obtain Opr-IL18(117-157) (IL18-Seg4) as a white solid. The isolated yield based on the resin loading was 106 mg (21%). MS (ESI): C₂₂₂H₃₄₆N₅₆O₇₃S; Average isotope calculated 4998.4861 Da [M]; found: 4999.5126 Da [M].

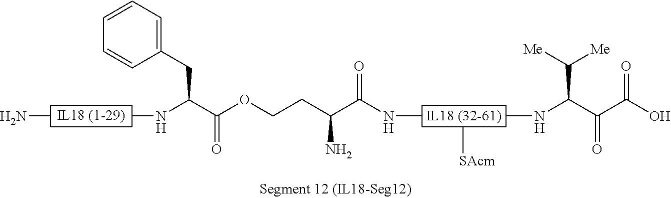

IL18-Seg12A preparation: Peptide ketoacid IL18-Seg1 (80.8 mg; 22.8 μmol; 1.2 eq) and hydroxylamine peptide IL18-Seg2 (72.6 mg; 19 μmol; 1.0 eq) were in dissolved in a 9.75:0.25 DMSO/H₂O solution containing 0.1 M oxalic acid (950 μL). A very homogeneous liquid solution was obtained. The ligation vial was protected from light by wrapping the vial in aluminum foil and gently agitated overnight at 60° C. After completion of the ligation, the mixture was diluted with DMSO (3550 μL) and irradiated at a wavelength of 365 nm for 3 h to allow photo deprotection of the C-terminal ketoacid. The mixture was further diluted with 1:1 CH₃CN/H₂O (q.s. 10 mL) with TFA (0.1%, v/v). The diluted mixture was filtered and injected into preparative HPLC. Crude ligated peptide was purified by preparative HPLC using Shiseido capcell Pak UG80 C18 column (5 μm, 250×50 mm) at a flow rate of 40 mL/min at 60° C., with a gradient of 20% to 50% CH₃CN with 0.1% TFA (v/v) in 30 min. The fractions containing the purified product were pooled and lyophilized to obtain IL18-Seg12A as a white solid in >98% purity. The isolated yield was 66.1 mg (48%). MS (ESI): C₃₂₁H₅₀₆N₈₈O₉₆S; m/z calculated: 7129.7248 Da [M]; found 7129.7177 Da [M].

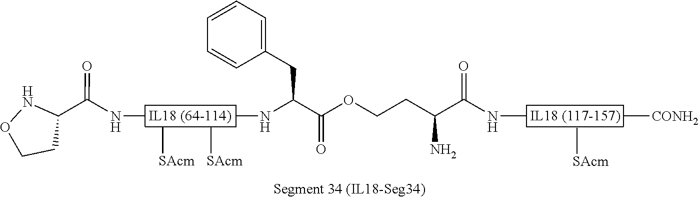

IL18-Seg34A preparation: Peptide ketoacid IL18-Seg3 (28 mg; 4.4 μmol; 1.1 eq) and hydroxylamine peptide IL18-Seg4 (20 mg; 4 μmol; 1 eq) were in dissolved in 97.5:2.5 DMSO/H₂O containing 0.1 M oxalic acid (200 μL). A very homogeneous liquid solution was obtained, which was gently agitated overnight at 60° C. Upon completion of the ligation reaction, the mixture was diluted with DMSO (400 μL). Fmoc deprotection was initiated by adding diethylamine (30 μL, 5%, v/v) and gently agitated at room temperature for 15 min. A second solution of diethylamine (30 μL) in DMSO (600 μL) was added to the reaction mixture and gently agitated at room temperature for another 15 min. Gel formation was expected. Trifluoroacetic acid (100 μL) was added to neutralize the reaction mixture. A homogeneous and colorless liquid solution was obtained, which was further diluted with 1:2 CH₃CN/H₂O (q.s. 10 mL) with TFA (0.1%, v/v). The diluted mixture was directly injected into preparative HPLC. The crude ligated peptide solution was filtered and purified by preparative HPLC using Shiseido Capcell Pak C18 column (5 μm, 250×20 mm) at a flow rate of 10 mL/min at 60° C. using CH₃CN/H₂O with 0.1% TFA (v/v) as mobile phase, with a gradient of 10 to 45% CH₃CN with 0.1% TFA (v/v) in 40 min. The fractions containing the purified product were pooled and lyophilized to obtain IL18-Seg34A as a white solid in >97% purity. The isolated yield was 20 mg (45%). MS (ESI): C₄₉₈H₇₈₉N₁₂₉O₁₅₇S₃; Average isotope calculated 11190.7044 Da [M]; found: 11190.7341 Da [M].

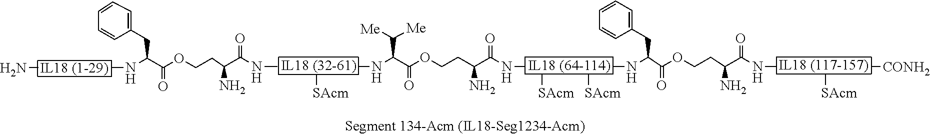

IL18-Seg1234A-Acm preparation: Peptide ketoacid IL18-Seg12 (7.5 mg; 1.1 μmol; 1.0 eq) and hydroxylamine peptide IL18-Seg34 (13 mg; 1.2 μmol; 1.1 eq) were dissolved in 97.5:2.5 DMSO/H₂O containing 0.1 M oxalic acid (69 μL). A homogeneous liquid solution was obtained, which was reacted overnight at 60° C. After completion of the ligation reaction, the mixture was diluted first with DMSO (140 μL). The mixture was further diluted with 1:1 H₂O/CH₃CN (q.s. 10 mL) containing TFA (0.1%, v/v). The diluted mixture was filtered and injected into preparative HPLC. Crude ligated peptide was purified by preparative HPLC using Shiseido Capcell Pak C18 column (5 μm, 250×20 mm) at a flow rate of 10 mL/min at 60° C. using CH₃CN/H₂O with 0.1% TFA (v/v) as mobile phase, with a gradient of 10 to 45% CH₃CN with 0.1% TFA (v/v) in 40 min. The fractions containing the purified product were pooled and lyophilized to obtain IL18-Seg1234A-Acm as a white solid in >92% purity. The isolated yield was 5.45 mg (26%). MS (ESI): C₈₁₅H₇₈₉N₁₂₉O₁₅₇S₃; Average isotope calculated: 18277.4418 Da [M]; found: 18278.5394 Da.

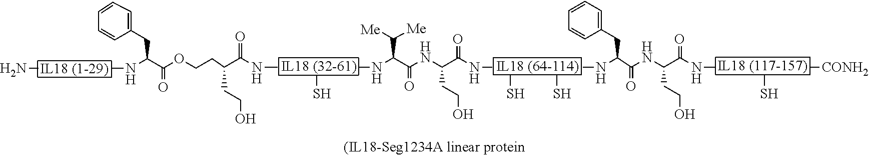

IL18-Seg1234A linear protein preparation: Rearrangement of linear protein: IL18-Seg1234A-Acm (3.61 mg, 0.198 μmol) was dissolved in aqueous 6 M Gu.HCl containing 0.1 M Tris (1.4 mL, 15 μM protein concentration) and the mixture was gently shaken at 50° C. for 2.5 hours. After completion of the rearrangement reaction, the mixture was diluted with 6 M Gu.HCl (q.s. 10 mL) containing TFA (0.1%, v/v). The diluted mixture was filtered and injected into preparative HPLC. Crude rearranged peptide was purified by preparative HPLC using Shiseido Capcell Pak C18 column (5 μm, 250×20 mm) at a flow rate of 10 mL/min at 60° C. using CH₃CN/H₂O with 0.1% TFA (v/v) as mobile phase, with a gradient of 10 to 45% CH₃CN with 0.1% TFA (v/v) in 40 min. The fractions containing the purified product were pooled and lyophilized to obtain IL18-Seg1234A-Acm-rearranged as a white solid in >97% purity. The isolated yield was 1.41 mg (38%) and this material was directly used in the Acm deprotection step with no further characterization.

Acm deprotection: IL18-Seg1234A-Acm-rearranged (1.41 mg; 0.077 μmol) was dissolved in 0.25 mM AcOH/H₂O (1:1) (310 μL, protein concentration) and silver acetate (3.1 mg, 1%, m/v) was added to the solution. The mixture was shaken for 2.5 hours at 50° C. protected from light. After completion of reaction, the sample was diluted with 6 mL of 1:2 CH₃CN/H₂O with 0.1% TFA (v/v). The sample was purified by preparative HPLC on a Shiseido Capcell Pak UG80 C18 column (5 μm, 250×20 mm) at a flow rate of 10 mL/min at at 60° C. using CH₃CN/H₂O with 0.1% TFA (v/v) as mobile phase, with a gradient of 10 to 45% CH₃CN with 0.1% TFA (v/v) in 40 min. The fractions containing the purified product were pooled and lyophilized to obtain 0.7 mg of IL18-Seg1234A linear protein as a white powder in 97% purity (50% yield). MS (ESI): C₈₀₃H₁₂₇₅N₂₁₃O₂₄₇S₄; Average isotope calculated: 17992.2908 Da [M]; found: 17993.3349 Da [M].

### Example 4B—Synthesis of a Modified IL-18 Polypeptide of SEQ ID NO: 28

For this variant, except segment 3, all the other segments are the same as the ones used for example 4A.

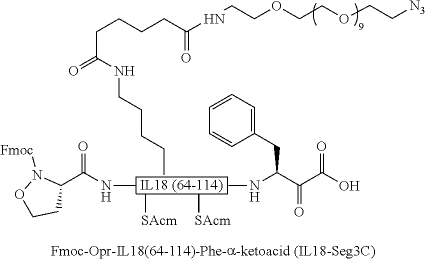

Segment 3B (Fmoc-Opr-IL18(64-114)-Phe-α-ketoacid): The Fmoc-Opr-IL18(64-114)-Phe-α-ketoacid segment was synthesized on a 0.2 mmol scale on Rink Amide ChemMatrix© resin pre-loaded with Fmoc-Phe-protected-α-ketoacid with a substitution capacity of −0.40 mmol/g.

Automated Fmoc-SPPS from position 96 to 114: The coupling reactions were performed at room temperature for 30 min by adding the Fmoc-amino acids dissolved in DMF (2.0 mL, 0.4 M, 4 eq), OxymaPure in DMF (2 mL, 0.4 M, 4 eq) and DIC in DMF (2 mL, 0.4 M, 4 eq) to the resin. Double couplings were required for each position. Capping was performed after each amino acid at room temperature for 6 min by adding a 20% (v/v) acetic anhydride solution in DMF (2 mL) and NMM in DMF (0.8 M, 2.0 mL). Fmoc deprotection reaction was performed using 20% (v/v) 4-methylpiperidine in DMF at room temperature for 8 min.

Manual coupling of Fmoc-L-Asp(tBu)-L-Thr[Ψ(Me,Me)Pro]-OH was then performed. A solution of Fmoc-L-Asp(tBu)-L-Thr[Ψ(Me,Me)Pro]-OH (332 mg, 0.6 mmol, 3 eq), HATU (228 mg, 0.6 mmol, 3 eq) and DIPEA (209 μL, 1.2 mmol, 6 eq) in 6 mL of DMF was prepared (3 min of pre-activation at room temperature) and added to the resin. The reaction was gently agitated at room temperature for 2 h.

Automated Fmoc-SPPS from position 73 to 93: The coupling reactions were performed using the conditions described above. Double couplings were required for each position. Fmoc deprotection reactions were performed using 20% (v/v) 4-methylpiperidine in DMF containing Cl-HOBt (0.1 M) at room temperature for 8 min.

Manual coupling of Fmoc-L-Ile-L-Ser[Ψ(Me,Me)Pro]-OH was then performed. A solution of Fmoc-L-Ile-L-Ser[Ψ(Me,Me)Pro]-OH (288 mg, 0.6 mmol, 3 eq), HATU (228 mg, 0.6 mmol, 3 eq) and DIPEA (209 μL, 1.2 mmol, 6 eq) in 6 ml of DMF was prepared (3 min of pre-activation at room temperature) and added to the resin. It was let to react at room temperature for 2 h. Capping was performed at room temperature for 10 min by adding a 20% (v/v) acetic anhydride solution in DMF (2 mL) and NMM in DMF (0.8 M, 2.0 mL) Fmoc deprotection reaction was performed using 20% (v/v) 4-methylpiperidine in DMF containing Cl-HOBt (0.1 M) at room temperature for 10 min.

Manual coupling of Fmoc-Lys(alloc)-OH was then performed. A solution of Fmoc-Lys(alloc)-OH (272 mg, 0.6 mmol, 3 eq), HATU (228 mg, 0.6 mmol, 3 eq) and DIPEA (209 μL, 1.2 mmol, 6 eq) in 6 ml of DMF was prepared (3 min of pre-activation at room temperature) and added to the resin. It was let to react at room temperature for 2 h.

Automated Fmoc-SPPS from position 64 to 69: The coupling reactions were performed using the same conditions used for position 73 to 93.

Manual coupling of Fmoc-5-(S)-oxaproline was then performed. A solution of Fmoc-5-(S)-oxaproline (204 mg, 0.6 mmol, 3 eq), HATU (228 mg, 0.6 mmol, 3 eq) and DIPEA (209 μL, 0.6 mmol, 6 eq) in 6 mL of DMF was prepared (3 min of pre-activation at room temperature) and added to the resin. The reaction was stirred at room temperature for 2 h.

Alloc deprotection: The resin was swollen in dry DCM for 15 min. A solution of phenyl silane (595 μL, 4.80 mmol, 24 eq.) in 3 ml of dry DCM purged with N₂, followed by a solution of palladium-tetrakis(triphenylphosphine) (116 mg, 100 μmol, 0.5 eq.) in 3 ml of dry DCM purged with N₂ were added to the resin which was left to stir for 30 min at room temperature. The resin was washed several times with DCM and DMF.

Manual coupling of glutaric anhydride was then performed. A solution of glutaric anhydride (172 μL, 2 mmol, 10 eq.) and NMM (220 μL, 2 mmol, 10 eq) in 6 ml of DMF was added to the resin which was left to stir for 30 min at room temperature. The resin was washed several times with DCM and DMF.

Manual coupling of O-(2-Aminoethyl)-O′-(2-azidoethyl) nonaethylene glycol was then performed. A solution of HATU (228 mg, 0.6 mmol, 3 eq) in 3 mL of DMF was added to the resin, followed by a solution of commercially available O-(2-Aminoethyl)-O′-(2-azidoethyl) nonaethylene glycol (338 mg, 0.6 mmol, 3 eq) in 2 mL of DMF and DIPEA (209 μL, 0.6 mmol, 6 eq) in 1 mL of DMF. The resin was then stirred at room temperature for 2 h.

The resin was washed with DCM and dried under vacuum. The mass of the dried peptidyl resin was 1.81 g. The peptide was cleaved from the resin by stirring the resin in a mixture of 95:2.5:2.5 TFA/DODT/H₂O (10 mL/g resin) at room temperature for 2 h. The resin was filtered off from the cleavage cocktail, and the filtrate was concentrated and diluted 20-fold with cold diethyl ether (20° C.), allowing the peptide to precipitate. After centrifugation, the ether layer was carefully decanted, and the peptide precipitate was resuspended in diethyl ether, triturated and centrifuged. Ether washings were repeated twice, and the resulting peptide precipitate was dried. Mass of crude peptide was 1.08 g. Purification of crude Fmoc-Opr-IL18(64-114)-Phe-α-ketoacid segment 3B was performed by preparative HPLC using Shiseido Capcell Pak C18 column (5 μm, 250×20 mm) at a flow rate of 10 mL/min at 60° C. using CH₃CN/H₂O with 0.1% TFA (v/v) as mobile phase, with a gradient of 10 to 45% CH₃CN with 0.1% TFA (v/v) in 40 min. The fractions containing the purified product were pooled and lyophilized to obtain the Fmoc-Opr-IL18(64-114)-Phe-α-ketoacid (IL18-Seg3B) as a white solid in >98% purity. The isolated yield based on the resin loading was 120 mg (8.5%). MS (ESI): C₃₁₉H₅₀₃N₇₇O₁₀₀S₂; [found: 7081.0 Da [M].

IL18-Seg34B preparation: Peptide ketoacid IL18-Seg3B (31 mg; 4.4 μmol; 1.1 eq) and hydroxylamine peptide IL18-Seg4A (20 mg; 4 μmol; 1 eq) were in dissolved in 97.5:2.5 DMSO/H₂O containing 0.1 M oxalic acid (200 μL). A very homogeneous liquid solution was obtained, which was gently agitated overnight at 60° C. Upon completion of the ligation reaction, the mixture was diluted with DMSO (200 μL) and further diluted with 1:1 CH₃CN/H₂O (q.s. 10 mL) with TFA (0.1%, v/v). The diluted mixture was directly injected into preparative HPLC. The crude ligated peptide solution was filtered and purified by preparative HPLC using Shiseido Capcell Pak C18 column (5 μm, 250×20 mm) at a flow rate of 10 mL/min at 60° C. using CH₃CN/H₂O with 0.1% TFA (v/v) as mobile phase, with a gradient of 10 to 45% CH₃CN with 0.1% TFA (v/v) in 40 min. The fractions containing the purified product were pooled and lyophilized to obtain 30.3 mg of Fmoc protected IL18-Seg34B as a white solid in >98% purity. IN The Fmoc protected IL18-Seg34B was dissolved in 300 μL of DMSO. Fmoc deprotection was initiated by addition of diethylamine (15 μL, 5%, v/v) and gently agitated at room temperature for 15 min. A second solution of diethylamine (15 μL) in DMSO (300 μL) was added to the reaction mixture and gently agitated at room temperature for another 15 min. Gel formation was expected. Trifluoroacetic acid (50 μL) was added to neutralize the reaction mixture. A homogeneous and colorless liquid solution was obtained, which was further diluted with 1:1 CH₃CN/H₂O (q.s. 10 mL) with TFA (0.1%, v/v). The diluted mixture was directly injected into preparative HPLC. The crude ligated peptide solution was filtered and purified by preparative HPLC using Shiseido Capcell Pak C18 column (5 μm, 250×20 mm) at a flow rate of 10 mL/min at 60° C. using CH₃CN/H₂O with 0.1% TFA (v/v) as mobile phase, with a gradient of 10 to 45% CH₃CN with 0.1% TFA (v/v) in 40 min. The fractions containing the purified product were pooled and lyophilized to obtain IL18-Seg34B as a white solid in >94% purity. The isolated yield was 12.5 mg (26%). MS (ESI): C₅₂₅H₈₃₉N₁₃₃O₁₆₉S₃; found: 11815.0 Da [M].

IL18-Seg1234B-Acm preparation: Peptide ketoacid IL18-Seg12A (7.2 mg; 1.01 μmol; 1.0 eq) and hydroxylamine peptide IL18-Seg34B (12.5 mg; 1.06 μmol; 1.05 eq) were dissolved in 97.5:2.5 DMSO/H₂O containing 0.1 M oxalic acid (68 μL). A homogeneous liquid solution was obtained, which was reacted overnight at 60° C. After completion of the ligation reaction, the mixture was diluted first with DMSO (150 μL). The mixture was further diluted with 2:1 H₂O/CH₃CN (q.s. 10 mL) containing TFA (0.1%, v/v). The diluted mixture was filtered and injected into preparative HPLC. Crude ligated peptide was purified by preparative HPLC using Shiseido Capcell Pak C18 column (5 μm, 250×20 mm) at a flow rate of 10 mL/min at 60° C. using CH₃CN/H₂O with 0.1% TFA (v/v) as mobile phase, with a gradient of 10 to 45% CH₃CN with 0.1% TFA (v/v) in 40 min. The fractions containing the purified product were pooled and lyophilized to obtain IL18-Seg1234B-Acm as a white solid in >98% purity. The isolated yield was 5.8 mg (30.4%). This material was directly used in the rearrangement step with no further characterization.

IL18-Seg1234B Linear Protein Preparation:

Rearrangement of linear protein: IL18-Seg1234B-Acm (5.81 mg, 0.307 μmol) was dissolved in aqueous 6 M Gu.HCl containing 0.1 M Tris (2.2 mL, 15 μM protein concentration) and the mixture was gently shaken at 50° C. for 2.5 hours. After completion of the rearrangement reaction, the mixture was diluted with 6 M Gu.HCl (q.s. 10 mL) containing TFA (0.1%, v/v). The diluted mixture was filtered and injected into preparative HPLC. Crude rearranged peptide was purified by preparative HPLC using Shiseido Capcell Pak C18 column (5 μm, 250×20 mm) at a flow rate of 10 mL/min at 60° C. using CH₃CN/H₂O with 0.1% TFA (v/v) as mobile phase, with a gradient of 10 to 45% CH₃CN with 0.1% TFA (v/v) in 40 min. The fractions containing the purified product were pooled and lyophilized to obtain IL18-Seg1234B-Acm-rearranged as a white solid in >90% purity. The isolated yield was 1.2 mg (21%). This material was directly used in the Acm deprotection step with no further characterization.

Acm deprotection: IL18-Seg1234B-Acm-rearranged (1.2 mg; 0.063 μmol) was dissolved in 0.20 mM AcOH/H₂O (1:1) (320 μL, protein concentration) and silver acetate (3.2 mg, 1%, m/v) was added to the solution. The mixture was shaken for 2.5 hours at 50° C. protected from light. After completion of reaction, the sample was diluted with 6 mL of 1:2 CH₃CN/H₂O with 0.1% TFA (v/v). The sample was purified by preparative HPLC on a Shiseido Capcell Pak UG80 C18 column (5 μm, 250×20 mm) at a flow rate of 10 mL/min at at 60° C. using CH₃CN/H₂O with 0.1% TFA (v/v) as mobile phase, with a gradient of 10 to 45% CH₃CN with 0.1% TFA (v/v) in 40 min. The fractions containing the purified product were pooled and lyophilized to obtain 0.2 mg of IL18-Seg1234B linear protein as a white powder in 86% purity (20% yield). MS (ESI): C₈₃₀H₁₃₂₅N₂₁₇O₂₅₉S₄; Mass found: 18617.00 Da [M+H⁺].

### Example 4C—Synthesis of a Modified IL-18 Polypeptide of SEQ ID NO: 63

The following protocol was used to prepare a modified IL-18 polypeptide of SEQ ID NO: 63, wherein residue 68 comprises an aspartate residue modified to comprise an azide functionality. For this variant, segment 1A is the same as the one used for example 4A.

Segment 2B (Opr-IL18(32-61)-Val-photoprotected-α-ketoacid): Opr-IL18(32-61)-Val-photoprotected-α-ketoacid segment B was synthesized on a 0.2 mmol scale on Rink Amide MBHA resin pre-loaded with Fmoc-Val-photoprotected-α-ketoacid with a substitution capacity of ˜0.307 mmol/g.

Automated Fmoc-SPPS for position 61: The coupling reaction was performed at room temperature for 30 min by adding the Fmoc-amino acid dissolved in DMF (2.0 mL, 0.4 M, 4 eq), OxymaPure in DMF (2 mL, 0.4 M, 4 eq) and DIC in DMF (2 mL, 0.4 M, 4 eq) to the resin. Capping was performed at room temperature for 6 min by adding a 20% (v/v) acetic anhydride solution in DMF (2 mL) and NMM in DMF (0.8 M, 2.0 mL). Fmoc deprotection reaction was performed using 20% (v/v) 4-methylpiperidine in DMF at room temperature for 8 min.

Manual coupling of Fmoc-O-methyl-L-homoserine was then performed. A solution of Fmoc-O-methyl-L-homoserine (177 mg, 0.5 mmol, 2.5 eq), HATU (190 mg, 0.5 mmol, 2.5 eq) and DIPEA (174 μL, 1.0 mmol, 5 eq) in 6 mL of DMF was prepared (3 min of pre-activation at room temperature) and added to the resin. The reaction was gently agitated at room temperature for 2 h. Capping was performed at room temperature by adding acetic anhydride (150 μL, 1.6 mmol, 8 eq) and DIPEA (278 μL, 1.6 mmol, 8 eq) in DMF (4 mL) at room temperature for 10 min. Fmoc deprotection reaction was performed using 20% (v/v) 4-methylpiperidine in DMF at room temperature for 10 min.

Automated Fmoc-SPPS from position 59 to 56: The coupling reactions were performed at room temperature for 30 min by adding the Fmoc-amino acids dissolved in DMF (2.0 mL, 0.4 M, 4 eq), OxymaPure in DMF (2 mL, 0.4 M, 4 eq) and DIC in DMF (2 mL, 0.4 M, 4 eq) to the resin. Capping was performed after each amino acid at room temperature for 6 min by adding a 20% (v/v) acetic anhydride solution in DMF (2 mL) and NMM in DMF (0.8 M, 2.0 mL). Fmoc deprotection reaction was performed using 20% (v/v) 4-methylpiperidine in DMF at room temperature for 8 min.

Manual coupling of Fmoc-L-Asp(tBu)-L-Ser[Ψ(Me,Me)Pro]-OH was then performed. A solution of Fmoc-L-Asp(tBu)-L-Ser[Ψ(Me,Me)Pro]-OH (322 mg, 0.6 mmol, 3 eq), HATU (228 mg, 0.6 mmol, 3 eq) and DIPEA (209 μL, 1.2 mmol, 6 eq) in 6 mL of DMF was prepared (3 min of pre-activation at room temperature) and added to the resin. The reaction was gently agitated at room temperature for 2 h.

Automated Fmoc-SPPS from position 53 to 52: The coupling reactions were performed using the same conditions as previously mentioned for the beginning of the sequence.

Manual coupling of Fmoc-O-methyl-L-homoserine was then performed. The Fmoc-O-methyl-L-homoserine manual coupling reaction was performed using the same conditions as previously mentioned.

Manual coupling of Fmoc-L-Ile-L-Ser[Ψ(Me,Me)Pro]-0H was then performed. A solution of Fmoc-L-Ile-L-Ser[Ψ(Me,Me)Pro]-OH (288 mg, 0.6 mmol, 3 eq), HATU (228 mg, 0.6 mmol, 3 eq) and DIPEA (209 μL, 1.2 mmol, 6 eq) in 6 mL of DMF was prepared (3 min of pre-activation at room temperature) and added to the resin. The reaction was gently agitated at room temperature for 2 h.

Automated SPPS from position 48 to 37: The coupling reactions were performed using the conditions described above. Double couplings were required for each position.

Manual coupling of Fmoc-L-Asp(tBu)-L-Ser[Ψ(Me,Me)Pro]-OH was then performed. A solution of Fmoc-L-Asp(tBu)-L-Ser[Ψ(Me,Me)Pro]-OH (322 mg, 0.6 mmol, 3 eq), HATU (228 mg, 0.6 mmol, 3 eq) and DIPEA (209 μL, 1.2 mmol, 6 eq) in 6 mL of DMF was prepared (3 min of pre-activation at room temperature) and added to the resin. The reaction was gently agitated at room temperature for 2 h.

Automated Fmoc-SPPS for position 34: The coupling reaction was performed using the conditions described above. Double couplings were required.

Manual coupling of Fmoc-O-methyl-L-homoserine was then performed. The Fmoc-O-methyl-L-homoserine_manual coupling reactions was performed using the same conditions as previously mentioned.

Automated Fmoc-SPPS for position 32: The coupling reaction was performed using the conditions described above. Double couplings were required.

Manual coupling of Boc-5-(S)-oxaproline was then performed. A solution of Boc-5-(S)-oxaproline (130 mg, 0.6 mmol, 3 eq), HATU (228 mg, 0.6 mmol, 3 eq) and DIPEA (209 μL, 1.2 mmol, 6 eq) in 6 mL of DMF was prepared (3 min of pre-activation at room temperature) and added to the resin. The reaction was gently agitated at room temperature for 2 h. The resin was washed with DCM and diethyl ether and dried under vacuum. The mass of the dried peptidyl resin was 1.4 g.

The peptide was cleaved from the resin using a mixture of 95:2.5:2.5 TFA/DODT/H₂O (10 mL/g resin) and gently agitating the mixture at room temperature for 2.0 h. The resin was filtered off from the cleavage cocktail, and the filtrate was concentrated and diluted 20-fold with cold diethyl ether (20° C.), allowing the peptide to precipitate. After centrifugation, the ether layer was carefully decanted, and the peptide precipitate was resuspended in diethyl ether, triturated and centrifuged. Ether washings were repeated twice, and the resulting peptide precipitate was dried. The mass of crude peptide was 940 mg. Purification of the crude Opr-IL18(32-61)-Val-photoprotected-α-ketoacid segment 2C was performed by preparative HPLC using Shiseido Capcell Pak C18 column (5 μm, 250×20 mm) at a flow rate of 10 mL/min at 60° C. using CH₃CN/H₂O with 0.1% TFA (v/v) as mobile phase, with a gradient of 10 to 70% CH₃CN with 0.1% TFA (v/v) in 40 min. The fractions containing the purified product were pooled and lyophilized to obtain Opr-IL18(32-61)-Val-photoprotected-α-ketoacid (IL18-Seg2) as a white solid in >90% purity. The isolated yield based on the resin loading was 340 mg (40%). C₁₆₃H₂₅₃N₄₅O₆₀S; Average isotope calculated: 1278.6023 Da [M+3H⁺]; found: 1278.6020 Da [M+3H⁺].

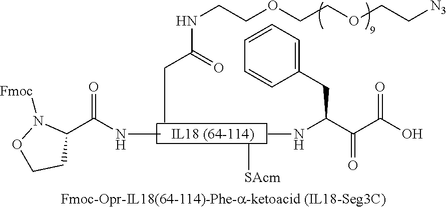

Segment 3C (Fmoc-Opr-IL18(64-114)-Phe-α-ketoacid): The Fmoc-Opr-IL18(64-114)-Phe-α-ketoacid segment was synthesized on a 0.2 mmol scale on Rink Amide ChemMatrix© resin pre-loaded with Fmoc-Phe-protected-α-ketoacid with a substitution capacity of ˜0.40 mmol/g.

Automated Fmoc-SPPS at position 114: The coupling reaction was performed at room temperature for 30 min by adding the Fmoc-amino acid dissolved in DMF (2.0 mL, 0.4 M, 4 eq), OxymaPure in DMF (2 mL, 0.4 M, 4 eq) and DIC in DMF (2 mL, 0.4 M, 4 eq) to the resin. Double couplings were required. Capping was performed at room temperature for 6 min by adding a 20% (v/v) acetic anhydride solution in DMF (2 mL) and NMM in DMF (0.8 M, 2.0 mL). Fmoc deprotection reaction was performed using 20% (v/v) 4-methylpiperidine in DMF at room temperature for 8 min.

Manual coupling of Fmoc-O-methyl-L-homoserine was then performed. A solution of Fmoc-O-methyl-L-homoserine (177 mg, 0.5 mmol, 2.5 eq), HATU (190 mg, 0.5 mmol, 2.5 eq) and DIPEA (174 μL, 1.0 mmol, 5 eq) in 6 mL of DMF was prepared (3 min of pre-activation at room temperature) and added to the resin. The reaction was gently agitated at room temperature for 2 h. Capping was performed at room temperature by adding acetic anhydride (150 μL, 1.6 mmol, 8 eq) and DIPEA (278 μL, 1.6 mmol, 8 eq) in DMF (4 mL) at room temperature for 10 min. Fmoc deprotection reaction was performed using 20% (v/v) 4-methylpiperidine in DMF at room temperature for 10 min.

Automated Fmoc-SPPS from position 96 to 112: The coupling reactions were performed at room temperature for 30 min by adding the Fmoc-amino acids dissolved in DMF (2.0 mL, 0.4 M, 4 eq), OxymaPure in DMF (2 mL, 0.4 M, 4 eq) and DIC in DMF (2 mL, 0.4 M, 4 eq) to the resin. Double couplings were required for each position. Capping was performed after each amino acid at room temperature for 6 min by adding a 20% (v/v) acetic anhydride solution in DMF (2 mL) and NMM in DMF (0.8 M, 2.0 mL). Fmoc deprotection reaction was performed using 20% (v/v) 4-methylpiperidine in DMF at room temperature for 8 min.

Manual coupling of Fmoc-L-Asp(tBu)-L-Thr[Ψ(Me,Me)Pro]-OH was then performed. A solution of Fmoc-L-Asp(tBu)-L-Thr[Ψ(Me,Me)Pro]-OH (332 mg, 0.6 mmol, 3 eq), HATU (228 mg, 0.6 mmol, 3 eq) and DIPEA (209 μL, 1.2 mmol, 6 eq) in 6 mL of DMF was prepared (3 min of pre-activation at room temperature) and added to the resin. The reaction was gently agitated at room temperature for 2 h.

Automated Fmoc-SPPS from position 87 to 93: The coupling reactions were performed using the conditions described above. Double couplings were required for each position. Fmoc deprotection reactions were performed using 20% (v/v) 4-methylpiperidine in DMF containing Cl-HOBt (0.1 M) at room temperature for 8 min.

Manual coupling of Fmoc-O-methyl-L-homoserine was then performed. The Fmoc-O-methyl-L-homoserine_manual coupling reactions was performed using the same conditions as previously mentioned.

Automated Fmoc-SPPS from position 73 to 85: The coupling reactions were performed using the same conditions used for position 87 to 93.

Manual coupling of Fmoc-L-Ile-L-Ser[Ψ(Me,Me)Pro]-OH was then performed. A solution of Fmoc-L-Ile-L-Ser[Ψ(Me,Me)Pro]-OH (288 mg, 0.6 mmol, 3 equiv), HATU (228 mg, 0.6 mmol, 3 equiv) and DIPEA (209 μL, 1.2 mmol, 6 equiv) in 6 ml of DMF was prepared (3 min of pre-activation at r.t.) and added to the resin. It was let to react at r.t. for 2 h.

Automated Fmoc-SPPS from position 69 to 70: The coupling reactions were performed using the same conditions used for position 73 to 85.

Manual coupling of Fmoc-Asp(alloc)-OH was then performed. A solution of Fmoc-Asp(alloc)-OH (237 mg, 0.6 mmol, 3 equiv), HATU (228 mg, 0.6 mmol, 3 equiv) and DIPEA (209 μL, 1.2 mmol, 6 equiv) in 6 ml of DMF was prepared (3 min of pre-activation at r.t.) and added to the resin. It was let to react at r.t. for 2 h.

Automated Fmoc-SPPS from position 64 to 67: The coupling reactions were performed using the same conditions used for position 69 to 70.

Manual coupling of Fmoc-5-(S)-oxaproline was then performed. A solution of Fmoc-5-(S)-oxaproline (204 mg, 0.6 mmol, 3 eq), HATU (228 mg, 0.6 mmol, 3 eq) and DIPEA (209 μL, 0.6 mmol, 6 eq) in 6 mL of DMF was prepared (3 min of pre-activation at room temperature) and added to the resin. The reaction was gently agitated at room temperature for 2 h.

Alloc deprotection: The resin was swollen in dry DCM for 15 min. A solution of phenyl silane (595 μL, 4.80 mmol, 24 equiv.) in 3 ml of dry DCM purged with N₂, followed by a solution of palladium-tetrakis(triphenylphosphine) (116 mg, 100 μmol, 0.5 eq.) in 3 ml of dry DCM purged with N₂ were added to the resin which was left to stir for 30 min at room temperature. The resin was washed several times with DCM and DMF.

Manual coupling of O-(2-Aminoethyl)-O′-(2-azidoethyl) nonaethylene glycol was then performed. A solution of HATU (228 mg, 0.6 mmol, 3 eq) in 3 mL of DMF was added to the resin, followed by a solution of commercially available O-(2-Aminoethyl)-O′-(2-azidoethyl) nonaethylene glycol (338 mg, 0.6 mmol, 3 eq) in 2 mL of DMF and DIPEA (209 μL, 0.6 mmol, 6 eq) in 1 mL of DMF. The resin was then stirred at room temperature for 2 h.

The resin was washed with DCM and dried under vacuum. The mass of the dried peptidyl resin was 2 g. The peptide was cleaved from the resin by stirring the resin in a mixture of 95:2.5:2.5 TFA/DODT/H₂O (10 mL/g resin) at room temperature for 2.0 h. The resin was filtered off from the cleavage cocktail, and the filtrate was concentrated and diluted 20-fold with cold diethyl ether (20° C.), allowing the peptide to precipitate. After centrifugation, the ether layer was carefully decanted, and the peptide precipitate was resuspended in diethyl ether, triturated and centrifuged. Ether washings were repeated twice, and the resulting peptide precipitate was dried. Mass of crude peptide was 1.5 g. Purification of crude Fmoc-Opr-IL18(64-114)-Phe-α-ketoacid segment 3C was performed by preparative HPLC using Shiseido Capcell Pak C18 column (5 μm, 250×20 mm) at a flow rate of 10 mL/min at 60° C. using CH₃CN/H₂O with 0.1% TFA (v/v) as mobile phase, with a gradient of 10 to 50% CH₃CN with 0.1% TFA (v/v) in 40 min. The fractions containing the purified product were pooled and lyophilized to obtain the Fmoc-Opr-IL18(64-114)-Phe-α-ketoacid (IL18-Seg3C) as a white solid in >80% purity. The isolated yield based on the resin loading was 104 mg (6%). MS (MALDI-TOF): C₃₁₀H₄₈₈N₇₆O₁₀₀S; calculated 6907.5157 Da [M]; found: 6902.4980 Da.

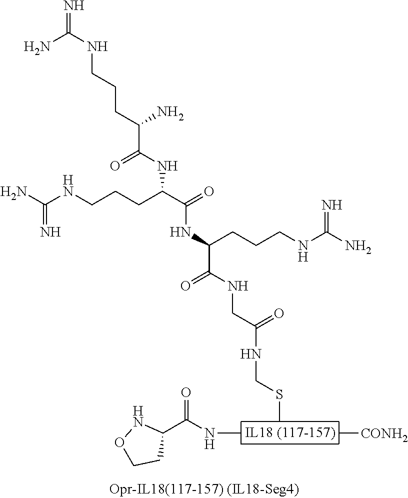

Segment 4C (Opr-IL18 (117-157)): Opr-IL18(117-157) Synthesis of SEQ ID NO: 63-Seg4 was started on a 0.5 mmol scale using Rink Amide MBHA resin pre-loaded with Fmoc-Asp(OtBu)-OH with a substitution capacity of ˜0.35 mmol/g (1.43 g).

Manual Fmoc-SPPS from position 153 to 156: Fmoc-protected amino acids (5.0 equiv., 2.5 mmol) and HCTU (4.8 equiv., 2.4 mmol) were dissolved in NMP (6.0 mL). Pre-activation was performed for 2 min by addition of DIPEA (0.5 mL, 2.2 mmol). The reaction mixture was poured onto the resin and allowed to react for 45 min under gentle manual stirring. The resin was rinsed thoroughly with DMF and DCM. Capping was performed at r.t. for 6 min at each position by addition of acetic anhydride (0.5 mL) and DIPEA (0.5 mL) in DMF (6 mL). For Fmoc deprotection, the resin was rinsed once with 20% 4-methylpiperidine in DMF. The resin was treated by the same solution for 15 min under gentle manual stirring. The resin was rinsed thoroughly with DMF and DCM.

Manual coupling of Fmoc-Phe-Thr[Ψ(Me,Me)Pro]-OH: A solution of Fmoc-Phe-Thr[Ψ(Me,Me)Pro]-OH (793 mg, 1.5 mmol, 3.0 equiv.), HATU (570 mg, 1.5 mmol, 3.0 equiv.) and DIPEA (500 μL, 2.9 mmol, 5.8 equiv.) in 6 mL of NMP was prepared (3 min of pre-activation at r.t.) and added to the resin. The reaction was gently agitated at room temperature for 2 h. Capping was performed at r.t. for 6 min at each position by addition of acetic anhydride (0.5 mL) and DIPEA (0.5 mL) in DMF (6 mL). For Fmoc deprotection, the resin was rinsed once with 20% (v/v) 4-methylpiperidine in DMF. The resin was treated by the same solution for 15 min under gentle manual stirring. The resin was rinsed thoroughly with DMF and DCM.

Manual coupling of Fmoc-O-methylhomoserine: A solution of Fmoc-Hse(Me)-OH (533 mg, 1.5 mmol, 3.0 equiv.), HATU (570 mg, 1.5 mmol, 3.0 equiv.) and DIPEA (500 μL, 2.9 mmol, 5.8 equiv.) in 6 mL of NMP was prepared (3 min of pre-activation at room temperature) and added to the resin. The reaction was gently agitated at room temperature for 2 h. Capping was performed at r.t. for 6 min at each position by addition of acetic anhydride (0.5 mL) and DIPEA (0.5 mL) in DMF (6 mL). For Fmoc deprotection, the resin was rinsed once with 20% (v/v) 4-methylpiperidine in DMF. The resin was treated by the same solution for 15 min under gentle manual stirring. The resin was rinsed thoroughly with DMF and DCM.

Manual Fmoc-SPPS from position 146 to 149: The coupling, acetylation and deprotection reactions were performed using the same conditions used for position 151 to 156.

Manual coupling of Fmoc-Leu-(Dmb)Gly-OH: A solution of Fmoc-Leu-(Dmb)Gly-OH (842 mg, 1.5 mmol, 3.0 equiv.), HATU (570 mg, 1.5 mmol, 3.0 equiv.) and DIPEA (500 μL, 2.9 mmol, 5.8 equiv.) in 6 mL of NMP was prepared (3 min of pre-activation at room temperature) and added to the resin. The reaction was gently agitated at room temperature for 2 h. Capping was performed at r.t. for 6 min at each position by addition of acetic anhydride (0.5 mL) and DIPEA (0.5 mL) in DMF (6 mL). For Fmoc deprotection, the resin was rinsed once with 20% (v/v) 4-methylpiperidine in DMF. The resin was treated by the same solution for 15 min under gentle manual stirring. The resin was rinsed thoroughly with DMF and DCM.

Synthesis of SEQ ID NO: 63 Seg4 was continued on a 0.15 mmol scale.

Automated Fmoc-SPPS from position 128 to 143: The coupling reactions were performed at room temperature for 30 min by adding a solution of Fmoc-amino acids dissolved in DMF (2.0 mL, 0.4 M, 5.3 equiv.), HCTU (2 mL, 0.4 M, 5.3 equiv.) and NMM in DMF (2 mL, 0.8 M, 10.7 equiv.) to the resin. Double coupling was performed for all positions. Capping was performed after each amino acid at room temperature for 6 min by adding a 20% (v/v) acetic anhydride solution in DMF (2 mL) and NMM in DMF (0.8 M, 2.0 mL). The Fmoc deprotection reaction was performed using 20% (v/v) 4-methylpiperidine in DMF at room temperature for 10 min.

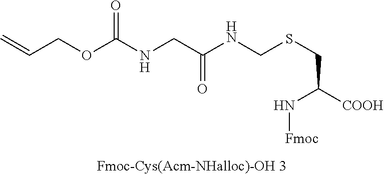

Manual coupling of Fmoc-Cys(Acm-NHalloc)-OH 3: A solution of Fmoc-Cys(Acm-NHalloc)-OH (308 mg, 0.75 mmol, 5.0 equiv.), HATU (285 mg, 0.75 mmol, 5.0 equiv.) and DIPEA (210 μL, 1.2 mmol, 8 equiv.) in 4 mL of NMP was prepared (3 min of pre-activation at room temperature) and added to the resin. The reaction was gently agitated at room temperature for 2 h. Capping was performed at r.t. for 6 min at each position by addition of acetic anhydride (0.2 mL) and DIPEA (0.2 mL) in DMF (4 mL). For Fmoc deprotection, the resin was rinsed once with 20% (v/v) 4-methylpiperidine in DMF. The resin was treated by the same solution for 15 min under gentle manual stirring. The resin was rinsed thoroughly with DMF and DCM.

Automated Fmoc-SPPS from position 123 to 126: The coupling, acetylation and deprotection reactions were performed using the same conditions used for position 123 to 143.

Manual coupling of Fmoc-Glu(OtBu)-(Dmb)Gly-OH: A solution of Fmoc-Glu(OtBu)-(Dmb)Gly-OH (475 mg, 0.75 mmol, 5.0 equiv.), HATU (342 mg, 0.75 mmol, 5.0 equiv.) and DIPEA (210 μL, 1.2 mmol, 8 equiv.) in 4 mL of NMP was prepared (3 min of pre-activation at room temperature) and added to the resin. The reaction was gently agitated at room temperature for 2 h. Capping was performed at r.t. for 6 min at each position by addition of acetic anhydride (0.2 mL) and DIPEA (0.2 mL) in DMF (4 mL). For Fmoc deprotection, the resin was rinsed once with 20% (v/v) 4-methylpiperidine in DMF. The resin was treated by the same solution for 15 min under gentle manual stirring. The resin was rinsed thoroughly with DMF and DCM.

Automated Fmoc-SPPS from position 117 to 120: The coupling, acetylation and deprotection reactions were performed using the same conditions used for position 123 to 143.

Manual coupling of Boc-Opr-OH: A solution of Boc-Opr-OH (163 mg, 0.75 mmol, 5.0 equiv.), HATU (342 mg, 0.75 mmol, 5.0 equiv.) and DIPEA (210 μL, 1.2 mmol, 8 equiv.) in 4 mL of NMP was prepared (3 min of pre-activation at room temperature) and added to the resin. The reaction was gently agitated at room temperature for 2 h. The resin was rinsed thoroughly with DMF and DCM.

For Alloc deprotection of residue 127: The resin was swollen in DCM purged with nitrogen. A solution of phenylsilane (444 μL, 3.6 mmol, 24 equiv) in DCM (3 mL) purged with nitrogen was added to the resin. Alloc deprotection was carried out upon the addition of a solution of Pd(PPh3)4 (58 mg, 0.075 mmol, 0.5 equiv) in DCM (2 mL) purged with nitrogen. It was let to react at r.t. for 30 min with manual stirring. The side-chain was then functionalized with a solubilizing Tag composed of three arginine residues. They were coupled in an automated fashion using the same conditions used for position 123 to 143.

The peptide was cleaved from the resin using a mixture of 92.5:2.5:2.5:2.5 TFA/TIPS/DODT/H₂O (10 mL/g resin) at room temperature for 2 h. The resin was filtered off from the cleavage cocktail, and the filtrate was concentrated and diluted 20-fold with cold diethyl ether (−20° C.), allowing the peptide to precipitate. After centrifugation, the ether layer was carefully decanted, and the peptide precipitate was resuspended in diethyl ether, triturated and centrifuged. Ether washings were repeated twice, and the resulting peptide precipitate was dried. Purification of crude Segment 4C was performed by preparative HPLC using Shiseido Proteonavi column (5 μm, 250×50 mm) at a flow rate of 40 mL/min at 60° C. using CH₃CN/H₂O with 0.1% TFA (v/v) as mobile phase, with a gradient of 5 to 50% CH₃CN with 0.1% TFA (v/v) in 45 min. The fractions containing the purified product were pooled and lyophilized to obtain Segment 4C as a white solid. The isolated yield based on the resin loading was 116 mg (14%). MS (MALDI-TOF): C₂₃₉H₃₈₁N₆₉O₇₇S; Average isotope calculated 5485.14 Da [M]; found: 5481.28

IL18-Seg12C preparation: Peptide ketoacid IL18-Seg1A (56.2 mg; 15.8 μmol; 1.2 eq) and hydroxylamine peptide IL18-Seg2C (50.6 mg; 13.2 μmol; 1.0 eq) were in dissolved in a 9.75:0.25 DMSO/H₂O solution containing 0.1 M oxalic acid (660 μL). A very homogeneous liquid solution was obtained. The ligation vial was protected from light by wrapping the vial in aluminum foil and gently agitated overnight at 60° C. After completion of the ligation, the mixture was diluted with DMSO (1920 μL) and irradiated at a wavelength of 365 nm for 2 h to allow photo deprotection of the C-terminal ketoacid. The mixture was further diluted with 1:1 CH₃CN/H₂O (q.s. 20 mL) with TFA (0.1%, v/v). The diluted mixture was filtered and injected into preparative HPLC. Crude ligated peptide was purified by preparative HPLC using Shiseido capcell Pak C18 column (5 μm, 250×50 mm) at a flow rate of 40 mL/min at 60° C., with a gradient of 10% to 70% CH₃CN with 0.1% TFA (v/v) in 40 min. The fractions containing the purified product were pooled and lyophilized to obtain IL18-Seg12C as a white solid in >98% purity. The isolated yield was 40 mg (42%). MS (MALDI-TOF): C₃₂₁H₅₀₆N₈₈O₉₆S; Average calculated 7131.6516 Da [M]; found: 7125.8340.

IL18-Seg34C preparation: Peptide ketoacid IL18-Seg3C (25 mg; 3.6 μmol; 1.0 eq) and hydroxylamine peptide IL18-Seg4C (22 mg; 4 μmol; 1.1 eq) were in dissolved in 97.5:2.5 DMSO/H₂O containing 0.1 M oxalic acid (180 μL). A very homogeneous liquid solution was obtained, which was gently agitated overnight at 60° C. Upon completion of the ligation reaction, the mixture was diluted with DMSO (320 μL) and further diluted with 1:2 CH₃CN/H₂O (q.s. 10 mL) with TFA (0.1%, v/v). The diluted mixture was directly injected into preparative HPLC. The crude ligated peptide solution was filtered and purified by preparative HPLC using Shiseido Capcell Pak C18 column (5 μm, 250×20 mm) at a flow rate of 10 mL/min at 60° C. using CH₃CN/H₂O with 0.1% TFA (v/v) as mobile phase, with a gradient of 10 to 45% CH₃CN with 0.1% TFA (v/v) in 40 min. The fractions containing the product were pooled and lyophilized to obtain 8 mg of Fmoc protected IL18-Seg34C as a white solid.

The Fmoc protected IL18-Seg34C was dissolved in DMSO (200 μL). Fmoc deprotection was initiated by adding diethylamine (10 μL, 5%, v/v) and gently agitated at room temperature for 15 min. A second solution of diethylamine (10 μL) in DMSO (200 μL) was added to the reaction mixture and gently agitated at room temperature for another 15 min. Gel formation was expected. Trifluoroacetic acid (40 μL) was added to neutralize the reaction mixture. A homogeneous and colorless liquid solution was obtained, which was further diluted with 1:2 CH₃CN/H₂O (q.s. 10 mL) with TFA (0.1%, v/v). The diluted mixture was directly injected into preparative HPLC. The crude ligated peptide solution was filtered and purified by preparative HPLC using Shiseido Capcell Pak C18 column (5 μm, 250×20 mm) at a flow rate of 10 mL/min at 60° C. using CH₃CN/H₂O with 0.1% TFA (v/v) as mobile phase, with a gradient of 10 to 45% CH₃CN with 0.1% TFA (v/v) in 40 min. The fractions containing the purified product were pooled and lyophilized to obtain IL18-Seg34C as a white solid in >97% purity. The isolated yield was 5.5 mg (26%). MS (MALDI-TOF): C₅₃₀H₈₅₄N₁₄₄O₁₇₂S₂; Average calculated 12059.62 Da [M]; found: 12119.56 [M+adduct].

IL18-Seg1234C Linear Protein Preparation:

Final peptide ligation: Peptide ketoacid IL18-Seg12C (3.6 mg; 0.5 μmol; 1.1 eq) and hydroxylamine peptide IL18-Seg34C (5.5 mg; 0.46 μmol; 1.0 eq) were dissolved in 97.5:2.5 DMSO/H₂O containing 0.1 M oxalic acid (23 μL). A homogeneous liquid solution was obtained, which was reacted overnight at 65° C. After completion of the ligation reaction, the mixture was diluted first with DMSO (0.95 mL).
Rearrangement: The mixture was further diluted with 6 M Gu.HCl containing 0.1 M Tris (1.0 mL) and the mixture was gently shaken at 50° C. for 2 h. After completion of the rearrangement reaction, the mixture was diluted with 6 M Gu.HCl (q.s. 10.0 mL) containing TFA (0.1%, v/v). The diluted mixture was filtered and injected into preparative HPLC. Crude rearranged peptide was purified by preparative HPLC using Shiseido Capcell Pak C18 column (5 μm, 250×20 mm) at a flow rate of 10 mL/min at 60° C. using CH₃CN/H₂O with 0.1% TFA (v/v) as mobile phase, with a gradient of 10 to 45% CH₃CN with 0.1% TFA (v/v) in 40 min. The fractions containing the purified product were pooled and lyophilized to obtain IL18-Seg1234C-Acm-rearranged as a white solid in >97% purity. The isolated yield was 1.9 mg (22%). This material was directly used in the Acm deprotection step with no further characterization.

Acm deprotection: IL18-Seg1234C-Acm-rearranged (1.9 mg; 0.099 μmop was dissolved in 0.20 mM AcOH/H₂O (1:1) (500 μL) and silver acetate (5.0 mg, 1%, m/v) was added to the solution. The mixture was shaken for 3 hours at 50° C. protected from light. After completion of reaction, the sample was diluted with 9.5 mL of 1:2 CH₃CN/H₂O with 0.1% TFA (v/v). The sample was purified by preparative HPLC on a Shiseido Capcell Pak UG80 C18 column (5 μm, 250×20 mm) at a flow rate of 10 mL/min at at 60° C. using CH₃CN/H₂O with 0.1% TFA (v/v) as mobile phase, with a gradient of 10 to 45% CH₃CN with 0.1% TFA (v/v) in 40 min. The fractions containing the purified product were pooled and lyophilized to obtain 0.5 mg of IL18-Seg1234C linear protein as a white powder in 97% purity (30% yield). MS (MALDI-TOF): C₈₁₉H₁₃₀₅N₂₁₇O₂₆₄S₃; Average calculated 18511.88; found: 18513.3270 Da.

### Folding and Formulation of IL18-Seg1234C Linear Protein:

The protein powder (0.22 mg, lyophilized as TFA salt) is solubilized in 110 μL of Buffer A Tris buffer (50 mM, pH 8.0) containing 8 M urea, 2 mM DTT and 0.02% (m/v) Tween 80. A 2 mg/mL clear solution is obtained. It is incubated at r.t. for 30 min. The protein solution is slowly diluted at 20° C. in a dropwise fashion with Tris buffer (50 mM, pH 7.8) containing 2 mM EDTA, 137 mM NaCl, 2.7 mM KCl, 400 mM arginine.HCl, 2 mM DTT and 0.02% (m/v) Tween 80 (Buffer B). The mixture is gently shaken (400 RPM) to allow efficient diffusion. A 0.2 mg/mL clear solution is obtained. It is incubated at 20° C. for 20 h. It is then centrifuged at 10 000 RPM at r.t. for 5 min. The protein solution is dialyzed against 650 mL of PBS (pH 7.4) containing 6% sucrose and 0.02% Tween 80 at r.t. for 2 h. This step is repeated a second time. The protein solution is then dialyzed a third time against 650 mL of PBS (pH 7.4) containing 6% sucrose and 0.02% Tween 80 at r.t. for 18 h. Finally, it is centrifuged at 10 000 RPM at r.t. for 5 min and stored at −80° C.

The above folding protocol describes an exemplary protocol which can be tested in order to prepare a properly folded IL-18 polypeptide from the linear Seg1234 polypeptide precursor. In some embodiments, the folding protocol described above is modified by using alternative Buffer A and B as outlined in Example 2 (See Table 14). These folding protocols can be tested until a desired folding outcome is determined.

### Example 5—Structure of Composition A and Composition B

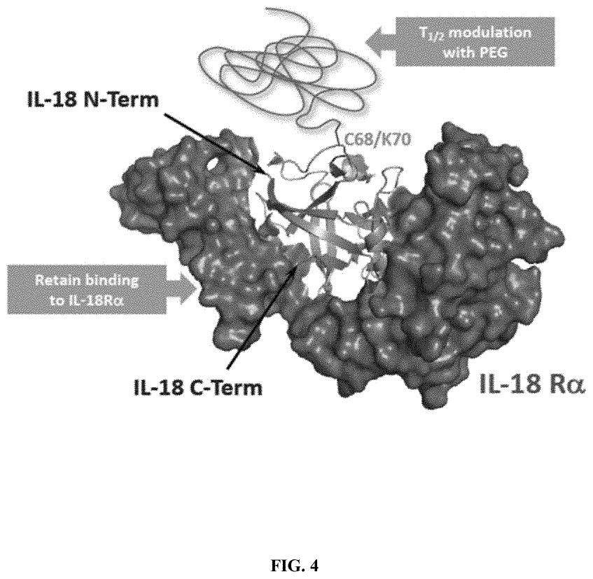

FIG. 4 shows the synthesis of a modified IL-18 polypeptide, Composition A. Composition A comprises a 30 kDa PEG functionality attached to residue C68 on the end of the short PEG polymer. Optionally, this 30 kDa PEG functionality is covalently attached to the short PEG polymer by means of a copper-free click chemical reaction.

Further provided herein is a modified IL-18 polypeptide Composition B. Composition B comprises a 30 kDa PEG functionality attached to residue K70 on the end of the short PEG polymer. Optionally, this 30 kDa PEG functionality is covalently attached to the short PEG polymer by means of a copper-free click chemical reaction.

Further provided herein is a modified IL-18 polypeptide Composition C. Composition C comprises a 30 kDa PEG functionality attached to residue E69 on the end of the short PEG polymer. Optionally, this 30 kDa PEG functionality is covalently attached to the short PEG polymer by means of a copper-free click chemical reaction

### Example 6—Characterization of Composition a and Composition B

Composition A and Composition B are subject to a series of analytical experiments to characterize the compositions. The modified IL-18 polypeptides are analyzed by HPLC to determine the degree of uniformity in the compositions. The modified IL-18 polypeptide compositions are also analyzed by MALDI-MS to determine the MW and distribution of molecular weights of the compositions. The modified IL-18 polypeptide compositions are further analyzed by circular dichroism to compare the folding of the modified IL-18 polypeptide compositions compared to wild type IL-18.

### Example 7—Formulation of Modified IL-18 Polypeptides

The lyophilized modified IL-18 polypeptides were suspended in a solution comprising PBS buffer (pH 7.4) with 50 mg/mL mannitol.

### Example 8—IL-18 SPR Measurements

The interaction of the wild type and of modified IL-18 polypeptides with human IL-18 receptor subunits were measured with Surface Plasmon Resonance (SPR) technology. Anti-human IgG antibodies were bound by amine coupling onto a CM5 chip to capture 6 μg/mL of Fc fused human IL-18Rα, 6 μg/mL of Fc fused human IL-18Rβ, or 2 μg/mL of Fc fused human IL-18BP isoform a (IL-18BPa) for 30 min before capture. In other settings, 6 μg/mL of alpha and beta IL-18 receptors were mixed and pre-incubated for 30 min before capture of the alpha/beta heterodimer IL-18 receptor.

The kinetic binding of the IL-18 analytes were measured with a Biacore 8K instrument in two-fold serial dilutions starting at 1 μM down to 0.98 nM. Regeneration of the surface back to amine coupled anti IgG antibody was done after every concentration of analyte. To measure the protein association to the receptors, the samples were injected with a flow rate of 50 μL/min for 60 s, followed by 300 s buffer only to detect the dissociation. The used running buffer was 1×PBS with 0.05% Tween20. The relative response units (RU, Y-axis) are plotted against time (s, X-axis) and analyzed in a kinetic 1:1 binding model for the monomer receptor binding and for the binding to the IL-18BP. A kinetic heterogenous ligand fit model was applied for the alpha/beta heterodimer binding.

### Example 9—IL-18BP Binding alphaLISA Assay

A human IL-18BP AlphaLISA Assay Kit was used to determine the binding affinity of each IL-18 variant for IL-18BP, which detected the presence of free form IL-18BP.

Sixteen three-fold serial dilutions of IL-18 analytes were prepared in aMEM medium supplemented with 20% FCS, Glutamax, and 25 μM β-mercaptoethanol in the presence of 5 ng/mL of His-tagged human IL-18BP. Final IL-18 analytes concentration ranged from 2778 nM to 0.2 pM.

After 1 hr incubation at room temperature, free IL-18BP levels were measured using a Human IFNγ AlphaLISA Assay Kit. In a 384 well OPTIplate, 5 μL of 5× Anti-IL-18BP acceptor beads were added to 7.5 μL of an IL-18/IL-18BP mix. After 30 min incubation at room temperature with shaking, 5 μL of biotinylated Anti-IL-18BP antibodies were added to each well. The plate was incubated further for 1 hr at room temperature. Under subdued light, 12.5 μL of 2× streptavidin (SA) donor beads were pipetted into each well, and the wells were incubated with shaking for an additional 30 min at room temperature. The AlphaLisa signal was then measured on an Enspire plate reader with 680 and 615 nm as excitation and emission wavelengths, respectively. The dissociation constant (K_{D}) was calculated based on a variable slope, four parameter analysis using GraphPad PRISM software.

### Example 10—Binding of IL-18 Variants to IL-18Rα Monomer

Table 5 shows results of the dissociation constants (K_{D}) observed for the IL-18 variants described to IL-18Rα using the protocol as set forth in Example 8. The results show that modified IL-18 polypeptides of SEQ ID NO: 6, 7, 8, and 20 had K_{D} values that were similar to, or lower than that of, WT IL-18 of SEQ ID NO: 1. Modified IL-18 polypeptides having sequence modifications E06K, K53A, S55A, and T63A maintained binding affinity to IL-18Rα.

TABLE 5
Binding of IL-18Ra monomer

| SEQ ID | | Kₒₙ | K_{off} | K_{D} |
|---|---|---|---|---|
| NO: | Sequence modifications | (1/Ms) | (1/s) | (nM) |
| 1 | Native sequence | 669000 | 0.00484 | 7.24 |
| 2 | E06K, K53A, S55A | 31800 | 0.0627 | 2000 |
| 3 | Y01G, F02A, E06K, M51G, | No | No | >1000 |
| | K53A, D54A, S55A, T63A | binding | binding | |
| 4 | K53A | 160000 | 0.032 | 200 |
| 5 | S55A | 90800 | 0.0121 | 136 |
| 6 | E06K | 824000 | 0.00111 | 1.37 |
| 7 | E06K, K53A | 816000 | 0.0071 | 8.88 |
| 8 | E06K, S55A | 265000 | 0.00393 | 14.8 |
| 9 | K53A, S55A | | | >1000 |
| 10 | E06K, K53A, S55A, T63A | 337000 | 0.0496 | 147 |
| 11 | E06K, K53A, S55A, Y01G | | | |
| 12 | E06K, K53A, S55A, F02A | No | No | >1000 |
| | | binding | binding | |
| 13 | E06K, K53A, S55A, D54A | No | No | >1000 |
| | | binding | binding | |
| 14 | E06K, K53A, S55A, M51G | No | No | >1000 |
| | | binding | binding | |
| 15 | C38S, C68S, C76S, C127S | 77500 | 0.0179 | 231 |
| 16 | C38S, C68S, C76S, C127S, | 51900 | 0.0158 | 304 |
| | K70C | | | |
| 17 | E06K, K53A, S55A, C38S, | No | No | >1000 |
| | C68S, C76S, C127S, K70C | binding | binding | |
| 18 | E06K, K53A, T63A | 151000 | 0.00573 | 38 |
| 19 | T63A | 95200 | 0.00359 | 37.8 |
| 20 | E06K, T63A | 648000 | 0.00151 | 2.33 |
| 21 | K53A, T63A | 16600 | 0.0133 | 800 |
| 22 | E06K, K53A, C38S, C68S, | 8970 | 0.0199 | 2210 |
| | C76S, C127S, K70C | | | |
| 23 | K53A, T63A, C38S, C68S, | No | No | >1000 |
| | C76S, C127S, K70C | binding | binding | |
| 70 | E6K, K53A, C38S, C76S, | 92600 | 0.0369 | 379 |
| | C127S | | | |
| 71 + PEG | E6K, C38S, K53A, | 31800 | 0.0217 | 682 |
| | C68-30kDPEG | | | |
| 71 | E6K, C38S, K53A | 208000 | 0.0151 | 72.4 |

### Example 11—Binding of IL-18 Variants to IL-18Rα/β Heterodimer

Table 6 shows results of the dissociation constants (K_{D}) observed for the IL-18 variants described to IL-18Rα/β heterodimer using the experimental as described in Example 8. The results show that modified IL-18 polypeptides of SEQ ID NO: 6, 7, 8, 18, 19, and 20 had K_{D} values similar to wild type IL-18 of SEQ ID NO: 1. In some cases, the modified IL-18 polypeptides displayed lower K_{D} values than wild type IL-18. Modified IL-18 polypeptides bearing sequence modifications E06K, K53A, S55A, and T63A maintained binding affinity to IL-18Rα/β heterodimer. The data show that some IL-18 variants of the disclosure had decreased dissociation constants, which reflected stabilization of the IL-18/IL-18R complex.

TABLE 6
Binding of IL-1812α/β heterodimer

| SEQ ID | | Kₒₙ | K_{off} | K_{D} |
|---|---|---|---|---|
| NO: | Sequence modifications | (1/Ms) | (1/s) | (nM) |
| 1 | Native sequence | 5800000 | 0.010900 | 1.88 |
| 2 | E06K, K53A, S55A | 39200 | 0.073400 | 1930 |
| 3 | Y01G, F02A, E06K, M51G, | No | No | >1000 |
| | K53A, D54A, S55A, T63A | binding | binding | |
| 4 | K53A | 142000 | 0.035400 | 249 |
| 5 | S55A | 152000 | 0.009980 | 96.4 |
| 6 | E06K | 1300000 | 0.000391 | 0.335 |
| 7 | E06K, K53A | 582000 | 0.000856 | 1.47 |
| 8 | E06K, S55A | 63000000 | 0.933000 | 14.8 |
| 9 | K53A, S55A | Weak | Weak | Weak |
| | | binding | binding | binding |
| 10 | E06K, K53A, S55A, T63A | 409000 | 0.054000 | 132 |
| 11 | E06K, K53A, S55A, Y01G | | | |
| 12 | E06K, K53A, S55A, F02A | No | No | >1000 |
| | | binding | binding | |
| 13 | E06K, K53A, S55A, D54A | No | No | >1000 |
| | | binding | binding | |
| 14 | E06K, K53A, S55A, M51G | Weak | Weak | Weak |
| | | binding | binding | binding |
| 15 | C38S, C68S, C76S, C127S | 69800 | 0.022000 | 315 |
| 16 | C38S, C68S, C76S, C127S, | 159000 | 0.031700 | 200 |
| | K70C | | | |
| 17 | E06K, K53A, S55A, C38S, | No | No | >1000 |
| | C68S, C76S, C127S, K70C | binding | binding | |
| 18 | E06K, K53A, T63A | 136000 | 0.000955 | 7.01 |
| 19 | T63A | 68600 | 0.000624 | 9.09 |
| 20 | E06K, T63A | 693000 | 0.000456 | 0.658 |
| 21 | K53A, T63A | 16700 | 0.001050 | 63 |
| 22 | E06K, K53A, C38S, C68S, | 15800 | 0.000761 | 48.2 |
| | C76S, C127S, K70C | | | |
| 23 | K53A, T63A, C38S, C68S, | No | No | >1000 |
| | C76S, C127S, K70C | binding | binding | |
| 70 | E6K, K53A, C38S, C76S, | 43650 | 0.0006 | 13.19 |
| | C127S | | | |
| 71 + PEG | E6K, C38S, K53A, | 32200 | 0.0008 | 23.9 |
| | C68-30kDPEG | | | |
| 71 | E6K, C38S, K53A | 279000 | 0.0008 | 2.92 |

### Example 12—Binding Assay to IL-18BP Monomer

Table 7 shows results of the dissociation constants (K_{D}) observed for the IL-18 variants described to IL-18BP using an analogous protocol to that described in Example 8. The results show that modified IL-18 polypeptides of SEQ ID NOs: 4, 5, 6, 7, 8, 15, 16, 19, 20, and 21 had K_{D} values similar to, or higher than, wild type IL-18 of SEQ ID NO: 1. Modified IL-18 polypeptides bearing sequence modifications K53A, S55A, E06K, C38S, C68S, C76S, C127S, and/or K70C maintained binding affinity to IL-18BP.

Many of the modified IL-18 variants did not substantially modify the association to IL-18BP (Kₒₙ) but only destabilized the complex, as shown by 10-fold higher dissociation constants observed by some of the modified IL-18 variants of the disclosure. For these modified IL-18 variants, stabilization of IL-18/IL-18R complexes and destabilization of IL-18/IL-18BP complexes resulted in a shifted equilibrium in the competition of IL-18R and IL-18BP for IL-18. For many of the variants, binding to IL-18BP was not abolished, yet they exhibited similar or slightly improved binding to IL18R compared to IL-18BP.

TABLE 7
Binding of IL-18BP monomer determined by SPR

| SEQ ID | | Kₒₙ | K_{off} | K_{D} |
|---|---|---|---|---|
| NO: | Sequence modifications | (1/Ms) | (1/s) | (nM) |
| 1 | Native sequence | 1.49E+06 | 3.83E−04 | 0.270 |
| 2 | E06K, K53A, S55A | 4.78E+04 | 1.54E−02 | 322.7 |
| 3 | Y01G, F02A, E06K, M51G, | No | No | >1000 |
| | K53A, D54A, S55A, T63A | binding | binding | |
| 4 | K53A | 2.06E+05 | 5.38E−04 | 1.33 |
| 5 | S55A | 2.70E+05 | 2.98E−04 | 1.13 |
| 6 | E06K | 1.46E+06 | 1.41E−04 | 0.101 |
| 7 | E06K, K53A | 1.36E+06 | 3.04E−03 | 2.26 |
| 8 | E06K, S55A | 7.10E+05 | 2.58E−04 | 0.364 |
| 9 | K53A, S55A | 6.74E+04 | 2.01E−03 | 29.8 |
| 10 | E06K, K53A, S55A, T63A | 3.70E+05 | 1.81E−02 | 49 |
| 11 | E06K, K53A, S55A, YO1G | | | |
| 12 | E06K, K53A, S55A, FO2A | No | No | >1000 |
| | | binding | binding | |
| 13 | E06K, K53A, S55A, D54A | No | No | >1000 |
| | | binding | binding | |
| 14 | E06K, K53A, S55A, M51G | No | No | >1000 |
| | | binding | binding | |
| 15 | C38S, C68S, C76S, C127S | 1.27E+06 | 2.53E−04 | 0.199 |
| 16 | C38S, C68S, C76S, C127S, | 7.08E+05 | 2.65E−04 | 0.374 |
| | K70C | | | |
| 17 | E06K, K53A, S55A, C38S, | | | 1000 |
| | C68S, C76S, C127S, K70C | | | |
| 18 | E06K, K53A, T63A | 2.79E+05 | 2.91E−03 | 10.4 |
| 19 | T63A | 2.79E+05 | 8.98E−06 | 0.0321 |
| 20 | E06K, T63A | 1.87E+06 | 3.28E−04 | 0.175 |
| 21 | K53A, T63A | 1.05E+05 | 9.66E−04 | 9.22 |
| 22 | E06K, K53A, C38S, C68S, | 1.01E+05 | 2.35E−03 | 23.2 |
| | C76S, C127S, K70C | | | |
| 23 | K53A, T63A, C38S, C68S, | 2.24E+04 | 1.18E−03 | 52.8 |
| | C76S, C127S, K70C | | | |
| 70 | E6K, K53A, C38S, C76S, | 128000 | 0.0037 | 29.1 |
| | C127S | | | |
| 71 + PEG | E6K, C38S, K53A, | 158000 | 0.0039 | 24.5 |
| | C68-301cDPEG | | | |
| 71 | E6K, C38S, K53A | 1140000 | 0.0031 | 2.72 |

Table 8 shows results of the dissociation constants (K_{D}) observed for the IL-18 variants described to IL-18BP as measured using the protocol described in Example 9. The results show that modified IL-18 polypeptides of SEQ ID NO: 5, 6, 8, 15, and 16 had K_{D} values similar to, or higher than, wild type IL-18 of SEQ ID NO: 1.

TABLE 8
Binding of IL-18BP monomer determined by alphaLISA

| SEQ ID | | |
|---|---|---|
| NO: | Sequence modifications | KD (nM) |
| 1 | Native Sequence | 0.67 |
| 2 | E06K, K53A, S55A | >1500 |
| 3 | Y01G, F02A, E06K, M51G, K53A, | 969.0 |
| | D54A, 555A, T63A | |
| 4 | K53A | 513.8 |
| 5 | S55A | 10.7 |
| 6 | E06K | 0.13 |
| 7 | E06K, K53A | 130.3 |
| 8 | E06K, 555A | 12.3 |
| 9 | K53A, 555A | 500.0 |
| 10 | E06K, K53A, S55A, T63A | 822.0 |
| 11 | E06K, K53A, S55A, Y01G | |
| 12 | E06K, K53A, S55A, FO2A | >1000 |
| 13 | E06K, K53A, S55A, D54A | >1000 |
| 14 | E06K, K53A, S55A, M51G | >1000 |
| 15 | C38S, C68S, C76S, C127S | 0.03 |
| 16 | C38S, C68S, C76S, C127S, K70C | 0.21 |
| 17 | E06K, K53A, S55A, C38S, C68S, C76S, | >1000 |
| | C127S, K70C | |
| 18 | E06K, K53A, T63A | 339.8 |
| 19 | T63A | 2.59 |
| 20 | E06K, T63A | 0.83 |
| 21 | K53A, T63A | 198 |
| 22 | E06K, K53A, C38S, C68S, C76S, C127S, | 446.0 |
| | K70C | |
| 23 | K53A, T63A, C38S, C68S, C76S, C127S, | 913 |
| | K70C | |
| 70 | E6K, K53A, C38S, C76S, C127S | 435.5 |
| 71 | E6K, K53A, C38S | 50.2 |
| 71 + PEG | E6K, C38S, K53A, C68-301cDPEG | 59.1 |

### Example 13—IFNγ Induction Cellular Assay

The ability of IL-18 polypeptides provided herein were assessed for ability to induce IFN ⋅ in a cellular assay according to the protocol below.

The NK cell line NK-92 derived from a patient with lymphoma (ATCC® CRL-2407™) was cultured in aMEM medium supplemented with 20% FCS, Glutamax, 25 μM B-mercaptoethanol, and 100 IU/mL of recombinant human IL-2.

On the day of experiment, cells were harvested and washed with aMEM medium without IL-2 and containing 1 ng/mL of recombinant human IL-12. After counting, cells were seeded at 100,000 cells/well in a 384 well titer plate and incubated at 37° C./5% CO₂. Sixteen 4-fold serial dilutions of IL-18 analytes were prepared in aMEM medium, and 1 ng/mL of IL-12 were added to the NK-92 cells. Final IL-18 analyte concentrations ranged from 56 nM to 5×10⁻⁵ pM.

After incubating the cells for 16-20 hr at 37° C./5% CO₂, 5 μL of supernatant was carefully transferred to a 384 microwell OptiPlate. IFNγ levels were measured using a human IFNγ AlphaLISA Assay Kit. Briefly, 10 μL of 2.5× AlphaLISA Anti-IFNγ acceptor beads and biotinylated antibody anti-IFNγ mix were added to the 50, of NK-92 supernatants. The mixtures were incubated for 1 hr at room temperature with shaking. Under subdued light, 2.5 μl, of 2× streptavidin (SA) donor beads were pipetted into each well, and the wells were incubated for 30 min at room temperature with shaking. AlphaLISA signals were then measured on an EnSpire™ plate reader using 680 nm and 615 nm as excitation and emission wavelengths, respectively. Half maximal effective concentrations (EC₅₀) were calculated based on a variable slope and four parameter analysis using GraphPad PRISM software.

Results of this experiment for various IL-18 polypeptides is shown below in Table 9 (EC₅₀ data).

### Example 14—IL-18 Binding Protein Inhibition Cellular Assay

The NK cell line NK-92 derived from a patient with lymphoma (ATCC® CRL-2407™) was cultured in aMEM medium supplemented with 20% FCS-Glutamax, 25 μM B-mercaptoethanol, and 100 IU/mL of recombinant human IL-2.

On the day of experiment, cells were harvested and washed with aMEM medium without IL-2 and containing 1 ng/mL of recombinant human IL-12. After counting, the cells were seeded at 100,000 cells/well in a 384 well titer plate and incubated at 37° C./5% CO₂. Sixteen 2-fold serial dilutions of Fc-fused human IL-18 binding protein isoform a (IL-18BPa) were prepared in aMEM medium. 1 ng/mL of IL-12 containing 2 nM of each modified IL-18 polypeptide variant was added to the NK-92 cells. The final IL-18 analyte concentration was 1 nM, and the final IL-18BPa concentration ranged from 566 nM to 17 pM.

After incubating the cells for 16-20 hr at 37° C./5% CO₂, 5 μL of the supernatant was carefully transferred to a 384 microwell OptiPlate. IFNγ levels were measured using a human IFNγ AlphaLISA Assay Kit. Briefly, 10 μL of 2.5× AlphaLISA anti-IFNγ acceptor beads and biotinylated antibody anti-IFNγ mix were added to 5 μL of NK-92 supernatants. The mixtures were incubated for 1 hr at room temperature with shaking. Under subdued light, 2.5 μL of 2×SA donor beads were pipetted in each well and incubated for 30 min at room temperature with shaking. AlphaLISA signals were then measured on an EnSpire™ plate reader using 680 nm and 615 nm as excitation and emission wavelengths, respectively. Half maximal inhibitory concentrations (IC₅₀) were calculated based on a variable slope and four parameter analysis using GraphPad PRISM software.

Modified IL-18 variants of the disclosure are active and able to induce IFNγ secretion in vitro. Table 9 shows the ability of many of the tested IL-18 variants to induce IFNγ production while some IL-18 variants are significantly less sensitive to inhibition by IL-18BP, as measured by EC₅₀ and IC₅₀, respectively.

TABLE 9
IC₅₀/EC₅₀ in NK92 IFN_{γ} Release Assay Data

| | | | | | Potency |
|---|---|---|---|---|---|
| SEQ | | | | | loss |
| ID | | IC₅₀ | EC₅₀ | IC₅₀/ | Mutein/ |
| NO: | Sequence modifications | (nM) | (nM) | EC₅₀ | WT |
| 1 | Native sequence | 1.47 | 0.276 | 7.59 | 1.00 |
| 2 | E06K, K53A, S55A | 229 | 0.824 | 1420 | 2.99 |
| 3 | Y01G, F02A, E06K, M51G, | >55.0 | >55.0 | <=>1.00 | >200 |
| | K53A, D54A, S55A, T63A | | | | |
| 4 | K53A | 27.3 | 0.444 | 825 | 1.61 |
| 5 | S55A | 4.46 | 0.108 | 41.1 | 0.39 |
| 6 | E06K | 7.79 | 0.0567 | 345 | 0.21 |
| 7 | E06K, K53A | >703 | 0.0192 | >298000 | 0.07 |
| 8 | E06K, S55A | 15 | 0.067 | 1060 | 0.24 |
| 9 | K53A, S55A | 37.3 | 1.58 | 180 | 5.72 |
| 10 | E06K, K53A, S55A, T63A | 1060 | 0.144 | 50600 | 0.52 |
| 11 | E06K, K53A, S55A, Y01G | 27.8 | 6.12 | 11.8 | 22.17 |
| 12 | E06K, K53A, S55A, F02A | NT | >1000 | NT | >3600 |
| 13 | E06K, K53A, S55A, D54A | NT | 30 | NT | 108.70 |
| 14 | E06K, K53A, S55A, M51G | 0.189 | 7.4 | 0.013 | 26.81 |
| 15 | C38S, C68S, C76S, C127S | 0.444 | 0.115 | 3.1 | 0.42 |
| 16 | C38S, C68S, C76S, C127S, | 0.114 | 0.488 | 0.21 | 1.77 |
| | K70C | | | | |
| 17 | E06K, K53A, S55A, C38S, | NT | 58.5 | NT | 211.96 |
| | C68S, C76S, C127S, K70C | | | | |
| 18 | E06K, K53A, T63A | >1000 | 0.0268 | >87000 | 0.10 |
| 19 | T63A | 0.239 | 0.449 | 0.625 | 1.63 |
| 20 | E06K, T63A | 47.1 | 0.011 | 8220 | 0.04 |
| 21 | K53A, T63A | 18.2 | 0.155 | 185 | 0.56 |
| 22 | E06K, K53A, C38S, C68S, | 23.5 | 0.962 | 24.4 | 3.49 |
| | C76S, C127S, K70C | | | | |
| 23 | K53A, T63A, C38S, C68S, | >1000 | 17.2 | >84.2 | 62.32 |
| | C76S, C127S, K70C | | | | |
| 71 | E6K, K53A, C38S | | | | |
| 71 + | E6K, C38S, K53A, | 0.66 | 19.1 | 29.1 | 0.86 |
| PEG | C68-301cDPEG | | | | |

### Example 15—HEK-Blue IL18R Reporter Assay

An IL-18R positive HEK-Blue reporter cell line was used to determine binding of IL-18 variants to IL-18R and subsequent downstream signaling. The general protocol is outlined below.

5×10⁴ cells HEK-Blue IL18R reporter cells (InvivoGen, #hkb-hmil18) were seeded into each well of a 96 well plate and stimulated with 0-100 nM of IL-18 polypeptide variants at 37° C. and 5% CO₂. After 20h incubation, 20 μL of cell culture supernatant was then taken from each well and mixed with 180 μL QUANTI-Blue media in a 96 well plate, incubated for 1 hour at 37° C. and 5% CO₂. The absorbance signal at 620 nm was then measured on an Enspire plate reader with 680 and 615 nm as excitation and emission wavelengths, respectively. Half Maximal Effective dose (EC50) was calculated based on a variable slope, four parameter analysis using GraphPad PRISM software.

Results of this experiment for select variants are shown below in Table 12.

TABLE 12
EC₅₀ in HEK-Blue IL18R Reporter Assay Data

| | | | Potency |
|---|---|---|---|
| | | | loss |
| SEQ ID | | EC₅₀ | Mutant/ |
| NO: | Sequence modifications | (pM) | NWT |
| 1 | Native sequence | 3.33 | 1.00 |
| 2 | E6K, K53A, S55A | 272.5 | 81.8 |
| 7 | E6K, K53A | 0.72 | 0.22 |
| 10 | E6K, K53A, S55A, T63A | 0.79 | 0.24 |
| 18 | E6K, K53A, T63A | 1.77 | 0.53 |
| 22 | E6K, C38S, K53A, C68S, | 9.12 | 2.74 |
| | K70C, C76S, C127S | | |
| 70 | E6K, K53A, C38S, C76S, | 3.73 | 1.11 |
| | C127S | | |
| 71 + PEG | E6K, C38S, K53A, C68-301(DPEG | 1.85 | 0.56 |
| 71 | E6K, C38S, K53A | 0.86 | 0.26 |

### Example 16A—Pharmacokinetic and Pharmacodynamic Properties of Modified IL-18 Polypeptide Variants

The pharmacokinetic (PK) and pharmacodynamic (PD) properties of select IL-18 polypeptide variants were measured. Three C57BL/6 mice were tested per group and per time point. IL-18 variants were applied via single intravenous injections. Mice were divided into four dose groups: 0.5 mg/kg, 0.1 mg/kg, 0.02 mg/kg, 0.004 mg/kg; and four time point groups: 5 min, 6 hr, 24 hr, 48 hr.

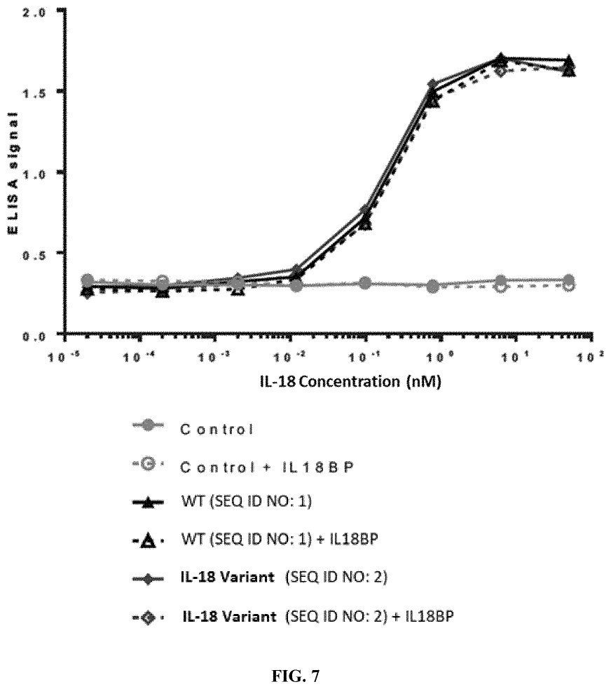

Immune-related PD effects were determined by analyzing cytokine levels in plasma. The following plasma cytokines were measured: IFNγ, CXCL9, CXCL10, GM-CSF, IL-1a, FasL, and IL-18BP. The activation status of leukocytes was determined by monitoring surface markers: ICOS, PD-1, CD25, CD69, and Fas. Bioanalysis was conducted by detecting the total amount of IL-18 variants (free and IL-18BP-complexed, see FIG. 7). Corning high-binding half-area plates (Fisher Scientific, Reinach, Switzerland) were coated overnight at 4° C. with 25 μl of anti-IL18 monoclonal antibody (MBL, cat #D043-3, Clone 25-2G) at 2 μg/ml in PBS. Plates were then washed four times with 100 μl of PBS-0.02% Tween20. Plates surfaces were blocked with 25 μl of PBS-0.02% Tween20-1% BSA at 37° C. during 1 h. Plates were then washed four times with 100 μl of PBS-0.02% Tween20. Twenty-five microliters of IL-18 variants (or of mouse plasma) were added in eight-fold serial dilutions starting at 50 nM down to 0.02 nM into PBS-0.02% Tween20-0.1% BSA and incubated at 37° C. during 2h. Plates were then washed four times with 100 μl of PBS-0.02% Tween20 and 25 μl of of biotinylated anti-IL18 monoclonal antibody (MBL, cat #D045-6, Clone 159-12B) at 2 μg/ml in PBS. Plates were incubated during 2h at 37° C. and were then washed four times with 100 μl of PBS-0.02% Tween20. Twenty-five microliters of Streptavidin-Horseradish peroxidase (#RABHRP3, Merck, Buchs, Switzerland) diluted at 1:500 into PBS-0.02% Tween20-0.1% BSA were added to each well and incubated at Room Temperature during 30 min. Plates were then washed four times with 100 μl of PBS-0.02% Tween20. Fifty microliters of TMB substrate reagent (#CL07, Merck, Buchs, Switzerland) were added to each well and incubated at 37° C. during 5 min. After 5 min at 37° C., Horseradish peroxidase reaction was stopped by adding 50 μl/well of 0.5M H25O4 stop solution. ELISA signal was then measured at 450 nm on an EnSpire plate reader from Perkin Elmer (Schwerzenbach, Switzerland)

FIG. 7 shows ELISA results of the following groups: control; control+IL-18BP; WT IL-18; WT IL-18+IL-18BP; a modified IL-18 polypeptide of SEQ ID NO: 2; a modified IL-18 polypeptide of SEQ ID NO: 2+IL-18BP.

### Example 16B—PK/PD of a Modified IL-18 Harboring E6K and K53A Amino Acid Substitutions

In a separate experiment, three C57BL/6 mice were tested per group and per time point. WT IL-18 (SEQ ID NO: 1) was applied via a single intravenous injection of 0.3 mg/kg. A E6K, K53A variant IL-18 (SEQ ID NO: 7) was applied via two intravenous injections of 0.3 mg/kg, at t=0 hr and t=24 hr. Mice were divided into seven time point groups: 5 min, 1 hr, 2 hr, 4 hr, 8 hr, 24 hr, and 48 hr.

Immune-related PD effects were determined by analyzing cytokine levels in plasma. The following plasma cytokines were measured: GM-CSF, IFNγ, IL-4, IL-5, IL-6, IL-12, TNFα, IL-22, MCP-1, MCP-3, MIP-1a, MIP-1b and CXCL1. The activation status of leukocytes was determined by monitoring surface markers: PD-1 and CD25. Bioanalysis was conducted by detecting the total amount of IL-18 variants (free and IL-18BP-complexed).

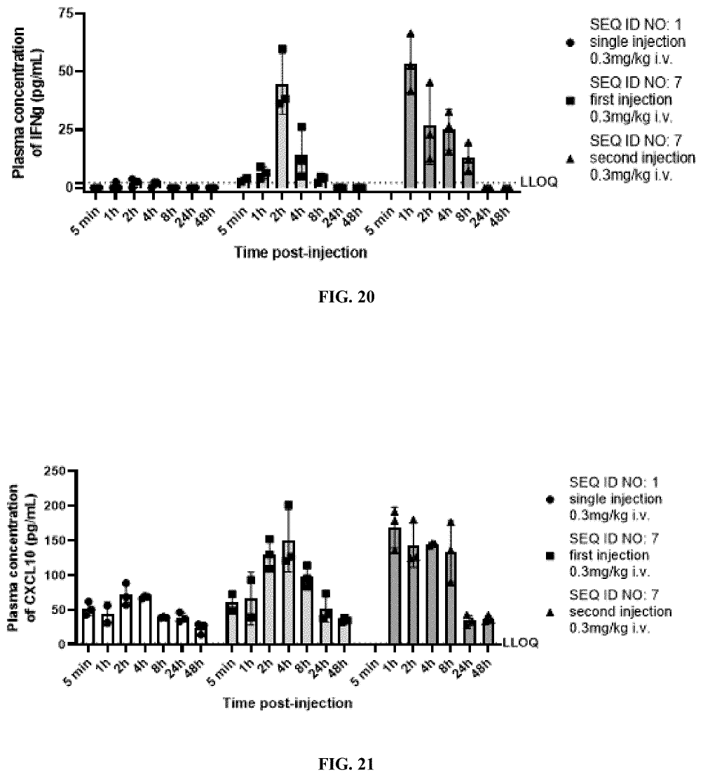

Human IL-18 polypeptide variant (SEQ ID NO: 7) increased cytokine production in vivo compared to wild-type human IL-18. FIG. 20 shows the plasma concentration of IFNγ and FIG. 21 shows the plasma concentration of CXCL10 at various time points post I.v. injection of either wild-type or variant IL-18. The most robust response were found with IFNγ,- and MCP-1, MCP-3, MIP-1a, MIP-1b, CXCL10 chemokines, with significant increases observed between 2 hr and 8 hr post-injection (data for MCP-1, MCP-3, MIP-1a and MIO-1b not shown). Repeated i.v. injection of human IL-18 polypeptide variant (SEQ ID NO: 7) led to a stronger and more rapid response, with plasma cytokine levels increasing between 1 hr and 8 hr post-injection.

### Example 17—rIL18 Expression and Purification

Recombinant IL-18 variants provided herein can be prepared according to the protocols provided below

Soluble his-SUMO-IL18 Variants

E. coli BL21 (DE3) harboring a plasmid encoding a N-His-SUMO tagged IL-18 variant fusion is inoculated into 3 L LB culture medium and induced with 0.4 mM IPTG at 30° C. for 6h. Cells are pelleted and cell lysis is done by sonication in lysis buffer: PBS, pH 7.4. Soluble protein is purified via Ni-NTA beads 6FF (wash 1 with: PBS, 20 mM imidazole, pH7.4; wash 2 with PBS, 50 mM Imidazole, pH7.4; elution with PBS, 500 mM imidazole, pH7.4).

Fractions containing the protein are pooled, dialyzed into PBS pH 7.4 and followed by SUMO digestion. Then the protein is two-step purified with Ni-NTA beads (continue with flow through sample) and gel filtration. Fractions containing the protein are pooled and QC is performed using analytical techniques, such as SDS-PAGE and analytical SEC.

Insoluble his-SUMO-IL18 Variants

E. coli BL21 (DE3) harboring a plasmid encoding a N-His-SUMO tagged IL-18 variant fusion are inoculated into 10 L LB culture medium and induced with 0.4 mM IPTG at 30° C. for 6h. Cells are pelleted and cell lysis is done by sonication in lysis buffer: PBS, 8 M urea, pH 7.4. Protein is purified via Ni-NTA beads 6FF (wash 1 with: PBS, 8 M urea, 20 mM imidazole, pH7.4; wash 2 with PBS, 8 M urea, 50 mM Imidazole, pH7.4; elution with PBS, 8 M urea, 500 mM imidazole, pH7.4).

Fractions containing the protein are pooled, dialyzed into PBS pH 7.4 and followed by SUMO digestion. Then the protein is purified with Ni-NTA beads (equilibrate column with PBS, 8 M urea, pH 7.4, wash with PBS, 8 M urea, pH 7.4, elution with PBS, 8 M urea, pH 7.4). Fractions containing the protein are pooled, dialyzed into PBS pH 7.4 and QC is performed using analytical techniques, such as SDS-PAGE and analytical SEC.

### Insoluble Tagless IL18 Variants

E. coli BL21 (DE3) harboring a plasmid encoding mIL-18 is inoculated into 2 L LB culture medium and induced with 0.4 mM IPTG at 30° C. for 6h. Cells are pelleted and cell lysis was done by sonication in lysis buffer: 110 mM Tris, 1.1 M guanidine HCl, 5 mM DTT, pH 8.9. Protein as purified via Q Sepharose FF (balance buffer 20 mM MES, pH 7.0, elution with an increasing gradient from 0 to 1 M NaCl).

### Bicistronic System

A single colony of E. coli BL21 containing the plasmid (e.g., SEQ ID: 71) is used as an inoculum for 10 mL LB containing 25 μg/mL kanamycin sulfate and incubated overnight at 37° C. and 200 rpm. 1 mL of the preculture are used to inoculate 1 L autoinducing terrific broth containing 100 μg/mL kanamycin sulfate. The culture is incubated at 37° C. and 110 rpm for 4 h and then transferred to 15° C. for another 15 h. Cells are resuspended in 10-15 mL lysis buffer (100 mM Hepes, 1 mM EDTA, 5 mM DTT, 20 μg/mL lysozyme, 0.1 mg/mL DNase I, 1 mM PMSF, pH 7.5) and gently shaken at 4° C. for 1 h. Then the cells are lysed with sonication and the soluble protein fraction is obtained by centrifugation (16,000×g, 30 min, 4° C.) and filtration (0.2 μm membrane).

The supernatant is adjusted to ca. pH 7 and loaded on a tandem column system (2×SP CIEX+1×HiPrep DEAE FF 16/10, all from cytiva) using a 50 mL superloop (loading less than 30 mL lysate per run). The system is run with wash buffer (25 mM Hepes, 1 mM EDTA, 5 mM DTT, pH 7.0) and fractions containing the protein (second main peak) are collected and pooled.

The tandem columns are separated into their respective types. The DEAE columns were eluted with buffers E1 and E2 (25 mM Bis-Tris Propane HCl, pH 9.5 and 25 mM Bis-Tris Propane HCl, 1 M NaCl, pH 9.5 respectively) with a stepwise gradient. First, 100% E1 was run for 8 CV, followed by a gradient from 0% to 12% E2 over 5 CV and then keeping it at 12% for another 10 CV. This is followed by a gradient from 12% to 40% E2 over 5 CV and keeping it at 40% for another 5 CV. Fractions containing the protein (second main peak) are collected and pooled with the previous fractions. The SP columns are washed with the same method and discard, as no protein should be found in this elution.

The pooled samples are adjusted to pH 9.5 and loaded on a Mono Q (small scale) or Hitrap Q (large scale) column. Buffers used are E2 and E3 (25 mM Bis-Tris Propane HCl, 1.5 M Ammonium Sulfate, pH 9.5). The stepwise elution gradient starts at 8% E3 for 15 CV, increasing to 16% E3 over 5 CV and the increasing to 50% E3 over 3 CV. Fractions containing the protein are found in the second main peak.

The fractions containing the target protein are pooled and concentrated by diafiltration (10 kDa MWCO, less than 3500×g, 4° C.). The concentrated sample is loaded on a Superdex 75 equilibrated with buffer (20 mM potassium phosphate, 150 mM KCl, 1 mM DTT, pH 6.0). Fractions containing the target protein are collected, pooled and concentrated

### Example 18—Conjugation of Modified IL-18 Polypeptides

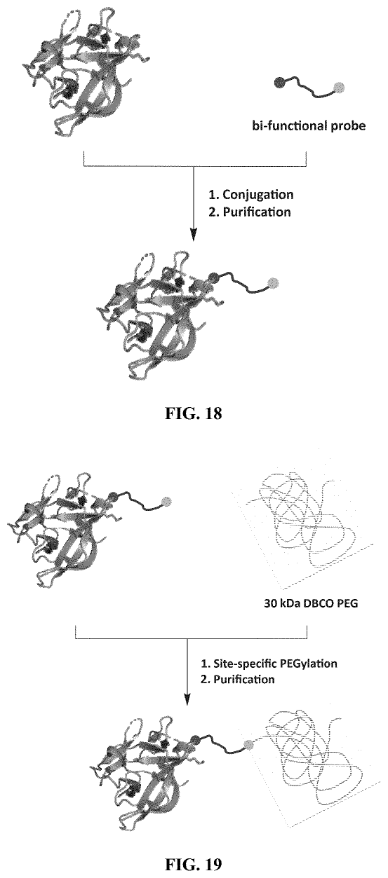

In some instances, a modified IL-18 polypeptide as provided herein is conjugated to a PEG functionality. In some cases, the PEG is attached via a bifunctional linker which first attaches to a desired residue of the modified IL-18 polypeptide (e.g., C68 or another suitable naturally occurring cysteine or a cysteine residue which has been incorporated at a desired site, such as residue 69 or 70). Once attached to the IL-18 polypeptide, the second functionality of the bifunctional linker is used to attach the PEG moiety. An exemplary schematic of such a process is shown in FIG. 18. An exemplary protocol on a recombinant IL-18 variant provided herein is described below.

Conjugation—Recombinant IL-18 was stored at a concentration of 2.4 mg/mL at −80° C. in potassium phosphate buffer (pH 7.0) containing 50 mM KCl and 1 mM DTT. The sample was thawed on ice yielding a clear solution. The protein solution was diluted in PBS, pH 7.4. A clear solution was obtained at a concentration of 0.4 mg/mL.

The protein solution was dialyzed against PBS, pH 7.4 (twice against 600 mL for 2 h and once against 800 mL for 18 h). After dialysis, a clear solution was obtained with no sign of precipitation. Protein concentration was obtained using UV absorbance at 280 nm and by BCA protein assay.

A stock solution of bi-functional probe (bromoacetamido-PEG5-azide, CAS: 1415800-37-1) in water was prepared at a concentration of 20 mM. 500 μL of the protein solution were mixed with 25 μL of probe solution. pH was adjusted to 7.5 and it was let to react for 3 h at 20° C.

The progress of the synthesis was monitored by reverse-phase HPLC using a gradient of 5 to 30% (2.5 min) and 30 to 75% (7.5 min) CH₃CN with 0.1% TFA (v/v) on a Aeris WIDEPORE C18 200 Å column (3.6 μm, 150×4.6 mm) at a flow rate of 1 mL/min at 40° C. and by MALDI-TOF MS

Purification—In some cases, ion-exchange chromatography was used to purify the conjugated protein. To remove the excess of probe, the reaction mixture (volume is around 500 μL) was flowed through a Hi-Trap-G-FF-1 mL column using 25 mM Tris (pH 7.4) as the buffer. The column was eluted with a linear gradient of 0-0.35 M NaCl in the same buffer. The fractions containing the target protein were gathered, buffer exchanged (25 mM Tris, pH 7.4, 75 mM NaCl, 5% glycerol) and concentrated at 0.4 mg/mL. The concentration of purified protein was determined by UV absorbance at 280 nm and by BCA protein assay. The protein solution was kept at −80° C.

Characterization—The purity and identity of the recombinant protein from commercial source and the conjugated protein was confirmed by aSEC, HPLC and MALDI-TOF MS.

### Example 19—PEGylation of Modified IL-18 Polypeptides

After conjugation of the bifunctional linker as described in Example 19 and as shown in FIG. 18, the modified IL-18 polypeptide can be covalently linked with a PEG group. An exemplary schematic of this process is shown in FIG. 19. An exemplary protocol of the conjugation reaction between a PEG and a suitably activated IL-18 polypeptide is provided below. Additionally, the protocol below can be used to covalently link a desired PEG group to a modified IL-18 polypeptide which incorporates a conjugation handle directly during the preparation of the modified IL-18 polypeptide (e.g., during the synthesis of a synthetic IL-18 polypeptide). An exemplary schematic of such a process is shown in FIG. 3.

Conjugation—Recombinant modified IL-18 polypeptide of SEQ ID NO: 71 was stored at −80° C. in PBS (pH 7.4) containing 75 mM NaCl and 5% (v/v) glycerol. Prior to PEGylation reaction, the sample was thawed on ice yielding a clear solution. 200 μL of the protein solution (0.4 mg/mL) were mixed with 2.0 mg of 30 kDa DBCO-polyethylene glycol polymer. It was let to react overnight at 20° C.

The progress of the synthesis was monitored by reverse-phase HPLC using a gradient of 5 to 30% (2.5 min) and 30 to 75% (7.5 min) CH₃CN with 0.1% TFA (v/v) on a Aeris WIDEPORE C4 200 Å column (3.6 μm, 150×4.6 mm) at a flow rate of 1 mL/min at 40° C. and by MALDI-TOF MS.

Purification—To remove the excess of PEG, the reaction mixture was diluted with Tris buffer (25 mM, pH 7.4) and flowed through a Hi-Trap-Q-FF column using 25 mM Tris (pH 7.4) as the buffer. The column was eluted with a linear gradient of 0-0.35 M NaCl in the same buffer. The fractions containing the target protein were gathered, buffer exchanged (25 mM Tris, pH 7.4, 75 mM NaCl, 5% glycerol) and concentrated at 0.04 mg/mL. The concentration of purified protein was determined by BCA protein assay. The protein solution was kept at −80° C.

Characterization—The purity and identity of the conjugated protein was confirmed by HPLC and MALDI-TOF MS.

### Example 20—PBMC Stimulation Assay

Ability of IL-18 variants to stimulate peripheral blood mononuclear cells (PBMCs) was assessed according to the following protocol.

Isolation of lymphocytes: Blood from Buffy Coats of healthy volunteers was diluted with equal volume of PBS and slowly poured on top of SepMate tube prefilled with 15 mL Histopaque-1077. Tubes were centrifuged for 10 minutes at 1200 g, the top layer was collected and washed 3 times with PBS containing 2% of Fetal Bovine Serum. PBMCs were counted and cryopreserved as aliquots of 20×10⁶ cells.

Cryopreserved PBMCs were thawed and stimulated with gradient of human IL-18 variants ranging from 0.2 μM to 1 μM in RPMI containing 10% Fetal Bovine Serum.

Cytokine production after 24 hr stimulation is measured by Legendplex (Biolegend #740930) on a multicolor flow cytometer. Half maximal effective concentrations (EC₅₀) of IFNγ released in culture supernatant are calculated based on a variable slope and four parameter analysis using GraphPad PRISM software.

Surface expression of FcγRIII on NK cells is measured by flow cytometry (Mouse IgG1 clone 3G8) after 72 hr stimulation.

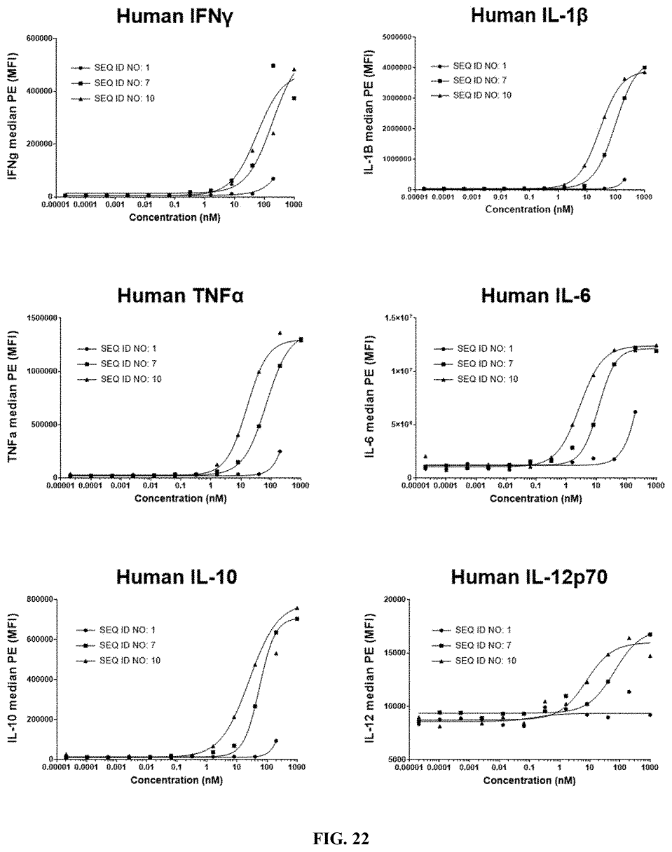

FIG. 22 shows the induction of (clockwise from top left) human IFNγ, IL-1β, IL-6, IL-12p70, IL-10, and TNFα upon administration of wild type IL-18 (SEQ ID NO: 1) and modified IL-18 polypeptides of SEQ ID NO: 7 and SEQ ID NO: 10. In each individual graph, the x-axis displays the concentration of the indicated IL-18 polypeptide (nM) and the y-axis indicates the mean fluorescence intensity (MFI) of the indicated biomarker. In the graphs, wild type IL-18 is represented as circles, modified IL-18 polypeptide of SEQ ID NO: 7 is represented as squares, and modified IL-18 polypeptide of SEQ ID NO: 10 is represented as triangles. For each biomarker, the modified IL-18 polypeptides induced production of the indicated cytokines at lower concentrations than wild type. Between the modified IL-18 polypeptides of SEQ ID NO: 7 and SEQ ID NO: 10, the SEQ ID NO: 10 induced cytokine production at lower concentrations for each cytokine.

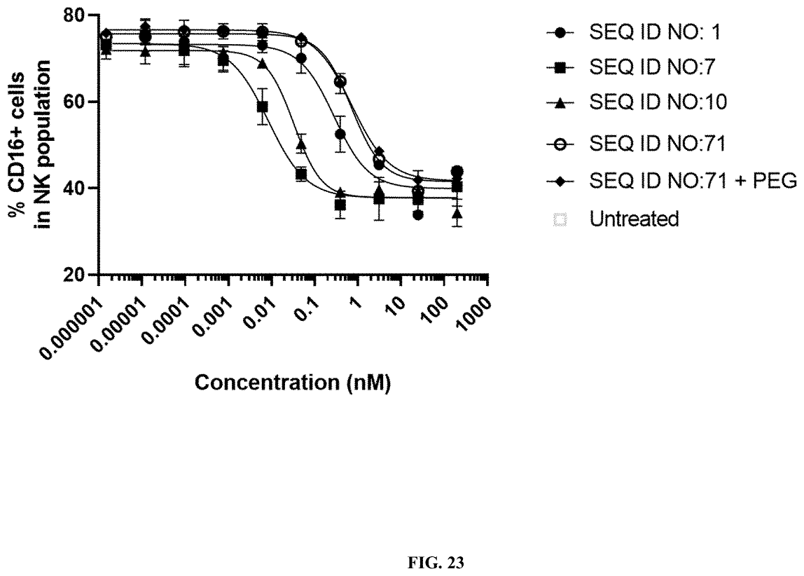

FIG. 23 shows the percentage of CD16⁺ cells in the NK cell population of PBMCs stimulated with modified IL-18 polypeptides (y-axis, express as a % of the total population). The x-axis displays the concentration of the relavant IL-18 polypeptide (nM). Wild type IL-18 is represented as a full circle, SEQ ID NO: 7 is represented as a full square, SEQ ID NO: 10 is represented as a full triangle, SEQ ID NO: 71 is represented as an empty circle, and SEQ ID NO: 71 with a 30 kDa PEG attached at residue C68 is represented by a filled diamond. SEQ ID NOS: 7 and 10 displayed activity at lower concentrations compared to wild type, whereas SEQ ID NO: 71 and SEQ ID NO: 71 with PEG displayed activity at similar but slightly higher concentrations than wild type.

What is claimed is:

1. A modified interleukin-18 (IL-18) polypeptide, comprising:
a modified IL-18 polypeptide comprising E06K and K53A, wherein residue position numbering of the modified IL-18 polypeptide is based on SEQ ID NO: 1 as a reference sequence.

2. The modified IL-18 polypeptide of claim 1, wherein the modified IL-18 polypeptide further comprises T63A.

3. The modified IL-18 polypeptide of claim 1 or 2, wherein the modified IL-18 polypeptide further comprises at least one of Y01X, S55X, F02X, D54X, C38X, C68X, E69X, C76X, C127X, or K70X, wherein X is an amino acid or an amino acid derivative.

4. The modified IL-18 polypeptide of claim 3, wherein the modified IL-18 polypeptide comprises at least one of Y01G, S55A, F02A, D54A, C38S, C38A, C68S, C68A, E69C, C76S, C76A, C127S, C127A, or K70C.

5. The modified IL-18 polypeptide of any one of claims 1 to 4, wherein the modified IL-18 polypeptide comprises a polymer covalently attached at residue 65, residue 66, residue 67, residue 68, residue 69, residue 70, residue 71, residue 72, residue 73, residue 74 or residue 75.

6. The modified IL-18 polypeptide of any one of claims 1 to 5, wherein the modified IL-18 polypeptide comprises a polymer covalently attached at residue C68.

7. The modified IL-18 polypeptide of any one of claims 1 to 6, wherein the modified IL-18 polypeptide comprises a polymer covalently attached at residue E69 or E69C.

8. The modified IL-18 polypeptide of any one of claims 1 to 7, wherein the modified IL-18 polypeptide comprises a polymer covalently attached at residue K70 or K70C.

9. The modified IL-18 polypeptide of any one of claims 5 to 8, wherein the polymer has a weight average molecular weight of at most about 50,000 Daltons, at most about 25,000 Daltons, at most about 10,000 Daltons, at most about 6,000 Daltons or at most about 2,000 Daltons.

10. The modified IL-18 polypeptide of any one of claims 5 to 9, wherein the polymer has a weight average molecular weight of at least about 120 Daltons, at least about 250 Daltons, at least about 300 Daltons, at least about 400 Daltons, or at least about 500 Daltons.

11. The modified IL-18 polypeptide of any one of claims 5-10, wherein the polymer comprises a conjugation handle or a reaction product of a conjugation handle with a complementary conjugation handle.

12. The modified IL-18 polypeptide of any one of claims 5 to 11, wherein the polymer comprises an azide moiety, an alkyne moiety, or reaction product of an azide-alkyne cycloaddition reaction.

13. The modified IL-18 polypeptide of any one of claims 5 to 12, wherein the polymer is a water-soluble polymer.

14. The modified IL-18 polypeptide of claim 13, wherein the water-soluble polymer comprises poly(alkylene oxide), polysaccharide, poly(vinyl pyrrolidone), poly(vinyl alcohol), polyoxazoline, poly(acryloylmorpholine), or a combination thereof.

15. The modified IL-18 polypeptide of claim 13, wherein the water-soluble polymer comprises poly(alkylene oxide).

16. The modified IL-18 polypeptide of claim 14 or 15, wherein the poly(alkylene oxide) is polyethylene glycol (PEG).

17. The modified IL-18 polypeptide of claim 16, wherein the polyethylene glycol has a weight average molecular weight of about 10 kDa to about 50 kDa.

18. The modified IL-18 polypeptide of claim 16, wherein the polyethylene glycol has a weight average molecular weight of about 10 kDa, about 20 kDa, or about 30 kDa.

19. The modified IL-18 polypeptide of claim 16, wherein the polyethylene glycol has a weight average molecular weight of about 30 kDa.

20. The modified IL-18 polypeptide of any one of claims 5-19, wherein a half-life of the modified IL-18 polypeptide is at least 10% longer than a half-life of a corresponding wild-type IL-18 polypeptide.

21. The modified IL-18 polypeptide of claim 20, wherein the half-life of the modified IL-18 polypeptide is at least 30% longer than the half-life of the corresponding wild-type IL-18 polypeptide.

22. The modified IL-18 polypeptide of any one of claims 1-21, wherein the modified IL-18 polypeptide comprises an N-terminal extension.

23. The modified IL-18 polypeptide of any one of claims 1-21, comprising an N-terminal truncation.

24. The modified IL-18 polypeptide of any one of claims 1-23, wherein the modified IL-18 polypeptide comprises a polypeptide sequence having at least about 80% sequence identity to any one of SEQ ID NOs: 2-83.

25. The modified IL-18 polypeptide of claim 24, wherein the polypeptide sequence is at least about 80% identical to SEQ ID NO: 2 or SEQ ID NO: 18.

26. The modified IL-18 polypeptide of claim 25, wherein the polypeptide sequence is at least about 80% identical to SEQ ID NO: 2.

27. The modified IL-18 polypeptide of claim 25, wherein the polypeptide sequence is at least about 90% identical to SEQ ID NO: 2.

28. The modified IL-18 polypeptide of claim 25, wherein the polypeptide sequence is at least about 95% identical to SEQ ID NO: 2.

29. The modified IL-18 polypeptide of claim 25, wherein the polypeptide sequence is at least about 80% identical to SEQ ID NO: 18.

30. The modified IL-18 polypeptide of claim 25, wherein the polypeptide sequence is at least about 90% identical to SEQ ID NO: 18.

31. The modified IL-18 polypeptide of claim 25, wherein the polypeptide sequence is at least about 95% identical to SEQ ID NO: 18.

32. The modified IL-18 polypeptide of any one of claims 1-31, wherein the modified IL-18 polypeptide is recombinant.

33. The modified IL-18 polypeptide of any one of claims 1-31, comprising one or more amino acid substitutions selected from:
(a) a homoserine residue located at any one of residues 26-36;
(b) a homoserine residue located at any one of residues 60-80;
(c) a homoserine residue located at any one of residues 110-120;
(d) a norleucine or O-methyl-homoserine residue located at any one of residues 28-38;
(d) a norleucine or O-methyl-homoserine residue located at any one of residues 46-56;
(e) a norleucine or O-methyl-homoserine residue located at any one of residues 54-64;
(f) a norleucine or O-methyl-homoserine residue located at any one of residues 80-90;
(g) a norleucine or O-methyl-homoserine residue located at any one of residues 108-118; and
(h) a norleucine or O-methyl-homoserine residue located at any one of residues 145-155;
wherein residue position numbering of the modified IL-18 polypeptide is based on SEQ ID NO: 1 as a reference sequence.

34. The modified IL-18 polypeptide of claim 33, comprising one or more amino acid substitutions selected from homoserine (Hse) 31, norleucine (Nle) 33, O-methyl-homoserine (Omh) 33, Nle51, Omh51, Nle60, Omh60, Hse75, Nle86, Omh86, Hse106, Nle113, Omh113, Nle150, and Omh150.

35. The modified IL-18 polypeptide of any one of claims 1-34, wherein the modified IL-18 polypeptide modulates IFNγ production, and wherein an EC₅₀ (nM) of the modified IL-18 polypeptide's ability to induce IFNγ is less than 10-fold higher than, less than 5-fold higher than, or less than an EC₅₀ (nM) of an IL-18 polypeptide of SEQ ID NO: 1.

36. The modified IL-18 polypeptide of claim 35, wherein the EC₅₀ (nM) of the modified IL-18 polypeptide's ability to induce IFNγ is less than 5-fold greater than the EC₅₀ (nM) of SEQ ID NO: 1.

37. The modified IL-18 polypeptide of claim 35, wherein the EC₅₀ (nM) of the modified IL-18 polypeptide's ability to induce IFNγ is less than the EC₅₀ (nM) an IL-18 polypeptide of SEQ ID NO: 1.

38. The modified IL-18 polypeptide of claim 35, wherein the EC₅₀ (nM) of the modified IL-18 polypeptide's ability to induce IFNγ is at least about 10-fold less than the EC₅₀ (nM) of SEQ ID NO: 1.

39. A modified IL-18 polypeptide comprising a polypeptide sequence having at least about 80% sequence identity to any one of SEQ ID NOS: 2-83.

40. The modified IL-18 polypeptide of claim 39, wherein the polypeptide sequence is at least about 80% identical to SEQ ID NO: 2 or SEQ ID NO: 18.

41. The modified IL-18 polypeptide of claim 39, wherein the polypeptide sequence is at least about 80% identical to SEQ ID NO: 2.

42. The modified IL-18 polypeptide of claim 39, wherein the polypeptide sequence is at least about 90% identical to SEQ ID NO: 2.

43. The modified IL-18 polypeptide of claim 39, wherein the polypeptide sequence is at least about 95% identical to SEQ ID NO: 2.

44. The modified IL-18 polypeptide of claim 39, wherein the polypeptide sequence is at least about 80% identical to SEQ ID NO: 18.

45. The modified IL-18 polypeptide of claim 39, wherein the polypeptide sequence is at least about 90% identical to SEQ ID NO: 18.

46. The modified IL-18 polypeptide of claim 39, wherein the polypeptide sequence is at least about 95% identical to SEQ ID NO: 18.

47. The modified IL-18 polypeptide of any one of claims 1-46, wherein the modified IL-18 polypeptide exhibits less than a 10-fold lower affinity, less than a 5-fold lower affinity, or a greater affinity to an IL-18 receptor alpha subunit (IL-18Rα) than to IL-18 binding protein (IL-18BP) as measured by K_{D}, and wherein [K_{D} IL-18Rα]/[K_{D} IL-18BP] is greater than 0.1.

48. The modified IL-18 polypeptide of claim 47, wherein the modified IL-18 polypeptide binds to IL-18 receptor alpha (IL-18Rα).

49. The modified IL-18 polypeptide of claim 47, wherein the modified IL-18 polypeptide binds to IL-18Rα with a K_{D} of less than about 200 nM, less than about 100 nM, less than about 80 nM, less than about 70 nM, less than about 60 nM, or less than about 50 nM.

50. The modified IL-18 polypeptide of claim 47, wherein the modified IL-18 polypeptide binds to IL-18Rα with a K_{D} of less than about 50 nM.

51. The modified IL-18 polypeptide of any one of claims 47-50, wherein the modified IL-18 polypeptide binds to an IL-18 receptor alpha/beta (IL-18Rα/β) heterodimer.

52. The modified IL-18 polypeptide of claim 51, wherein the modified IL-18 polypeptide binds to the IL-18Rα/β heterodimer with a K_{D} of less than about 25 nM.

53. The modified IL-18 polypeptide of claim 51, wherein the modified IL-18 polypeptide binds to the IL-18Rα/β heterodimer with a K_{D} of less than about 10 nM.

54. The modified IL-18 polypeptide of any one of claims 1-53, wherein the modified IL-18 polypeptide is conjugated to an additional peptide.

55. A population of modified interleukin-18 (IL-18) polypeptides, comprising:
a) a plurality of modified IL-18 polypeptides; and
b) at least one polymer moiety, wherein at least one polymer moiety is covalently linked to the modified IL-18 polypeptides and attached at residue 65, residue 66, residue 67, residue 68, residue 69, residue 70, residue 71, residue 72, residue 73, residue 74 or residue 75, wherein the amino acid residue position is based on SEQ ID NO: 1 as a reference sequence;
wherein at least 90% of the modified IL-18 polypeptides have a molecular weight that is within ±500 Da of the peak molecular weight of the plurality of the modified IL-18 polypeptides as determined by high resolution electrospray ionization mass spectrometry (ESI-HRMS).

56. A population of modified interleukin-18 (IL-18) polypeptides, comprising:
a) a plurality of modified IL-18 polypeptides; and
b) a plurality of polymer moieties, wherein the plurality of polymer moieties are covalently linked to the modified IL-18 polypeptides and attached at residue 65, residue 66, residue 67, residue 68, residue 69, residue 70, residue 71, residue 72, residue 73, residue 74 or residue 75 of the modified IL-18 polypeptide, wherein the amino acid residue position is based on SEQ ID NO: 1 as a reference sequence;
wherein at least 90% of the plurality of polymer moieties have a molecular weight that is within ±500 Da of the peak molecular weight of the plurality of the polymer moieties as determined by high resolution electrospray ionization mass spectrometry (ESI-HRMS).

57. The population of modified IL-18 polypeptides of claim 55 or 56, wherein at least one polymer moiety or the plurality of polymer moieties is covalently linked to the modified IL-18 polypeptides at amino acid residue 68, wherein the amino acid residue numbering of the modified IL-18 polypeptides is based on SEQ ID NO: 1 as a reference sequence.

58. The population of modified IL-18 polypeptides of claim 55 or 56, wherein at least one polymer moiety or the plurality of polymer moieties is covalently linked to the modified IL-18 polypeptides at amino acid residue 69, wherein the amino acid residue numbering of the modified IL-18 polypeptides is based on SEQ ID NO: 1 as a reference sequence.

59. The population of modified IL-18 polypeptides of claim 55 or 56, wherein at least one polymer moiety or the plurality of polymer moieties is covalently linked to the modified IL-18 polypeptides at amino acid residue 70, wherein the amino acid residue numbering of the modified IL-18 polypeptides is based on SEQ ID NO: 1 as a reference sequence.

60. The population of modified IL-18 polypeptides of claim 55 or 56, wherein at least one polymer moiety or the plurality of polymer moieties is covalently linked to the modified IL-18 polypeptides at C68, wherein the amino acid residue numbering of the modified IL-18 polypeptides is based on SEQ ID NO: 1 as a reference sequence.

61. The population of modified IL-18 polypeptides of claim 55 or 56, wherein at least one polymer moiety or the plurality of polymer moieties is covalently linked to the modified IL-18 polypeptides at E69 or E69C, wherein the amino acid residue numbering of the modified IL-18 polypeptides is based on SEQ ID NO: 1 as a reference sequence.

62. The population of modified IL-18 polypeptides of claim 55 or 56, wherein at least one polymer moiety or the plurality of polymer moieties is covalently linked to the modified IL-18 polypeptides at K70 or K70C, wherein the amino acid residue numbering of the modified IL-18 polypeptides is based on SEQ ID NO: 1 as a reference sequence.

63. The population of modified IL-18 polypeptides of any one of claims 55-62, wherein each modified IL-18 polypeptide of the plurality of modified IL-18 polypeptides comprises one or more mutations.

64. The population of modified IL-18 polypeptides of claim 63, wherein the one or more mutations are located at residue positions selected from E06, K53, Y01, S55, F02, D54, C38, T63, C68, E69, C76, C127, and K70, wherein residue position numbering of the modified IL-18 polypeptides are based on SEQ ID NO: 1 as a reference sequence.

65. The population of modified IL-18 polypeptides of claim 64, wherein the one or more mutations are selected from E06K, K53A, Y01G, S55A, F02A, D54A, C38S, C38A, T63A, C68S, C68A, E69C, C76S, C76A, C127S, C127A, and K70C.

66. The population of modified IL-18 polypeptides of claim 65, wherein one or more mutations comprise E06K and K53A.

67. The population of modified IL-18 polypeptides of claim 65, wherein one or more mutations comprise E06K, K53A, and T63A.

68. The population of modified IL-18 polypeptides of any one of claims 55-67, wherein the population comprises at least 1 μg, at least 10 μg, or at least 1 mg of the modified IL-18 polypeptides.

69. The population of modified IL-18 polypeptides of any one of claims 55-67, wherein the population comprises at least 100, at least 1000, or at least 10000 of the modified IL-18 polypeptides.

70. The population of modified IL-18 polypeptides of any one of claims 55-69, wherein a ratio of weight average molecular weight over number average molecular weight for the population of the modified IL-18 polypeptide is at most 1.1.

71. The population of modified IL-18 polypeptides of any one of claims 55-70, wherein each of the plurality of polymers comprises a water-soluble polymer.

72. The population of modified IL-18 polypeptides of claim 71, wherein the water-soluble polymer comprises poly(alkylene oxide), polysaccharide, poly(vinyl pyrrolidone), poly(vinyl alcohol), polyoxazoline, poly(acryloylmorpholine), or a combination thereof.

73. The population of modified IL-18 polypeptides of claim 71, wherein the water-soluble polymer comprises polyethylene glycol.

74. The population of modified IL-18 polypeptides of any one of claims 55-73, wherein a weight average molecular weight of the plurality of polymers is from about 200 Da to about 50,000 Da.

75. The population of modified IL-18 polypeptides of any one of claims 55-74, wherein a weight average molecular weight of the plurality of polymers is from about 10,000 Da to about 30,000 Da.

76. A host cell comprising a modified IL-18 polypeptide of any one of claims 1-54.

77. A method of producing a modified IL-18 polypeptide of any one of claims 1-54, comprising expressing the modified IL-18 polypeptide in a host cell.

78. The host cell of claim 76 or 77, wherein the host cell is a prokaryotic cell or a eukaryotic cell.

79. The host cell of claim 76 or 77, wherein the host cell is a mammalian cell, an avian cell, a fungal cell, or an insect cell.

80. The host cell of claim 79, wherein the host cell is a CHO cell, a COS cell, or a yeast cell.

81. A pharmaceutical composition comprising:
a) a modified IL-18 polypeptide of any one of claims 1-54 or the population of modified IL-18 polypeptides of any one of claims 55-75; and
b) a pharmaceutically acceptable carrier or excipient.

82. The pharmaceutical composition of claim 81, wherein the pharmaceutical composition is in a lyophilized form.

83. A method of treating cancer in a subject in need thereof, comprising:
administering to the subject a pharmaceutically effective amount of a modified IL-18 polypeptide of any one of claims 1-54 or a pharmaceutical composition of claim 81 or 82.

84. The method of claim 83, wherein the cancer is a solid cancer.

85. The method of claim 84, wherein the solid cancer is kidney cancer, skin cancer, bladder cancer, bone cancer, brain cancer, breast cancer, colorectal cancer, esophageal cancer, eye cancer, head and neck cancer, lung cancer, ovarian cancer, pancreatic cancer, or prostate cancer.

86. The method of claim 84, wherein the solid cancer is metastatic renal cell carcinoma or melanoma.

87. The method of claim 84, wherein the solid cancer is a carcinoma or a sarcoma.

88. The method of claim 83, wherein the cancer is a blood cancer.

89. The method of claim 88, wherein the blood cancer is leukemia, non-Hodgkin lymphoma, Hodgkin lymphoma, or multiple myeloma.

90. The method of any one of claims 83-89, further comprising reconstituting a lyophilized form of the modified IL-18 polypeptide or the pharmaceutical composition.

91. A synthetic IL-18 polypeptide, comprising:
a synthetic IL-18 polypeptide comprising a homoserine (Hse) residue at a position selected from the region of residues 21-41, residues 60-80, and residues 106-126, wherein residue position numbering of the modified IL-18 polypeptide is based on SEQ ID NO: 1 as a reference sequence.

92. The synthetic IL-18 polypeptide of claim 91, wherein the synthetic IL-18 polypeptide comprises a Hse residue in each of the regions of residues 21-41, residues 60-80, and residues 106-126.

93. The synthetic IL-18 polypeptide of claim 91 or 92, wherein the synthetic IL-18 polypeptide comprises a Hse residue at position 31.

94. The synthetic IL-18 polypeptide of any one of claims 91-93, wherein the synthetic IL-18 polypeptide comprises a Hse residue at position 63 or position 75.

95. The synthetic IL-18 polypeptide of claim 94, wherein the synthetic IL-18 polypeptide comprises a Hse residue at position 63.

96. The synthetic IL-18 polypeptide of claim 94, wherein the synthetic IL-18 polypeptide comprises a Hse residue at position 75.

97. The synthetic IL-18 polypeptide of any one of claims 91-96, wherein the synthetic IL-18 polypeptide comprises a Hse residue at position 116.

98. The synthetic IL-18 polypeptide of any one of claims 91-97, wherein the synthetic IL-18 polypeptide comprises a Hse residue at position 31, 116, and at least one of positions 63 and 75.

99. The synthetic IL-18 polypeptide of any one of claims 91-98, wherein the synthetic IL-18 polypeptide comprises an amino acid substitution of at least one methionine residue in SEQ ID NO: 1.

100. The synthetic IL-18 polypeptide of claim 99, wherein the amino acid substitution of at least one methionine residue in SEQ ID NO: 1 comprises a substitution at M33, M51, M60, M86, M113, or M150.

101. The synthetic IL-18 polypeptide of claim 99 or 100, wherein the synthetic IL-18 polypeptide comprises substitutions of at least three methionine residues.

102. The synthetic IL-18 polypeptide of any one of claims 99-101, wherein the synthetic IL-18 polypeptide comprises substitutions of at least five methionine residues.

103. The synthetic IL-18 polypeptide of any one of claims 99-102, wherein the synthetic IL-18 polypeptide comprises substitution of at least six methionine residues.

104. The synthetic IL-18 polypeptide of any one of claims 99-103, wherein at least one methionine residue is substituted for an O-methyl-homoserine (Omh) residue.

105. The synthetic IL-18 polypeptide of any one of claims 99-104, wherein at least three methionine residues are substituted for Omh residues.

106. The synthetic IL-18 polypeptide of any one of claims 99-105, wherein at least five methionine residues are substituted for Omh residues.

107. The synthetic IL-18 polypeptide of any one of claims 99-106, wherein each methionine substitution is for a norleucine or Omh residue.

108. The synthetic IL-18 polypeptide of any one of claims 99-107, wherein each methionine substitution is for an Omh residue.

109. The synthetic IL-18 polypeptide of any one of claims 99-108, wherein each methionine residue of SEQ ID NO: 1 is substituted for an Omh residue.

110. The synthetic IL-18 polypeptide of any one of claims 91-109, wherein the synthetic IL-18 polypeptide comprises an additional mutation to SEQ ID NO: 1.

111. The synthetic IL-18 polypeptide of any one of claims 91-110, wherein the synthetic IL-18 polypeptide comprises an amino acid sequence at least about 80% identical to that of SEQ ID NO: 1.

112. The synthetic IL-18 polypeptide of any one of claims 91-111, wherein the synthetic IL-18 polypeptide comprises a polymer covalently attached to a residue of the synthetic IL-18 polypeptide.

113. A method of making a modified IL-18 polypeptide, comprising:
a) synthesizing two or more fragments of the modified IL-18 polypeptide;
b) ligating the fragments; and
c) folding the ligated fragments.

114. The method of claim 113, wherein the two or more fragments comprise an N-terminal fragment, a C-terminal fragment, and optionally one or more interior fragments, wherein the N-terminal fragment comprises the N-terminus of the modified IL-18 polypeptide and the C-terminal fragment comprises the C-terminus of the modified IL-18 polypeptide.

115. The method of claim 114, wherein each of the N-terminal fragment and the one or more interior fragments comprise an alpha-keto amino acid as the C-terminal residue of each fragment.

116. The method of claim 115, wherein each alpha-keto amino acid is selected from alpha-keto-phenylalanine, alpha-keto-tyrosine, alpha-keto-valine, alpha-keto-leucine, alpha-keto-isoleucine, alpha-keto-norleucine, and alpha-keto-O-methylhomoserine.

117. The method of any one of claims 114-117, wherein each of the C-terminal fragment and the one or more interior fragments comprise a residue having a hydroxylamine or a cyclic hydroxylamine functionality as the N-terminal residue of each fragment.

118. The method of claim 117, wherein each residue having the hydroxylamine or the cyclic hydroxylamine functionality is a 5-oxaproline residue.

119. The method of any one of claims 113-118, wherein synthesizing two or more fragments of the modified IL-18 polypeptide comprises synthesizing four fragments.

120. The method of claim 119, wherein the four fragments comprise an N-terminal fragment, a first interior fragment, a second interior fragment, and a C-terminal fragment.

121. The method of claim 120, wherein the N-terminal fragment comprises residues which correspond to amino acids 1-30 of the modified IL-18 polypeptide, wherein residue position numbering of the modified IL-18 polypeptide is based on SEQ ID NO: 1 as a reference sequence.

122. The method of claim 120 or 121, wherein the N-terminal fragment comprises an N-terminal extension as compared to the sequence of SEQ ID NO: 1.

123. The method of any one of claims 120-122, wherein the N-terminal fragment comprises an amino acid sequence having at least 80% sequence identity with the amino acid sequence as set forth in SEQ ID NO: 201.

124. The method of any one of claims 120-123, wherein the N-terminal fragment comprises an amino acid sequence as set forth in any one of SEQ ID NOS: 201-209.

125. The method of any one of claims 120-124, wherein the first interior fragment comprises residues which correspond to amino acids 31-62 of the modified IL-18 polypeptide, wherein residue position numbering of the modified IL-18 polypeptide is based on SEQ ID NO: 1 as a reference sequence.

126. The method of any one of claims 120-125, wherein the first interior fragment comprises an amino acid sequence having at least 80% sequence identity with the amino acid sequence as set forth in SEQ ID NO: 210.

127. The method of any one of claims 120-126, wherein the first interior fragment comprises an amino acid sequence as set forth in any one of SEQ ID NOS: 210-217.

128. The method of any one of claims 120-127, wherein the second interior fragment comprises residues which correspond to amino acids 63-115 of the modified IL-18 polypeptide, wherein residue position numbering of the modified IL-18 polypeptide is based on SEQ ID NO: 1 as a reference sequence.

129. The method of any one of claims 120-128, wherein the second interior fragment comprises an amino acid sequence having at least 80% sequence identity with the amino acid sequence as set forth in SEQ ID NO: 227.

130. The method of any one of claims 120-129, wherein the second interior fragment comprises an amino acid sequence as set forth in any one of SEQ ID NOs: 227-236.

131. The method of any one of claims 120-124, wherein the first interior fragment comprises residues which correspond to amino acids 31-74 of the modified IL-18 polypeptide, wherein residue position numbering of the modified IL-18 polypeptide is based on SEQ ID NO: 1 as a reference sequence.

132. The method of any one of claim 120-124 or 131, wherein the first interior fragment comprises an amino acid sequence having at least 80% sequence identity with the amino acid sequence as set forth in SEQ ID NO: 218.

133. The method of any one of claim 120-124, 131 or 132, wherein the first interior fragment comprises an amino acid sequence as set forth in any one of SEQ ID NOS: 218-226.

134. The method of any one of claim 120-124 or 131-133, wherein the second interior fragment comprises residues which correspond to amino acids 75-115 of the modified IL-18 polypeptide, wherein residue position numbering of the modified IL-18 polypeptide is based on SEQ ID NO: 1 as a reference sequence.

135. The method of any one of claim 120-124 or 131-134, wherein the second interior fragment comprises an amino acid sequence having at least 80% sequence identity with the amino acid sequence as set forth in SEQ ID NO: 237.

136. The method of any one of claim 120-124 or 131-135, wherein the second interior fragment comprises an amino acid sequence as set forth in any one of SEQ ID NOs: 237-242.

137. The method of any one of claims 120-136, wherein the C-terminal fragment comprises residues which correspond to amino acids 116-157 of the modified IL-18 polypeptide, wherein residue position numbering of the modified IL-18 polypeptide is based on SEQ ID NO: 1 as a reference sequence.

138. The method of any one of claims 120-137, wherein the C-terminal fragment comprises an amino acid sequence having at least 80% sequence identity with the amino acid sequence as set forth in SEQ ID NO: 243.

139. The method of any one of claims 120-138, wherein the C-terminal fragment comprises an amino acid sequence as set forth in any one of SEQ ID NOS: 243-248.

140. The method of any one of claims 120-139, wherein the N-terminal fragment, the first interior fragment, the second interior fragment, and the C-terminal fragment are arranged from the N-terminus to the C-terminus, respectively, in the modified IL-18 polypeptide.

141. The method of any one of claims 113-140, wherein the method further comprises rearranging the ligated fragments.

142. The method of any one of claims 113-141, wherein the at least one of the fragments of the IL-18 polypeptide comprises a conjugation handle.

143. The method of any one of claims 113-142, further comprising attaching a water-soluble polymer to the folded, ligated fragments.

144. A fusion protein comprising a modified IL-18 polypeptide, wherein the modified IL-18 polypeptide comprises a sequence that is at least about 80% identical to any one of SEQ ID NOS: 2-83.

145. The fusion protein of claim 144, wherein the sequence is at least about 85% identical to SEQ ID NO: 2.

146. The fusion protein of claim 144, wherein the sequence is at least about 90% identical to SEQ ID NO: 2.

147. The fusion protein of claim 144, wherein the sequence is at least about 95% identical to SEQ ID NO: 2.

148. The fusion protein of claim 144, wherein the sequence is at least about 85% identical to SEQ ID NO: 18.

149. The fusion protein of claim 144, wherein the sequence is at least about 90% identical to SEQ ID NO: 18.

150. The fusion protein of claim 144, wherein the sequence is at least about 95% identical to SEQ ID NO: 18.

151. The fusion protein of claim 144, wherein the sequence is identical to SEQ ID NO: 18.
